(19) United States
(12) Patent Application Publication
(10) Pub. No.: US 2024/0124529 A1
(43) Pub. Date: Apr. 18, 2024

(54) ANTIVIRAL STRUCTURALLY-STABILIZED SARS-CoV-2 PEPTIDES AND USES THEREOF

(71) Applicant: Dana-Farber Cancer Institute, Inc., Boston, MA (US)

(72) Inventors: Loren D. Walensky, Newton, MA (US); Gregory H. Bird, Pelham, NH (US)

(21) Appl. No.: 17/908,825

(22) PCT Filed: Mar. 4, 2021
(86) PCT No.: PCT/US2021/020940

§ 371 Date: Sep. 1, 2022

Related U.S. Application Data
(60) Provisional application No. 62/985,100, filed on Mar. 4, 2020.

(51) Int. Cl. C07K 14/005; A61P 31/14

(52) U.S. Cl. CPC C07K 14/005; A61P 31/14; A61K 38/00; C12N 2770/20022; C12N 2770/20033

(57) ABSTRACT

Disclosed herein are cross-linked peptides useful for interfering with and inhibiting coronavirus infection (e.g., infection by SARS-CoV-2). Also disclosed are methods of treating and/or preventing a coronavirus infection (e.g., COVID-19).

### CROSS-REFERENCE TO RELATED APPLICATION

This application claims the benefit of priority to U.S. Provisional Appl. No. 62/985,100, filed Mar. 4, 2020, the contents of which are incorporated by reference in their entirety herein.

### SEQUENCE LISTING

The instant application contains a Sequence Listing which has been submitted electronically in ASCII format and is hereby incorporated by reference in its entirety. Said ASCII copy, created on Mar. 4, 2021, is named 00530_0401WO1_2823_W01WO_SL.txt and is 163,968 bytes in size.

### TECHNICAL FIELD

This disclosure relates to structurally-stabilized SARS-CoV-2 antiviral peptides and methods for using such peptides in the prevention and treatment of a coronavirus infection.

### BACKGROUND

No anti-viral therapeutic currently exists to prevent or treat infection by novel coronavirus (nCoV) outbreaks, such as COVID-19 caused by the Wuhan nCoV (also known as 2019-nCoV or SARS-CoV-2). COVID-19 has been declared a high-risk global health emergency by the World Health Organization (WHO) and has, as of March 2021, caused 114,857,764 cases of respiratory disease and 2,551,459 deaths worldwide.

SARS-CoV-2 contains a surface protein that undergoes a conformational change upon engagement with the host cell, resulting in formation of a six-helix bundle that brings the host and viral membranes together. Although peptide-based inhibition of viral fusion processes is mechanistically feasible and clinically effective (e.g., Fuzeon (i.e., enfurvirtide), approved by the FDA in 2003), the biophysical and pharmacologic liabilities of peptides, including loss of bioactive shape and rapid proteolysis in vivo (e.g., 100 mg self-injected twice daily), have limited broader application of this validated approach. Thus, new strategies for the prophylaxis and/or treatment of COVID-19 infection are urgently required to effectively mitigate the outbreak.

### SUMMARY

This application relates to compositions and methods disclosing peptide stabilizing technology (e.g., stapling, stitching) that recapitulates and fortifies the structure of bioactive helices to generate a targeted prophylactic and therapeutic agent for prevention and/or treatment of coronavirus (e.g., betacoronavirus such as SARS-CoV-2) infection. By inserting “staples” (e.g., all-hydrocarbon staples) or “stitches” into natural peptides, bioactive-helical structure can be restored and remarkable protease resistance can be conferred by burying the otherwise labile amide bonds at the core of the helical structure and/or restraining amide bonds in a manner that precludes their recognition and proteolysis by the body's proteases. Here, structurally-stabilized peptide inhibitors of coronavirus (e.g., betacoronavirus such as SARS-CoV-2) are disclosed. These structurally-stabilized peptide inhibitors are used to prevent and/or treat coronavirus (e.g., betacoronavirus such as SARS-CoV-2) infection such as COVID-19.

The disclosure provides, in part, structurally-stabilized peptides of an amino acid sequence comprising a sequence that is at least 50%, 55%, 60%, 65%, 70%, 75%, 80%, 85%, 90%, 94%, 95%, or 100% identical to the sequence of any one of SEQ ID NO:10 or 258 (core template sequences of SARS-CoV-2 HR2 and EK1, respectively) or SEQ ID NOs.: 133, 40, 136, 42, 30, 113, 34, 36, 134, 39, 135, 42, 137, 50, 52, 51, 31-33, 37, 41, 44-49, 177, and 179, wherein the structurally-stabilized peptide has at least one (1, 2, 3, 4, 5, 6) of these properties: (i) binds the 5-helix bundle of SARS-CoV-2 S protein; (ii) disrupts the interaction between the 5 helix bundle of SARS-CoV-2 S protein and a peptide of SEQ ID NO:10 or 258; (iii) is alpha-helical; (iv) is protease resistant; (v) inhibits fusion of SARS-CoV-2 with a host cell; and/or (vi) inhibits infection of a cell by SARS-CoV-2. The disclosure also provides, in part, structurally-stabilized peptides of an amino acid sequence comprising a sequence of any one of SEQ ID NO:10 or 258, or SEQ ID NOs.: 133, 40, 136, 42, 30, 113, 34, 36, 134, 39, 135, 42, 137, 50, 52, 51, 31-33, 37, 41, 44-49, 177, and 179 with 0 to 10 (0, 1, 2, 3, 4, 5, 6, 7, 8, 9, or 10) amino acid substitutions, insertions, and/or deletions, wherein the structurally-stabilized peptide has at least one (1, 2, 3, 4, 5, 6) of these properties: (i) binds the 5-helix bundle of SARS-CoV-2 S protein; (ii) disrupts the interaction between the 5 helix bundle of SARS-CoV-2 S protein and a peptide of SEQ ID NO:10 or 258; (iii) is alpha-helical; (iv) is protease resistant; (v) inhibits fusion of SARS-CoV-2 with a host cell; and/or (vi) inhibits infection of a cell by SARS-CoV-2. In some instances, one or more of positions 1, 3, 5, 6, 8, 10, 12, 13, 15, 17, and 19 of SEQ ID NO: 10 or 258 are not substituted, or are substituted by a conservative amino acid substitution. In some instances, one or more (1, 2, 3, 4, 5, 6) of positions 2, 4, 7, 9, 11, 14, 16, or 18 of SEQ ID NOs.: 10 or 258 are substituted by an α, α-disubstituted non-natural amino acids with olefinic side chains. In certain instances, one or more of positions 4, 8, 10, 13, 15, 17 and 18 of SEQ ID NO:10 or 258 are not substituted, or if substituted are substituted by a conservative amino acid. In certain instances, one or more of positions 1, 5, 7, 11, or 12 of SEQ ID NO:10 or 258 if substituted are substituted by a conservative amino acid. The guiding feature of varying the amino acid sequence of SEQ ID NO:10 or 258 is that it should still bind the 5 helix bundle of SARS-CoV-2 and be able to inhibit or disrupt the association of the 5 helix bundle with a peptide of SEQ ID NO: 10 or 258. In some instances, the structurally-stabilized peptide comprises the sequence of any one of SEQ ID NOs.: 133, 40, 136, 42, 30, 113, 34, 36, 134, 39, 135, 42, 137, 50, 52, 51, 31-33, 37, 41, 44-49, 177, and 179. The above-described peptides can be 19 to 100 (e.g., at least 19, 25, 30, 35, 40, 45, 50, 55, 60, 65, 70, 75, 80, 85, 90, 95) amino acids in length, These peptides can be lipidated. The peptides can also be modified to be conjugated to polyethylene glycol (PEG). In addition, these peptides can be modified to include additional N-terminal (e.g., either SEQ ID NO: 250 or 251) and/or C-terminal (e.g., any one of SEQ ID NO: 252-255) sequences of the corresponding SARS-CoV-2 HR2 peptide. In some cases, these peptides can be modified to include the amino acid sequence GSGSGC (SEQ ID NO:256) appended at the C-terminus of the amino acid sequence. In some cases, the amino acid sequence further comprises a C-terminal peptide/PEG spacer conjugated cholesterol as in GSGSGC (SEQ ID NO:256)-Ac-PEG4-Cholesterol. In some cases, these peptides can be modified to include the GSGSGC (SEQ ID NO:256)-(PEG4-chol)-carboxamide appended at the C-terminus of the amino acid sequence. These structurally-stabilized peptides are useful for treatment or prevention of a coronavirus infection (e.g., COVID-19). The disclosure also relates to methods of making the structurally stabilized peptides described above. For example, a peptide of any one of SEQ ID NOs.: 133, 40, 136, 42, 30, 113, 34, 36, 134, 39, 135, 42, 137, 50, 52, 51, 31-33, 37, 41, 44-49, 177, and 179 is subject to cross-linking (e.g., by a ruthenium mediated ring closing metathesis reaction). The method can further including formulating the cross-linked peptide as a sterile pharmaceutical composition useful for administration to a human subject in need thereof (e.g., intravenous, subcutaneous, topical, intranasal).

In one aspect, this disclosure features a structurally-stabilized polypeptide comprising an amino acid sequence that is at least 55%, 60%, 65%, 70%, 75%, 80%, 85%, 90%, or 94% identical to sequence set forth in SEQ ID NO:10 (IQKEIDRLNEVAKNLNESL). In some instances, amino acids at positions of SEQ ID NO:10 selected from (wherein position 1 is the N-terminal Isoleucine and position 19 is the C-terminal Leucine of SEQ ID NO:10):
(i) positions 7 and 11;
(ii) positions 10 and 14;
(iii) positions 12 and 16;
(iv) positions 14 and 18
(v) positions 2 and 9;
(vi) positions 4 and 11;
(vii) positions 9 and 16;
(viii) positions 2 and 6;
(ix) positions 8 and 12;
(x) positions 9 and 13;
(xi) positions 11 and 15;
(xii) positions 14 and 18;
(xiii) positions 15 and 19;
(xiv) positions 7 and 14;
(xv) positions 3 and 10;
(xvi) positions 6 and 13;
(xvii) positions 13 and 17;
(xiii) positions 3 and 7;
(xix) positions 3, 7, 13, and 17;
(xx) positions 3, 7, 14, and 18;
(xxi) positions 2, 6, 14, and 18;
(xxii) positions 2, 6, 13, and 17;
(xxiii) positions 3, 10, and 17;
(xiv) positions 2, 9, and 13;
(xv) positions 3, 10, and 14;
(xvi) positions 6, 13, and 17; or
(xvii) positions 7, 14, and 18,
are replaced by α, α-disubstituted non-natural amino acids with olefinic side chains. In some instances, if the amino acid sequence includes additional substitution(s), those substitution(s) are based on either (A) or (B):
(A) wherein positions 4, 8, 10, 13, 15, 17, and 18 of SEQ ID NO:10, if not substituted by an α, α-disubstituted non-natural amino acid with olefinic side chains, are not substituted or substituted by a conservative amino acid substitution;
wherein positions 1, 5, 7, and 11 if substituted are substituted by conservative amino acid substitutions or an α, α-disubstituted non-natural amino acid with olefinic side chains; and
wherein the remaining positions in SEQ ID NO:10 can be substituted by any amino acid or an α, α-disubstituted non-natural amino acid with olefinic side chains; or
(B) wherein one or more of positions 1, 3, 5, 6, 8, 10, 12, 13, 15, 17, and 19 of SEQ ID NO:10, are not substituted, or if substituted are replaced by a conservative amino acid substitution.

In some instances, the structurally-stabilized polypeptide at one or more of positions 2, 4, 7, 9, 11, 14, 16, and 18 of SEQ ID NO:10 can be replaced by any amino acid or an α, α-disubstituted non-natural amino acid with olefinic side chains. In some instances, the structurally-stabilized peptide is 15 to 100 amino acids in length, optionally 19 to 45 amino acids in length. In some instances, the structurally-stabilized peptide has one or more of the properties listed below: (i) binds a recombinant 5-helix bundle of SARS-CoV-2 S protein; (ii) disrupts the interaction between the 5 helix bundle and SEQ ID NO:10; (iii) is alpha-helical; (iv) is protease resistant; (v) inhibits fusion of SARS-CoV-2 with a host cell; and/or (vi) inhibits infection of a cell by SARS-CoV-2.

In some instances, the amino acid sequence of the structurally-stabilized polypeptide is at least 70% (70%, 75%, 80%, 85%, 90%, 95%) identical to the sequence set forth in SEQ ID NO:10. In some instances, the amino acid sequence of the structurally-stabilized polypeptide is at least 80% (80%, 85%, 90%, 95%) identical to the sequence set forth in SEQ ID NO:10. In some instances, the amino acid sequence of the structurally-stabilized polypeptide comprises the sequence of SEQ ID NO:50. In some instances, the amino acid sequence of the structurally-stabilized polypeptide comprises the sequence of SEQ ID NO:52. In some instances, the amino acid sequence of the structurally-stabilized polypeptide comprises the sequence of SEQ ID NO:51. In some instances, the amino acid sequence comprises the sequence of any one of the sequences of SEQ ID NOs.: 133, 40, 136, 42, 30, 113, 34, 36, 134, 39, 135, 42, and 137.

In some instances, the structurally-stabilized further comprises the amino acid sequence ISGINASVVN (SEQ ID NO:250) appended at the N-terminus of the amino acid sequence. In some instances, the structurally-stabilized further comprises the amino acid sequence DISGINASVVN (SEQ ID NO:251) appended at the N-terminus of the amino acid sequence. In some instances, the structurally-stabilized further comprises the amino acid sequence IDLQEL (SEQ ID NO:252) appended at the C-terminus of the amino acid sequence. In some instances, the structurally-stabilized further comprises the amino acid sequence IDLQELGKYEQYI (SEQ ID NO:253) appended at the C-terminus of the amino acid sequence. In some instances, the structurally-stabilized further comprises the amino acid sequence IDLQELGSGSGC (SEQ ID NO:254) appended at the C-terminus of the amino acid sequence. In some instances, the structurally-stabilized further comprises the amino acid sequence IDLQELGKYEQYIGSGSGC (SEQ ID NO:255) appended at the C-terminus of the amino acid sequence.

In some instances, the structurally-stabilized further comprises polyethylene glycol. In some instances, the structurally-stabilized further comprises cholesterol.

In some instances, the structurally-stabilized further comprises the GSGSGC(SEQ ID NO:256)-(PEG4-chol)-carboxamide.

In another aspect the disclosure features a structurally-stabilized polypeptide comprising an amino acid sequence that is at least 55%, 60%, 65%, 70%, 75%, 80%, 85%, 90%, or 94% identical to sequence set forth in SEQ ID NO:258 (LEYEBKKLEEAIKKLEESY, wherein amino acids at positions of SEQ ID NO:258 selected from (wherein position 1 is the N-terminal Leucine and position 19 is the C-terminal Tyrosine):
(i) positions 2, 9, and 15;
(ii) positions 3, 10, and 16;
(iii) positions 2, 6, 13, and 17;
(iv) positions 3, 7, 13, and 17;
(v) positions 2, 6, 14, and 18; or
(vi) positions 3, 7, 14, and 18,
   are replaced by α, α-disubstituted non-natural amino acids with olefinic side chains. In some cases, if the amino acid sequence has additional substitution(s) they are as follows: wherein if one or more of positions 2, 4, 7, 9, 11, 14, 16, or 18 of SEQ ID NO:258 if not substituted by an α, α-disubstituted non-natural amino acid with olefinic side chains are substituted by any amino acid; and wherein one or more of positions 1, 3, 5, 6, 8, 10, 12, 13, 15, 17 and 19 of SEQ ID NO:110, are not substituted, or if substituted, are substituted by conservative amino acid substitutions. In some cases, if the amino acid sequence has additional substitution(s) they are at one or more of positions, 2, 9, 11, 14, or 16 of SEQ ID NO:258 and the substitution can be to any amino acid including a conservative substitution. In some cases, if the amino acid sequence has additional substitution(s) they are at one or more of positions, 1, 5, 7, 11, or 12 of SEQ ID NO:258, then the substitution is a conservative amino acid substitution. In some cases, the peptide is 19 to 100 amino acids in length. Finally, the structurally-stabilized peptide has one or more of the properties listed below: (i) binds the 5-helix bundle of SARS-CoV-2 S protein; (ii) disrupts the interaction between the 5 helix bundle and SEQ ID NO:258; (iii) is alpha-helical; (iv) is protease resistant; (v) inhibits fusion of SARS-CoV-2 with a host cell; and/or (vi) inhibits infection of a cell by SARS-CoV-2.

In another aspect, this disclosure features a structurally-stabilized polypeptide comprising an amino acid sequence that is at least 55%, 60%, 65%, 70%, 75%, 80%, 85%, 90%, or 94% identical to sequence set forth in SEQ ID NO:110 (SLDQINVTFLDLEYEMKKLEEAIKKLEESYIDLKEL), wherein amino acids at positions of SEQ ID NO:110 selected from (position 1 is the N-terminal Serine and position 36 is the C-terminal Leucine):
(i) positions 13, 20, and 27
(ii) positions 14, 21, and 28;
(iii) positions 13, 17, 24, and 28;
(iv) positions 14, 18, 24, and 28;
(v) positions 13, 17, 25, and 29; or
(vi) positions 14, 18, 25, and 29,
are replaced by α, α-disubstituted non-natural amino acids with olefinic side chains,
and if the amino acid sequence has additional substitution(s) they are based on (A)
(A) wherein if one or more of positions 4, 8, 10, 13, 15, 17, and 18 of SEQ ID NO:110 if not substituted by an α, α-disubstituted non-natural amino acid with olefinic side chains are not substituted or substituted by a conservative amino acid substitution;
wherein one or more of positions 1, 5, 7, and 11 of SEQ ID NO:110 if substituted are substituted by conservative amino acid substitutions;
wherein one or more of the remaining positions in SEQ ID NO:110 can be substituted by any amino acid;
and
wherein the peptide is 15 to 100 amino acids in length; and
wherein the structurally-stabilized peptide has one or more of the properties listed below: (i) binds a recombinant 5-helix bundle of SARS-CoV-2 S protein; (ii) disrupts the interaction between the 5 helix bundle and SEQ ID NO:258; (iii) is alpha-helical; (iv) is protease resistant; (v) inhibits fusion of SARS-CoV-2 with a host cell; and/or (vi) inhibits infection of a cell by SARS-CoV-2.

In some instances, the structurally-stabilized polypeptide comprises the amino acid sequence is at least 70% (70%, 75%, 80%, 85%, 90%, 95%) identical to the sequence set forth in SEQ ID NO:177. In some instances, the structurally-stabilized polypeptide comprises the amino acid sequence is identical to the sequence set forth in SEQ ID NO:177. In some instances, the structurally-stabilized polypeptide comprises the amino acid sequence is at least 70% (70%, 75%, 80%, 85%, 90%, 95%) identical to the sequence set forth in SEQ ID NO:179. In some instances, the structurally-stabilized polypeptide comprises the amino acid sequence is identical to the sequence set forth in SEQ ID NO: 179. In some instances, the structurally-stabilized polypeptide further comprises the amino acid sequence GSGSGC (SEQ ID NO:256) appended at the C-terminus of the amino acid sequence.

In some instances, the structurally-stabilized polypeptide further comprises polyethylene glycol. In some instances, the structurally-stabilized polypeptide further comprises cholesterol.

In some instances, the structurally-stabilized polypeptide further comprises GSGSGC(SEQ ID NO:256)-(PEG4-chol)-carboxamide).

In one aspect this disclosure features a peptide comprising at least 6, 7, 8, 9, 10, 11, 12, 13, 14, 15, 16, 17, 18, 19, 20, 21, 22, 23, 24, 25, 26, 27, 28, 29, or 30 contiguous amino acids of the amino acid sequence set forth in SEQ ID NO:9 with at least two (e.g., 2, 3, 4, 5) amino acids separated by 2, 3, or 6 amino acids replaced by α, α-disubstituted non-natural amino acids with olefinic side chains. In some instances, the SARS CoV-2 HR2 peptide template sequence is no greater than 45 amino acids in length (e.g., 42, 43, 44, or 45) but it should of course be understood that the SARS CoV-2 HR2 peptide template sequence can be extended at the N- or C-terminus (with or without chemical derivatizations) to maintain or optimize activity. The peptide binds a recombinant SARS-CoV-2 5-helix bundle S protein. The peptide can also inhibit or disrupt the interaction between a SARS CoV-2 HR2 sequence (e.g., SEQ ID NOs:9, 10, 103, 104, 106, or 108) and the recombinant SARS-CoV-2 5-helix bundle S protein.

In some instances, the peptide comprises or consists of at least 6, 7, 8, 9, 10, 11, 12, 13, 14, 15, 16, 17, 19, 20, 21, 22, 23, 24, 25, 26, 27, 28, 29, or 30 contiguous amino acids of the amino acid sequence set forth in any one of SEQ ID NOs:11 to 29. In some instances, the peptide comprises or consists of at least 6, 7, 8, 9, 10, 11, 12, 13, 14, 15, 16, 17, 18, 19, 20, 21, 22, 23, 24, 25, 26, 27, 28, 29, or 30 contiguous amino acids of the amino acid sequence set forth in any one of SEQ ID NOs:11 to 29 with 1, 2, 3, 4, or 5 amino acid substitutions either in the non-interacting surface when tolerated, or homologous substitutions on the interacting face so as to avoid disruption of key binding interactions between the stapled peptide and the recombinant 5-helix bundle target of SARS-CoV-2. In some instances, the peptide comprises or consists of the amino acid sequence set forth in any one of SEQ ID NOs:11 to 29 with 1, 2, 3, 4, or 5 amino acid substitutions. These peptides have one or more (e.g., 1, 2, 3, 4) of the properties listed below: (i) binds the recombinant SARS-CoV-2 5-helix bundle S protein; (ii) inhibits or disrupts interaction between a SARS CoV-2 HR2 sequence (e.g., SEQ ID NOs:9, 10, 103, 104, 106, or 108) and the recombinant SARS-CoV-2 5-helix bundle S protein; (iii) inhibits fusion of SARS-CoV-2 with a host cell; and/or (iv) inhibits infection of a cell by SARS-CoV-2.

In another aspect the disclosure features a structurally-stabilized peptide comprising at least 6, 7, 8, 9, 10, 11, 12, 13, 14, 15, 16, 17, 18, 19, 20, 21, 22, 23, 24, 25, 26, 27, 28, 29, or 30 contiguous amino acids of the amino acid sequence set forth in SEQ ID NO:9 with at least two (e.g., 2, 3, 4, 5) amino acids separated by 2, 3, or 6 amino acids replaced by α, α-disubstituted non-natural amino acids with olefinic side chains. The side chains of the α, α-disubstituted non-natural amino acids with olefinic side chains are cross-linked. In some instances, the SARS CoV-2 HR2 peptide template sequence is no greater than 45 amino acids in length (e.g., 42, 43, 44, or 45) but can be extended at the N- or C-terminus (with or without chemical derivatizations) to maintain or optimize activity. The structurally-stabilized peptide has one or more (e.g., 1, 2, 3, 4, 5, 6) of the properties listed below: (i) binds the recombinant SARS-CoV-2 5-helix bundle S protein; (ii) inhibits or disrupts the interactions between the 5 helix bundle and SARS-CoV-2 HR2 peptide (e.g., SEQ ID NOs:9, 10, 103, 104, 106, or 108); (iii) is alpha-helical; (iv) is protease resistant; (v) inhibits fusion of SARS-CoV-2 with a host cell; and/or (vi) inhibits infection of a cell by SARS-CoV-2. In some instances, the structurally-stabilized peptide is 42 to 45 (e.g., 42, 43, 44, 45) amino acids in length.

In some instances, the structurally-stabilized peptide comprises or consists of at least 6, 7, 8, 9, 10, 11, 12, 13, 14, 15, 16, 17, 18, 19, 20, 21, 22, 23, 24, 25, 26, 27, 28, 29, or 30 contiguous amino acids of the amino acid sequence set forth in any one of SEQ ID NOs:11 to 29, wherein the side chains of the α, α-disubstituted non-natural amino acids with olefinic side chains are cross-linked (e.g., stapled and/or stitched). In some instances, the structurally-stabilized peptide comprises or consists of at least 6, 7, 8, 9, 10, 11, 12, 13, 14, 15, 16, 17, 18, 19, 20, 21, 22, 23, 24, 25, 26, 27, 28, 29, or 30 contiguous amino acids of the amino acid sequence set forth in any one of SEQ ID NOs:11 to 29 with 1, 2, 3, 4, or 5 amino acid substitutions, wherein the side chains of the α, α-disubstituted non-natural amino acids with olefinic side chains are cross-linked (e.g., stapled and/or stitched). In some instances, the structurally-stabilized peptide comprises or consists of the amino acid sequence set forth in any one of SEQ ID NOs:11 to 29 with 1, 2, 3, 4, or 5 amino acid substitutions, wherein the side chains of the α, α-disubstituted non-natural amino acids with olefinic side chains are cross-linked (e.g., stapled and/or stitched). In some instances, the structurally-stabilized peptide is 42 to 45 (e.g., 42, 43, 44, 45) amino acids in length.

In another aspect the disclosure provides a peptide comprising at least 6, 7, 8, 9, 10, 11, 12, 13, 14, 15, 16, or 17 contiguous amino acids of the amino acid sequence set forth in SEQ ID NO:10 with at least two (e.g., 2, 3, 4, 5) amino acids separated by 2, 3, or 6 amino acids replaced by α, α-disubstituted non-natural amino acids with olefinic side chains. In some instances, the peptide sequence template is at most 45 amino acids in length (e.g., 19, 20, 21, 22, 23, 24, 25, 26, 27, 28, 29, 30, 31, 32, 33, 34, 35, 36, 37, 38, 39, 40, 41, 42, 43, 44, or 45) but in some instances can be extended at the N- or C-terminus (with or without chemical derivatizations) to maintain or optimize activity. The peptide binds the recombinant SARS-CoV-2 5-helix bundle S protein. The peptide can also inhibit or disrupt the interaction between a SARS CoV-2 HR2 sequence (e.g., SEQ ID NOs:9, 10, 103, 104, 106, or 108) and the recombinant SARS-CoV-2 5-helix bundle S protein.

In some instances, the peptide comprises or consists of at least 6, 7, 8, 9, 10, 11, 12, 13, 14, 15, 16, or 17 contiguous amino acids of the amino acid sequence set forth in any one of SEQ ID NOs: 30-52. In some instances, the peptide comprises or consists of at least 6, 7, 8, 9, 10, 11, 12, 13, 14, 15, 16, or 17 contiguous amino acids of the amino acid sequence set forth in any one of SEQ ID NOs:30-52 with 1, 2, 3, 4, or 5 amino acid substitutions either in the non-interacting surface when tolerated, or homologous substitutions on the interacting face so as to avoid disruption of key binding interactions between the stapled peptide and the recombinant 5-helix bundle target of SARS-CoV-2.

In some instances, the peptide comprises or consists of the amino acid sequence set forth in any one of SEQ ID NOs:30-52 with 1, 2, 3, 4, or 5 amino acid substitutions. These peptides have one or more (e.g., 1, 2, 3, 4) of the properties listed below: (i) binds the recombinant SARS-CoV-2 5-helix bundle S protein; (ii) inhibits the interactions between the 5 helix bundle and SARS-CoV-2 HR2 peptide (SEQ ID NO:9); (iii) inhibits fusion of SARS-CoV-2 with a host cell; and/or (iv) inhibits infection of a cell by SARS-CoV-2.

In another aspect the disclosure features a structurally-stabilized peptide comprising at least 6, 7, 8, 9, 10, 11, 12, 13, 14, 15, 16, or 17 contiguous amino acids of the amino acid sequence set forth in SEQ ID NO:10 with at least two (e.g., 2, 3, 4, 5) amino acids separated by 2, 3, or 6 amino acids replaced by α, α-disubstituted non-natural amino acids with olefinic side chains. The side chains of the α, α-disubstituted non-natural amino acids with olefinic side chains are cross-linked. The peptide is no longer than 45 amino acids in length (e.g., 42, 43, 44, or 45) but can be extended at the N- or C-terminus (with or without chemical derivatizations) to maintain or optimize activity. The structurally-stabilized peptide has one or more (e.g., 1, 2, 3, 4, 5) of the properties listed below: (i) binds the recombinant SARS-CoV-2 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2. In some instances, the structurally-stabilized peptide is 19 to 45 (e.g., 19, 20, 21, 22, 23, 24, 25, 26, 27, 28, 29, 30, 31, 32, 33, 34, 35, 36, 37, 38, 39, 40, 41, 42, 43, 44, 45) amino acids in length.

In some instances, the structurally-stabilized peptide comprises or consists of at least 6, 7, 8, 9, 10, 11, 12, 13, 14, 15, 16, or 17 contiguous amino acids of the amino acid sequence set forth in any one of SEQ ID NOs:30 to 52, wherein the side chains of the α, α-disubstituted non-natural amino acids with olefinic side chains are cross-linked (e.g., stapled and/or stitched). In some instances, the structurally-stabilized peptide comprises or consists of at least 6, 7, 8, 9, 10, 11, 12, 13, 14, 15, 16, or 17 contiguous amino acids of the amino acid sequence set forth in any one of SEQ ID NOs:30 to 52 with 1, 2, 3, 4, or 5 amino acid substitutions, wherein the side chains of the α, α-disubstituted non-natural amino acids with olefinic side chains are cross-linked (e.g., stapled and/or stitched). In some instances, the structurally-stabilized peptide comprises or consists of the amino acid sequence set forth in any one of SEQ ID NOs:30 to 52 with 1, 2, 3, 4, or 5 amino acid substitutions, wherein the side chains of the α, α-disubstituted non-natural amino acids with olefinic side chains are cross-linked (e.g., stapled and/or stitched). In some instances, the structurally-stabilized peptide is 19 to 45 (e.g., 19, 20, 21, 22, 23, 24, 25, 26, 27, 28, 29, 30, 31, 32, 33, 34, 35, 36, 37, 38, 39, 40, 41, 42, 43, 44, 45) amino acids in length.

In some instances, the peptide or structurally-stabilized (e.g., stapled, stitched) peptide described above and in this disclosure has 1, 2, 3, 4, 5 or all 6 of these properties: (i) binds the recombinant SARS-CoV-2 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2.

In one aspect, the disclosure relates to a structurally-stabilized peptide comprising or consisting of the formula:

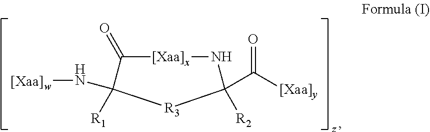

or a pharmaceutically acceptable salt thereof.

In some instances, each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted. In some instances, each R₃ is independently alkylene, alkenylene, or alkynylene, any of which is substituted or unsubstituted. In some instances, z is 1, 2, 3, 4, 5, 6, 7, 8, 9, or 10; and each [Xaa]_{w} is one of SEQ ID NOs: 53; 56, 59, 62, 65, 68, 74, 77, 80, 82, 86, or 87, or is one of I or IQ; each [Xaa]ₓ is one of SEQ ID NOs: 54, 57, 60, 63, 66, 69, 70, 72, 75, 78, 81, or 83, or is one or KEI, EID, RLN, EVA, VAK, NLN, or LNE; and each [Xaa]_{y} is one of SEQ ID NO:55; 58, 61, 64, 67, 71, 73, 76, 79, 84, 85, or is one of YI, ESL, SL, or L. In some instances, the structurally-stabilized peptide has one or more (1, 2, 3, 4, 5, 6) of the properties listed below: (i) binds the recombinant SARS-CoV-2 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2.

In some instances, the R₁ is an alkyl or a methyl group. In some instances, the R₂ is an alkenyl. In some instances, the R₃ is an alkyl or a methyl group.

In one aspect, the disclosure relates to a structurally-stabilized peptide comprising or consisting of the formula:

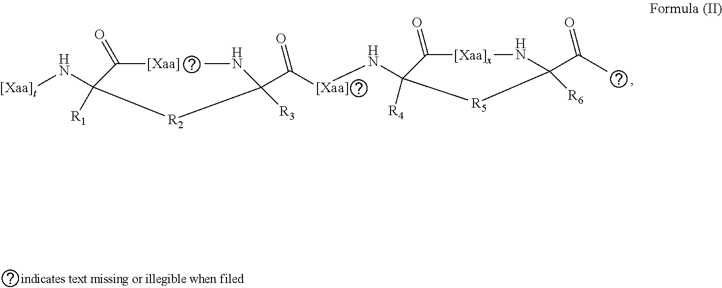

or a pharmaceutically acceptable salt thereof.

In some instances, each R₁, R₃, R₄, and R₆ is H or a C₁ to C₁₀ alkyl, alkenyl, alkynyl, arylalkyl, cycloalkylalkyl, heteroarylalkyl, or heterocyclylalkyl, any of which is substituted or unsubstituted. In some instances, each R₃ is independently alkylene, alkenylene, or alkynylene, any of which is substituted or unsubstituted. In some instances, [Xaa]ₜ is one of SEQ ID NOs: 53, 56, 59, or is one of I or IQ. In some instances, [Xaa]ᵤ is one of SEQ ID NOs: 54, 57, 60, or is one of KEI or EID. In some instances, [Xaa]ᵥ is one of SEQ ID NOs: 88-100. In some instances, [Xaa]ₓ is one of SEQ ID NOs: 63, 66, 69, or is one of NLN or LNE. In some instances, [Xaa]_{y} is one of SEQ ID NO: 64 or 67, or is one of YI, SL, or L. In some instances, the structurally-stabilized peptide has one or more (1, 2, 3, 4, 5, 6) of the properties listed below: (i) binds the recombinant SARS-CoV-2 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2.

In one aspect this disclosure features a structurally-stabilized peptide comprising the formula:

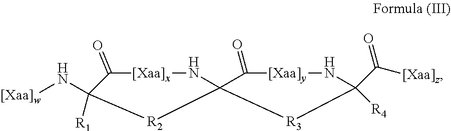

or a pharmaceutically acceptable salt thereof.

In some instances [Xaa]_{w} is one of SEQ ID NOs: 62, 74, or 77, or is I or IQ. In some instances, [Xaa]ₓ is one of SEQ ID NOs: 63, 70, 72, 75, or 78. In some instances, [Xaa]_{y} is one of SEQ ID NOs: 69, 81, or 83, or is one of EVA, VAK, NLN, or LNE. In some instances, [Xaa]_{z} is SEQ ID NO: 76 or 79 or is one of YI, ESL, SL, or L. In some instances, each R₁ and R₄ is independently H, alkyl, alkenyl, alkynyl, arylalkyl, cycloalkylalkyl, heteroarylalkyl, or heterocyclylalkyl, any of which is substituted or unsubstituted. In some instances, each R₂ and R₃ is independently alkylene, alkenylene, or alkynylene, any of which is substituted or unsubstituted. In some instances, the structurally-stabilized peptide has one or more (1, 2, 4, 5, 6) of the properties listed below: (i) binds the recombinant SARS-CoV-2 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2. In some instances, R₁ is an alkyl or a methyl group. In some instances, R₂ is an alkenyl. In some instances, R₃ is an alkenyl. In some instances, R₄ is an alkyl or a methyl group.

In one aspect this disclosure features a structurally-stabilized peptide comprising the formula:

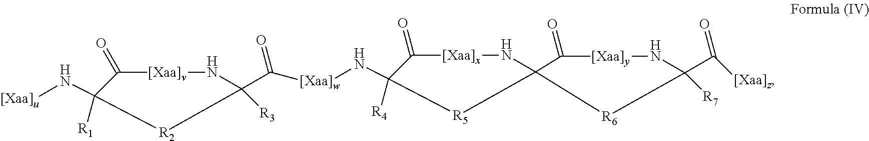

or a pharmaceutically acceptable salt thereof.

In some instances [Xaa]ᵤ is one of SEQ ID NOs: 53, 59, or 59. In some instances [Xaa]ᵥ is one of SEQ ID NOs: 54, 57, or 60. In some instances [Xaa]_{w} is one of SEQ ID NOs: 88, 91, or 94. In some instances [Xaa]ₓ is SEQ ID NO:63. In some instances [Xaa]_{y} is SEQ ID NO:69. In some instances [Xaa]_{z} is YI. In some instances, R₁, R₃, R₄, and R₇ is independently H, alkyl, alkenyl, alkynyl, arylalkyl, cycloalkylalkyl, heteroarylalkyl, or heterocyclylalkyl, any of which is substituted or unsubstituted; In some instances, R₂, R₅, and R₆ is independently alkylene, alkenylene, or alkynylene, any of which is substituted or unsubstituted. In some instances, the structurally-stabilized peptide has one or more (1, 2, 4, 5, 6) of the properties listed below: (i) binds the recombinant SARS-CoV-2 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2.

In some instances, the structurally-stabilized peptide or pharmaceutically acceptable salt thereof disclosed herein is at most 45 amino acids in length. In some cases, the structurally-stabilized peptide is 19, 20, 21, 22, 3, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids in length.

In one aspect this disclosure features a pharmaceutical composition comprising one of the peptides disclosed herein. In one aspect this disclosure features a pharmaceutical compound comprising the one of the structurally-stabilized peptides disclosed herein. In some instances, the pharmaceutical compound includes a pharmaceutically acceptable carrier.

In one aspect this disclosure features a method of treating a coronavirus infection (e.g., COVID-19) in a human subject in need thereof, the method comprising administering to the human subject a therapeutically-effective amount of any one of the peptides disclosed herein. In another aspect this disclosure features a method of treating a coronavirus infection (e.g., COVID-19) in a human subject in need thereof, the method comprising administering to the human subject a therapeutically-effective amount of any one of the structurally-stabilized peptides disclosed herein.

In one aspect this disclosure features a method of preventing a coronavirus infection (e.g., COVID-19) in a human subject in need thereof, the method comprising administering to the human subject a therapeutically-effective amount of any one of the peptides disclosed herein. In another aspect this disclosure features a method of preventing a coronavirus infection (e.g., COVID-19) in a human subject in need thereof, the method comprising administering to the human subject a therapeutically-effective amount of any one of the structurally-stabilized peptides disclosed herein.

In some instances, the methods herein are methods of treating or preventing a coronavirus infection (e.g., COVID-19). In some instances, the coronavirus infection is by a betacoronavirus. In some instances, the coronavirus infection is caused by an infection by SARS-CoV-2.

In one aspect this disclosure features a method of making a structurally-stabilized peptide, the method comprising (a) providing a peptide (e.g., SEQ ID NOT 1-52 or 112-180) as disclosed herein, and (b) cross-linking the peptide. In some instances, cross-linking the peptide is by a ruthenium catalyzed metathesis reaction.

In one aspect this disclosure features a nanoparticle-comprising composition comprising one of the structurally-stabilized peptides disclosed herein. In some instances, the peptide or structurally-stabilized peptide includes one or more of 8, 8₁, and 82. In some instances, 8, 8₁, and 82 is (R)-α-(7′-octenyl)alanine or (R)-α-(4′-pentenyl)alanine. In some instances, the peptide or structurally-stabilized peptide includes one or more of X, X₁, X₂, X₃, and X₄. In some instances, X, X₁, X₂, X₃, and X₄ each is (S)-α-(4′-pentenyl)alanine. In some instances, the peptide or structurally-stabilized peptide includes a #, which is α,α-Bis(4′-pentenyl)glycine or α,α-Bis(7′-octenyl)glycine. In some instances, the peptide or structurally-stabilized peptide includes a %, which is (S)-α-(7′-octenyl)alanine or (S)-α-(4′-pentenyl)alanine. In some instances, the nanoparticle is a PLGA nanoparticle. In certain cases, the lactic acid:glycolic acid ratio of the PLGA nanoparticle is in the range of 2:98 to 100:0.

In one aspect this disclosure features a structurally-stabilized peptide, wherein 8, 8₁, and 8₂=(R)-α-(7′-octenyl)alanine or (R)-α-(4′-pentenyl)alanine; X, X₁, X₂, X₃, and X₄=(S)-α-(4′-pentenyl)alanine; #=α,α-Bis(4′-pentenyl)glycine or α,α-Bis(7′-octenyl)glycine; and %=(S)-α-(7′-octenyl)alanine or (S)-α-(4′-pentenyl)alanine. In another aspect, the structurally-stabilized comprises peptides, wherein 8, 8₁, and 8₂=(R)-α-(7′-octenyl)alanine; X, X₁, X₂, X₃, and X₄=(S)-α-(4′-pentenyl)alanine; #=α,α-Bis(4′-pentenyl)glycine; and %=(S)-α-(7′-octenyl)alanine.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the disclosure will be apparent from the following detailed description and from the claims.

### BRIEF DESCRIPTION OF DRAWINGS

FIGS. 1A-1B provide the amino acid sequence of the S protein (SEQ ID NO: 1) (FIG. 1A) and three generated sequences of helical bundles (SEQ ID NOs: 261-263) (FIG. 1B) of SARS-CoV-2. Underlined sequences in FIG. 1B represent HR1 sequences and boxed sequences in FIG. 1B represent HR2 FIG. 2 is a schematic representation of the SARS-CoV-2 spike (S) protein, including the sequence composition of the heptad repeat domain 1 (HR1) (SEQ ID NO: 2) and heptad repeat domain 2 (HR2) (SEQ ID NO: 3) fusion domains.

FIG. 3 depicts the mechanism of action of SARS-CoV-2 S fusion inhibitor peptides.

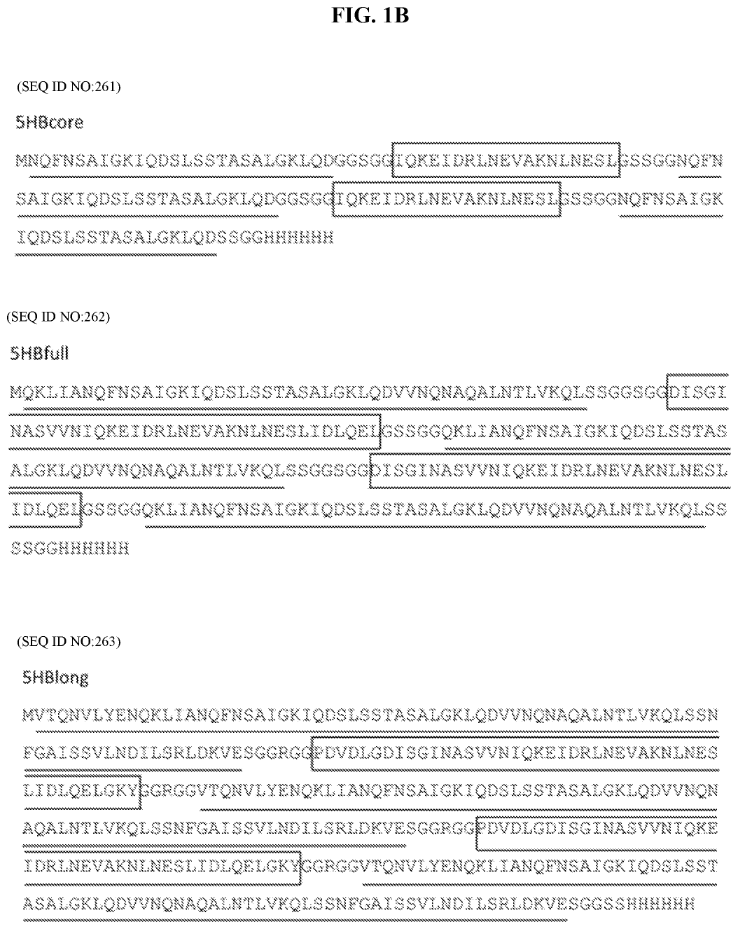

FIG. 4 is a helical wheel depiction of a portion of the SARS-CoV-2 S HR2₍₁₁₆₉₋₁₂₁₀₎ domain amphipathic alpha-helix (SEQ ID NOs: 4-6), illustrating the predominantly hydrophobic binding interface, with flanking charged or polar residues at the perimeter of the binding interface and at the non-interacting face. The arrows refer to the hydrophobic moment.

FIG. 5 is a helical wheel depiction of a portion of the SARS-CoV-2 S HR2₍₁₁₇₉₋₁₁₉₇₎ domain amphipathic alpha-helix (SEQ ID NO: 7), illustrating the predominantly hydrophobic binding interface, with flanking charged or polar residues at the perimeter of the binding interface and at the non-interacting face. The arrow refers to the hydrophobic moment.

FIGS. 6A-6B show an alignment of the HR1 and HR2 regions of SARS-CoV-2 and SARS-CoV-1 (“SARS”) (FIG. 6A) and an alignment of HR2 sequences from SARS-CoV-2, MERS, and an alternate HR2-type sequence (“EK1”) (FIG. 6B). In FIG. 6A, the SARS HR1 sequence is set forth in SEQ ID NO: 8. The SARS-CoV-2 HR1 sequence is set forth in SEQ ID NO: 2. The SARS-CoV-1 and SARS-CoV-2 HR2 sequence is set forth in SEQ ID NO: 3. In FIG. 6B, the SARS-CoV2 sequence is set forth in SEQ ID NO:108, the MERS sequence is set forth in SEQ ID NO: 259; and the EK1 sequence is set forth in SEQ ID NO:110. The core template helical sequences of SARS-CoV-2 HR2 and its two homologs are underlined and the core template sequences from SARS-CoV-2 HR2 and EK1 are set forth in SEQ ID NO:10 and SEQ ID NO:258, respectively. The alignment in FIG. 6B allows identification of possible residues in the SARS-CoV2 HR2 sequence that can be modified and to what amino acids.

FIG. 7 shows a variety of non-natural amino acids containing olefinic tethers that can be used to generate hydrocarbon stapled SARS-CoV-2 S peptides bearing staples spanning i, i+3; i, i+4, and i, i+7 positions. Single staple scanning is used to generate a library of singly stapled COVID-19-S peptides.

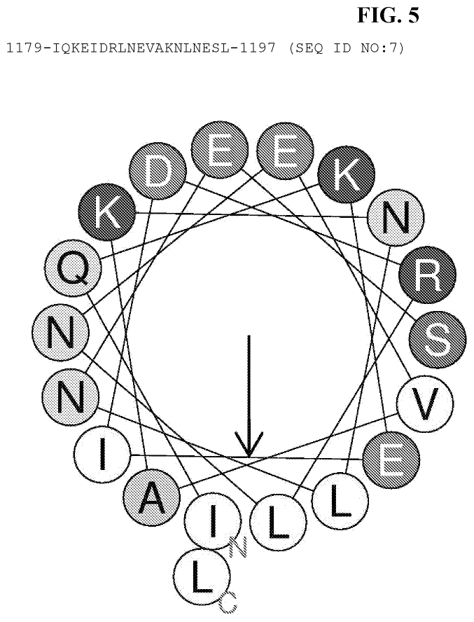

FIG. 8 shows a variety of staple compositions in multiply stapled peptides and staple scanning to generate a library of multiply stapled SARS-CoV-2 S peptides.

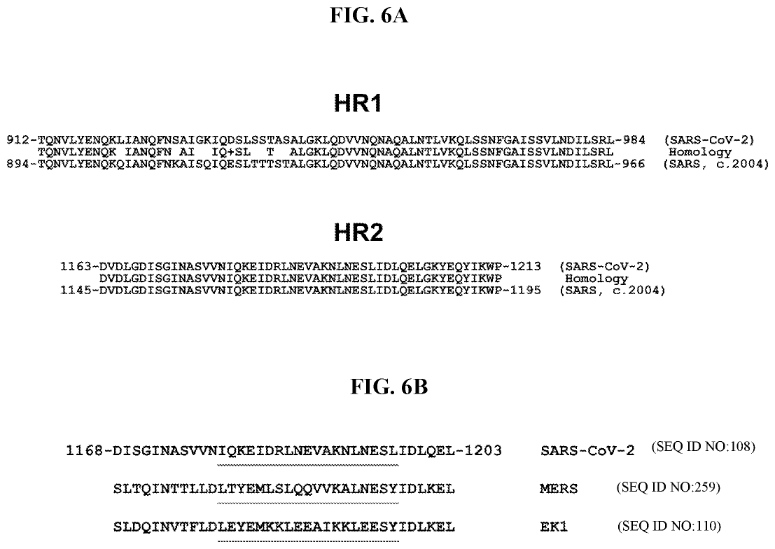

FIG. 9 shows a variety of staple compositions in tandem stitched peptides to generate a library of stitched SARS-CoV-2 S peptides.

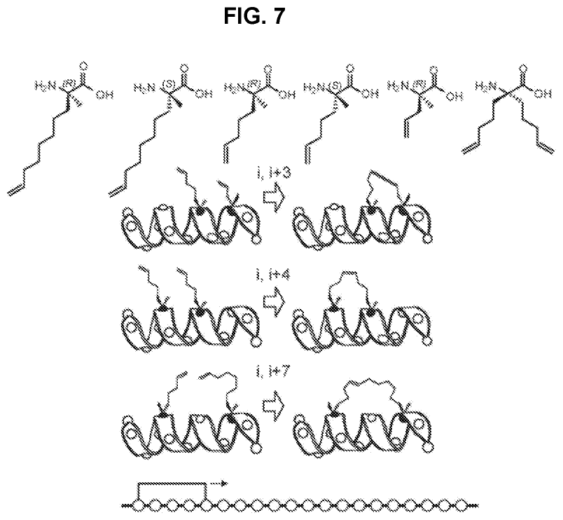

FIG. 10 is an illustration of an exemplary approach to designing, synthesizing, and identifying optimal stapled peptide constructs to target the SARS-CoV-2 fusion apparatus, including the generation of Ala scan, staple scan, and variable N- and C-terminal deletion, addition, and derivatization libraries. Singly and doubly stapled and stitched constructs, including alanine and staple and stitch scans, are used to identify optimal stapled peptides for in vitro and in vivo analyses.

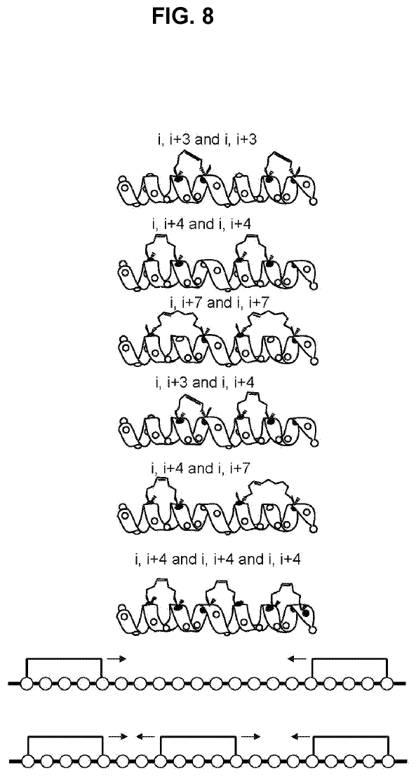

FIG. 11 shows exemplary structurally-stabilized SARS-CoV-2 HR2 peptide sequences generated by i, i+4 and i, i+7 staple scanning of a core template sequence (aa 1169-1197) and variations thereof characterized by N- and C-terminal unstapled sequence extensions, terminal derivatizations (e.g. PEG4-cholesterol), incorporation of double staples and stitches, and application of staples to an alternate HR2-type sequence. The letter designation next to the SEQ ID NO is a key for the location of the staples in the sequence.

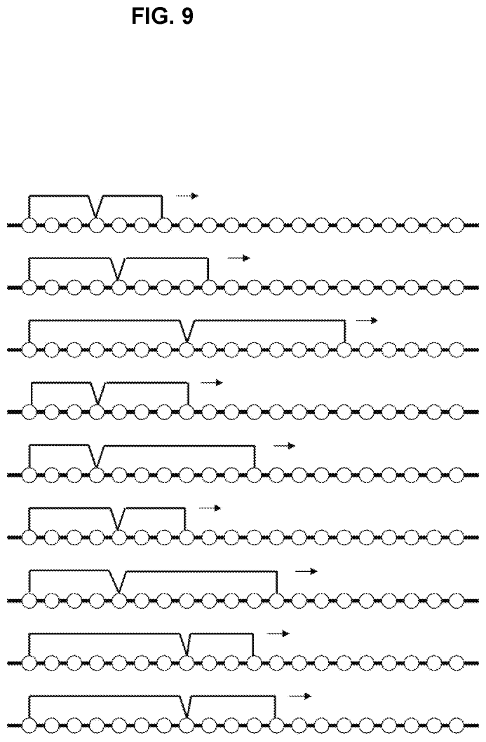

FIGS. 12A-12B show that insertion of staples into the core template sequence (aa 1169-1197) confers striking alpha-helical structure compared to the unstapled sequence, and this structural benefit is preserved upon appending N- and/or C-terminal unstapled sequences. FIG. 12A compares the circular dichroism spectra of unstapled core template sequence (SEQ ID NO:10) with that containing stitches J,S (SEQ ID NO:47) or K,T (SEQ ID NO:48), or double staples N,S (SEQ ID NO:49) or N,T (SEQ ID NO:51). FIG. 12B compares the circular dichroism spectra of longer unstapled HR2 sequences (SEQ ID NOs: 9, 108, 110) with those containing double staples O,S (SEQ ID NO:158) and N,S (SEQ ID NO:177).

FIGS. 13A-13B show that insertion of double staples or stitches into the core template sequence (aa 1169-1197) confers striking protease resistance compared to the unstapled sequence, depending on the sequence, staple type, and staple location. FIG. 13A shows that double staples or a stitch (SEQ ID NOs: 48 and 52) both confer marked resistance to Proteinase K treatment (half-lives of >1000 min), whereas the unstapled sequence (SEQ ID NO:10) is rapidly digested (half-life of 35 min). FIG. 13B shows that the longer unstapled HR2 sequence (SEQ ID NO:9) is rapidly digested by Proteinase K (half-life of 25 min) and insertion of double staples 0, S (SEQ ID NO:158) only mildly enhances proteolytic resistance (half-life of 33 min), whereas insertion of double staples N,S (SEQ ID NO: 177) into an alternate HR2-type sequence (SEQ ID NO:110) confers marked proteolytic resistance to Proteinase K (half-life of 840 min).

FIGS. 14A-14B show mouse plasma stability (no degradation) of two doubly stapled peptides of the core template sequence (aa 1169-1197), including SEQ ID NO:51 (Staples N,T) in FIG. 14A and SEQ ID NO:52 (Staples 0, T) in FIG. 14B.

FIGS. 15A-15B show the results of a direct fluorescence polarization binding assay using the recombinant SARS-CoV-2 5-helix binding protein and an N-terminal FITC derivatized i, i+4 staple scanning library of the core template sequence (aa 1179-1197, SEQ ID NO: 10). FIG. 15A illustrates the differential binding activities of the stapled peptides based on staple location, as reflected by the change in fluorescence polarization (ΔmP) at 4 μM 5-HB protein concentration. FIG. 15B shows the dose-response curves for the fluorescent i, i+4 staple scanning library to the 5-HB protein, highlighting that depending on the particular staple position, the i, i+4 stapled peptides bind either better, similar to, or worse than the unstapled core template sequence. In both FIGS. 15A and 15B, the sequences from top to bottom have SEQ ID NOs.: 130, 36, 37, 131, 132, 38, 133, 134, 39, 40, 135, 136, 41, 42, 137, and 10.

FIGS. 16A-16D show the results of a direct fluorescence polarization binding assay using the recombinant SARS-CoV-2 5-helix binding protein and an N-terminal FITC derivatized i, i+7 staple scanning library of the core template sequence (aa 1179-1197, SEQ ID NO:10). FIG. 16A illustrates the differential binding activities of the stapled peptides based on staple location, as reflected by the change in fluorescence polarization (ΔmP) at 4 μM 5-HB protein concentration. FIG. 16B shows the dose-response curves for the fluorescent i, i+7 staple scanning library to the 5-HB protein, highlighting that depending on the particular staple position, the i, i+7 stapled peptides bind either better, similar to, or worse than the unstapled core template sequence. In both FIGS. 16A and 16B the sequences from top to bottom have SEQ ID NOs.: 112, 30, 31, 113, 114, 32, 33, 115, 34, 35, 116, 117, and 10. FIG. 16C shows a helical wheel diagram depicting residues that participate in a favorable (light grey), unfavorable (dark grey), and intermediate (medium grey) i, i+7 staple. Residues that participate in two staples are shown as bisected circles with the leftward semicircle representative of the residue's incorporation at the N-terminal position of a staple and the rightward semicircle representative of the residue's incorporation at the C-terminal position of a staple; when a semicircle is colored white, the indicated residue position does not participate in either an N- or C-terminal staple position. Staple positions located at the hydrophobic surface disrupt 5-HB binding activity and, unexpectedly, staple positions located at the hydrophilic surface opposite to the 5-HB binding surface are also disfavored (dark grey-colored residues; marked by X's). In contrast, select staple positions at the boundary between the hydrophobic binding surface and the hydrophilic surface are favored (light grey-colored residues; marked by stars). FIG. 16D shows the SARS CoV-2 HR2 sequence, highlighting the roles of particular amino acids in engaging the HR1 heptad repeat and the tolerance or intolerance of staples at particular positions, informing which residues are more or less amenable to amino acid substitution.

FIGS. 17A-17B show the results of a direct fluorescence polarization binding assay using the recombinant SARS-CoV-2 5-helix binding protein and N-terminal FITC derivatized double i, i+4 stapled peptides (SEQ ID NOs: 51 (N, T) and 52 (O,T)) of the core template sequence (aa 1179-1197, SEQ ID NO:10). FIG. 17A illustrates the differential binding activities of the stapled peptides based on double staple locations, as reflected by the change in fluorescence polarization (ΔmP) at 4 M 5-HB protein concentration. FIG. 17B shows the dose-response curves for the fluorescent double stapled peptides to the 5-HB protein, highlighting that in each example, insertion of the double staples leads to enhanced binding activity compared to the unstapled core template sequence.

FIG. 18 shows the results of a direct fluorescence polarization binding assay using the recombinant SARS-CoV-2 5-helix binding protein and N-terminal FITC derivatized double i, i+4 stapled peptides (from top to bottom, SEQ ID NOs.: 156, 158, 160, 162, 179, and 180) of core template sequences, SEQ ID NO:10 or LEYEBKKLEEAIKKLEESY (SEQ ID NO: 258), within the context of the longer HR2 (SEQ ID NO:9) and alternate HR2-type (SEQ ID NO:110) sequences, respectively. The plot demonstrates the comparative dose-responsive binding activity of the double stapled peptides for the 5-HB of SARS-CoV-2.

FIGS. 19A-19C show the results of a competitive ELISA binding assay in which the interaction between SARS-CoV-2 5-HB protein and the SARS-CoV-2 unstapled HR2 sequence corresponding to SEQ ID NO:9 is competed by a serial dilution of an i, i+4 staple scanning library (SEQ ID NOs.: 138-152, top to bottom) of the core template sequence (SEQ ID NO:10) with an N-terminal extension (aa 1169-1197). FIG. 19A shows the full dose-response competitive binding curves and FIG. 19B and FIG. 19C highlight the comparative, competitive binding activity for each construct at 3 μM and 10 μM dosing, respectively.

FIG. 20 shows the results of a competitive ELISA binding assay in which the interaction between SARS-CoV-2 5-HB protein and the SARS-CoV-2 unstapled HR2 sequence corresponding to SEQ ID NO:9 is competed by a fixed dose (10 μM) of double stapled and stitched peptides (SEQ ID NOs.: 10, 52, 51, 50, 49, 48, 47, 44, and 43, top to bottom) of the core template SARS-CoV-2 HR2 sequence corresponding to SEQ ID:NO 10. Whereas the unstapled core template sequence (SEQ ID NO:10) is unable to compete with the longer HR2 template sequence (SEQ ID NO:9) for binding to the 5-HB, select double stapled (staple combinations O,S and K,T) and stitched (staple combination H,L) peptides of the core template sequence are capable of partially disrupting the binding interaction at 10 μM dosing.

FIG. 21 shows the results of a competitive ELISA binding assay in which the interaction between SARS-CoV-2 5-HB protein and the SARS-CoV-2 unstapled HR2 sequence corresponding to SEQ ID NO:9 is competed by dose-responsive treatment with double stapled and stitched peptides (SEQ ID NOs.: 9, 153, 154, 156, 158, 160, and 162, top to bottom) of the longer HR2 sequence corresponding to SEQ ID NO:9. The effectiveness at disrupting the 5-HB/HR2 interaction depends upon the staple type and staple positioning of the double staples and stitches within the core template sequence (SEQ ID NO: 10) within the context of the longer HR2 peptide (SEQ ID NO:9).

FIG. 22 shows the results of a competitive ELISA binding assay in which the interaction between SARS-CoV-2 5-HB protein and the SARS-CoV-2 unstapled HR2 sequence corresponding to SEQ ID NO:9 is competed by dose-responsive treatment with double stapled and stitched peptides (SEQ ID NOs. 110 and 175-180, top to bottom) of an alternative HR2 sequence corresponding to SEQ ID NO:110. The effectiveness at disrupting the 5-HB/HR2 interaction depends upon the staple type and staple positioning of the double staples and stitches of the core template sequence within the context of the longer HR2-type peptide (SEQ ID NO:110), with double staples N,S producing the most potent competitive inhibitor of this group.

FIG. 23 shows the antiviral activity of exemplary double stapled and stitched peptides (SEQ ID NOs.: 43, 49, 48, 52, and 22, top to bottom) of the core template sequence SEQ ID NO:10 and a double stapled peptide of the longer HR2 sequence corresponding to SEQ ID NO:9. Peptides were screened at 25 μM for the capacity to block infection of Vero E6 cells by live, wild-type SARS-CoV-2 virus, with fraction infected cells plotted. In each case, the stapled peptides inhibit infection as compared to treatment with the vehicle control.

FIG. 24 shows that hits from the peptide screen in SARS-CoV-2-exposed Vero E6 cells subjected to SARS-CoV-2 infection were then subjected to further dose-response testing, as exemplified by the double stapled core template sequence bearing staples O,T (SEQ ID NO:52), which has an IC₅₀ below 6 μM for blocking SARS-CoV-2 infection in the assay.

FIG. 25 shows the differential anti-viral activity of double stapled and stitched peptides (SEQ ID NOs.:10, 43, 44, 47-52, from left to right) of the core template sequence (SEQ ID NO:10), as assessed in high-throughput by an antibody-based SARS-CoV-2 detection platform in infected Vero E6 cells.

FIG. 26 shows that double i, i+7 stapling and stitching in the indicated positions outside of the core template sequence (SEQ ID NO:10) within the context of the longer HR2 peptide sequence (SEQ ID NO:9) did not yield compounds with anti-viral activity, as assessed in high-throughput by an antibody-based SARS-CoV-2 detection platform in infected Vero E6 cells. The sequences from top to bottom have SEQ ID NOs.: 9, 26-28, 19, 22, 23, 25, and 24.

FIG. 27 shows the differential anti-viral activity of exemplary double stapled and stitched peptides of the core template sequence (SEQ ID NO:10) within the context of the longer HR2 peptide sequence corresponding to SEQ ID NO:9, as assessed in high-throughput by an antibody-based SARS-CoV-2 detection platform in infected Vero E6 cells. The construct bearing double i, i+4 staples O,S had the most potent antiviral activity, followed by compounds bearing the O,T; I,R; and N,S staples, whereas the N,T and H,L constructs showed no effect in this assay across the indicated dosing range. The sequences from top to bottom have SEQ ID NOs:9, 156, 158, 160, 162, 154, and 153.

FIG. 28 shows the differential anti-viral activity of exemplary double stapled and stitched peptides of an alternate core template sequence (SEQ ID NO:258) in the context of its longer HR2-type peptide sequence corresponding to SEQ ID NO:110, as assessed in high-throughput by an antibody-based SARS-CoV-2 detection platform in infected Vero E6 cells. The construct bearing double i, i+4 staples N,S had the most potent antiviral activity, followed by the peptide containing the N,T staples, whereas the other compounds in this group showed no significant effect in this assay across the indicated dosing range. The sequences from top to bottom have SEQ ID NOs.:110 and 175-180.

FIG. 29 shows the differential anti-viral activity of double stapled and stitched peptides of the core template sequence (SEQ ID NO:10) compared to the unstapled core template sequence that shows no anti-viral activity, as assessed by a SARS-CoV-2 pseudoviral assay in which the number of infected cells is counted by IXM microscopy based on the fluorescence of ACE2-expressing 293T cells infected by the GFP-expressing pseudovirus. The sequences from top to bottom have SEQ ID NOs.:10, 43, 44, and 47-52.

FIG. 30 shows the differential anti-viral activity of double stapled and stitched peptides of the core template sequence (SEQ ID NO:10) in the context of its longer HR2 sequence (SEQ ID NO:9), as assessed by a SARS-CoV-2 pseudoviral assay in which the number of infected cells is counted by IXM microscopy based on the fluorescence of ACE2-expressing 293T cells infected by the GFP-expressing pseudovirus. The sequences from top to bottom have SEQ ID NOs.:153, 154, 156, 158, 160, and 162.

FIG. 31 shows the differential anti-viral activity of double stapled peptides of the core template sequence (SEQ ID NO:10) with or without an N-terminal peptide extension (aa 1168-1176) and bearing a C-terminal derivatization with GSGSGC(SEQ ID NO:256)-(PEG4-chol)-carboxamide, as assessed by a SARS-CoV-2 pseudoviral assay in which the number of infected cells is counted by IXM microscopy based on the fluorescence of ACE2-expressing 293T cells infected by the GFP-expressing pseudovirus. The sequences from top to bottom have SEQ ID NOs.:155, 159, 161, and 167-170.

FIG. 32 shows the differential anti-viral activity of double stapled and stitched peptides of an alternate core template sequence (SEQ ID NO:258) in the context of its longer HR2-type sequence (SEQ ID NO:110), as assessed by a SARS-CoV-2 pseudoviral assay in which the number of infected cells is counted by IXM microscopy based on the fluorescence of ACE2-expressing 293T cells infected by the GFP-expressing pseudovirus. The sequences from top to bottom have SEQ ID NOs.: 175-180.

### DETAILED DESCRIPTION

The present disclosure is based, inter alia, on the discovery that stabilized (e.g., stapled, double stapled, stitched, stapled and stitched) peptides may be designed to selectively bind to one or more coronaviruses (e.g., betacoronaviruses such as SARS-CoV-2). Accordingly, the present disclosure provides novel methods and compositions (e.g., peptides, stabilized peptides, combinations of peptides; combinations of stabilized peptides; combinations of peptides and stabilized peptides) for treating, for developing treatments for, and for preventing infection with one or more coronaviruses (e.g., betacoronaviruses such as SARS-CoV-2). Thus, the peptides and composition disclosed herein can be used to prevent and/or treat COVID-19.

### Coronavirus Peptides

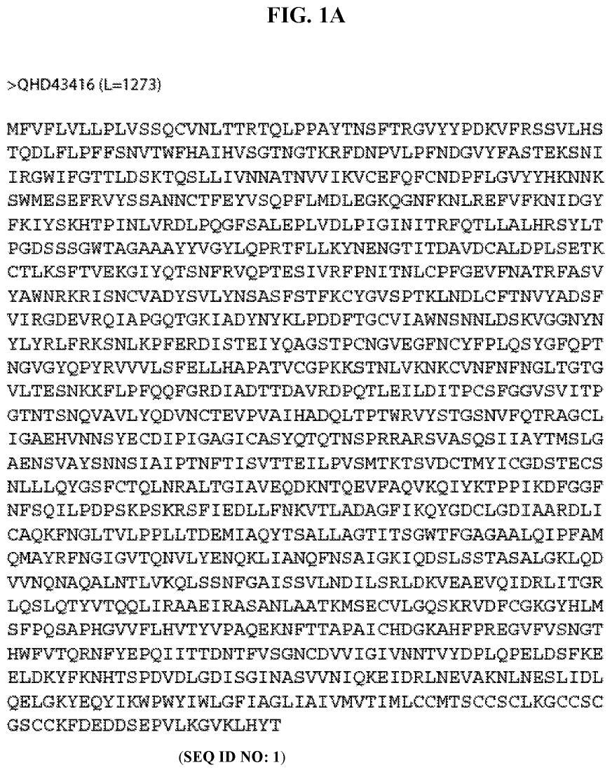

The amino acid sequence of an exemplary coronavirus surface glycoprotein is provided in FIG. 1. (See also, GenBank Accession No. QHD43416.1.) An exemplary amino acid sequence of the heptad repeat domain 1 (HR1) in SARS-CoV-2 S is shown in FIG. 2. An exemplary amino acid sequence of the heptad repeat domain 2 (HR2) in SARS-CoV-2 S is also shown in FIG. 2.

Other exemplary amino acid sequences of the HR2 in SARS-CoV-2 S are provided as SEQ ID NOs: 9, 10, 103, 104, 106, 108, and 110 (an alternate HR2 region (EK1)) in Table 1.

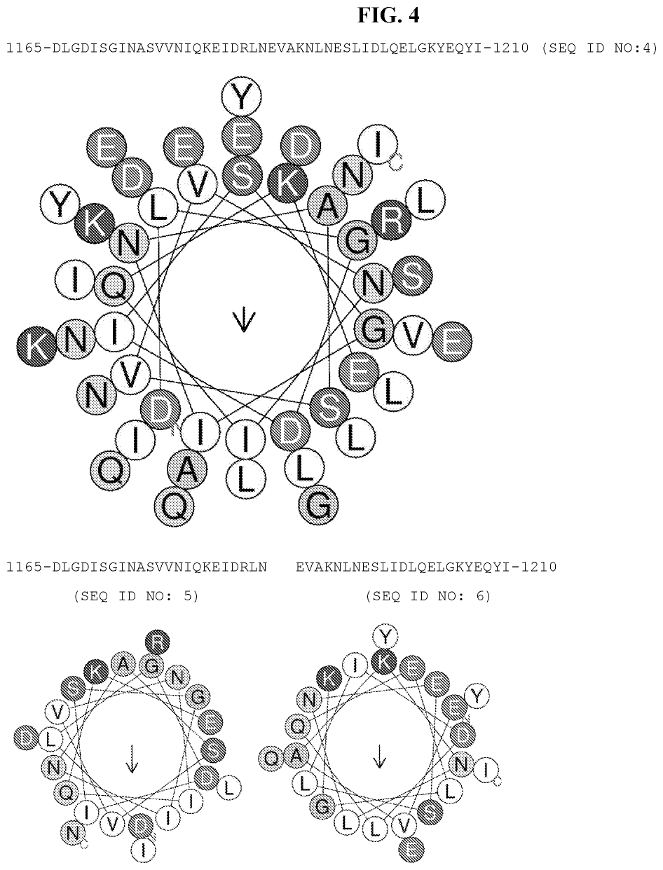

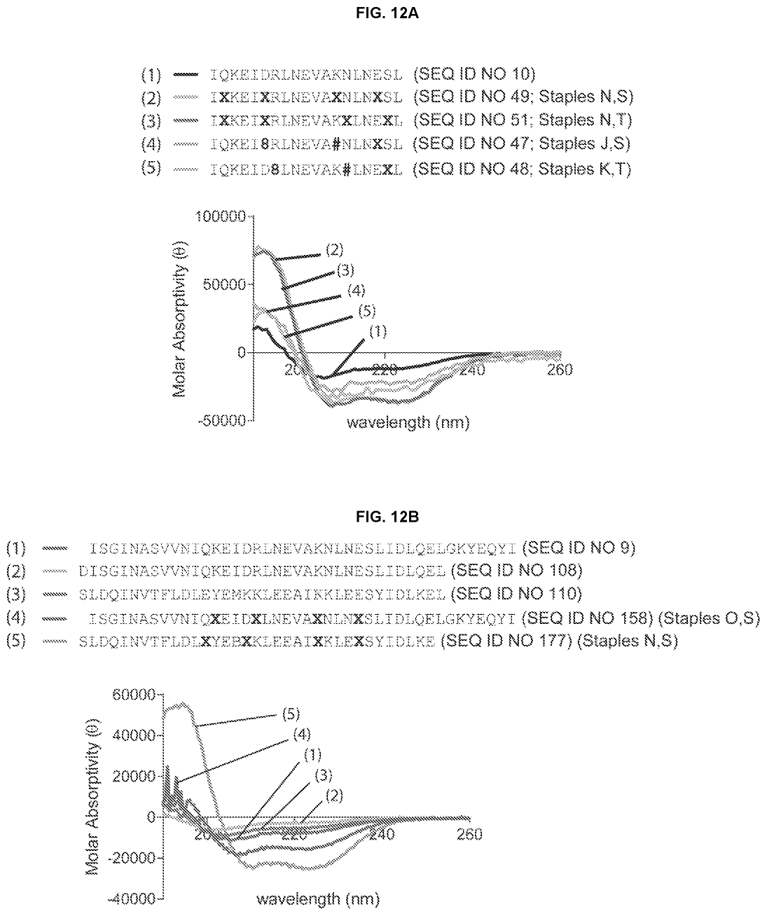

In certain instances, the SARS-CoV-2 HR1 or HR2 peptides described herein (e.g., SEQ ID NO: 2, 3, 9, 10, 103, 104, 106, 108, and 110) may also contain one or more (e.g., 1, 2, 3, 4, 5, 6, 7, 8, 9, 10) amino acid substitutions (relative to an amino acid sequence set forth in any one of SEQ ID NOs: 2, 3, 9, 10, 103, 104, 106, 108, or 110), e.g., one or more (e.g., 1, 2, 3, 4, 5, 6, 7, 8, 9, 10) conservative and/or non-conservative amino acid substitutions. In addition, in some instances at least two (e.g., 2, 3, 4, 5, or 6) amino acids of SEQ ID NOs: 2, 3, 9, 10, 103, 104, 106, 108, or 110 may be substituted by α, α-disubstituted non-natural amino acids with olefinic side chains. The type of substitutions that are made can, e.g., be guided by an alignment of the HR2-like region of SARS, MERS, and the EK1 peptide (FIG. 6B) and the guidance provided by FIG. 16D. The guidance provided in the Structurally-Stabilized Peptides section below regarding the amino acids that can be varied is equally relevant for the peptides described herein. Residues that are unchanged between SARS, MERS, and EK1 in such an alignment are either unmodified or substituted with a non-natural amino acid or a conservative amino acid. Residues in the alignment that are found replaced by conservative substitutions (e.g., Isoleucine in SARS replaced by Leucine or Methionine) in the HR2-like region of MERS or EK1 are either not replaced or replaced by conservative amino acid substitutions. Residues that are not conserved between the HR2-like region of SARS, MERS, and EK1 can be replaced by any amino acid.

A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), and acidic side chains and their amides (e.g., aspartic acid, glutamic acid, asparagine, glutamine).

In some instances, the SARS-CoV-2 HRT or HR2 peptides described herein (e.g., SEQ ID NO: 2, 3, 9, 10, 103, 104, 106, 108, or 110) may also contain at least one, at least 2, at least 3, at least 4, or at least 5 amino acids added to the N-terminus of the peptide. In some instances, the SARS-CoV-2 HRT or HR2 peptides described herein (e.g., SEQ ID NO: 2 or 3, 9, 10, 103, 104, 106, 108, or 110) may also contain at least one, at least 2, at least 3, at least 4, or at least 5 amino acids added to the C-terminus of the peptide. In some instances, the SARS-CoV-2 HRT or HR2 peptides described herein (e.g., SEQ ID NO: 2 or 3, 9, 10, 103, 104, 106, 108, or 110) may also contain at least one, at least 2, at least 3, at least 4, or at least 5 amino acids deleted at the N-terminus of the peptide. In some instances, the SARS-CoV-2 HRT or HR2 peptides described herein (e.g., SEQ ID NO: 2 or 3, 9, 10, 103, 104, 106, 108, or 110) may also contain at least one, at least 2, at least 3, at least 4, or at least 5 amino acids deleted at the C-terminus of the peptide.

In some cases, the peptides are lipidated. In some cases, the peptides are modified to comprise polyethylene glycol and/or cholesterol. In some cases, the peptides (e.g., SEQ ID NOs.: 3, 9, 10, 103, 104, 106, 108, or 110) include the GSGSGC (SEQ ID NO:256) sequence appended at the C-terminus of the peptide. In some cases, the peptides (e.g., SEQ ID NOs.: 3, 9, 10, 103, 104, 106, 108, or 110) include the GSGSGC (SEQ ID NO:256)-(PEG4-chol)-carboxamide appended at the C-terminus of the peptide. In some instances, the peptide is any one of SEQ ID NOs.: 102, 105, 107, and 109, or a peptide that differs from these sequences at 1, 2, 3, 4, 5, 6, 7, 8, 9, or 10 positions within SEQ ID NOs.: 102, 105, 107, and 109.

In some instances, the peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length.

In some instances, the peptides described above bind the recombinant 5-helix bundle of SARS-CoV-2 S protein; and/or inhibits or disrupts interaction between the recombinant 5-helix bundle and a SARS CoV-2 HR2 peptide (e.g., one of those in SEQ ID NO: 9, 10, 103, 104, 106, 108); and/or inhibits fusion of SARS-CoV-2 with a host cell; and/or inhibits infection of a cell by SARS-CoV-2.

### Structurally-Stabilized Peptides

Disclosed herein are stapled or stitched SARS-CoV-2 peptides based on a portion of the HR2 region or an alternate HR2 region (EK1). In some instances, the stapled or stitched SARS-CoV-2 peptides are derived from SARS-CoV-2 HR2₍₁₁₆₉₋₁₂₁₀₎ (ISGINASVVNIQKEIDRLNEVAKNLNESLIDLQELGKYEQYI (SEQ ID NO:9)). In some instances, the stapled or stitched SARS-CoV-2 peptides derived from SEQ ID NO:9 include SAH-SARS-CoV-2-A; SAH-SARS-CoV-2-B; SAH-SARS-CoV-2-C; SAH-SARS-CoV-2-D; SAH-SARS-CoV-2-E; SAH-SARS-CoV-2-F; SAH-SARS-CoV-2-G; SAH-SARS-CoV-2-A,D; SAH-SARS-CoV-2-A,E; SAH-SARS-CoV-2-A,F; SAH-SARS-CoV-2-A,G; SAH-SARS-CoV-2-B,D; SAH-SARS-CoV-2-B,E; SAH-SARS-CoV-2-B,F; SAH-SARS-CoV-2-B,G; SAH-SARS-CoV-2-C,D; SAH-SARS-CoV-2-C,E; SAH-SARS-CoV-2-C,F; or SAH-SARS-CoV-2-C,G (e.g., SEQ ID NOs: 11-29), as shown in Table 1 below. Additional sequences are provided in Table 1.

In some instances, the stapled or stitched SARS-CoV-2 peptides are derived from SARS-CoV-2 HR2₍₁₁₇₉₋₁₁₉₇₎ (IQKEIDRLNEVAKNLNESL (SEQ ID NO: 10)).

In some instances, the stapled or stitched SARS-CoV-2 peptides derived from SEQ ID NO: 10 include SAH-SARS-CoV-2-H; SAH-SARS-CoV-2-I; SAH-SARS-CoV-2-J; SAH-SARS-CoV-2-K; SAH-SARS-CoV-2-L; SAH-SARS-CoV-2-M; SAH-SARS-CoV-2-N; SAH-SARS-CoV-2-O; SAH-SARS-CoV-2-P; SAH-SARS-CoV-2-Q; SAH-SARS-CoV-2-R; SAH-SARS-CoV-2-S; SAH-SARS-CoV-2-T; SAH-SARS-CoV-2-H-L; SAH-SARS-CoV-2-I-M; SAH-SARS-CoV-2-H-Q; SAH-SARS-CoV-2-I-R; SAH-SARS-CoV-2-J-S; SAH-SARS-CoV-2-K-T; SAH-SARS-CoV-2-N,S; SAH-SARS-CoV-2-O,S; SAH-SARS-CoV-2-N,T; and SAH-SARS-CoV-2-O,T (e.g., SEQ ID NOs: 30-52), as shown in Table 1 below. Additional sequences are provided in Table 1.

In some instances, the stapled or stitched SARS-CoV-2 peptides derived from SARS-CoV-2 HR2₍₁₁₇₉₋₁₁₉₇₎ (IQKEIDRLNEVAKNLNESL (SEQ ID NO: 10) further includes the amino acid sequence ISGINASVVN (SEQ ID NO:250) appended at the N-terminus of the amino acid sequence. In some instances, the stapled or stitched SARS-CoV-2 peptides derived from SARS-CoV-2 HR2₍₁₁₇₉₋₁₁₉₇₎ (IQKEIDRLNEVAKNLNESL (SEQ ID NO: 10) further includes the amino acid sequence DISGINASVVN (SEQ ID NO:251) appended at the N-terminus of the amino acid sequence. In some instances, the stapled or stitched SARS-CoV-2 peptides derived from SARS-CoV-2 HR2₍₁₁₇₉₋₁₁₉₇₎ (IQKEIDRLNEVAKNLNESL (SEQ ID NO: 10) further includes the amino acid sequence IDLQEL (SEQ ID NO:252) appended at the C-terminus of the amino acid sequence. In some instances, the stapled or stitched SARS-CoV-2 peptides derived from SARS-CoV-2 HR2₍₁₁₇₉₋₁₁₉₇₎ (IQKEIDRLNEVAKNLNESL (SEQ ID NO: 10) further includes the amino acid sequence IDLQELGKYEQYI (SEQ ID NO:253) appended at the C-terminus of the amino acid sequence. In some instances, the stapled or stitched SARS-CoV-2 peptides derived from SARS-CoV-2 HR2₍₁₁₇₉₋₁₁₉₇₎ (IQKEIDRLNEVAKNLNESL (SEQ ID NO: 10) further includes the amino acid sequence IDLQELGSGSGC (SEQ ID NO:254) appended at the C-terminus of the amino acid sequence. In some instances, the stapled or stitched SARS-CoV-2 peptides derived from SARS-CoV-2 HR2₍₁₁₇₉₋₁₁₉₇₎ (IQKEIDRLNEVAKNLNESL (SEQ ID NO: 10) further includes the amino acid sequence IDLQELGKYEQYIGSGSGC (SEQ ID NO:255) appended at the C-terminus of the amino acid sequence.

In some instances, the stapled or stitched SARS-CoV-2 peptides are derived from SARS-CoV-2 HR2_{(1179-1197)*} (IQKEIDRLNEVAKNLNESL* (SEQ ID NO: 102), wherein *=GSGSGC(SEQ ID NO:256)-(PEG4-chol)-carboxamide). In some instances, the stapled or stitched SARS-CoV-2 peptides are derived from COVID19 HR2₍₁₁₆₉₋₁₁₉₇₎ (ISGINASVVNIQKEIDRLNEVAKNLNESL (SEQ ID NO: 103)). In some instances, the stapled or stitched SARS-CoV-2 peptides are derived from COVID19 HR2₍₁₁₇₉₋₁₂₀₃₎ (IQKEIDRLNEVAKNLNESLIDLQEL (SEQ ID NO: 104)). In some instances, the stapled or stitched SARS-CoV-2 peptides are derived from COVID19 HR2_{(1179-1203)*} (IQKEIDRLNEVAKNLNESLIDLQEL* (SEQ ID NO: 105)). In some instances, the stapled or stitched SARS-CoV-2 peptides are derived from COVID19 HR2₍₁₁₆₈₋₁₁₉₇₎ (DISGINASVVNIQKEIDRLNEVAKNLNESL (SEQ ID NO: 106)). In some instances, the stapled or stitched SARS-CoV-2 peptides are derived from COVID19 HR2_{(1168-1197)*} (DISGINASVVNIQKEIDRLNEVAKNLNESL* (SEQ ID NO: 107)).

In some instances, the stapled or stitched SARS-CoV-2 peptides are derived from COVID19 HR2₍₁₁₆₈₋₁₂₀₃₎ (DISGINASVVNIQKEIDRLNEVAKNLNESLIDLQEL (SEQ ID NO: 108)). In some instances, the stapled or stitched SARS-CoV-2 peptides are derived from COVID19 HR2_{(1168-1203)*} (DISGINASVVNIQKEIDRLNEVAKNLNESLIDLQEL* (SEQ ID NO: 109)). In some instances, the stapled or stitched SARS-CoV-2 peptides are derived from EK1 (SLDQINVTFLDLEYEMKKLEEAIKKLEESYIDLKEL (SEQ ID NO: 110)). In some instances, the stapled or stitched SARS-CoV-2 peptides are derived from EK1* (SLDQINVTFLDLEYEMKKLEEAIKKLEESYIDLKEL* (SEQ ID NO: 111)).

In some instances, the SARS-CoV-2 HR2 stabilized peptide comprises any one of SEQ ID NOs: 11-52 or 112-180. In some instances, the SARS-CoV-2 HR2 stabilized peptide consists of any one of SEQ ID NOs: 11-52 or 112-180. In some instances, the stapled and/or stitched SARS-CoV-2 peptides are derived from SEQ ID NOs:9, 10, 103, 104, 106, 108, and 110 are listed in Table 1.

TABLE 1
Stapled SARS-COV-2 HR2 Peptides.

| | Sequence (X, #, %, and/or 8 refer to α, α- disubstituted non- | |
|---|---|---|
| | natural amino acids with olefinic side chains whose side | |
| | chains are cross-linked to form an internally cross-linked | SEQ ID |
| Name | peptide) | NO |
| SARS- | ISGINASVVNIQKEIDRLNEVAKNLNESLIDLQELGKYEQY | 9 |
| CoV-2 | I | |
| HR2₍₁₁₆₉₋₁₂₁₀₎ | | |
| SARS- | IQKEIDRLNEVAKNLNESL | 10 |
| CoV-2 | | |
| HR2₍₁₁₇₉₋₁₁₉₇₎ | | |
| SARS- | IQKEIDRLNEVAKNLNESL* | 102 |
| CoV-2 | | |
| HR2_{(1179-1197)*} | | |
| SARS- | ISGINASVVNIQKEIDRLNEVAKNLNESL | 103 |
| CoV-2 | | |
| HR2₍₁₁₆₉₋₁₁₉₇₎ | | |
| SARS- | IQKEIDRLNEVAKNLNESLIDLQEL | 104 |
| CoV-2 | | |
| HR2₍₁₁₇₉₋₁₂₀₃₎ | | |
| SARS- | IQKEIDRLNEVAKNLNESLIDLQEL* | 105 |
| CoV-2 | | |
| HR2_{(1179-1203)*} | | |
| SARS- | DISGINASVVNIQKEIDRLNEVAKNLNESL | 106 |
| CoV-2 | | |
| HR2₍₁₁₆₈₋₁₁₉₇₎ | | |
| SARS- | DISGINASVVNIQKEIDRLNEVAKNLNESL* | 107 |
| CoV-2 | | |
| HR2_{(1168-1197)*} | | |
| SARS- | DISGINASVVNIQKEIDRLNEVAKNLNESLIDLQEL | 108 |
| CoV-2 | | |
| HR2₍₁₁₆₈₋₁₂₀₃₎ | | |
| SARS- | DISGINASVVNIQKEIDRLNEVAKNLNESLIDLQEL* | 109 |
| CoV-2 | | |
| HR2_{(1168-1203)*} | | |
| EK1 | LEYEBKKLEEAIKKLEESY | 258 |
| Core | | |
| EK1 | SLDQINVTFLDLEYEMKKLEEAIKKLEESYIDLKEL | 110 |
| EK1* | SLDQINVTFLDLEYEMKKLEEAIKKLEESYIDLKEL* | 111 |
| SAH- | ISGI8ASVVNIXKEIDRLNEVAKNLNESLIDLQELGKYEQY | 11 |
| SARS- | I | |
| CoV-2- | | |
| A | | |
| SAH- | ISGIN8SVVNIQXEIDRLNEVAKNLNESLIDLQELGKYEQY | 12 |
| SARS- | I | |
| CoV-2 | | |
| B | | |
| SAH- | ISGINA8VVNIQKXIDRLNEVAKNLNESLIDLQELGKYEQ | 13 |
| SARS- | YI | |
| CoV-2 | | |
| C | | |
| SAH- | ISGINASVVNIQKEIDRLNEVAKNL8ESLIDLXELGKYEQY | 14 |
| SARS- | I | |
| CoV-2 | | |
| D | | |
| SAH- | ISGINASVVNIQKEIDRLNEVAKNLN8SLIDLQXLGKYEQ | 15 |
| SARS- | YI | |
| CoV-2 | | |
| E | | |
| SAH- | ISGINASVVNIQKEIDRLNEVAKNLNESLIDL8ELGKYEX | 16 |
| SARS- | YI | |
| CoV-2 | | |
| F | | |
| SAH- | ISGINASVVNIQKEIDRLNEVAKNL8ESLIDL#ELGKYE% | 17 |
| SARS- | YI | |
| CoV-2 | | |
| G | | |
| SAH- | ISGI8₁ASVVNIX₁KEIDRLNEVAKNLN8₂ESLIDLX₂ELGKYE | 18 |
| SARS- | QYI | |
| CoV-2 | | |
| A, D | | |
| SAH- | ISGI8₁ASVVNIX₁KEIDRLNEVAKNLN8₂SLIDLQ | 19 |
| SARS- | X₂LGKYEQYI | |
| CoV-2 | | |
| A, E | | |
| SAH- | ISGI8₁ASVVNIX₁KEIDRLNEVAKNLNESLIDL8₂ELGKYE | 20 |
| SARS- | X₂YI | |
| CoV-2 | | |
| A, F | | |
| SAH- | ISGI8₁ASVVNI | 21 |
| SARS- | XKEIDRLNEVAKNL8₂ESLIDL#ELGKYE%YI | |
| CoV-2 | | |
| A, G | | |
| SAH- | ISGIN8₁SVVNIQX₁EIDRLNEVAKNL8₂ESLIDL | 22 |
| SARS- | X₂ELGKYEQYI | |
| CoV-2 | | |
| B, D | | |
| SAH- | ISGIN8₁SVVNIQX₁EIDRLNEVAKNLN8₂SLIDLQ | 23 |
| SARS- | X₂LGKYEQYI | |
| CoV-2 | | |
| B, E | | |
| SAH- | ISGIN8₁SVVNIQX₁EIDRLNEVAKNLNESLIDL8ELGKYE | 24 |
| SARS- | X₂YI | |
| CoV-2 | | |
| B ,F | | |
| SAH- | ISGIN8₁SVVNIQ | 25 |
| SARS- | XEIDRLNEVAKNL8₂ESLIDL#ELGKYE%YI | |
| CoV-2 | | |
| B, G | | |
| SAH- | ISGINA8₁VVNIQKX₁IDRLNEVAKNL8₂ESLIDL | 26 |
| SARS- | X₂ELGKYEQYI | |
| CoV-2 | | |
| C, D | | |
| SAH- | ISGINA8₁VVNIQKX₁IDRLNEVAKNLN8₂SLIDLQ | 27 |
| SARS- | X₂LGKYEQYI | |
| CoV-2 | | |
| C, E | | |
| SAH- | ISGINA8₁VVNIQK | 28 |
| SARS- | X₁IDRLNEVAKNLNESLIDL8₂ELGKYEX₂YI | |
| CoV-2 | | |
| C, F | | |
| SAH- | ISGINA8₁VVNIQK | 29 |
| SARS- | XIDRLNEVAKNL8₂ESLIDL#ELGKYE%YI | |
| CoV-2 | | |
| C, G | | |
| SAH- | 8QKEIDRXNEVAKNLNESL | 112 |
| SARS- | | |
| CoV-2 | | |
| U | | |
| SAH- | I8KEIDRLXEVAKNLNESL | 30 |
| SARS- | | |
| CoV-2 | | |
| H | | |
| SAH- | IQ8EIDRLNXVAKNLNESL | 31 |
| SARS- | | |
| CoV-2 | | |
| I | | |
| SAH- | IQK8IDRLNEXAKNLNESL | 113 |
| SARS- | | |
| CoV-2- | | |
| V | | |
| SAH- | IQKE8DRLNEVXKNLNESL | 114 |
| SARS- | | |
| CoV-2- | | |
| W | | |
| SAH- | IQKEI8RLNEVAXNLNESL | 32 |
| SARS- | | |
| CoV-2- | | |
| J | | |
| SAH- | IQKEID8LNEVAKXLNESL | 33 |
| SARS- | | |
| CoV-2 | | |
| K | | |
| SAH- | IQKEIDR8NEVAKNXNESL | 115 |
| SARS- | | |
| CoV-2 | | |
| X | | |
| SAH- | IQKEIDRL8EVAKNLXESL | 34 |
| SARS- | | |
| CoV-2 | | |
| L | | |
| SAH- | IQKEIDRLN8VAKNLNXSL | 35 |
| SARS- | | |
| CoV-2 | | |
| M | | |
| SAH- | IQKEIDRLNE8AKNLNEXL | 116 |
| SARS- | | |
| CoV-2 | | |
| Y | | |
| SAH- | IQKEIDRLNEV8KNLNESX | 117 |
| SARS- | | |
| CoV-2 | | |
| Z | | |
| SAH- | ISGINASVVN8QKEIDRXNEVAKNLNESL | 118 |
| SARS- | | |
| CoV-2 | | |
| L-U | | |
| SAH- | ISGINASVVNI8KEIDRLXEVAKNLNESL | 119 |
| SARS- | | |
| CoV-2 | | |
| L-H | | |
| SAH- | ISGINASVVNIQ8EIDRLNXVAKNLNESL | 120 |
| SARS- | | |
| CoV-2 | | |
| L-I | | |
| SAH- | ISGINASVVNIQK8IDRLNEXAKNLNESL | 121 |
| SARS- | | |
| CoV-2 | | |
| L-V | | |
| SAH- | ISGINASVVNIQKE8DRLNEVXKNLNESL | 122 |
| SARS- | | |
| CoV-2 | | |
| L-W | | |
| SAH- | ISGINASVVNIQKEI8RLNEVAXNLNESL | 123 |
| SARS- | | |
| CoV-2 | | |
| L-J | | |
| SAH- | ISGINASVVNIQKEID8LNEVAKXLNESL | 124 |
| SARS- | | |
| CoV-2 | | |
| L-K | | |
| SAH- | ISGINASVVNIQKEIDR8NEVAKNXNESL | 125 |
| SARS- | | |
| CoV-2 | | |
| L-X | | |
| SAH- | ISGINASVVNIQKEIDRL8EVAKNLXESL | 126 |
| SARS- | | |
| CoV-2 | | |
| L-L | | |
| SAH- | ISGINASVVNIQKEIDRLN8VAKNLNXSL | 127 |
| SARS- | | |
| CoV-2 | | |
| L-M | | |
| SAH- | ISGINASVVNIQKEIDRLNE8AKNLNEXL | 128 |
| SARS- | | |
| CoV-2 | | |
| L-Y | | |
| SAH- | ISGINASVVNIQKEIDRLNEV8KNLNESX | 129 |
| SARS- | | |
| CoV-2 | | |
| L-Z | | |
| SAH- | X₁QKEX₂DRLNEVAKNLNESL | 130 |
| SARS- | | |
| CoV-2- | | |
| a | | |
| SAH- | IX₁KEIX₂RLNEVAKNLNESL | 36 |
| SARS- | | |
| CoV-2- | | |
| N | | |
| SAH- | IQX₁EIDX₂LNEVAKNLNESL | 37 |
| SARS- | | |
| CoV-2- | | |
| O | | |
| SAH- | IQKX₁IDRX₂NEVAKNLNESL | 131 |
| SARS- | | |
| CoV-2- | | |
| b | | |
| SAH- | IQKEX₁DRLX₂EVAKNLNESL | 132 |
| SARS- | | |
| CoV-2- | | |
| c | | |
| SAH- | IQKEIX₁RLNX₂VAKNLNESL | 38 |
| SARS- | | |
| CoV-2- | | |
| P | | |
| SAH- | IQKEIDX₁LNEX₂AKNLNESL | 133 |
| SARS- | | |
| CoV-2- | | |
| d | | |
| SAH- | IQKEIDRX₁NEVX₂KNLNESL | 134 |
| SARS- | | |
| CoV-2- | | |
| e | | |
| SAH- | IQKEIDRLX₁EVAX₂NLNESL | 39 |
| SARS- | | |
| CoV-2- | | |
| Q | | |
| SAH- | IQKEIDRLNX₁VAKX₂LNESL | 40 |
| SARS- | | |
| CoV-2- | | |
| R | | |
| SAH- | IQKEIDRLNEX₁AKNX₂NESL | 135 |
| SARS- | | |
| CoV-2-f | | |
| SAH- | IQKEIDRLNEVX₁KNLX₂ESL | 136 |
| SARS- | | |
| CoV-2- | | |
| g | | |
| SAH- | IQKEIDRLNEVAX₁NLNX₂SL | 41 |
| SARS- | | |
| CoV-2- | | |
| S | | |
| SAH- | IQKEIDRLNEVAKX₁LNEX₂L | 42 |
| SARS- | | |
| CoV-2- | | |
| T | | |
| SAH- | IQKEIDRLNEVAKNX₁NESX₂ | 137 |
| SARS- | | |
| CoV-2- | | |
| h | | |
| SAH- | ISGINASVVNX₁QKEX₂DRLNEVAKNLNESL | 138 |
| SARS- | | |
| CoV-2 | | |
| L-a | | |
| SAH- | ISGINASVVNIX₁KEIX₂RLNEVAKNLNESL | 139 |
| SARS- | | |
| CoV-2 | | |
| L-N | | |
| SAH- | ISGINASVVNIQX₁EIDX₂LNEVAKNLNESL | 140 |
| SARS- | | |
| CoV-2 | | |
| L-O | | |
| SAH- | ISGINASVVNIQKX₁IDRX₂NEVAKNLNESL | 141 |
| SARS- | | |
| CoV-2 | | |
| L-b | | |
| SAH- | ISGINASVVNIQKEX₁DRLX₂EVAKNLNESL | 142 |
| SARS- | | |
| CoV-2 | | |
| L-c | | |
| SAH- | ISGINASVVNIQKEIX₁RLNX₂VAKNLNESL | 143 |
| SARS- | | |
| CoV-2 | | |
| L-P | | |
| SAH- | ISGINASVVNIQKEIDX₁LNEX₂AKNLNESL | 144 |
| SARS- | | |
| CoV-2 | | |
| L-d | | |
| SAH- | ISGINASVVNIQKEIDRX₁NEVX₂KNLNESL | 145 |
| SARS- | | |
| CoV-2 | | |
| L-e | | |
| SAH- | ISGINASVVNIQKEIDRLX₁EVAX₂NLNESL | 146 |
| SARS- | | |
| CoV-2 | | |
| L-Q | | |
| SAH- | ISGINASVVNIQKEIDRLNX₁VAKX₂LNESL | 147 |
| SARS- | | |
| CoV-2 | | |
| L-R | | |
| SAH- | ISGINASVVNIQKEIDRLNEX₁AKNX₂NESL | 148 |
| SARS- | | |
| CoV-2 | | |
| L-f | | |
| SAH- | ISGINASVVNIQKEIDRLNEVX₁KNLX₂ESL | 149 |
| SARS- | | |
| CoV-2 | | |
| L-g | | |
| SAH- | ISGINASVVNIQKEIDRLNEVAX₁NLNX₂SL | 150 |
| SARS- | | |
| CoV-2 | | |
| L-S | | |
| SAH- | ISGINASVVNIQKEIDRLNEVAKX₁LNEX₂L | 151 |
| SARS- | | |
| CoV-2 | | |
| L-T | | |
| SAH- | ISGINASVVNIQKEIDRLNEVAKNX₁NESX₂ | 152 |
| SARS- | | |
| CoV-2 | | |
| L-h | | |
| SAH- | I8KEIDRL#EVAKNL%ESL | 43 |
| SARS- | | |
| CoV-2 | | |
| H-L | | |
| SAH- | ISGINASVVNI8KEIDRL#EVAKNL%ESLIDLQELGKYEQ | 153 |
| SARS- | YI | |
| CoV-2 | | |
| L-H-L | | |
| SAH- | IQ8EIDRLN#VAKNLN%SL | 44 |
| SARS- | | |
| CoV-2 | | |
| I-M | | |
| SAH- | I8KEIDRL#EVAXNLNESL | 45 |
| SARS- | | |
| CoV-2 | | |
| H-Q | | |
| SAH- | IQ8EIDRLN#VAKXLNESL | 46 |
| SARS- | | |
| CoV-2 | | |
| I-R | | |
| SAH- | ISGINASVVNIQ8EIDRLN#VAKXLNESLIDLQELGKYEQY | 154 |
| SARS- | I | |
| CoV-2 | | |
| L-I-R | | |
| SAH- | IQKEI8RLNEVA#NLNXSL | 47 |
| SARS- | | |
| CoV-2 | | |
| J-S | | |
| SAH- | IQKEID8LNEVAK#LNEXL | 48 |
| SARS- | | |
| CoV-2 | | |
| K-T | | |
| SAH- | IX₁KEIX₂RLNEVAX₃NLNX₄SL | 49 |
| SARS- | | |
| CoV-2 | | |
| N, S | | |
| SAH- | IX₁KEIX₂RLNEVAX₃NLNX₄SL* | 155 |
| SARS- | | |
| CoV-2 | | |
| N, S* | | |
| SAH- | ISGINASVVNIX₁KEIX₂RLNEVAX₃NLNX₄SLIDLQELGKY | 156 |
| SARS- | EQYI | |
| CoV-2 | | |
| L-N, S | | |
| SAH- | IQX₁EIDX₂LNEVAX₃NLNX₄SL | 50 |
| SARS- | | |
| CoV-2 | | |
| O, S | | |
| SAH- | IQX₁EIDX₂LNEVAX₃NLNX₄SL* | 157 |
| SARS- | | |
| CoV-2 | | |
| O, S* | | |
| SAH- | ISGINASVVNIQX₁EIDX₂LNEVAX₃NLNX₄SLIDLQELGKY | 158 |
| SARS- | EQYI | |
| CoV-2 | | |
| L-O, S | | |
| SAH- | IX₁KEIX₂RLNEVAKX₃LNEX₄L | 51 |
| SARS- | | |
| CoV-2 | | |
| N, T | | |
| SAH- | IX₁KEIX₂RLNEVAKX₃LNEX₄L* | 159 |
| SARS- | | |
| CoV-2 | | |
| N, T* | | |
| SAH- | ISGINASVVNIX₁KEIX₂RLNEVAKX₃LNEX₄LIDLQELGKY | 160 |
| SARS- | EQYI | |
| CoV-2 | | |
| L-N, T | | |
| SAH- | IQX₁EIDX₂LNEVAKX₃LNEX₄L | 52 |
| SARS- | | |
| CoV-2 | | |
| O, T | | |
| SAH- | IQX₁EIDX₂LNEVAKX₃LNEX₄L* | 161 |
| SARS- | | |
| CoV-2 | | |
| O, T* | | |
| SAH- | ISGINASVVNIQX₁EIDX₂LNEVAKX₃LNEX₄LIDLQELGKY | 162 |
| SARS- | EQYI | |
| CoV-2 | | |
| L-O,T | | |
| SAH- | IX₁KEIX₂RLNEVAX₃NLNX₄SLIDLQEL* | 163 |
| SARS- | | |
| CoV-2 | | |
| L1- | | |
| N, S* | | |
| SAH- | IQX₁EIDX₂LNEVAX₃NLNX₄SLIDLQEL* | 164 |
| SARS- | | |
| CoV-2 | | |
| L1- | | |
| O, S* | | |
| SAH- | IX₁KEIX₂RLNEVAKX₃LNEX₄LIDLQEL* | 165 |
| SARS- | | |
| CoV-2 | | |
| L1- | | |
| N, T* | | |
| SAH- | IQX₁EIDX₂LNEVAKX₃LNEX₄LIDLQEL* | 166 |
| SARS- | | |
| CoV-2 | | |
| L1- | | |
| O, T* | | |
| SAH- | DISGINASVVNIX₁KEIX₂RLNEVAX₃NLNX₄SL* | 167 |
| SARS- | | |
| CoV-2 | | |
| L2- | | |
| N, S* | | |
| SAH- | DISGINASVVNIQX₁EIDX₂LNEVAX₃NLNX₄SL* | 168 |
| SARS- | | |
| CoV-2 | | |
| L2- | | |
| O, S* | | |
| SAH- | DISGINASVVNIX₁KEIX₂RLNEVAKX₃LNEX₄L* | 169 |
| SARS- | | |
| CoV-2 | | |
| L2- | | |
| N,T* | | |
| SAH- | DISGINASVVNIQX₁EIDX₂LNEVAKX₃LNEX₄L* | 170 |
| SARS- | | |
| CoV-2 | | |
| L2- | | |
| O,T* | | |
| SAH- | DISGINASVVNIX₁KEIX₂RLNEVAX₃NLNX₄SLIDLQEL* | 171 |
| SARS- | | |
| CoV-2 | | |
| L3- | | |
| N,S* | | |
| SAH- | DISGINASVVNIQX₁EIDX₂LNEVAX₃NLNX₄SLIDLQEL* | 172 |
| SARS- | | |
| CoV-2 | | |
| L3- | | |
| O,S* | | |
| SAH- | DISGINASVVNIX₁KEIX₂RLNEVAKX₃LNEX₄LIDLQEL* | 173 |
| SARS- | | |
| CoV-2 | | |
| L3- | | |
| N,T* | | |
| SAH- | DISGINASVVNIQX₁EIDX₂LNEVAKX₃LNEX₄LIDLQEL* | 174 |
| SARS- | | |
| CoV-2 | | |
| L3- | | |
| O,T* | | |
| SAH- | SLDQINVTFLDL8YEBKKL#EAIKKL%ESYIDLKE | 175 |
| EK1- | | |
| H, L | | |
| SAH- | SLDQINVTFLDLE8EBKKLE#AIKKLE%SYIDLKE | 176 |
| EK1- | | |
| I, M | | |
| SAH- | SLDQINVTFLDLX₁YEBX₂KLEEAIX₃KLEX₄SYIDLKE | 177 |
| EK1- | | |
| N, S | | |
| SAH- | SLDQINVTFLDLEX₁EBKX₂LEEAIX₃KLEX₄SYIDLKE | 178 |
| EK1- | | |
| O, S | | |
| SAH- | SLDQINVTFLDLX₁YEBX₂KLEEAIKX₃LEEX₄YIDLKE | 179 |
| EK1- | | |
| N, T | | |
| SAH- | SLDQINVTFLDLEX₁EBKX₂LEEAIKX₃LEEX₄YIDLKE | 180 |
| EK1- | | |
| O, T | | |

In Table 1, “8” is 8, 8₁, and 8₂=(R)-α-(7′-octenyl)alanine or (R)-α-(4′-pentenyl)alanine; X, X₁, X₂, X₃, and X₄=(S)-α-(4′-pentenyl)alanine; #=α,α-Bis(4′-pentenyl)glycine or α,α-Bis(7′-octenyl)glycine; %=(S)-α-(7′-octenyl)alanine or (S)-α-(4′-pentenyl)alanine; B=norleucine; and *=GSGSGC(SEQ ID NO:256)-(PEG4-chol)-carboxamide. It should be understood that the above peptides can be modified to include additional amino acids (e.g., 1, 2, 3, 4, 5, 6, 7, 8, 9, 10 amino acids added) at the N and/or C-terminus, and/or to have N and/or C terminal deletions (e.g., 1, 2, 3, 4, 5, 6, 7, 8, 9, 10 amino acids deleted).

Note that the bolded and underlined sequence used herein (e.g., in Table 1) identifies the stapling amino acids at the N- and C-termini and the intervening sequence between staples for each disclosed peptide. In some instances (e.g., SEQ ID NOs: 11-16, 30-42, and 112-152), the structurally-stabilized peptide is single-stapled peptide. In some instances (e.g., SEQ ID NOs: 18-20, 22-24, 26-28, 49-52, 155-174, and 177-180) the structurally-stabilized peptide is a double-stapled peptide. In some instances (e.g., SEQ ID NOs: 17, 43-48, 153, 154, 175, and 176), the structurally-stabilized peptide is a stitched peptide. In some instances (e.g., SEQ ID NOs: 21, 25, and 29), the structurally-stabilized peptide is both stapled and stitched.

The disclosure encompasses each and every peptide and structurally stabilized peptide listed in Table 1 as well as variants thereof. In some instances, the structurally stabilized peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length. In some instances, the structurally stabilized peptide described above have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9 or 10); (iii) is alpha-helical; (iv) is protease resistant; (v) inhibits fusion of SARS-CoV-2 with a host cell; and/or (vi) inhibits infection of a cell by SARS-CoV-2.

In some instances, disclosed herein are peptides that comprise 0-10 (0, 1, 2, 3, 4, 5, 6, 7, 8, 9, 10) amino acid substitutions compared to one of the single-stapled peptides (e.g., SEQ ID NOs: 11-16, 30-42, and 112-152) in Table 1. In some instances, disclosed herein are peptides that are at least 75% (e.g., at least 75%, at least 80%, at least 85%, at least 90%, at least 95% identical) to one of the single-stapled peptides (e.g., SEQ ID NOs: 11-16, 30-42, and 112-152) in Table 1. In some instances, the structurally stabilized peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length. In some instances, the structurally stabilized peptide described above have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9 or 10); (iii) is alpha-helical; (iv) is protease resistant; (v) inhibits fusion of SARS-CoV-2 with a host cell; and/or (vi) inhibits infection of a cell by SARS-CoV-2.

In some instances, disclosed herein are peptides that comprise 0-10 (0, 1, 2, 3, 4, 5, 6, 7, 8, 9, 10) amino acid substitutions compared to one of the double-stapled peptides (e.g., SEQ ID NOs: 18-20, 22-24, 26-28, 49-52, 155-174, and 177-180) in Table 1. In some instances, disclosed herein are peptides that are at least 75% (e.g., at least 75%, at least 80%, at least 85%, at least 90%, at least 95% identical) to one of the double-stapled peptides (e.g., SEQ ID NOs: 18-20, 22-24, 26-28, 49-52, 155-174, and 177-180) in Table 1. In some instances, the structurally stabilized peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length. In some instances, the structurally stabilized peptide described above have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9 or 10); (iii) is alpha-helical; (iv) is protease resistant; (v) inhibits fusion of SARS-CoV-2 with a host cell; and/or (vi) inhibits infection of a cell by SARS-CoV-2.

In some instances, disclosed herein are peptides that comprise 0-10 (0, 1, 2, 3, 4, 5, 6, 7, 8, 9, 10) amino acid substitutions compared to one of the stitched peptides (e.g., SEQ ID NOs: 17, 43-48, 153, 154, 175, and 176) in Table 1. In some instances, disclosed herein are peptides that are at least 75% (e.g., at least 75%, at least 80%, at least 85%, at least 90%, at least 95% identical) to one of the stitched peptides (e.g., SEQ ID NOs: 17, 43-48, 153, 154, 175, and 176) in Table 1. In some instances, the structurally stabilized peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length. In some instances, the structurally stabilized peptide described above have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9 or 10); (iii) is alpha-helical; (iv) is protease resistant; (v) inhibits fusion of SARS-CoV-2 with a host cell; and/or (vi) inhibits infection of a cell by SARS-CoV-2.

In some instances, disclosed herein are peptides that comprise 0-10 (0, 1, 2, 3, 4, 5, 6, 7, 8, 9, 10) amino acid substitutions compared to one of the peptides (e.g., SEQ ID NOs: 21, 25, and 29) in Table 1 that is both stapled and stitched. In some instances, disclosed herein are peptides that are at least 75% (e.g., at least 75%, at least 80%, at least 85%, at least 90%, at least 95% identical) to one of the peptides (e.g., SEQ ID NOs: 21, 25, and 29) in Table 1 that is both stapled and stitched. In some instances, these structurally stabilized peptides have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9 or 10); (iii) is alpha-helical; (iv) is protease resistant; (v) inhibits fusion of SARS-CoV-2 with a host cell; and/or (vi) inhibits infection of a cell by SARS-CoV-2.

In some instances, the structurally stabilized peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length. In some instances, the structurally stabilized peptide described above have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2.

In some instances, the stapled or stitched peptide is a peptide comprising or consisting of any one of the amino acids sequences of SEQ ID NOs: 9, 10, 103, 104, 106, 108, and 110, except that at least two (e.g., 2, 3, 4, 5, 6) amino acids of SEQ ID NOs: 9, 10, 103, 104, 106, 108, and 110 are replaced with a non-natural amino acid capable of forming a staple or stitch. In some instances, the non-natural amino acid is an α, α-disubstituted non-natural amino acids with olefinic side chains. In some instances, the stapled or stitched peptide is a peptide comprising or consisting of any one of the amino acids sequences of SEQ ID NOs: 10, 103, 104, 106, 108, and 110, except that at least two (e.g., 2, 3, 4, 5, 6) amino acids of SEQ ID NOs: 10, 103, 104, 106, 108, and 110 are replaced with a non-natural amino acid capable of forming a staple or stitch. In some instances, the non-natural amino acid is an α, α-disubstituted non-natural amino acids with olefinic side chains. In some instances, the structurally stabilized peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length. In some instances, the structurally stabilized peptide described above have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9 or 10); (iii) is alpha-helical; (iv) is protease resistant; (v) inhibits fusion of SARS-CoV-2 with a host cell; and/or (vi) inhibits infection of a cell by SARS-CoV-2.

In some instances, disclosed herein are peptides that comprise 0-10 (0, 1, 2, 3, 4, 5, 6, 7, 8, 9, 10) amino acid substitutions compared to one of the unmodified peptides (e.g., SEQ ID NOs: 9, 10, 103, 104, 106, 108, and 110) in Table 1. In some instances, disclosed herein are peptides that are at least 75% (e.g., at least 75%, at least 80%, at least 85%, at least 90%, at least 95% identical) to one of the unmodified peptides (e.g., SEQ ID NOs: 9, 10, 103, 104, 106, 108, and 110) in Table 1. In some instances, the substitution as described herein is a conservative substitution. In some instances, the structurally stabilized peptide is 19 to 100 (e.g., 19, 20, 21, 22, 23, 24, 25, 26, 27, 28, 29, 30, 31, 32, 33, 34, 345, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100) amino acids in length. In some instances, the structurally stabilized peptide described above have one or more (1, 2, 3, 4, 5, 6) of the properties listed below: (i) binds the recombinant 5-helix bundle protein; (ii) inhibits the interactions between the 5 helix bundle and SARS-CoV-2 HR2 peptide (SEQ ID NO:9 or 10); (iii) is alpha-helical; (iv) is protease resistant; (v) inhibits fusion of SARS-CoV-2 with a host cell; and/or (vi) inhibits infection of a cell by SARS-CoV-2.

In some instances, any substitution as described herein can be a conservative substitution. In some instances, any substitution as described herein is a non-conservative substitution.

In some instances, in any of the peptides comprising IQKEIDRLNEVAKNLNESL (SEQ ID NO:10) (i.e., in any of the peptides disclosed herein comprising IQKEIDRLNEVAKNLNESL (SEQ ID NO:10); e.g., peptides listed in Table 1), amino acid hydrophobic amino acid substitutions can be made at the following positions (shown in bold and underline):

(SEQ ID NO: 10)
1179 IQKEIDRLNEVAKNLNESL 1197.

So for example, I1179, I1183, L1186, A1190, L1193, and L1197 can be substituted with any of valine, isoleucine, leucine, phenylalanine, tryptophan, or cysteine. In some cases, these positions may be substituted with alanine or histidine.

In some instances, in any of the peptides comprising IQKEIDRLNEVAKNLNESL (SEQ ID NO:10) (i.e., in any of the peptides disclosed herein comprising IQKEIDRLNEVAKNLNESL (SEQ ID NO: 10); e.g., peptides listed in Table 1), amino acid substitutions can be made at the following positions (shown in bold and underline):

(SEQ ID NO: 10)
1179 IQKEIDRLNEVAKNLNESL 1197.

In some instances any of these bold and underlined positions (Q1180, E1182, R1185, N1187, V1189, N1192, N1194, or S1196) can be substituted with an α, α disubstituted non-natural amino acid with olefinic side chains. In some instances, the substation at these positions is a substitution to a nonpolar amino acid (e.g., G, A, P, V, L, I M, W, F, or C). In some instances, the substitution at these positions is to an alanine. In some instances, the substitution at these positions is a substitution that improves peptide binding (i.e., to the 5 helix bundle of SARS-CoV-2).

In some instances, in any of the peptides comprising IQKEIDRLNEVAKNLNESL (SEQ ID NO:10) (i.e., in any of the peptides disclosed herein comprising IQKEIDRLNEVAKNLNESL (SEQ ID NO:10); e.g., peptides listed in Table 1), substitutions are not made at one or more of the following positions (shown in bold and underlined):

(SEQ ID NO: 10)
1179 IQKEIDRLNEVAKNLNESL 1197.

In particular, these bold and underlined positions (i.e., K1181, D1184, E1188, K1191, E1195) are not substituted with a stapling amino acid (e.g., an α, α disubstituted non-natural amino acid with olefinic side chains).

In some instances, substitutions are made at one or more of the following positions: IQKEIDRLNEVAKNLNESL (SEQ ID NO:10). In these instances, the substitution is a substitution to a charged or polar amino acid (e.g., R, K, H, D, E, Q, Y, S, T, or N).

In some instances, with respect to SEQ ID NO:9, at the following positions (shown in bold and underlined; 11169, 11172, A1174, S1175, V1177, 11198, L1200, L1203)—which make direct contact with HR1—substitutions are not made, or if made, one or more of these positions can be substituted with conserved amino acid substitutions (e.g., for I, A, V, or L, a conservative substitution is one of G, A, V, L, I; and for S a conservative substitution is T, M, or C) for:

(SEQ ID NO: 9)
1169 ISGINASVVNIQKEIDRLNEVAKNLNESLIDLQELGKYEQYI
1210.

In some instances, if D1168 is also present as D1168 in the sequence, then it too should either not be substituted or only substituted with a conserved amino acid substitution (e.g., to E) In some instances, with respect to SEQ ID NO:9, at the following positions (S1170, G1171,N1173, V1176, N1178, D1199, Q1201, or E1202)—which are solvent exposed-one or more of these positions can be substituted with any amino acid substitutions (shown in bold and underline):

(SEQ ID NO: 9)
1169 ISGINASVVNIQKEIDRLNEVAKNLNESLIDLQELGKYEQYI
1210.

In some instances, the non-natural amino acids that may be used as stapling amino acids or stitching amino acids are: (R)-2-(2′-propenyl)alanine; (R)-2-(4′-pentenyl)alanine; (R)-α-(7′-octenyl)alanine; (S)-α-(2′-propenyl)alanine; (S)-α-(4′-pentenyl)alanine; (S)-2-(7′-octenyl)alanine; α,α-Bis(4′-pentenyl)glycine; and α,α-Bis(7′-octeny)gly cine.

In some embodiments, an internal staple replaces the side chains of 2 amino acids, i.e., each staple is between two amino acids separated by, for example, 2, 3, or 6 amino acids. In some embodiments, an internal stitch replaces the side chains of 3 amino acids, i.e., the stitch is a pair of crosslinks between three amino acids separated by, for example, 2, 3, or 6 amino acids. In some embodiments, the amino acids forming the staple or stitch are at each of positions i and i+3 of the staple. In some embodiments, the amino acids forming the staple or stitch are at each of positions i and i+4 of the staple. In some embodiments, the amino acids forming the staple or stitch are at each of positions i and i+7 of the staple. For example, where a peptide has the sequence . . . X1, X2, X3, X4, X5, X6, X7, X8, X9 . . . , cross-links between X1 and X4 (i and i+3), or between X1 and X5 (i and i+4), or between X1 and X8 (i and i+7) are useful hydrocarbon stapled forms of that peptide. The use of multiple cross-links (e.g., 2, 3, 4, or more) is also contemplated. Additional description regarding making and use of hydrocarbon-stapled peptides can be found, e.g., in U.S. Patent Publication Nos. 2012/0172285, 2010/0286057, and 2005/0250680, the contents of all of which are incorporated by reference herein in their entireties.

“Peptide stapling” is a term coined from a synthetic methodology wherein two olefin-containing side-chains (e.g., cross-linkable side chains) present in a peptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). The structural-stabilization may be by, e.g., stapling the peptide (see, e.g., Walensky, J. Med. Chem., 57:6275-6288 (2014), the contents of which are incorporated by reference herein in its entirety). In some cases, the staple is a hydrocarbon staple.

In some instances, the structural-stabilization is a stitch. The term “peptide stitching,” as used herein, refers to multiple and tandem stapling events in a single peptide chain to provide a “stitched” (e.g., tandem or multiply stapled) peptide, in which two staples, for example, are linked to a common residue. Peptide stitching is disclosed, e.g., in WO 2008/121767 and WO 2010/068684, which are both hereby incorporated by reference in their entirety.

In some instances, a staple or stitch used herein is a lactam staple or stitch; a UV-cycloaddition staple or stitch; an oxime staple or stitch; a thioether staple or stitch; a double-click staple or stitch; a bis-lactam staple or stitch; a bis-arylation staple or stitch; or a combination of any two or more thereof. Stabilized peptides as described herein include stapled peptides and stitched peptides as well as peptides containing multiple stitches, multiple staples or a mix of staples and stitches, or any other chemical strategies for structural reinforcement (see. e.g., Balaram P. Cur. Opin. Struct. Biol. 1992; 2:845; Kemp D S, et al., J. Am. Chem. Soc. 1996; 118:4240; Orner B P, et al., J. Am. Chem. Soc. 2001; 123:5382; Chin J W, et al., Int. Ed. 2001; 40:3806; Chapman R N, et al., J. Am. Chem. Soc. 2004; 126:12252; Home W S, et al., Chem., Int. Ed. 2008; 47:2853; Madden et al., Chem Commun (Camb). 2009 Oct. 7; (37): 5588-5590; Lau et al., Chem. Soc. Rev., 2015,44:91-102; and Gunnoo et al., Org. Biomol. Chem., 2016,14:8002-8013; each of which is incorporated by reference herein in its entirety).

A peptide is “structurally-stabilized” in that it maintains its native secondary structure. For example, stapling allows a peptide, predisposed to have an α-helical secondary structure, to maintain its native α-helical conformation. This secondary structure increases resistance of the peptide to proteolytic cleavage and heat, and may increase target binding affinity, hydrophobicity, and cell permeability. Accordingly, the stapled (cross-linked) peptides described herein have improved biological activity and pharmacology relative to a corresponding non-stapled (un-cross-linked) peptide.

In certain instances, the modification(s) to introduce structural stabilization (e.g., internal cross-linking, e.g., stapling, stitching) into the SARS-CoV-2 HR2 peptides described herein may be positioned on the face of the SARS-CoV-2 HR2 helix that does not interact with the recombinant 5-helix bundle of SARS-CoV-2. Alternatively, the modification(s) to introduce stabilization (e.g., internal cross-linking, e.g., stapling or stitching) into the SARS-CoV-2 HR2 peptides described herein may be positioned on the face of the SARS-CoV-2 HR2 helix that does interact with the 5 helix bundle of SARS-CoV-2. In some cases, a SARS-CoV-2 HR2 peptide described herein is stabilized by introducing a staple or stitch (e.g., a hydrocarbon staple or stitch) at the interface of the interacting and non-interacting helical faces of the SARS-CoV-2 HR2 protein.

In some instances, the modifications to introduce structural stabilization (e.g., internal cross-linking, e.g., stapling or stitching) into the SARS-CoV-2 HR2 peptides described herein are positioned at the amino acid positions in the SARS-CoV-2 HR2 peptide corresponding to residues:
(i) 5 and 12 of SEQ ID NO: 9;
(ii) 6 and 13 of SEQ ID NO: 9;
(iii) 7 and 14 of SEQ ID NO: 9;
(iv) 26 and 33 of SEQ ID NO: 9;
(v) 27 and 34 of SEQ ID NO: 9;
(vi) 33 and 40 of SEQ ID NO: 9;
(vii) 26, 33, and 40 of SEQ ID NO: 9;
(viii) 5, 12, 26, and 33 of SEQ ID NO: 9;
(ix) 5, 12, 27, and 34 of SEQ ID NO: 9;
(x) 5, 12, 33, and 40 of SEQ ID NO: 9;
(xi) 5, 12, 26, 33, and 40 of SEQ ID NO: 9;
(xii) 6, 13, 26, and 33 of SEQ ID NO: 9;
(xiii) 6, 13, 27, and 34 of SEQ ID NO: 9;
(xiv) 6, 13, 33, and 40 of SEQ ID NO: 9;
(xv) 6, 13, 26, 33, and 40 of SEQ ID NO: 9;
(xvi) 7, 14, 26, and 33 of SEQ ID NO: 9;
(xvii) 7, 14, 27, and 34 of SEQ ID NO: 9;
(xviii) 7, 14, 33, and 40 of SEQ ID NO: 9; or
(xix) 7, 14, 26, 33, and 40 of SEQ ID NO: 9;

In some instances, the modifications to introduce structural stabilization (e.g., internal cross-linking, e.g., stapling or stitching) into the SARS-CoV-2 HR2 peptides described herein are positioned at the amino acid positions in the SARS-CoV-2 HR2 peptide corresponding to residues:
(i) 1 and 8 of SEQ ID NO: 10;
(ii) 2 and 9 of SEQ ID NO: 10;
(iii) 3 and 10 of SEQ ID NO: 10;
(iv) 4 and 11 of SEQ ID NO: 10;
(v) 5 and 12 of SEQ ID NO: 10;
(vi) 6 and 13 of SEQ ID NO: 10;
(vii) 7 and 14 of SEQ ID NO: 10;
(viii) 8 and 15 of SEQ ID NO: 10;
(iv) 9 and 16 of SEQ ID NO: 10;
(x) 10 and 17 of SEQ ID NO: 10;
(xi) 11 and 18 of SEQ ID NO: 10;
(xi) 12 and 19 of SEQ ID NO: 10;
(xii) 1 and 5 of SEQ ID NO: 10;
(xiv) 2 and 6 of SEQ ID NO: 10;
(xv) 3 and 7 of SEQ ID NO: 10;
(xvi) 4 and 8 of SEQ ID NO: 10;
(xvii) 5 and 9 of SEQ ID NO: 10;
(xviii) 6 and 10 of SEQ ID NO: 10;
(xiv) 7 and 11 of SEQ ID NO: 10;
(xx) 8 and 12 of SEQ ID NO: 10;
(xxi) 9 and 13 of SEQ ID NO: 10;
(xxii) 10 and 14 of SEQ ID NO: 10;
(xxiii) 11 and 15 of SEQ ID NO: 10;
(xxiv) 12 and 16 of SEQ ID NO: 10;
(xxv) 13 and 17 of SEQ ID NO: 10;
(xxvi) 14 and 18 of SEQ ID NO: 10;
(xxvii) 15 and 19 of SEQ ID NO: 10;
(xxviii) 2, 9, and 16 of SEQ ID NO: 10;
(xxiv) 3, 10, and 17 of SEQ ID NO: 10;
(xxx) 2, 9, and 13 of SEQ ID NO: 10;
(xxxi) 3, 10, and 14 of SEQ ID NO: 10;
(xxxxii) 6, 13, and 17 of SEQ ID NO: 10;
(xxxiv) 7, 14, and 18 of SEQ ID NO: 10;
(xxxv) 2, 6, 13, and 17 of SEQ ID NO: 10;
(xxxvi) 3, 7, 13, and 17 of SEQ ID NO: 10;
(xxxvii) 2, 6, 14, and 18 of SEQ ID NO: 10; or
(xxxviii) 3, 7, 14, and 18 of SEQ ID NO: 10.

In certain instances, the SARS-CoV-2 HR2 peptides described herein (e.g., SEQ ID NOs: 11-52, 112-180, or 258) may also contain one or more (e.g., 1, 2, 3, 4, or 5) amino acid substitutions (relative to an amino acid sequence set forth in any one of SEQ ID NOs: 11-52, 112-180, or 258), e.g., one or more (e.g., 1, 2, 3, 4, or 5) conservative and/or non-conservative amino acid substitutions. In some instances, the SARS-CoV-2 HR2 peptides described herein (e.g., SEQ ID NOs: 11-52, 112-180, or 258) may also contain at least one, at least 2, at least 3, at least 4, or at least 5 amino acids added to the N-terminus of the peptide. In some instances, the SARS-CoV-2 HR2 peptides described herein (e.g., SEQ ID NOs: 11-52, 112-180, or 258) may also contain at least one, at least 2, at least 3, at least 4, or at least 5 amino acids added to the C-terminus of the peptide.

In one aspect, the structurally-stabilized SARS-CoV-2 HR2 peptide comprises Formula (I),

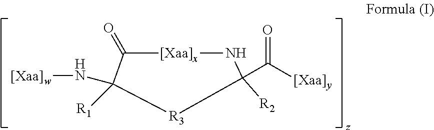

or a pharmaceutically acceptable salt thereof, wherein:
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄—K—R₄]ₙ; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

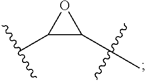 [SMILES: *C1OC1*]

R₆ is H, alkyl, or a therapeutic agent;
n is an integer from 1-4;
x is an integer from 2-10;
each y is independently an integer from 0-100;
z is an integer from 1-10 (e.g., 1, 2, 3, 4, 5, 6, 7, 8, 9, 10);
and each Xaa is independently an amino acid; and
wherein the structurally-stabilized peptide wherein the peptide binds the recombinant 5-helix bundle COVID-19 S protein.

In some embodiments, each of the [Xaa]w of Formula (I), the [Xaa]ₓ of Formula (I), and the [Xaa]_{y} of Formula (I) is as described for any one of constructs 1-60 of Table 2. For example, for a stabilized peptide comprising the [Xaa]_{w}, the [Xaa]ₓ, and the [Xaa]_{y} of construct 1 of Table 2, the [Xaa]_{w}, the [Xaa]ₓ, and the [Xaa]_{y} is: ISGI (SEQ ID NO: 53), ASVVNI (SEQ ID NO: 54), and KEIDRLNEVAKNLNESLIDLQELGKYEQYI (SEQ ID NO: 55), respectively. As another example, for a stabilized peptide comprising the [Xaa]_{w}, the [Xaa]ₓ, and the [Xaa]_{y} of construct 2 of Table 2, the [Xaa]_{w}, the [Xaa]ₓ, and the [Xaa]_{y} is: ISGIN (SEQ ID NO: 56), SVVNIQ (SEQ ID NO: 57), and EIDRLNEVAKNLNESLIDLQELGKYEQYI (SEQ ID NO: 58), respectively.

TABLE 2
[Xaa]_{w}, [Xaa]ₓ, and [Xaa]_{y} sequences for Formula (I) constructs 1-
60.

| Construct | [Xaa]_{w} | [Xaa]ₓ | [Xaa]_{y} |
|---|---|---|---|
| 1 | ISGI (SEQ ID NO: 53) | ASVVNI (SEQ ID | KEIDRLNEVAKNLNE |
| | | NO: 54) | SLIDLQELGKYEQYI |
| | | | (SEQ ID NO: 55) |
| 2 | ISGIN (SEQ ID NO: 56) | SVVNIQ (SEQ ID | EIDRLNEVAKNLNES |
| | | NO: 57) | LIDLQELGKYEQYI |
| | | | (SEQ ID NO: 58) |
| 3 | ISGINA (SEQ ID | VVNIQK (SEQ ID | IDRLNEVAKNLNESL |
| | NO: 59) | NO: 60) | IDLQELGKYEQYI |
| | | | (SEQ ID NO: 61) |
| 4 | ISGINASVVNIQKEIDR | ESLIDL (SEQ ID | ELGKYEQYI (SEQ ID |
| | LNEVAKNL (SEQ ID | NO: 63) | NO: 64) |
| | NO: 62) | | |
| 5 | ISGINASVVNIQKEIDR | SLIDLQ (SEQ ID | LGKYEQYI (SEQ ID |
| | LNEVAKNLN (SEQ ID | NO: 66) | NO: 67) |
| | NO: 65) | | |
| 6 | ISGINASVVNIQKEIDR | ELGKYE (SEQ ID | YI |
| | LNEVAKNLNESLIDL | NO: 69) | |
| | (SEQ ID NO: 68) | | |
| 7 | I | KEIDRL (SEQ ID | EVAKNLNESL (SEQ |
| | | NO: 70) | ID NO: 71) |
| 8 | IQ | EIDRLN (SEQ ID | VAKNLNESL (SEQ ID |
| | | NO: 72) | NO: 73) |
| 9 | IQKEI (SEQ ID NO: 74) | RLNEVA (SEQ ID | NLNESL (SEQ ID |
| | | NO: 75) | NO: 76) |
| 10 | IQKEID (SEQ ID | LNEVAK (SEQ ID | LNESL (SEQ ID |
| | NO: 77) | NO: 78) | NO: 79) |
| 11 | IQKEIDRL (SEQ ID | EVAKNL (SEQ ID | ESL |
| | NO: 80) | NO: 81) | |
| 12 | IQKEIDRLN (SEQ ID | VAKNLN (SEQ ID | SL |
| | NO: 82) | NO: 83) | |
| 13 | I | KEI | RLNEVAKNLNESL |
| | | | (SEQ ID NO: 84) |
| 14 | IQ | EID | LNEVAKNLNESL |
| | | | (SEQ ID NO: 85) |
| 15 | IQKEI (SEQ ID NO: 74) | RLN | VAKNLNESL (SEQ ID |
| | | | NO: 73) |
| 16 | IQKEIDRL (SEQ ID | EVA | NLNESL (SEQ ID |
| | NO: 80) | | NO: 76) |
| 17 | IQKEIDRLN (SEQ ID | VAK | LNESL (SEQ ID |
| | NO: 82) | | NO: 79) |
| 18 | IQKEIDRLNEVA (SEQ | NLN | SL |
| | ID NO: 86) | | |
| 19 | IQKEIDRLNEVAK | LNE | L |
| | (SEQ ID NO: 87) | | |
| 20 | | QKEIDR (SEQ ID | NEVAKNLNESL (SEQ |
| | | NO: 228) | ID NO: 229) |
| 21 | IQK | IDRLNE (SEQ ID | AKNLNESL (SEQ ID |
| | | NO: 230) | NO: 231) |
| 22 | IQKE (SEQ ID NO: 232) | DRLNEV (SEQ ID | KNLNESL (SEQ ID |
| | | NO: 181) | NO: 182) |
| 23 | IQKEIDR (SEQ ID | NEVAKN (SEQ ID | NESL (SEQ ID |
| | NO: 183) | NO: 184) | NO: 185) |
| 24 | IQKEIDRLNE (SEQ ID | AKNLNE (SEQ ID | L |
| | NO: 186) | NO: 187) | |
| 25 | IQKEIDRLNEV (SEQ | KNLNES (SEQ ID | |
| | ID NO: 188) | NO: 189) | |
| 26 | | QKE | DRLNEVAKNLNESL |
| | | | (SEQ ID NO: 190) |
| 27 | IQK | IDR | NEVAKNLNESL (SEQ |
| | | | ID NO: 229) |
| 28 | IQKE (SEQ ID NO: 232) | DRL | EVAKNLNESL (SEQ |
| | | | ID NO: 71) |
| 29 | IQKEID (SEQ ID | LNE | AKNLNESL (SEQ ID |
| | NO: 77) | | NO: 231) |
| 30 | IQKEIDR (SEQ ID | NEV | KNLNESL (SEQ ID |
| | NO: 183) | | NO: 182) |
| 31 | IQKEIDRLNE (SEQ ID | AKN | NESL (SEQ ID |
| | NO: 186) | | NO: 185) |
| 32 | IQKEIDRLNEV (SEQ | KNL | ESL |
| | ID NO: 188) | | |
| 33 | IQKEIDRLNEVAKN | NES | |
| | (SEQ ID NO: 191) | | |
| 34 | ISGINASVVN (SEQ ID | QKEIDR (SEQ ID NO: | NEVAKNLNESL (SEQ |
| | NO: 250) | 228) | ID NO: 229) |
| 35 | ISGINASVVNI (SEQ ID | KEIDRL (SEQ ID | EVAKNLNESL (SEQ |
| | NO: 193) | NO: 70) | ID NO: 71) |
| 36 | ISGINASVVNIQ (SEQ | EIDRLN (SEQ ID | VAKNLNESL (SEQ ID |
| | ID NO: 194) | NO: 72) | NO: 73) |
| 37 | ISGINASVVNIQK | IDRLNE (SEQ ID | AKNLNESL (SEQ ID |
| | (SEQ ID NO: 195) | NO: 230) | NO: 231) |
| 38 | ISGINASVVNIQKE | DRLNEV (SEQ ID | KNLNESL (SEQ ID |
| | (SEQ ID NO: 196) | NO: 181) | NO: 182) |
| 39 | ISGINASVVNIQKEI | RLNEVA (SEQ ID | NLNESL (SEQ ID |
| | (SEQ ID NO: 197) | NO: 75) | NO: 76) |
| 40 | ISGINASVVNIQKEID | LNEVAK (SEQ ID | LNESL (SEQ ID |
| | (SEQ ID NO: 198) | NO: 78) | NO: 79) |
| 41 | ISGINASVVNIQKEIDR | NEVAKN (SEQ ID | NESL (SEQ ID |
| | (SEQ ID NO: 199) | NO: 184) | NO: 185) |
| 42 | ISGINASVVNIQKEIDR | EVAKNL (SEQ ID | ESL |
| | L (SEQ ID NO: 200) | NO: 81) | |
| 43 | ISGINASVVNIQKEIDR | VAKNLN (SEQ ID | SL |
| | LN (SEQ ID NO: 201) | NO: 83) | |
| 44 | ISGINASVVNIQKEIDR | AKNLNE (SEQ ID | L |
| | LNE (SEQ ID NO: 202) | NO: 187) | |
| 45 | ISGINASVVNIQKEIDR | KNLNES (SEQ ID | |
| | LNEV (SEQ ID NO: 203) | NO: 189) | |
| 46 | ISGINASVVN (SEQ ID | QKE | DRLNEVAKNLNESL |
| | NO: 250) | | (SEQ ID NO: 190) |
| 47 | ISGINASVVNI (SEQ ID | KEI | RLNEVAKNLNESL |
| | NO: 193) | | (SEQ ID NO: 84) |
| 48 | ISGINASVVNIQ (SEQ | EID | LNEVAKNLNESL |
| | ID NO: 194) | | (SEQ ID NO: 85) |
| 49 | ISGINASVVNIQK | IDR | NEVAKNLNESL (SEQ |
| | (SEQ ID NO: 195) | | ID NO: 229) |
| 50 | ISGINASVVNIQKE | DRL | EVAKNLNESL (SEQ |
| | (SEQ ID NO: 196) | | ID NO: 71) |
| 51 | ISGINASVVNIQKEI | RLN | VAKNLNESL (SEQ ID |
| | (SEQ ID NO: 197) | | NO: 73) |
| 52 | ISGINASVVNIQKEID | LNE | AKNLNESL (SEQ ID |
| | (SEQ ID NO: 198) | | NO: 231) |
| 53 | ISGINASVVNIQKEIDR | NEV | KNLNESL (SEQ ID |
| | (SEQ ID NO: 199) | | NO: 182) |
| 54 | ISGINASVVNIQKEIDR | EVA | NLNESL (SEQ ID |
| | L (SEQ ID NO: 200) | | NO: 76) |
| 55 | ISGINASVVNIQKEIDR | VAK | LNESL (SEQ ID |
| | LN (SEQ ID NO: 201) | | NO: 79) |
| 56 | ISGINASVVNIQKEIDR | AKN | NESL (SEQ ID |
| | LNE (SEQ ID NO: 202) | | NO: 185) |
| 57 | ISGINASVVNIQKEIDR | KNL | ESL |
| | LNEV (SEQ ID NO: 203) | | |
| 58 | ISGINASVVNIQKEIDR | NLN | SL |
| | LNEVA (SEQ ID | | |
| | NO: 204) | | |
| 59 | ISGINASVVNIQKEIDR | LNE | L |
| | LNEVAK (SEQ ID | | |
| | NO: 205) | | |
| 60 | ISGINASVVNIQKEIDR | NES | |
| | LNEVAKN (SEQ ID | | |
| | NO: 206) | | |

In certain instances, the sequences set forth above in Table 2 can have at least one (e.g., 1, 2, 3, 4, 5, or 6) amino acid substitution or deletion. The SARS-CoV-2 HR2 peptides can include any amino acid sequence described herein.

In some instances, Formula (I) comprising the sequences set forth above in Table 2 can have one or more of the properties listed below: (i) binds the recombinant SARS-CoV-2 5-helix bundle S protein; (ii) is alpha-helical; (iii) is protease resistant; (iv) inhibits fusion of SARS-CoV-2 with a host cell; and/or (v) inhibits infection of a cell by SARS-CoV-2.

The tether of Formula (I) can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (I), x is 2, 3, or 6. In some instances of Formula (I), each y is independently an integer between 0 and 15, or 3 and 15. In some instances of Formula (I), R₁ and R₂ are each independently H or C₁-C₆ alkyl. In some instances of Formula (I), R₁ and R₂ are each independently C₁-C₃ alkyl. In some instances or Formula (I), at least one of R₁ and R₂ are methyl. For example, R₁ and R₂ can both be methyl. In some instances of Formula (I), R₃ is alkyl (e.g., C₈ alkyl) and x is 3. In some instances of Formula (I), R₃ is C₁₁ alkyl and x is 6. In some instances of Formula (I), R₃ is alkenyl (e.g., C₈ alkenyl) and x is 3. In some instances of Formula (I), x is 6 and R₃ is C₁₁ alkenyl. In some instances, R₃ is a straight chain alkyl, alkenyl, or alkynyl. In some instances, R₃ is —CH₂—CH₂—CH₂—CH═CH—CH₂—CH₂—CH₂—.

In one aspect, a structurally-stabilized COVID-19 HR2 peptide comprises Formula (I), or a pharmaceutically acceptable salt thereof, wherein:
each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
each R₃ is independently alkylene, alkenylene, or alkynylene, any of which is substituted or unsubstituted;
z is 1, 2, 3, 4, 5, 6, 7, 8, 9, or 10; and
(a) each [Xaa]_{w} is ISGI (SEQ ID NO:53), each [Xaa]ₓ is ASVVNI (SEQ ID NO:54), and each [Xaa]_{y} is KEIDRLNEVAKNLNESLIDLQELGKYEQYI (SEQ ID NO:55);
(b) each [Xaa]_{w} is ISGIN (SEQ ID NO:56), each [Xaa]ₓ is SVVNIQ (SEQ ID NO:57), and each [Xaa]_{y} is EIDRLNEVAKNLNESLIDLQELGKYEQYI (SEQ ID NO:58);
(c) each [Xaa]_{w} is ISGINA (SEQ ID NO:59), each [Xaa]ₓ is VVNIQK (SEQ ID NO:60), and each [Xaa]_{y} is IDRLNEVAKNLNESLIDLQELGKYEQYI (SEQ ID NO:61);
(d) each [Xaa]_{w} is ISGINASVVNIQKEIDRLNEVAKNL (SEQ ID NO:62), each [Xaa]ₓ is ESLIDL (SEQ ID NO:63), and each [Xaa]_{y} is ELGKYEQYI (SEQ ID NO:64);
(e) each [Xaa]_{w} is ISGINASVVNIQKEIDRLNEVAKNLN (SEQ ID NO:65), each [Xaa]ₓ is SLIDLQ (SEQ ID NO:66), and each [Xaa]_{y} is LGKYEQYI (SEQ ID NO:67);
(f) each [Xaa]_{w} is ISGINASVVNIQKEIDRLNEVAKNLNESLIDL (SEQ ID NO:68), each [Xaa]ₓ is ELGKYE (SEQ ID NO:69), and each [Xaa]_{y} is YI;
(g) each [Xaa]_{w} is I, each [Xaa]ₓ is KEIDRL (SEQ ID NO:70), and each [Xaa]_{y} is EVAKNLNESL (SEQ ID NO:71);
(h) each [Xaa]_{w} is IQ, each [Xaa]ₓ is EIDRLN (SEQ ID NO:72), and each [Xaa]_{y} is VAKNLNESL (SEQ ID NO:73);
(i) each [Xaa]_{w} is IQKEI (SEQ ID NO:74), each [Xaa]ₓ is RLNEVA (SEQ ID NO:75), and each [Xaa]_{y} is NLNESL (SEQ ID NO:76);
(j) each [Xaa]_{w} is IQKEID (SEQ ID NO:77), each [Xaa]ₓ is LNEVAK (SEQ ID NO:78), and each [Xaa]_{y} is LNESL (SEQ ID NO:79);
(k) each [Xaa]_{w} is IQKEIDRL (SEQ ID NO:80), each [Xaa]ₓ is EVAKNL (SEQ ID NO:81), and each [Xaa]_{y} is ESL;
(l) each [Xaa]_{w} is IQKEIDRLN (SEQ ID NO:82), each [Xaa]ₓ is VAKNLN (SEQ ID NO:83), and each [Xaa]_{y} is SL;
(m) each [Xaa]_{w} is I, each [Xaa]ₓ is KEI, and each [Xaa]_{y} is RLNEVAKNLNESL (SEQ ID NO:84);
(n) each [Xaa]_{w} is IQ, each [Xaa]ₓ is EID, and each [Xaa]_{y} is LNEVAKNLNESL (SEQ ID NO:85);
(o) each [Xaa]_{w} is IQKEI (SEQ ID NO:74), each [Xaa]ₓ is RLN, and each [Xaa]_{y} is VAKNLNESL (SEQ ID NO:73);
(p) each [Xaa]_{w} is IQKEIDRL (SEQ ID NO:80), each [Xaa]ₓ is EVA, and each [Xaa]_{y} is NLNESL (SEQ ID NO:76);
(q) each [Xaa]_{w} is IQKEIDRLN (SEQ ID NO:82), each [Xaa]ₓ is VAK, and each [Xaa]_{y} is LNESL (SEQ ID NO:79);
(r) each [Xaa]_{w} is IQKEIDRLNEVA (SEQ ID NO:86), each [Xaa]ₓ is NLN, and each [Xaa]_{y} is SL;
(s) each [Xaa]_{w} is IQKEIDRLNEVAK (SEQ ID NO:87), each [Xaa]ₓ is LNE, and each [Xaa]_{y} is L;
(t) each [Xaa]w is missing, each [Xaa]x is QKEIDR (SEQ ID NO:228), and each [Xaa]y is NEVAKNLNESL (SEQ ID NO:229);
(u) each [Xaa]w is IQK, each [Xaa]x is IDRLNE (SEQ ID NO:230), and each [Xaa]y is AKNLNESL (SEQ ID NO:231);
(v) each [Xaa]w is IQKE (SEQ ID NO:232), each [Xaa]x is DRLNEV (SEQ ID NO:181), and each [Xaa]y is KNLNESL (SEQ ID NO:182);
(w) each [Xaa]w is IQKEIDR (SEQ ID NO:183), each [Xaa]x is NEVAKN (SEQ ID NO:184), and each [Xaa]y is NESL (SEQ ID NO:185);
(x) each [Xaa]w is IQKEIDRLNE (SEQ ID NO:186), each [Xaa]x is AKNLNE (SEQ ID NO:187), and each [Xaa]y is L;
(y) each [Xaa]w is IQKEIDRLNEV (SEQ ID NO:188), each [Xaa]x is KNLNES (SEQ ID NO:189), and each [Xaa]y is missing;
(z) each [Xaa]w is QKE, each [Xaa]x is DRLNEVAKNLNESL (SEQ ID NO:190), and each [Xaa]y is missing;
(aa) each [Xaa]w is IQK, each [Xaa]x is IDR, and each [Xaa]y is NEVAKNLNESL (SEQ ID NO:229);
(bb) each [Xaa]w is IQK, each [Xaa]x is IDR, and each [Xaa]y is NEVAKNLNESL (SEQ ID NO:229);
(cc) each [Xaa]w is IQKE (SEQ ID NO:232), each [Xaa]x is DRL, and each [Xaa]y is EVAKNLNESL (SEQ ID NO:71);
(dd) each [Xaa]w is IQKEID (SEQ ID NO:77), each [Xaa]x is LNE, and each [Xaa]y is AKNLNESL (SEQ ID NO:231);
(ee) each [Xaa]w is IQKEIDR (SEQ ID NO:183), each [Xaa]x is NEV, and each [Xaa]y is KNLNESL (SEQ ID NO:182);
(ff) each [Xaa]w is IQKEIDRLNE (SEQ ID NO:186), each [Xaa]x is AKN, and each [Xaa]y is NESL (SEQ ID NO:185);
(gg) each [Xaa]w is IQKEIDRLNEV (SEQ ID NO:188), each [Xaa]x is KNL, and each [Xaa]y is ESL;
(hh) each [Xaa]w is IQKEIDRLNEVAKN (SEQ ID NO:191), each [Xaa]x is NES, and each [Xaa]y is missing;
(ii) each [Xaa]w is ISGINASVVN (SEQ ID NO:250), each [Xaa]x is QKEIDR (SEQ ID NO: 228), and each [Xaa]y is NEVAKNLNESL (SEQ ID NO:229);
(jj) each [Xaa]w is ISGINASVVNI (SEQ ID NO:193), each [Xaa]x is KEIDRL (SEQ ID NO:70), and each [Xaa]y is EVAKNLNESL (SEQ ID NO:71);
(kk) each [Xaa]w is ISGINASVVNIQ (SEQ ID NO:194), each [Xaa]x is EIDRLN (SEQ ID NO:72), and each [Xaa]y is VAKNLNESL (SEQ ID NO:73);
(ll) each [Xaa]w is ISGINASVVNIQK (SEQ ID NO:195), each [Xaa]x is IDRLNE (SEQ ID NO:230), and each [Xaa]y is AKNLNESL (SEQ ID NO:231);
(mm) each [Xaa]w is ISGINASVVNIQKE (SEQ ID NO:196), each [Xaa]x is DRLNEV (SEQ ID NO:181), and each [Xaa]y is KNLNESL (SEQ ID NO: 182);
(nn) each [Xaa]w is ISGINASVVNIQKEI (SEQ ID NO:197), each [Xaa]x is RLNEVA (SEQ ID NO:75), and each [Xaa]y is NLNESL (SEQ ID NO:76);
(oo) each [Xaa]w is ISGINASVVNIQKEID (SEQ ID NO:198), each [Xaa]x is LNEVAK (SEQ ID NO:78), and each [Xaa]y is LNESL (SEQ ID NO:79);
(pp) each [Xaa]w is ISGINASVVNIQKEIDR (SEQ ID NO:199), each [Xaa]x is NEVAKN (SEQ ID NO:184), and each [Xaa]y is NESL (SEQ ID NO:185);
(qq) each [Xaa]w is ISGINASVVNIQKEIDRL (SEQ ID NO:200), each [Xaa]x is EVAKNL (SEQ ID NO:81), and each [Xaa]y is ESL;
(rr) each [Xaa]w is ISGINASVVNIQKEIDRLN (SEQ ID NO:201), each [Xaa]x is VAKNLN (SEQ ID NO:83), and each [Xaa]y is SL;
(ss) each [Xaa]w is ISGINASVVNIQKEIDRLNE (SEQ ID NO:202), each [Xaa]x is AKNLNE (SEQ ID NO:187), and each [Xaa]y is L;
(tt) each [Xaa]w is ISGINASVVNIQKEIDRLNEV (SEQ ID NO:203), each [Xaa]x is KNLNES (SEQ ID NO:189), and each [Xaa]y is missing;
(uu) each [Xaa]w is ISGINASVVN (SEQ ID NO:250), each [Xaa]x is QKE, and each [Xaa]y is DRLNEVAKNLNESL (SEQ ID NO:190);
(vv) each [Xaa]w is ISGINASVVNI (SEQ ID NO:193), each [Xaa]x is KEI, and each [Xaa]y is RLNEVAKNLNESL (SEQ ID NO:84);
(ww) each [Xaa]w is ISGINASVVNIQ (SEQ ID NO:194), each [Xaa]x is EID, and each [Xaa]y is LNEVAKNLNESL (SEQ ID NO:85);
(xx) each [Xaa]w is ISGINASVVNIQK (SEQ ID NO:195), each [Xaa]x is IDR, and each [Xaa]y is NEVAKNLNESL (SEQ ID NO:229);
(yy) each [Xaa]w is ISGINASVVNIQKE (SEQ ID NO:196), each [Xaa]x is DRL, and each [Xaa]y is EVAKNLNESL (SEQ ID NO:71);
(zz) each [Xaa]w is ISGINASVVNIQKEI (SEQ ID NO:197), each [Xaa]x is RLN, and each [Xaa]y is VAKNLNESL (SEQ ID NO:73);
(aaa) each [Xaa]w is ISGINASVVNIQKEID (SEQ ID NO:198), each [Xaa]x is LNE, and each [Xaa]y is AKNLNESL (SEQ ID NO:231);
(bbb) each [Xaa]w is ISGINASVVNIQKEIDR (SEQ ID NO:199), each [Xaa]x is NEV, and each [Xaa]y is KNLNESL (SEQ ID NO:182);
(ccc) each [Xaa]w is ISGINASVVNIQKEIDRL (SEQ ID NO:200), each [Xaa]x is EVA, and each [Xaa]y is NLNESL (SEQ ID NO:76);
(ddd) each [Xaa]w is ISGINASVVNIQKEIDRLN (SEQ ID NO:201), each [Xaa]x is VAK, and each [Xaa]y is LNESL (SEQ ID NO:79);
(eee) each [Xaa]w is ISGINASVVNIQKEIDRLNE (SEQ ID NO:202), each [Xaa]x is AKN, and each [Xaa]y is NESL (SEQ ID NO:185);
(fff) each [Xaa]w is ISGINASVVNIQKEIDRLNEV (SEQ ID NO:203), each [Xaa]x is KNL, and each [Xaa]y is ESL;
(ggg) each [Xaa]w is ISGINASVVNIQKEIDRLNEVA (SEQ ID NO:204), each [Xaa]x is NLN, and each [Xaa]y is SL;
(hhh) each [Xaa]w is ISGINASVVNIQKEIDRLNEVAK (SEQ ID NO:205), each [Xaa]x is LNE, and each [Xaa]y is L; or
(iii) each [Xaa]w is ISGINASVVNIQKEIDRLNEVAKN (SEQ ID NO:206), each [Xaa]x is NES, and each [Xaa]y is missing,
   wherein the structurally-stabilized SARS-CoV-2 HR2 peptide binds the recombinant SARS-CoV-2 5-helix bundle S protein. In some instances, wherein R₁ is an alkyl. In some instances, R₁ is a methyl group. In some instances, R₃ is an alkyl. In some instances, R₃ is a methyl group. In some instances, R₂ is an alkenyl. In some instances, z is 1.

In another aspect of Formula (I), the two alpha, alpha disubstituted stereocenters are both in the R configuration or S configuration (e.g., i, i+4 cross-link), or one stereocenter is R and the other is S (e.g., i, i+7 cross-link). Thus, where Formula (I) is depicted as:

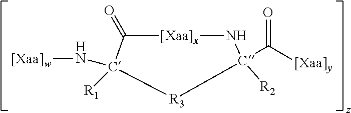

The C′ and C″ disubstituted stereocenters can both be in the R configuration or they can both be in the S configuration, e.g., when x is 3. When x is 6 in Formula (I), the C′ disubstituted stereocenter is in the R configuration and the C″ disubstituted stereocenter is in the S configuration. The R₃ double bond of Formula (I) can be in the E or Z stereochemical configuration.

In some instances of Formula (I), R₃ is [R₄—K—R₄]ₙ; and R₄ is a straight chain alkyl, alkenyl, or alkynyl.

In some instances, “z” of Formula (I) is greater than one. In some instances, z=2, as shown in Formula (II). In this instance, the peptide includes more than one staple. In some instances, the peptide includes two staples (i.e., the peptide is double stapled), as shown in Formula (II). In some instances, a double stapled peptide includes multiple staples in the same construct, creating a construct having [Xaa]ₜ and [Xaa]ᵤ, [Xaa]_{w}, [Xaa]ₓ, and [Xaa]_{y}. Double stapled peptides are provided in Table 3 as constructs 61-97.

Formula II provides the structure of a double stapled peptide:

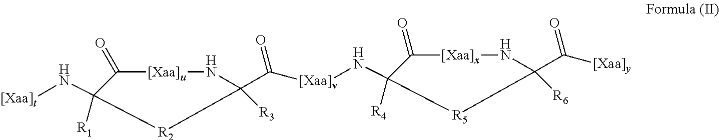

For example, for a stabilized peptide comprising the [Xaa]ₜ, the [Xaa]ᵤ, the [Xaa]ᵥ, the [Xaa]ₓ, and the [Xaa]_{y} of construct 61 of Table 3, the [Xaa]ₜ, the [Xaa]ᵤ, the [Xaa]ᵥ, the [Xaa]ₓ, and the [Xaa]_{y} is: ISGI (SEQ ID NO: 53), ASVVNI (SEQ ID NO: 54), and KEIDRLNEVAKNL (SEQ ID NO: 88), ESLIDL (SEQ ID NO: 63), and ELGKYEQYI (SEQ ID NO: 64), respectively. As another example, for a stabilized peptide comprising the [Xaa]ₜ, the [Xaa]ᵤ, the [Xaa]ᵥ, the [Xaa]ₓ, and the [Xaa]_{y} of construct 62 of Table 3, the [Xaa]ₜ, the [Xaa]ᵤ, the [Xaa]ᵥ, the [Xaa]ₓ, and the [Xaa]_{y} is: ISGI (SEQ ID NO: 53), ASVVNI (SEQ ID NO: 54), and KEIDRLNEVAKNLN (SEQ ID NO: 89), SLIDLQ (SEQ ID NO: 66), and LGKYEQYI (SEQ ID NO: 67), respectively.

TABLE 3
[Xaa]ₜ, [Xaa]ᵤ [Xaa]ᵥ, [Xaa]ₓ, and [Xaa]_{y} sequences for Formula
(II) constructs 61-97.

| Construct | [Xaa]ₜ | [Xaa]ᵤ | [Xaa]ᵥ | [Xaa]ₓ | [Xaa]_{y} |
|---|---|---|---|---|---|
| 61 | ISGI (SEQ | ASVVNI | KEIDRLN | ESLIDL | ELGKYEQ |
| | ID NO: 53) | (SEQ ID | EVAKNL | (SEQ ID | YI (SEQ |
| | | NO: 54) | (SEQ ID | NO: 63) | ID NO: 64) |
| | | | NO: 88) | | |
| 62 | ISGI (SEQ | ASVVNI | KEIDRLN | SLIDLQ | LGKYEQ |
| | ID NO: 53) | (SEQ ID | EVAKNL | (SEQ ID | YI (SEQ |
| | | NO: 54) | N (SEQ ID | NO: 66) | ID NO: 67) |
| | | | NO: 89) | | |
| 63 | ISGI (SEQ | ASVVNI | KEIDRLN | ELGKYE | YI |
| | ID NO: 53) | (SEQ ID | EVAKNL | (SEQ ID | |
| | | NO: 54) | NESLIDL | NO: 69) | |
| | | | (SEQ ID | | |
| | | | NO: 90) | | |
| 64 | ISGIN | SVVNIQ | EIDRLNE | ESLIDL | ELGKYEQ |
| | (SEQ ID | (SEQ ID | VAKNL | (SEQ ID | YI (SEQ |
| | NO: 56) | NO: 57) | (SEQ ID | NO: 63) | ID NO: 64) |
| | | | NO: 91) | | |
| 65 | ISGIN | SVVNIQ | EIDRLNE | SLIDLQ | LGKYEQ |
| | (SEQ ID | (SEQ ID | VAKNLN | (SEQ ID | YI (SEQ |
| | NO: 56) | NO: 57) | (SEQ ID | NO: 66) | ID NO: 67) |
| | | | NO: 92) | | |
| 66 | ISGIN | SVVNIQ | EIDRLNE | ELGKYE | YI |
| | (SEQ ID | (SEQ ID | VAKNLN | (SEQ ID | |
| | NO: 56) | NO: 57) | ESLIDL | NO: 69) | |
| | | | (SEQ ID | | |
| | | | NO: 93) | | |
| 67 | ISGINA | VVNIQK | IDRLNEV | ESLIDL | ELGKYEQ |
| | (SEQ ID | (SEQ ID | AKNL | (SEQ ID | YI (SEQ |
| | NO: 59) | NO: 60) | (SEQ ID | NO: 63) | ID NO: 64) |
| | | | NO: 94) | | |
| 68 | ISGINA | VVNIQK | IDRLNEV | SLIDLQ | LGKYEQ |
| | (SEQ ID | (SEQ ID | AKNLN | (SEQ ID | YI (SEQ |
| | NO: 59) | NO: 60) | (SEQ ID | NO: 66) | ID NO: 67) |
| | | | NO: 95) | | |
| 69 | ISGINA | VVNIQK | IDRLNEV | ELGKYE | YI |
| | (SEQ ID | (SEQ ID | AKNLNES | (SEQ ID | |
| | NO: 59) | NO: 60) | LIDL (SEQ | NO: 69) | |
| | | | ID NO: 96) | | |
| 70 | I | KEI | RLNEVA | NLN | SL |
| | | | (SEQ ID | | |
| | | | NO: 97) | | |
| 71 | IQ | EID | LNEVA | NLN | SL |
| | | | (SEQ ID | | |
| | | | NO: 98) | | |
| 72 | I | KEI | RLNEVA | LNE | L |
| | | | K (SEQ ID | | |
| | | | NO: 99) | | |
| 73 | IQ | EID | LNEVAK | LNE | L |
| | | | (SEQ ID | | |
| | | | NO: 100) | | |
| 74 | I | KEI | RLNEVA | NLN | SL |
| | | | (SEQ ID | | |
| | | | NO: 75) | | |
| 75 | IQ | EID | LNEVA | NLN | SL |
| | | | (SEQ ID | | |
| | | | NO: 98) | | |
| 76 | I | KEI | RLNEVA | LNE | L |
| | | | K (SEQ ID | | |
| | | | NO: 99) | | |
| 77 | IQ | EID | LNEVAK | LNE | L |
| | | | (SEQ ID | | |
| | | | NO: 78) | | |
| 78 | I | KEI | RLNEVA | NLN | SLIDLQE |
| | | | (SEQ ID | | L (SEQ ID |
| | | | NO: 75) | | NO: 207) |
| 79 | IQ | EID | LNEVA | NLN | SLIDLQE |
| | | | (SEQ ID | | L (SEQ ID |
| | | | NO: 98) | | NO: 207) |
| 80 | I | KEI | RLNEVA | LNE | LIDLQEL |
| | | | K (SEQ ID | | (SEQ ID |
| | | | NO: 99) | | NO: 208) |
| 81 | IQ | EID | LNEVAK | LNE | LIDLQEL |
| | | | (SEQ ID | | (SEQ ID |
| | | | NO: 78) | | NO: 208) |
| 82 | DISGINAS | KEI | RLNEVA | NLN | SL |
| | VVNI | | (SEQ ID | | |
| | (SEQ ID | | NO: 75) | | |
| | NO: 209) | | | | |
| 83 | DISGINAS | EID | LNEVA | NLN | SL |
| | VVNIQ | | (SEQ ID | | |
| | (SEQ ID | | NO: 98) | | |
| | NO: 210) | | | | |
| 84 | DISGINAS | KEI | RLNEVA | LNE | L |
| | VVNI | | K (SEQ ID | | |
| | (SEQ ID | | NO: 99) | | |
| | NO: 209) | | | | |
| 85 | DISGINAS | EID | LNEVAK | LNE | L |
| | VVNIQ | | (SEQ ID | | |
| | (SEQ ID | | NO: 78) | | |
| | NO: 210) | | | | |
| 86 | DISGINAS | KEI | RLNEVA | NLN | SLIDLQE |
| | VVNI | | (SEQ ID | | L (SEQ ID |
| | (SEQ ID | | NO: 75) | | NO: 207) |
| | NO: 209) | | | | |
| 87 | DISGINAS | EID | LNEVA | NLN | SLIDLQE |
| | VVNIQ | | (SEQ ID | | L (SEQ ID |
| | (SEQ ID | | NO: 98) | | NO: 207) |
| | NO: 210) | | | | |
| 88 | DISGINAS | KEI | RLNEVA | LNE | LIDLQEL |
| | VVNI | | K (SEQ ID | | (SEQ ID |
| | (SEQ ID | | NO: 99) | | NO: 208) |
| | NO: 209) | | | | |
| 89 | DISGINAS | EID | LNEVAK | LNE | LIDLQEL |
| | VVNIQ | | (SEQ ID | | (SEQ ID |
| | (SEQ ID | | NO: 78) | | NO: 208) |
| | NO: 210) | | | | |
| 90 | ISGINASV | KEI | RLNEVA | NLN | SLIDLQE |
| | VNI (SEQ | | (SEQ ID | | LGKYEQ |
| | ID | | NO: 75) | | YI (SEQ |
| | NO: 193) | | | | ID |
| | | | | | NO: 211) |
| 91 | ISGINASV | EID | LNEVA | NLN | SLIDLQE |
| | VNIQ | | (SEQ ID | | LGKYEQ |
| | (SEQ ID | | NO: 98) | | YI (SEQ |
| | NO: 194) | | | | ID |
| | | | | | NO: 211) |
| 92 | ISGINASV | KEI | RLNEVA | LNE | LIDLQEL |
| | VNI (SEQ | | K (SEQ ID | | GKYEQYI |
| | ID | | NO: 99) | | (SEQ ID |
| | NO: 193) | | | | NO: 212) |
| 93 | ISGINASV | EID | LNEVAK | LNE | LIDLQEL |
| | VNIQ | | (SEQ ID | | GKYEQYI |
| | (SEQ ID | | NO: 78) | | (SEQ ID |
| | NO: 194) | | | | NO: 212) |
| 94 | SLDQINV | YEB | KLEEAI | KLE | SYIDLKE |
| | TFLDL | | (SEQ ID | | (SEQ ID |
| | (SEQ ID | | NO: 214) | | NO: 215) |
| | NO: 213) | | | | |
| 95 | SLDQINV | EBK | LEEAI | KLE | SYIDLKE |
| | TFLDLE | | (SEQ ID | | (SEQ ID |
| | (SEQ ID | | NO: 217) | | NO: 215) |
| | NO: 216) | | | | |
| 96 | SLDQINV | YEB | KLEEAIK | LEE | YIDLKE |
| | TFLDL | | (SEQ ID | | (SEQ ID |
| | (SEQ ID | | NO: 218) | | NO: 219) |
| | NO: 213) | | | | |
| 97 | SLDQINV | EBK | LEEAIK | LEE | YIDLKE |
| | TFLDLE | | (SEQ ID | | (SEQ ID |
| | (SEQ ID | | NO: 220) | | NO: 219) |
| | NO: 216) | | | | |

In one aspect, a structurally-stabilized (stitched) SARS-CoV-2 HR2 peptide comprises Formula (III):

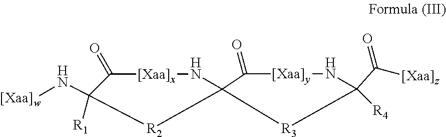

or a pharmaceutically acceptable salt thereof, wherein:
each R₁ and R₄ is independently H or a C₁₋₁₀ alkyl, alkenyl, alkynyl, arylalkyl, cycloalkylalkyl, heteroarylalkyl, or heterocyclylalkyl, any of which is substituted or unsubstituted;
each of R₂ and R₃ is independently a C₅₋₂₀ alkyl, alkenyl, alkynyl; [R₄—K—R₄]ₙ;
each of which is substituted with 0-6 R₅;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

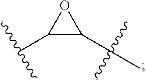 [SMILES: *C1OC1*]

R₆ is H, alkyl, or a therapeutic agent;
n is an integer from 1-4; and
[Xaa]_{w}; [Xaa]ₓ; [Xaa]_{y}; and [Xaa]_{z} are provided in Table 4.

In some embodiments, each of the [Xaa]_{w} of Formula (III), the [Xaa]ₓ of Formula (III), the [Xaa]_{y} of Formula (III), [Xaa]_{z} of Formula (III) is as described for any one of constructs 98-108 of Table 4. For example, for a stabilized peptide comprising the [Xaa]_{w}, the [Xaa]ₓ, the [Xaa]_{y}, and the [Xaa]_{z} of construct 98 of Table 4, the [Xaa]_{w}, the [Xaa]ₓ, the [Xaa]_{y}, and the [Xaa]_{z} is: ISGINASVVNIQKEIDRLNEVAKNL (SEQ ID NO: 62), ESLIDL (SEQ ID NO: 63), ELGKYE (SEQ ID NO: 69), and YI, respectively. As another example, for a stabilized peptide comprising the [Xaa]_{w}, the [Xaa]ₓ, the [Xaa]_{y}, and the [Xaa]_{z} of construct 99 of Table 4, the [Xaa]_{w}, the [Xaa]ₓ, the [Xaa]_{y}, and the [Xaa]_{z} is: I, KEIDRL (SEQ ID NO: 70), EVAKNL (SEQ ID NO: 81), and ESL, respectively.

TABLE 4
[Xaa]_{w}, [Xaa]ₓ, [Xaa]_{y}, and [Xaa]_{z} sequences for Formula (III)
constructs 98-108.

| Construct | [Xaa]_{w} | [Xaa]ₓ | [Xaa]_{y} | [Xaa]_{z} |
|---|---|---|---|---|
| 98 | ISGINASVV | ESLIDL (SEQ | ELGKYE (SEQ | YI |
| | NIQKEIDRL | ID NO: 63) | ID NO: 69) | |
| | NEVAKNL | | | |
| | (SEQ ID | | | |
| | NO: 62) | | | |
| 99 | I | KEIDRL (SEQ | EVAKNL (SEQ | ESL |
| | | ID NO: 70) | ID NO: 81) | |
| 100 | IQ | EIDRLN (SEQ | VAKNLN (SEQ | SL |
| | | ID NO: 72) | ID NO: 83) | |
| 101 | I | KEIDRL (SEQ | EVA | NLNESL |
| | | ID NO: 70) | | (SEQ ID |
| | | | | NO: 76) |
| 102 | IQ | EIDRLN (SEQ | VAK | LNESL (SEQ |
| | | ID NO: 72) | | ID NO: 79) |
| 103 | IQKEI (SEQ | RLNEVA (SEQ | NLN | SL |
| | ID NO: 74) | ID NO: 75) | | |
| 104 | IQKEID | LNEVAK (SEQ | LNE | L |
| | (SEQ ID | ID NO: 78) | | |
| | NO: 77) | | | |
| 105 | ISGINASVV | KEIDRL (SEQ | EVAKNL (SEQ | ESLIDLQEL |
| | NI (SEQ ID | ID NO: 70) | ID NO: 81) | GKYEQYI |
| | NO: 193) | | | (SEQ ID |
| | | | | NO: 221) |
| 106 | ISGINASVV | EIDRLN (SEQ | VAK | LNESLIDLQ |
| | NIQ (SEQ ID | ID NO: 72) | | ELGKYEQYI |
| | NO: 194) | | | (SEQ ID |
| | | | | NO: 222) |
| 107 | SLDQINVTF | YEBKKL (SEQ | EAIKKL (SEQ | ESYIDLKE |
| | LDL (SEQ | ID NO: 223) | ID NO: 224) | (SEQ ID |
| | ID NO: 213) | | | NO: 225) |
| 108 | SLDQINVTF | EBKKLE (SEQ | AIKKLE (SEQ | SYIDLKE |
| | LDLE (SEQ | ID NO: 226) | ID NO: 227) | (SEQ ID |
| | ID NO: 216) | | | NO: 215) |

In some instances, Formula (III) comprising the sequences set forth above in Table 4 can have one or more of the properties listed below: (i) binds the recombinant SARS-CoV-2 5-helix bundle S protein; (ii) is alpha-helical; (iii) is protease resistant; (iv) inhibits fusion of SARS-CoV-2 with a host cell; and/or (v) inhibits infection of a cell by SARS-CoV-2.

In some instances of Formula (III), R₁ and R₄ are each independently H or C₁-C₆ alkyl. In some instances of Formula (III), R₁ and R₄ are each independently C₁-C₃ alkyl. In some instances of Formula (III), at least one of R₁ and R₄ are methyl. For example, R₁ and R₄ can both be methyl. In some instances of Formula (III), R₂ and R₃ are each independently alkyl (e.g., C₁₂ alkyl). In some instances of Formula (III), R₂ and R₃ are each independently a C₁₂ alkyl. In some instances of Formula (III), R₂ and R₃ are each independently a straight chain alkyl, alkenyl, or alkynyl (e.g., a straight chain C₁₂ alkyl, alkenyl, or alkynyl. In some instances of Formula (III), R₂ is —CH₂—CH₂—CH₂—CH₂—CH₂—CH₂—CH═CH—CH₂—CH₂—CH₂—CH₂—. In some instances of Formula (III), R₃ is —CH₂—CH₂—CH₂—CH₂—CH═CH—CH₂—CH₂—CH₂—CH₂—CH₂—CH₂—.

In some instances, the structurally-stabilized SARS-CoV-2 HR2 peptide comprises Formula (III), or a pharmaceutically acceptable salt thereof, wherein: [Xaa]_{w}; [Xaa]ₓ; [Xaa]_{y}; and [Xaa]_{z} are provided in Table 4;
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted; and wherein the structurally-stabilized SARS-CoV-2 HR2 peptide wherein the peptide binds the recombinant SARS-CoV-2 5-helix bundle S protein. In some instances, R₁ is an alkyl. In some instances, R₁ is a methyl group. In some instances, R₄ is an alkyl. In some instances, R₄ is a methyl group. In some instances, R₂ is an alkenyl. In some instances, R₃ is an alkenyl.

In another aspect of Formula (III), of the three alpha, alpha disubstituted stereocenters: (i) two stereocenters are in the R configuration and one stereocenter is in the S configuration; or (ii) two stereocenters are in the S configuration and one stereocenter is in the R configuration. Thus, where Formula (III) is depicted as:

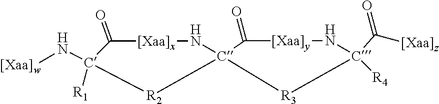

The C′ and C′″ disubstituted stereocenters can both be in the R configuration or they can both be in the S configuration. When both C′ and C′″ are in the R configuration, C″ is in the S configuration. When both C′ and C′″ are in the S configuration, C″ is in the R configuration. The double bond in each of R₂ and R₃ of Formula (III) can be in the E or Z stereochemical configuration.

In some instances of Formula (III), R₃ is [R₄—K—R₄]ₙ; and R₄ is a straight chain alkyl, alkenyl, or alkynyl.

In another aspect, the structurally-stabilized peptide may be both stapled and stitched as shown in the structure below:

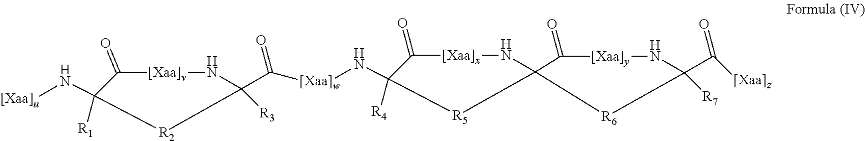

or a pharmaceutically acceptable salt thereof, wherein:
each R₁, R₃, R₄, and R₇ is independently H or a C₁₋₁₀ alkyl, alkenyl, alkynyl, arylalkyl, cycloalkylalkyl, heteroarylalkyl, or heterocyclylalkyl, any of which is substituted or unsubstituted;
each of R₂, R₅, and R₆ is independently a C₅₋₂₀ alkyl, alkenyl, alkynyl;
[R₄—K—R₄]ₙ; each of which is substituted with 0-6 R₅;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

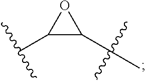 [SMILES: *C1OC1*]

R₆ is H, alkyl, or a therapeutic agent;
n is an integer from 1-4; and
[Xaa]ᵤ, [Xaa]ᵥ, [Xaa]_{w}, [Xaa]ₓ, [Xaa]_{y}, and [Xaa]_{z} are provided in Table 5.

In some embodiments, each of the [Xaa]ᵤ of Formula (IV), the [Xaa]ᵥ of Formula (IV), the [Xaa]_{w} of Formula (IV), the [Xaa]ₓ of Formula (IV), the [Xaa]_{y} of Formula (IV), and the [Xaa]_{z} of Formula (IV) is as described for any one of constructs 109-111 of Table 5. For example, for a stabilized peptide comprising the [Xaa]ᵤ, the [Xaa]ᵥ, the [Xaa]_{w}, the [Xaa]ₓ, the [Xaa]y, and the [Xaa]_{z} of construct 109 of Table 5, the [Xaa]ᵤ, the [Xaa]ᵥ, the [Xaa]_{w}, the [Xaa]ₓ, the [Xaa]_{y}, and the [Xaa]_{z} is: ISGI (SEQ ID NO:53); ASVVNI (SEQ ID NO:54); KEIDRLNEVAKNL (SEQ ID NO:88); ESLIDL (SEQ ID NO:63); ELGKYE (SEQ ID NO:69); and YI, respectively. As another example, for a stabilized peptide comprising the [Xaa]ᵤ, the [Xaa]ᵥ, the [Xaa]_{w}, the [Xaa]ₓ, the [Xaa]_{y}, and the [Xaa]_{z} of construct 110 of Table 5, the [Xaa]ᵤ, the [Xaa]ᵥ, the [Xaa]_{w}, the [Xaa]ₓ, the [Xaa]_{y}, and the [Xaa]_{z} is: ISGIN (SEQ ID NO:56); SVVNIQ (SEQ ID NO:57); EIDRLNEVAKNL (SEQ ID NO:91); ESLIDL (SEQ ID NO:63); ELGKYE (SEQ ID NO:69); and YI, respectively.

TABLE 5
[Xaa]ᵤ, [Xaa]ᵥ, [Xaa]_{w}, [Xaa]ₓ, [Xaa]_{y}, and [Xaa]_{z} sequences for
Formula (IV) constructs 109-111.

| Construct | [Xaa]ᵤ | [Xaa]ᵥ | [Xaa]_{w} | [Xaa]ₓ | [Xaa]_{y} | [Xaa]_{z} |
|---|---|---|---|---|---|---|
| 109 | ISGI | ASVVNI | KEIDRL | ESLIDL | ELGKYE | YI |
| | (SEQ ID | (SEQ ID | NEVAK | (SEQ ID | (SEQ ID | |
| | NO: 53) | NO: 54) | NL (SEQ | NO: 63) | NO: 69) | |
| | | | ID | | | |
| | | | NO: 88) | | | |
| 110 | ISGIN | SVVNIQ | EIDRLN | ESLIDL | ELGKYE | YI |
| | (SEQ ID | (SEQ ID | EVAKN | (SEQ ID | (SEQ ID | |
| | NO: 56) | NO: 57) | L (SEQ | NO: 63) | NO: 69) | |
| | | | ID | | | |
| | | | NO: 91) | | | |
| 111 | ISGINA | VVNIQ | IDRLNE | ESLIDL | ELGKYE | YI |
| | (SEQ ID | K (SEQ | VAKNL | (SEQ ID | (SEQ ID | |
| | NO: 59) | ID | (SEQ ID | NO: 63) | NO: 69) | |
| | | NO: 60) | NO: 94) | | | |

In some instances, Formula (IV) comprising the sequences set forth above in Table 5 can have one or more of the properties listed below: (i) binds the recombinant SARS-CoV-2 5-helix bundle S protein; (ii) is alpha-helical; (iii) is protease resistant; (iv) inhibits fusion of SARS-CoV-2 with a host cell; and/or (v) inhibits infection of a cell by SARS-CoV-2.

In some instances of Formula (IV), R₁, R₃, R₄, and R₇ are each independently H or C₁-C₆ alkyl. In some instances of Formula (IV), R₂, R₅, and R₆ are each independently C₁-C₃ alkyl. In some instances of Formula (IV), at least one of R₁, R₃, R₄, and R₇ are methyl. For example, R₁, R₃, R₄, and R₇ can both be methyl. In some instances of Formula (IV), R₂, R₅, and R₆ are each independently alkyl (e.g., C₁₂ alkyl). In some instances of Formula (IV), R₂, R₅, and R₆ are each independently a C₁₂ alkyl. In some instances of Formula (IV), R₂, R₅, and R₆ are each independently a straight chain alkyl, alkenyl, or alkynyl (e.g., a straight chain C₁₂ alkyl, alkenyl, or alkynyl. In some instances of Formula (IV), R₂ is —CH₂—CH₂—CH₂—CH₂—CH₂—CH₂—CH═CH—CH₂—CH₂—CH₂—CH₂—. In some instances of Formula (IV), R₅ is —CH₂—CH₂—CH₂—CH₂—CH═CH—CH₂—CH₂—CH₂—CH₂—CH₂—CH₂—. In some instances of Formula (IV), R₆ is —CH₂—CH₂—CH₂—CH₂—CH═CH—CH₂—CH₂—CH₂—CH₂—CH₂—CH₂—.

In some instances, the structurally-stabilized SARS-CoV-2 HR2 peptide comprises Formula (IV), or a pharmaceutically acceptable salt thereof, wherein: [Xaa]ᵤ; [Xaa]ᵥ; [Xaa]_{w}; [Xaa]ₓ; [Xaa]_{y}; and [Xaa]_{z} are provided in Table 5;
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted; and wherein the structurally-stabilized SARS-CoV-2 HR2 peptide wherein the peptide binds the recombinant SARS-CoV-2 5-helix bundle S protein. In some instances, R₁ is an alkyl. In some instances, R₁ is a methyl group. In some instances, R₄ is an alkyl. In some instances, R₄ is a methyl group. In some instances, R₂ is an alkenyl. In some instances, R₃ is an alkenyl. As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to 10 ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to 10 carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some embodiments, halo is F or Cl.

In some embodiments, the disclosure features structurally-stabilized (e.g., stapled or stitched) peptides comprising the amino acid sequence of any one of SEQ ID NOs: 9, 10, 103, 104, 106, 108, or 110 (or a modified version thereof), wherein: the side chains of two amino acids separated by two, three, or six amino acids are replaced by an internal staple, the side chains of three amino acids are replaced by an internal stitch, the side chains of four amino acids are replaced by two internal staples, or the side chains of five amino acids are replaced by the combination of an internal staple and an internal stitch. In some embodiments, the disclosure features structurally-stabilized (e.g., stapled or stitched) peptides comprising the amino acid sequence of any one of SEQ ID NOs: 9, 10, 103, 104, 106, 108, or 110 (or a modified version thereof), wherein the side chains of two amino acids separated by two, three, or six amino acids are replaced by an internal staple. In some embodiments, the disclosure features structurally-stabilized (e.g., stapled or stitched) peptides comprising the amino acid sequence of any one of SEQ ID NOs: 9, 10, 103, 104, 106, 108, or 110 (or a modified version thereof), wherein the side chains of two amino acids separated by three amino acids are replaced by an internal staple. In some embodiments, the disclosure features structurally-stabilized (e.g., stapled or stitched) peptides comprising the amino acid sequence of any one of SEQ ID NOs: 9, 10, 103, 104, 106, 108, or 110 (or a modified version thereof), wherein the side chains of two amino acids separated by six amino acids are replaced by an internal staple. In some embodiments, the disclosure features structurally-stabilized (e.g., stapled or stitched) peptides comprising the amino acid sequence of any one of SEQ ID NOs: 9, 10, 103, 104, 106, 108, or 110 (or a modified version thereof), wherein the side chains of three amino acids are replaced by an internal stitch.

The stapled or stitched peptide can be 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids in length. In a specific embodiment, the stapled or stitched peptide is 19-45 amino acids (i.e., 19, 20, 21, 25, 30, 31, 32, 33, 34, 35, 36, 37, 38, 39, 40, 41, 42, 43, 44, 45) in length. In a specific embodiment, the stapled or stitched peptide is 19-35 amino acids (i.e., 19, 20, 21, 22, 23, 34, 235, 26, 27, 28, 29, 30, 31, 32, 33, 34, 35) amino acids in length. In a specific embodiment, the stapled or stitched peptide is 19-42 amino acids (i.e., 19, 20, 21, 22, 23, 34, 235, 26, 27, 28, 29, 30, 31, 32, 33, 34, 35, 36, 37, 38, 39, 40, 41. 42) amino acids in length. In a specific embodiment, the stapled or stitched peptide is 19 amino acids in length. In another specific embodiment, the stapled or stitched peptide is 42 amino acids in length. Exemplary COVID-19 HR2 stapled or stitched peptides are shown in Tables 1-5 and described in Formulae (I)-(IV). In one embodiment, the COVID-19 HR2 stapled or stitched peptide comprises or consists of a stapled or stitched version of the amino acid sequence of any one of SEQ ID NOs: 11-52 or 112-180 (e.g., the product of a ring-closing metathesis reaction performed on a peptide comprising the amino acid sequence of any one of SEQ ID NOs: 11-52 or 112-180, respectively). In one embodiment, the SARS-CoV-2 HR2 stapled or stitched peptide comprises or consists of a stapled or stitched version of the amino acid sequence of SEQ ID NO: 9 (e.g., the product of a ring-closing metathesis reaction performed on a peptide comprising the amino acid sequence of SEQ ID NO:9). In one embodiment, the SARS-CoV-2 HR2 stapled or stitched peptide comprises or consists of a stapled or stitched version of the amino acid sequence of SEQ ID NO: 10 (e.g., the product of a ring-closing metathesis reaction performed on a peptide comprising the amino acid sequence of SEQ ID NO: 10).

In certain embodiments, the stapled peptide comprises or consists of a variant of the amino acid sequence set forth in any one of SEQ ID NOs: 9, 10, 103, 104, 106, 108, or 110, wherein two amino acids each separated by 3 amino acids (i.e., positions i and i+4) are modified to structurally stabilize the peptide (e.g., by substituting them with non-natural amino acids to permit hydrocarbon stitching, i.e., stapling amino acids). In certain embodiments, the stapled peptide comprises or consists of a variant of the amino acid sequence set forth in any one of SEQ ID NOs: 9, 10, 103, 104, 106, 108, or 110, wherein two amino acids each separated by 6 amino acids (i.e., positions i and i+7) are modified to structurally stabilize the peptide (e.g., by substituting them with non-natural amino acids to permit hydrocarbon stapling, i.e., with stapling amino acids).

In certain embodiments, the two amino acids each separated by six amino acids are at the amino acid positions in the SARS-CoV-2 HR2 peptide corresponding to positions 5 and 12 of SEQ ID NO:9. In certain embodiments, the two amino acids each separated by six amino acids are at the amino acid positions in the SARS-CoV-2 HR2 peptide corresponding to positions 6 and 13 of SEQ ID NO:9. In certain embodiments, the two amino acids each separated by six amino acids are at the amino acid positions in the SARS-CoV-2 HR2 peptide corresponding to positions 7 and 14 of SEQ ID NO:9. In certain embodiments, the two amino acids each separated by six amino acids are at the amino acid positions in the SARS-CoV-2 HR2 peptide corresponding to positions 26 and 33 of SEQ ID NO:9. In certain embodiments, the two amino acids each separated by six amino acids are at the amino acid positions in the SARS-CoV-2 HR2 peptide corresponding to positions 27 and 34 of SEQ ID NO:9. In certain embodiments, the two amino acids each separated by six amino acids are at the amino acid positions in the SARS-CoV-2 HR2 peptide corresponding to positions 33 and 40 of SEQ ID NO:9. In certain embodiments, the three amino acids each separated by six amino acids are at the amino acid positions in the SARS-CoV-2 HR2 peptide corresponding to positions 26, 33, and 40 of SEQ ID NO:9. In certain embodiments, the two amino acids each separated by six amino acids are at the amino acid positions in the SARS-CoV-2 HR2 peptide corresponding to positions 5, 12, 26, and 33 of SEQ ID NO:9. In certain embodiments, the two amino acids each separated by six amino acids are at the amino acid positions in the SARS-CoV-2 HR2 peptide corresponding to positions 5, 12, 27 and 34 of SEQ ID NO:9. In certain embodiments, the two amino acids each separated by six amino acids are at the amino acid positions in the SARS-CoV-2 HR2 peptide corresponding to positions 5 and 12, 33 and 40 of SEQ ID NO:9. In certain embodiments, the two amino acids each separated by six amino acids are at the amino acid positions in the SARS-CoV-2 HR2 peptide corresponding to positions 5, 12, 26, 33, and 40 of SEQ ID NO:9. In certain embodiments, the two amino acids each separated by six amino acids are at the amino acid positions in the SARS-CoV-2 HR2 peptide corresponding to positions 6, 13, 26, and 33 of SEQ ID NO:9. In certain embodiments, the two amino acids each separated by six amino acids are at the amino acid positions in the SARS-CoV-2 HR2 peptide corresponding to positions 6, 13, 27, and 34 of SEQ ID NO:9. In certain embodiments, the two amino acids each separated by six amino acids are at the amino acid positions in the SARS-CoV-2 HR2 peptide corresponding to positions 6, 13, 33, and 40 of SEQ ID NO:9. In certain embodiments, the two amino acids each separated by six amino acids are at the amino acid positions in the SARS-CoV-2 HR2 peptide corresponding to positions 6, 13, 26, 33, and 40 of SEQ ID NO:9. In certain embodiments, the two amino acids each separated by six amino acids are at the amino acid positions in the SARS-CoV-2 HR2 peptide corresponding to positions 7, 14, 26, and 33 of SEQ ID NO:9. In certain embodiments, the two amino acids each separated by six amino acids are at the amino acid positions in the SARS-CoV-2 HR2 peptide corresponding to positions 7, 14, 27, and 34 of SEQ ID NO:9. In certain embodiments, the two amino acids each separated by six amino acids are at the amino acid positions in the SARS-CoV-2 HR2 peptide corresponding to positions 7, 14, 33, and 40 of SEQ ID NO:9. In certain embodiments, the two amino acids each separated by six amino acids are at the amino acid positions in the SARS-CoV-2 HR2 peptide corresponding to positions 7, 14, 26, 33, and 40 of SEQ ID NO:9.

In certain embodiments, the two amino acids each separated by six amino acids are at the amino acid positions in the SARS-CoV-2 HR2 peptide corresponding to positions 2 and 9 of SEQ ID NO:10. In certain embodiments, the two amino acids each separated by six amino acids are at the amino acid positions in the SARS-CoV-2 HR2 peptide corresponding to positions 3 and 10 of SEQ ID NO:10. In certain embodiments, the two amino acids each separated by six amino acids are at the amino acid positions in the SARS-CoV-2 HR2 peptide corresponding to positions 6 and 13 of SEQ ID NO:10. In certain embodiments, the two amino acids each separated by six amino acids are at the amino acid positions in the SARS-CoV-2 HR2 peptide corresponding to positions 7 and 14 of SEQ ID NO:10. In certain embodiments, the two amino acids each separated by six amino acids are at the amino acid positions in the SARS-CoV-2 HR2 peptide corresponding to positions 9 and 16 of SEQ ID NO:10. In certain embodiments, the two amino acids each separated by six amino acids are at the amino acid positions in the SARS-CoV-2 HR2 peptide corresponding to positions 10 and 17 of SEQ ID NO:10. In certain embodiments, the two amino acids each separated by three amino acids are at the amino acid positions in the SARS-CoV-2 HR2 peptide corresponding to positions 2 and 6 of SEQ ID NO:10. In certain embodiments, the two amino acids each separated by three amino acids are at the amino acid positions in the SARS-CoV-2 HR2 peptide corresponding to positions 3 and 7 of SEQ ID NO: 10. In certain embodiments, the two amino acids each separated by three amino acids are at the amino acid positions in the SARS-CoV-2 HR2 peptide corresponding to positions 6 and 10 of SEQ ID NO:10. In certain embodiments, the two amino acids each separated by three amino acids are at the amino acid positions in the SARS-CoV-2 HR2 peptide corresponding to positions 9 and 13 of SEQ ID NO: 10. In certain embodiments, the two amino acids each separated by three amino acids are at the amino acid positions in the SARS-CoV-2 HR2 peptide corresponding to positions 10 and 14 of SEQ ID NO:10. In certain embodiments, the two amino acids each separated by three amino acids are at the amino acid positions in the SARS-CoV-2 HR2 peptide corresponding to positions 13 and 17 of SEQ ID NO: 10. In certain embodiments, the two amino acids each separated by three amino acids are at the amino acid positions in the SARS-CoV-2 HR2 peptide corresponding to positions 14 and 18 of SEQ ID NO:10. In certain embodiments, the two amino acids each separated by six amino acids are at the amino acid positions in the SARS-CoV-2 HR2 peptide corresponding to positions 2, 9, and 16 of SEQ ID NO: 10. In certain embodiments, the two amino acids each separated by six amino acids are at the amino acid positions in the SARS-CoV-2 HR2 peptide corresponding to positions 3, 10, and 17 of SEQ ID NO:10. In certain embodiments, the two amino acids each separated by six or three amino acids are at the amino acid positions in the SARS-CoV-2 HR2 peptide corresponding to positions 2, 9, and 13 of SEQ ID NO:10. In certain embodiments, the two amino acids each separated by six or three amino acids are at the amino acid positions in the SARS-CoV-2 HR2 peptide corresponding to positions 3, 10, and 14 of SEQ ID NO:10. In certain embodiments, the two amino acids each separated by six or three amino acids are at the amino acid positions in the SARS-CoV-2 HR2 peptide corresponding to positions 6, 13, and 17 of SEQ ID NO:10. In certain embodiments, the two amino acids each separated by six or three amino acids are at the amino acid positions in the SARS-CoV-2 HR2 peptide corresponding to positions 7, 14, and 18 of SEQ ID NO: 10. In certain embodiments, the two amino acids each separated by three or six amino acids are at the amino acid positions in the SARS-CoV-2 HR2 peptide corresponding to positions 2, 6, 13, and 17 of SEQ ID NO:10. In certain embodiments, the two amino acids each separated by three or six amino acids are at the amino acid positions in the SARS-CoV-2 HR2 peptide corresponding to positions 3, 7, 13, and 17 of SEQ ID NO:10. In certain embodiments, the two amino acids each separated by three or six amino acids are at the amino acid positions in the SARS-CoV-2 HR2 peptide corresponding to positions 2, 6, 14, and 18 of SEQ ID NO:10. In certain embodiments, the two amino acids each separated by three or six amino acids are at the amino acid positions in the SARS-CoV-2 HR2 peptide corresponding to positions 3, 7, 14, and 18 of SEQ ID NO: 10.

In certain embodiments, the stitched peptide comprises or consists of a variant of the amino acid sequence set forth in any one of SEQ ID NOs: 9, 10, 103, 104, 106, 108, or 110, wherein two, three, four, five amino acids, at positions such as i, i+3, i, i+4, and i+7, are substituted to structurally stabilize the peptide (e.g., by substituting them with non-natural amino acids to permit hydrocarbon stitching, i.e., with stitching amino acids).

While hydrocarbon tethers are common, other tethers can also be employed in the structurally-stabilized SARS-CoV-2 HR2 peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the SARS-CoV-2 HR2 peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc., 113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U.S.A., 97:3803-3808 (2000); Double-click stapling: Lau et al., Chem. Sci., 5:1804-1809 (2014); Bis-lactam stapling: J. C. Phelan et al., J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3, i to i+4, and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

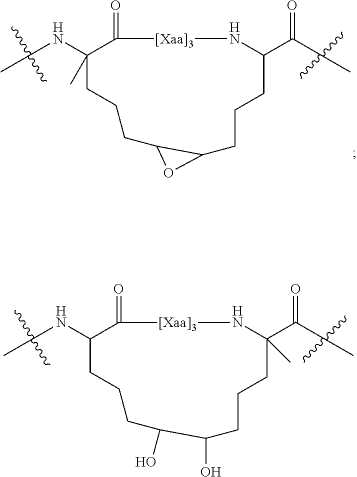

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the peptide or via the amino acid side chain. Other agents can be attached to the functionalized tether, e.g., an agent that facilitates entry of the peptide into cells.

In some instances, alpha disubstituted amino acids are used in the peptide to improve the stability of the alpha helical secondary structure. However, alpha disubstituted amino acids are not required, and instances using mono-alpha substituents (e.g., in the tethered amino acids) are also envisioned.

The structurally-stabilized (e.g., stapled or stitched) peptides can include a drug, a toxin, a derivative of polyethylene glycol; a second peptide; a carbohydrate, etc. Where a polymer or other agent is linked to the structurally-stabilized (e.g., stapled or stitched) peptide, it can be desirable for the composition to be substantially homogeneous.

The addition of polyethelene glycol (PEG) molecules can improve the pharmacokinetic and pharmacodynamic properties of the peptide. For example, PEGylation can reduce renal clearance and can result in a more stable plasma concentration. PEG is a water soluble polymer and can be represented as linked to the peptide as formula:

XO—(CH₂CH₂O)ₙ—CH₂CH₂—Y

where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the peptide. Y may also be a maleimide linkage to a thiol group (including but not limited to, the thiol group of cysteine). Other methods for linking PEG to a peptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain embodiments, macromolecular polymer (e.g., PEG) is attached to a structurally-stabilized (e.g., stapled or stitched) peptide described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH2, phenyl, etc. U.S. Pat. No. 5,446,090 describes a bifunctional PEG linker and its use in forming conjugates having a peptide at each of the PEG linker termini.

The structurally-stabilized (e.g., stapled or stitched) peptides can also be modified, e.g., to further facilitate cellular uptake or increase in vivo stability, in some embodiments. For example, acylating or PEGylating a structurally-stabilized peptide facilitates cellular uptake, increases bioavailability, increases blood circulation, alters pharmacokinetics, decreases immunogenicity and/or decreases the needed frequency of administration.

In some embodiments, the structurally-stabilized (e.g., stapled or stitched) peptides disclosed herein have an enhanced ability to penetrate cell membranes (e.g., relative to non-stabilized peptides). See, e.g., International Publication No. WO 2017/147283, which is incorporated by reference herein in its entirety.

### Methods of Treatment

The disclosure features methods of using any of the structurally-stabilized (e.g., stapled or stitched) peptides (or pharmaceutical compositions comprising said structurally-stabilized peptides) described herein for the prevention and/or treatment of a coronavirus (e.g., betacoronavirus such as SARS-CoV-2) infection or coronavirus disease (e.g., COVID-19). The terms “treat” or “treating,” as used herein, refers to alleviating, inhibiting, or ameliorating the disease or infection from which the subject (e.g., human) is suffering.

The structurally-stabilized (e.g., stapled or stitched) peptides (or compositions comprising the peptides) described herein can be useful for treating a subject (e.g., human subject) having a coronavirus (e.g., betacoronavirus) infection. The structurally-stabilized (e.g., stapled or stitched) peptides (or compositions comprising the peptides) described herein can also be useful for treating a human subject having a coronavirus disease. In certain embodiments, the coronavirus infection is an infection of one of 229E (alpha coronavirus); NL63 (alpha coronavirus); OC43 (beta coronavirus); HKU1 (beta coronavirus); Middle East respiratory syndrome (MERS); SARS-CoV; or SARS-CoV-2. In certain embodiments, the coronavirus disease is caused by a COVID-19 infection.

The structurally-stabilized (e.g., stapled or stitched) peptides (or compositions comprising the peptides) described herein can be useful for preventing a coronavirus (e.g., betacoronavirus) infection in a human subject. The peptides (or compositions comprising the peptides) described herein can also be useful for preventing a coronavirus disease in a subject (e.g., human subject). In certain embodiments, the coronavirus infection is an infection of one of 229E (alpha coronavirus); NL63 (alpha coronavirus); OC43 (beta coronavirus); HKU1 (beta coronavirus); Middle East respiratory syndrome (MERS); SARS-CoV; or SARS-COVID-19. In certain embodiments, the coronavirus disease is caused by a COVID-19 infection.

In certain embodiments, the human subject in need thereof is administered a peptide described in Tables 1-5, or a variant thereof. In certain embodiments, the human subject in need thereof is administered a stapled SARS-CoV-2 HR2 peptide comprising or consisting of SEQ ID NO:9 or a modified version thereof. In certain embodiments, the human subject in need thereof is administered a stapled SARS-CoV-2 HR2 peptide comprising or consisting of SEQ ID NO:10 or a modified version thereof.

In certain embodiments, the human subject in need thereof is administered any one of the peptides having SEQ ID NOs: 11-52, 102, 105, 107, 109, or 111-180 described in Table 1, or a variant thereof (as described herein). Possible variations in these peptides are described in the Structurally Stabilized Peptide section.

Additional guidance is provided in FIG. 6B and FIG. 16D. A variant of these sequences has at least one (e.g., 1, 2, 3, 4, 5) of these properties: (i) binds the recombinant 5-helix bundle protein; (ii) is alpha-helical; (iii) is protease resistant; (iv) inhibits fusion of SARS-CoV-2 with a host cell; and/or (v) inhibits infection of a cell by SARS-CoV-2. In certain embodiments, the human subject in need thereof is administered a peptide having at least 50%, 55%, 60%, 65%, 709%, 75%, 80%, 85%, 90%, 92%, 94%, 95%, identity to any one of the peptides having SEQ ID NOs: 11-52, 102, 105, 107, 109, or 111-180. In certain embodiments, the human subject in need thereof is administered any one of the peptides having SEQ ID NOs: 11-52, 102, 105, 107, 109, or 111-180 but having 1-10 (e.g., 1, 2, 3, 4, 5, 6, 7, 8, 9, 10) amino acid substitutions, insertions, and/or deletions.

In some embodiments, the human subject is infected with a coronavirus (e.g., betacoronavirus). In some embodiments, the human subject is at risk of being infected with a coronavirus (e.g., betacoronavirus). In some embodiments, the human subject is at risk of developing a coronavirus disease (e.g., betacoronavirus). In some instances, a human subject is at risk of being infected with a coronavirus or at risk of developing a coronavirus disease if he or she lives in an area (e.g., city, state, country) subject to an active coronavirus outbreak (e.g., an area where at least 1, at least 2, at least 3, at least 4, at least 5, at least 6, at least 7, at least 8, at least 9, at least 10, at least 20, at least 30, at least 40, or more people have been diagnosed as infected with a coronavirus). In some instances, a human subject is at risk of being infected with a coronavirus or developing a coronavirus disease if he or she lives in an area near (e.g., a bordering city, state, country) a second area (e.g., city, state, country) subject to an active coronavirus outbreak (e.g., an area near (e.g., bordering) a second area where at least 1, at least 2, at least 3, at least 4, at least 5, at least 6, at least 7, at least 8, at least 9, at least 10, at least 20, at least 30, at least 40, or more people have been diagnosed as infected with a coronavirus). In certain embodiments, the coronavirus disease is caused by a SARS-CoV-2 infection. In certain embodiments, the subject has or is at risk of developing COVID-19.

In general, methods include selecting a subject and administering to the subject an effective amount of one or more of the structurally-stabilized (e.g., stapled or stitched) peptides herein, e.g., in or as a pharmaceutical composition, and optionally repeating administration as required for the prevention or treatment of a coronavirus infection or a coronavirus disease and can be administered orally, intranasally, intravenously, subcutaneously, intramuscularly, or topically, including skin, nasal, sinus, respiratory tree, and lung administration. In some instances, the administration is by a topical respiratory application which includes application to the nasal mucosa, sinus mucosa, or respiratory tree, including the lungs. In some instances, topical application includes application to the skin. A subject can be selected for treatment based on, e.g., determining that the subject has a coronavirus (e.g., betacoronavirus such as SARS-CoV-2) infection. The peptides of this disclosure can be used to determine if a subject's is infected with a coronavirus.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### Pharmaceutical Compositions

One or more of any of the structurally-stabilized (e.g., stapled or stitched) peptides described herein can be formulated for use as or in pharmaceutical compositions. The pharmaceutical compositions may be used in the methods of treatment or prevention described herein (see above). In certain embodiments, the pharmaceutical composition comprises a structurally-stabilized (e.g., stapled or stitched) peptide comprising or consisting of an amino acid sequence that is identical to an amino acid sequence set forth in Table 1, except for 1 to 10, 1 to 9, 1 to 8, 1 to 7, 1 to 6, 1 to 5, 1 to 4, 1 to 3, 1 to 2, or 1 amino acid substitution, insertion, or deletion. These changes to the amino acid sequences can be made on the non-interacting alpha-helical face of these peptides (i.e., to the amino acids that do not interact with the coronavirus 5 helix bundle) and/or on the interacting alpha-helical face (i.e., to the amino acids that interact with the coronavirus 5 helix bundle). Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays and/or solutions).

In some instances, pharmaceutical compositions can include an effective amount of one or more structurally-stabilized (e.g., stapled or stitched) peptides. The terms “effective amount” and “effective to treat,” as used herein, refer to an amount or a concentration of one or more structurally-stabilized (e.g., stapled or stitched) peptides or a pharmaceutical composition described herein utilized for a period of time (including acute or chronic administration and periodic or continuous administration) that is effective within the context of its administration for causing an intended effect or physiological outcome (e.g., treatment of infection).

Pharmaceutical compositions of this invention can include one or more structurally-stabilized (e.g., stapled or stitched) peptides described herein and any pharmaceutically acceptable carrier and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

In some instances, one or more structurally-stabilized (e.g., stapled or stitched) peptides disclosed herein can be conjugated, for example, to a carrier protein. Such conjugated compositions can be monovalent or multivalent. For example, conjugated compositions can include one structurally-stabilized (e.g., stapled or stitched) peptide disclosed herein conjugated to a carrier protein. Alternatively, conjugated compositions can include two or more structurally-stabilized (e.g., stapled or stitched) peptides disclosed herein conjugated to a carrier.

As used herein, when two entities are “conjugated” to one another they are linked by a direct or indirect covalent or non-covalent interaction. In certain embodiments, the association is covalent. In other embodiments, the association is non-covalent. Non-covalent interactions include hydrogen bonding, van der Waals interactions, hydrophobic interactions, magnetic interactions, electrostatic interactions, etc. An indirect covalent interaction occurs when two entities are covalently connected, optionally through a linker group.

Carrier proteins can include any protein that increases or enhances immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561, 1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

### Methods of Making Stapled or Stitched Peptides

In one aspect this disclosure features a method of making a structurally-stabilized peptide. The method involves (a) providing a peptide comprising at least two non-natural amino acids with olefinic side chains (e.g., SEQ ID NO 11-52 or 112-180), and (b) cross-linking the peptide. In some instances, cross-linking the peptide is by a ruthenium catalyzed metathesis reaction.

Stapled peptide synthesis: Fmoc-based solid-phase peptide synthesis was used to synthesize stapled peptide fusion inhibitors in accordance with our reported methods for generating all-hydrocarbon stapled peptides (Bird et al., Curr. Protocol. Chem, Biol., 3(3):99-117 (2011; Bird et al., Methods Enzymol., 446:369-86(2008) To achieve the various staple lengths, α-methyl, α-alkenyl amino acids were installed in specific pairings at discrete positions, such as for i, i+4 positioning the use of two S-pentenyl alanine residues (S5). For the stapling reaction, Grubbs 1st generation ruthenium catalyst dissolved in dichloroethane was added to the resin-bound peptides. To ensure maximal conversion, three to five rounds of stapling were performed. The peptides were then cleaved off of the resin using trifluoroacetic acid, precipitated using a hexane:ether (1:1) mixture, air dried, and purified by LC-MS. All peptides were quantified by amino acid analysis.

Stitched peptide synthesis: Methods of synthesizing the stitched peptides described herein are known in the art. Nevertheless, the following exemplary method may be used. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Derivatization of stapled or stitched peptides with PEG4-cholesterol: 200 mg of Boc-PEG4-COOH (www.bichempeg.com/product/Boc-NH-PEG4-COOH.html) is dissolved in 10 mL THF. 400 mg cholesterol (Sigma) is then added with stirring, followed by 0.1 mL of diisopropylcarbodiimide and 7 mg dimethylamiopyridine. The reaction is monitored by LCMS on a C3 column and is typically complete at 1 hr. 10 mL of trifluoracetic acid is added and stirred for 15 min, again with monitoring by LCMS. The solvent is removed and the crude material dissolved in 5 mL of THF and purified by prep LCMS. Product fractions are pooled and lyophilized. The dry product is dissolved in 10 mL THF and 1.5 mL of diisopropylethylamine is added followed by dropwise addition of 0.36 mL bromoacetylbromide. LCMS is used to confirm that the reaction is complete, typically after 20 min. The product, bromoacetylated PEG-4 cholesterol, is purified by LCMS. Reaction of BrAc-PEG4-chol with cysteine containing peptides is then accomplished as follows: 5 mg of peptide (e.g. DISGINASVVNIQXEIDXLNEVAKXLNEXLIDLQELGSGSGC) is dissolved in 350 μL of DMF (5 mM) and 350 μL of a 10 mM solution of BrAc-PEG4-Chol in DMF is then added, followed by 35 μL of 50 mM TCEP in water, and finally 3.2 μL DIEA (10 eq relative to peptide) is added with stirring. The reaction is monitored by LCMS on a C3 column. The cholesterol-peptide adduct is purified by prep LCMS after overnight reaction.

The peptides of this invention can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH2 protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Insertion of a stitching amino acid may be performed as described in, e.g., Young and Schultz, J Biol Chem. 2010 Apr. 9; 285(15): 11039-11044. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from, e.g., Advanced Chemtech or Symphony X. Peptide bonds can be replaced, e.g., to increase physiological stability of the peptide, by: a retro-inverso bonds (C(O)—NH); a reduced amide bond (NH—CH₂); a thiomethylene bond (S—CH2 or CH2-S); an oxomethylene bond (O—CH2 or CH2-O); an ethylene bond (CH2-CH2); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH3; and a fluoro-ketomethylene bond (C(O)—CFR or CFR—C(O) wherein R is H or F or CH3.

The peptides can be further modified by: acetylation, amidation, biotinylation, cinnamoylation, famesylation, fluoresceination, formylation, myristoylation, palmitoylation, other lipidation (e.g. cholesterol), phosphorylation (Ser, Tyr or Thr), stearoylation, succinylation and sulfurylation. As indicated above, peptides can be conjugated to, for example, polyethylene glycol (PEG); alkyl groups (e.g., C₁-C₂₀ straight or branched alkyl groups); fatty acid radicals; and combinations thereof. α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). In some instances for peptides where an i linked to i+7, i+7 linked to i+14 stitch is used (four turns of the helix stabilized): one R-octenyl alanine (e.g., (R)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one bis-pentenyl glycine (e.g., α,α-Bis(4′-pentenyl)glycine), and one S-octenyl alanine (e.g., (S)-α-(7′-octenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. R-octenyl alanine is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids N-Fmoc-α,α-Bis(4′-pentenyl)glycine, (S)—N-Fmoc-α-(4′-pentenyl)alanine, (R)—N-Fmoc-α-(7′-octenyl)alanine, (R)—N-Fmoc-α-(7′-octenyl)alanine, and (R)—N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stitching, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some instances, the peptides are substantially free of non-stitched or non-stapled peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

Properties of the stitched or stapled peptides of the disclosure can be assayed, for example, using the methods described below and in the Examples.

### Assays to Determine Characteristics and Effectiveness of Stabilized Peptides

Assays to Determine α-Helicity: Compounds are dissolved in an aqueous solution (e.g. 5 μM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol., 1986).

Assays to Determine Melting Temperature (Tm): Cross-linked or the unmodified template peptides are dissolved in distilled H₂O or other buffer or solvent (e.g. at a final concentration of 50 μM) and Tm is determined by measuring the change in ellipticity over a temperature range (e.g. 4 to 95° C.) on a spectropolarimeter (e.g., Jasco J-710, Aviv) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In Vitro Protease Resistance Assays: The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The peptidomimetic macrocycles of the present invention may be subjected to in vitro enzymatic proteolysis (e.g. trypsin, chymotrypsin, pepsin) to assess for any change in degradation rate compared to a corresponding uncrosslinked or alternatively stapled polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Peptidomimetic macrocycles and/or a corresponding uncrosslinked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

Plasma Stability Assay: Stapled peptide stability can be tested in freshly drawn mouse plasma collected in lithium heparin tubes. Triplicate incubations are set up with 500 μl of plasma spiked with 10 μM of the individual peptides. Samples are gently shaken in an orbital shaker at 37° C. and 25 μl aliquots are removed at 0, 5, 15, 30, 60, 240, 360 and 480 min and added to 100 μl of a mixture containing 10% methanol:10% water:80% acetonitrile to stop further degradation of the peptides. The samples are allowed to sit on ice for the duration of the assay and then transferred to a MultiScreen Solvinert 0.45 μm low-binding hydrophilic PTFE plate (Millipore). The filtrate is directly analyzed by LC-MS/MS. The peptides are detected as double or triple charged ions using a Sciex 5500 mass spectrometer. The percentage of remaining peptide is determined by the decrease in chromatographic peak area and log transformed to calculate the half-life.

In Vivo Protease Resistance Assays: A key benefit of peptide stapling is the translation of in vitro protease resistance into markedly improved pharmacokinetics in vivo.

Liquid chromatography/mass spectrometry-based analytical assays are used to detect and quantitate SAH-SARS-CoV-2 levels in plasma. For pharmacokinetic analysis, peptides are dissolved in sterile aqueous 5% dextrose (1 mg/mL) and administered to C57BL/6 mice (Jackson Laboratory) by bolus tail vein or intraperitoneal injection (e.g. 5, 10, 25, 50 mg/kg). Blood is collected by retro-orbital puncture at 5, 30, 60, 120, and 240 minutes after dosing 5 animals at each time point. Plasma is harvested after centrifugation (2,500×g, 5 minutes, 4° C.) and stored at −70° C. until assayed. Peptide concentrations in plasma are determined by reversed-phase high performance liquid chromatography with electrospray ionization mass spectrometric detection (Aristoteli et al., Journal of Proteome Res., 2007; Walden et al., Analytical and Bioanalytical Chem., 2004). Study samples are assayed together with a series of 7 calibration standards of peptide in plasma at concentrations ranging from 1.0 to 50.0 μg/mL, drug-free plasma assayed with and without addition of an internal standard, and 3 quality control samples (e.g. 3.75, 15.0, and 45.0 μg/mL). Standard curves are constructed by plotting the analyte/internal standard chromatographic peak area ratio against the known drug concentration in each calibration standard. Linear least squares regression is performed with weighting in proportion to the reciprocal of the analyte concentration normalized to the number of calibration standards. Values of the slope and y-intercept of the best-fit line are used to calculate the drug concentration in study samples. Plasma concentration-time curves are analyzed by standard noncompartmental methods using WinNonlin Professional 5.0 software (Pharsight Corp., Cary, NC), yielding pharmacokinetic parameters such as initial and terminal phase plasma half-life, peak plasma levels, total plasma clearance, and apparent volume of distribution.

Persistence of stabilized alpha-helix of COVID-19 (SAH-SARS-CoV-2) peptides in the nasal mucosa after topical administration (i.e. nose drops) and in the respiratory mucosa after nebulization is examined in the context of pre- and post-infection blockade of viral fusion and dissemination. Mice are exposed to single SAH-SARS-CoV-2 treatment by nose drop or nebulizer at a series of intervals preceding intransal infection with rgCOVID-19, and the duration of protection from mucosal infection (assessed histologically as described above or by PCR as describe below) used to measure the relative mucosal stability and prophylactic efficacy of SAH-SARS-CoV-2 constructs.

In vitro Binding Assays: To assess the binding and affinity of peptidomimetic macrocycles and peptidomimetic precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules or peptides and then bound to proteins of high apparent molecular weights (e.g. FITC-labeled peptides bound to a large protein) emit higher levels of polarized fluorescence due to their slower rates of rotation upon protein binding as compared to fluorescent tracers attached to smaller molecules or peptides alone (e.g. FITC-labeled peptides that are free in solution).

In vitro Displacement Assays to Characterize Antagonists of Peptide-Protein Interactions: To assess the binding and affinity of compounds that antagonize the interaction between a peptide and an acceptor protein, a fluorescence polarization assay (FPA) utilizing a fluoresceinated peptide or peptidomimetic macrocycle derived from a template peptide sequence is used, for example. The FPA technique measures the molecular orientation and mobility using polarized light and fluorescent tracer. When excited with polarized light, fluorescent tracers (e.g., FITC) attached to molecules that are then bound to proteins with high apparent molecular weights (e.g. FITC-labeled peptides bound to a large protein) emit higher levels of polarized fluorescence due to their slower rates of rotation as compared to the FITC-derivatized molecules alone (e.g. FITC-labeled peptides that are free in solution). Compounds that antagonizes the interaction between the fluoresceinated peptide and an acceptor protein will be detected in a competitive binding FPA experiment and the differential potency of compounds in disrupting the interaction can be quantified and compared.

Five helix bundle protein production and fluorescence polarization assay: A C-terminal Hexa-His (SEQ ID NO: 101) tagged recombinant 5-helix bundle (5HB) protein is designed containing 5 of the 6 helices that comprise the core of the SARS-CoV-2 S trimer of hairpins, connected by short peptide linkers in accordance with the design of the gp41 5-HB (Root et al. Science, 291(5505):884-8 (2001); Bird et al., J. Clin Invest. 2014 May; 124(5):2113-24). The plasmid is transformed into Escherichia coli BL21 (DE3), cultured in Luria broth, and induced with 0.1 M isopropyl β-D-thiogalactoside overnight at 37° C. The cells are harvested by centrifugation for 20 minutes at 5,000 g, resuspended in buffer A (100 mM NaH2PO4, 20 mM Tris, 8 M urea; pH 7.4), and lysed by agitation at 4° C. overnight. The mixture is clarified by centrifugation (35,000 g for 30 minutes) before binding to a nickel-nitrilotriacetate (Ni-NTA) agarose (Qiagen) column at room temperature. The bound 5-HB is washed with buffer A (pH 6.3), eluted with buffer A (pH 4.5), renatured by diluting (1:2) with PBS (50 mM sodium phosphate, 100 mM NaCl; pH 7.5), and concentrated in a 10-kDa Amicon centricon (diluting and reconcentrating 7 times), yielding approximately 1 mg/ml protein solution. Purity of the protein is assessed by SDS-PAGE and determined to be >90%. Fluoresceinated peptides of the SARS-CoV-2 S HR2 (25 nM) are incubated with 5-HB protein at the indicated concentrations in room temperature binding buffer (50 mM sodium phosphate, 100 mM NaCl; pH 7.5). Direct Binding activity at equilibrium (e.g. 10 minutes) is measured by fluorescence polarization using a SpectraMax M5 microplate reader (BMG Labtech). For a competitive binding assay, A fixed concentration of FITC-peptide and 5-HB protein reflecting the EC90 for direct binding is then incubated with a serial dilution of acetylated SAH-SARS-CoV-2 peptides to generate competition curves for comparative analyses. Binding assays are run in triplicate, and Kis are calculated by nonlinear regression analysis of the competition binding isotherms using Prism software (GraphPad).

Assay to Screen for Binding Activity to the SARS-CoV-2 5 Helix Bundle:

In some instances, the methods disclosed herein include direct and competitive screening assays. For example, methods can include determining whether an agent alters (e.g., reduces) binding of one or more of the peptides disclosed herein to SARS-CoV-2 (e.g., to SARS-CoV-2 5-helix bundle). In some instances, methods include (i) determining a level of binding between one or more of the peptides disclosed herein and SARS-CoV-2 (e.g., to SARS-CoV-2 5-helix bundle) (e.g., in the absence of an agent); and (ii) detecting the level of binding between one or more peptides (e.g., the one or more peptides of (i)) and SARS-CoV-2 (e.g., to SARS-CoV-2 5-helix bundle) in the presence of an agent, wherein a change (e.g., reduction) in the level of binding between the one or more peptides and SARS-CoV-2 (e.g., to SARS-CoV-2 5-helix bundle) indicates that the agent is a candidate agent that binds to SARS-CoV-2; and (iii) selecting the candidate agent. In some instances, step (i) includes contacting one or more peptides with SARS-CoV-2 (e.g., to SARS-CoV-2 5-helix bundle) and detecting the level of binding between one or more peptides with SARS-CoV-2 (e.g., to SARS-CoV-2 5-helix bundle). In some instances, step (ii) includes contacting the one or more peptides and the agent with SARS-CoV-2 (e.g., to SARS-CoV-2 5-helix bundle) and detecting the level of binding between one or more peptides with SARS-CoV-2 (e.g., to SARS-CoV-2 5-helix bundle). SARS-CoV-2 (e.g., to SARS-CoV-2 5-helix bundle) can be contacted with the one or more peptides and the agent at the same time or at different times (e.g., the one or more peptides can be contacted with SARS-CoV-2 (e.g., to SARS-CoV-2 5-helix bundle) before or after the agent). In some embodiments, candidate agents are administered to a suitable animal model (e.g., an animal model of COVID-19) to determine if the agent reduces a level of COVID-19 infection in the animal.

In some instances, one or both of the peptide and the SARS-CoV-2 helix bundle can include a label, allowing detection of the peptide and/or the SARS-CoV-2 helix bundle. In some instances, the peptide includes a label. In some instances, the SARS-CoV-2 helix bundle includes a label. In some instances, both the peptide and the SARS-CoV-2 helix bundle include a label. A label can be any label known in the art, including but not limited to a fluorescent label, a radioisotope label, or an enzymatic label. In some instances, the label is directly detectable by itself (e.g., radioisotope labels or fluorescent labels). In some instances, (e.g., in the case of an enzymatic label), the label is indirectly detectable, e.g., by catalyzing chemical alterations of a chemical substrate compound or composition, which chemical substrate compound or composition is directly detectable.

Competitive SARS-CoV-2 5-HB binding assay by ELISA: Microwells are coated overnight at 4° C. with 50 μl of PBS containing neutravidin (4 μg/ml). Wells are washed twice with PBS containing 0.05% Tween 20 (PBS-T), and blocked with 4% BSA in PBS-T for 45 min at 37° C. Next, 50 μl of 250 nM biotinylated-PEG2-SARS-CoV-2 HR2 (SEQ ID NO: 9) is added in PBS-T with 1% BSA and incubated with shaking for 1 hr followed by 4× washes with 300 μl of PBS-T. Then, a 1:2 serial dilution of SARS-CoV-2 peptides starting at 10 μM containing 50 nM of recombinant 5-HB in 50 μL of PBS-T with 1% BSA is added to the plate and shaken at room temperature for 2 hr followed by 4× washes with 300 μl of PBS-T. Finally, 50 μL of a 1:5000 dilution of goat polyclonal to 6× His tag-HRP conjugated is added. Following incubation at RT for 40 min, the wells are washed five times, and developed by adding 50 μl of tetramethylbenzidine (TMB) solution. After 20 min, wells containing TMB solution are stopped by adding 50 μl of H₂SO₄ (2 M), and the absorbance at 450 nm is read on a microplate reader (Molecular Devices). The concentration of competitor peptide corresponding to a half-maximal signal (IC50) is determined by interpolation of the resulting binding curve using Prism software (Graphpad). Each peptide competitor is tested in triplicate in at least two separate experiments.

Cellular Penetrability Assays: To measure the cell penetrability of peptides or crosslinked polypeptides, intact cells are incubated with fluoresceinated crosslinked polypeptides (10 μM) for 4 hours in serum-free media or in media supplemented with human serum at 37° C., washed twice with media and incubated with trypsin (0.25%) for 10 min at 37° C. The cells are washed again and resuspended in PBS. Cellular fluorescence is analyzed, for example, by using either a FACSCalibur flow cytometer or Cellomics' KineticScan® HCS Reader.

Antiviral Efficacy Assays: The efficiency of SAH-SARS-CoV-2 peptides in preventing and treating COVID-19 infection are evaluated in monolayer cell cultures. A viral detection platform has been developed for SARS-CoV-2 based on previous screens against Ebolaviruses (see, Anantpadma M. et al., Antimicrob Agents Chemother. 2016; 60(8):4471-81. Epub 2016/05/11. doi: 10.1128/AAC.00543-16. PubMed PMID: 27161622; PMCID: PMC4958205). Vero E6 cells plated in 384-well format are treated for 1 hour with a serial dilution of stapled peptides (e.g. 10 μM starting dose), performed in triplicate, followed by 4 hour challenge with SARS-CoV-2 to achieve control infection of 10-20% cells (the pre-determined optimal infectivity to assess the dynamic range of test compounds in the assay). Infected cells are then washed, fixed with 4% paraformaldehyde, rewashed in PBS, immune-stained with anti-SARS-CoV-2 nucleocapsid monoclonal antibody followed by anti-Ig secondary antibody (Alexa Fluor 488; Life Technologies), and cell bodies counterstained with HCS CellMask blue. Cells are imaged across the z-plane on a Nikon Ti Eclipse automated microscope, analyzed by CellProfiler software, and infection efficiency calculated by dividing infected by total cells. Control cytotoxicity assays are performed using Cell-Titer Glo (Promega) and LDH release (Roche) assays.

In an alternative approach, qPCR based viral detection is used in natively-susceptible human-derived Huh770 and Calu-371 cells that express ACE2, and also MatTek Life Sciences primary lung epithelial and alveolar cell models, infected with SARS-CoV-2 virus (e.g. USA-WA1/2020; Hongkon VM20001061). Cultured cells are treated for 1 hour with a serial dilution of stapled peptides followed by challenge with SARS-CoV-2. Culture supernatants are sampled, virus lysed in the presence of RNAse inhibitor, and RT and qPCR performed as described. See Suzuki et al. J Vis Exp. 2018(141). Epub 2018/11/20. doi: 10.3791/58407. CDC-validated BHQ quenched dye pair primers are purchased from IDT and genome equivalents calculated from Ct values.

In yet another approach, antiviral activity of SAH-SARS-CoV-2 stapled peptides are assessed using pseudotyped virus. The 293T-hsACE2 stable cell line (Cat #C-HA101) and the pseudotyped SARS-CoV-2 (Wuhan-Hu-1 strain) particles with GFP (Cat #RVP-701G, Lot #CG-113A) reporters are used (Integral Molecular). The neutralization assay is carried out according to the manufacturers' protocols. In brief, 5 μL of a single dose of peptide (5 μM final dose) is incubated with 5 μL pseudotyped SARS-CoV-2-GFP for 1 hr at 37° C. in a 384 well black clear bottom plate followed by addition of 30 μL of 1,000 293T-hsACE2 cells in 10% FBS DMEM, phenol red free media and placed in a humidified incubator for 48 or 72 hrs. Hoechst 33342 and DRAQ7 dyes are added and the plate imaged on a Molecular Devices ImageXpress Micro Confocal Laser at 10× magnification. GFP (+) cells are counted and plotted using Prism software (Graphpad).

To evaluate the capacity of lead stapled peptides to prevent SARS-CoV-2 infection, K18-hACE2 (Jackson Laboratory) mice (n=10 per arm; 5 male, 5 female) are administered intranasally or by the oropharyngeal route with stapled peptide or vehicle and 24 hours later a viral dosage of 104 PFU is inoculated intranasally. Mice are euthanized 4 days later (peak of viremia) for evaluation by necropsy and viral load as quantitated by qPCR from supernatant samples of lung homogenates, prepared as described using a tissuelyzer (Qiagen). See Bao L et al. Nature. 2020. Epub 2020/05/08; doi: 10.1038/s41586-020-2312-y. To evaluate the capacity of lead stapled peptides to treat or mitigate established SARS-CoV-2 infection, K18-hACE2 mice (n=10 per arm; 5 male, 5 female) are inoculated intranasally at a viral dosage of 104 PFU on day 1, followed by daily oropharyngeal or intraperitoneal treatment with stapled peptide or vehicle for 10 days (days 2-12). In an alternate design, dosing is delayed until 3-5 days post-inoculation to simulate symptom- or positive test-driven initiation of therapy. Mice are continuously monitored to record body weights and clinical signs, with disease progression scored as >10% body weight loss, labored breathing, and/or failure to thrive. Doses for the most effective compound and route are then be refined in both prevention and treatment studies to determine the minimum dose to protect mice. The same experimental design is used except that the 4 treatment groups (n=10; 5 male, 5 female) receive the original dose and then 3 progressively lowered doses in 4-fold increments.

Clinical Trials: To determine the suitability of the cross-linked polypeptides of the invention for treatment of humans, clinical trials can be performed. For example, patients exposed to SARS-CoV-2 infection or diagnosed with SARS-CoV-2 infection are selected and separated in treatment and one or more control groups, wherein the treatment group is administered a crosslinked polypeptide of the invention, while the control groups receive a placebo or a known antiviral drug. The treatment safety and efficacy of the cross-linked polypeptides of the invention can thus be evaluated by performing comparisons of the patient groups with respect to factors such as prevention of symptoms, time to resolution of symptoms, and/or overall infection severity. In another example, uninfected patients are identified and are given either a cross-linked polypeptide or a placebo. After receiving treatment, patients are followed. In both examples, the SARS-CoV-2-exposed patient group treated with a cross-linked polypeptide would avoid the development of infection, or a patient group with SARS-CoV-2 infection would show resolution of or relief from symptoms compared to a patient control group treated with a placebo.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Design and Synthesis of SARS-CoV-2 HR2 Stapled Peptides

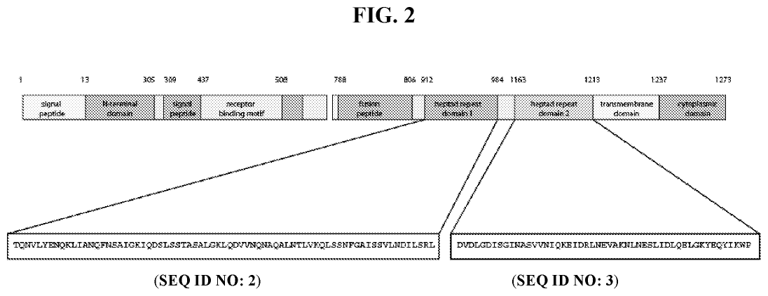

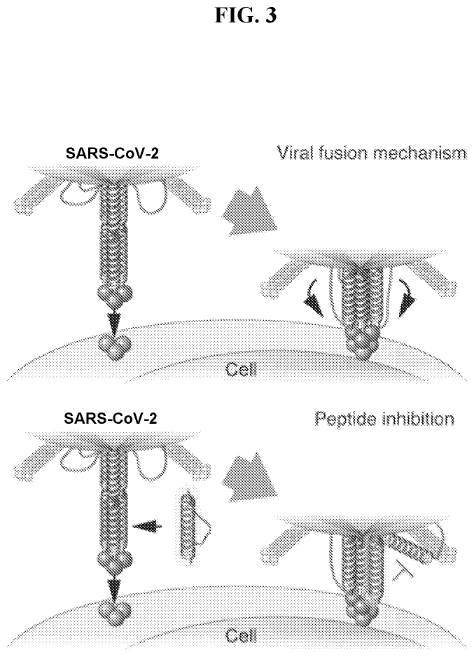

To design peptides that could block the fusion of the coronavirus to a host cell, a series of stapled peptides bearing differentially localized chemical staples were designed. The differentially localized chemical staples were located within the SARS-CoV-2 HR2 domain (i.e., amino acids 1169-1210 or 1179-1197) of the sequence of the surface glycoprotein [Severe acute respiratory syndrome coronavirus 2] (see, FIG. 1) by replacing native residues with α, α-disubstituted non-natural olefinic residues (“X”) at select (i, i+4) or (i, i+7) positions and combinations thereof in the form of double staples or stitches, followed by ruthenium-catalyzed olefin metathesis (see, Table 1). Some designs incorporate staples on the non-interacting amphiphilic face of the helix or at positions at the border of the hydrophobic interaction face with the amphiphilic face of the helix (FIGS. 4 and 5).

SAH-SARS-CoV-2 constructs were designed by replacing two naturally occurring amino acids with the non-natural S-2-(4′-pentenyl) alanine (S5) amino acids at i, i+4 positions (i.e. flanking 3 amino acids) to generate a staple spanning one α-helical turn, or a combination of (R)-2-(((9H-fluoren-9-yl)methoxy)carbonylamino)-2-methyl-dec-9-enoic acid (R8) and S5 at i, i+7 positions, respectively, to generate a staple spanning two α-helical turns. Asymmetric syntheses of α, α-disubstituted amino acids were performed as previously described in detail (Schafmeister et al., J. Am. Chem. Soc., 2000; Walensky et al., Science, 2004; Bird et al. Current Protocols in Chemical Biology, 2011, each of which is incorporated by reference in its entirety).

“Staple scanning” was performed to respectively identify residues and binding surfaces critical for interaction, which dictates the design of optimized constructs and negative control mutants. The N-termini of SAHs were capped with acetyl or a fluorophore (e.g. FITC, rhodamine), depending upon the experimental application.

Doubly stapled peptides were generated by installing two-S5-S5, two —R8-S5, or other combinations of crosslinking non-natural amino acids. Multiply stapled or stitched peptides are generated using similar principles.

Synthesis of the SAH-SARS-CoV-2 peptides shown in Table 1 was performed using solid phase Fmoc chemistry and ruthenium-catalyzed olefin metathesis, followed by peptide deprotection and cleavage, purification by reverse phase high performance liquid chromatography/mass spectrometry (LC/MS), and quantification by amino acid analysis (AAA) (Bird et al., Methods Enzymol., 2008).

### Example 2: Assessing Alpha-Helical Stabilization of SARS-CoV-2 HR2 Stapled Peptides

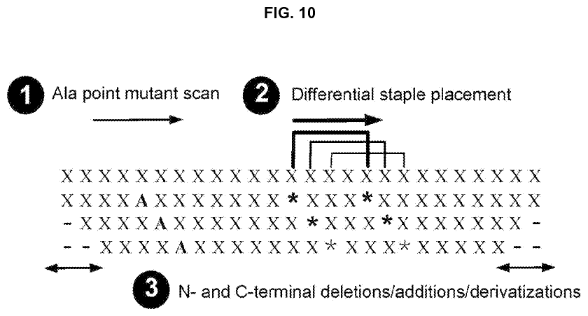

Generally, short peptides do not exhibit significant α-helical structure in solution. This is because the entropic cost of maintaining a conformationally-restricted structure is not overcome by the enthalpic gain from hydrogen bonding of the peptide backbone. To document secondary structure improvements of hydrocarbon-stapled peptides, circular dichroism (CD) spectra was recorded and analyzed on a Model 410 Aviv Biomedical spectrometer. Five scans from 190-260 nm in 0.5 nm increments with 0.5 sec averaging time were collectively averaged to obtain each spectrum using a 1 mm path length cell. The target peptide concentration for CD studies was 25-50 μM in 50 mM potassium phosphate (pH 7.5) or Milli-Q deionized water, and exact concentrations were confirmed by quantitative AAA of two CD sample dilutions. The CD spectra were initially plotted as wavelength versus millidegree. Once the precise peptide concentration was confirmed, the mean residue ellipticity [0], in units of degree·cm2·dmol-1·residue-1, was derived from the equation, [0]=millidegree/molar concentration/number of amino acid residues. After conversion to mean residue ellipticity, percent α-helicity was calculated using the equation, % helicity=100×[θ]222/max[θ]222, where max[θ]222=−40,000×[1−(2.5/number of amino acid residues)]. Stapled constructs that reinforce α-helical structure were advanced to protease-resistance testing, binding analyses, and antiviral activity assays. FIG. 12A and FIG. 12B show that unstapled HR2 peptides corresponding to SEQ ID NOs 10, 9, 106 and 110 exhibited little to no alpha-helical structure in solution by circular dichroism analyses, whereas insertion of double staples (i.e., in SEQ ID NOs: 49, 51, 158, and 177) and stitches (i.e., in SEQ ID NOs: 47 and 48) into such sequences effectively induced alpha-helicity, as evidenced by the progressive increase in absorption at [θ]222. Such stapled constructs that reinforced α-helical structure were advanced to protease-resistance testing, binding analyses, and antiviral activity assays.

### Example 3: Determining Protease Resistance of SARS-CoV-2 HR2 Stapled Peptides

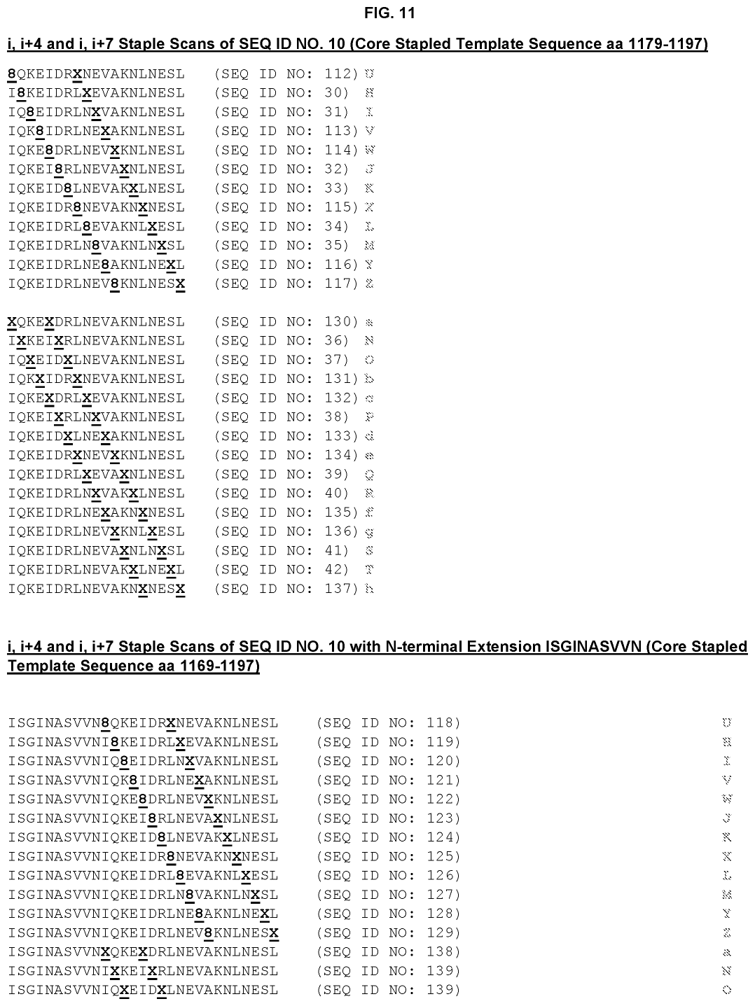

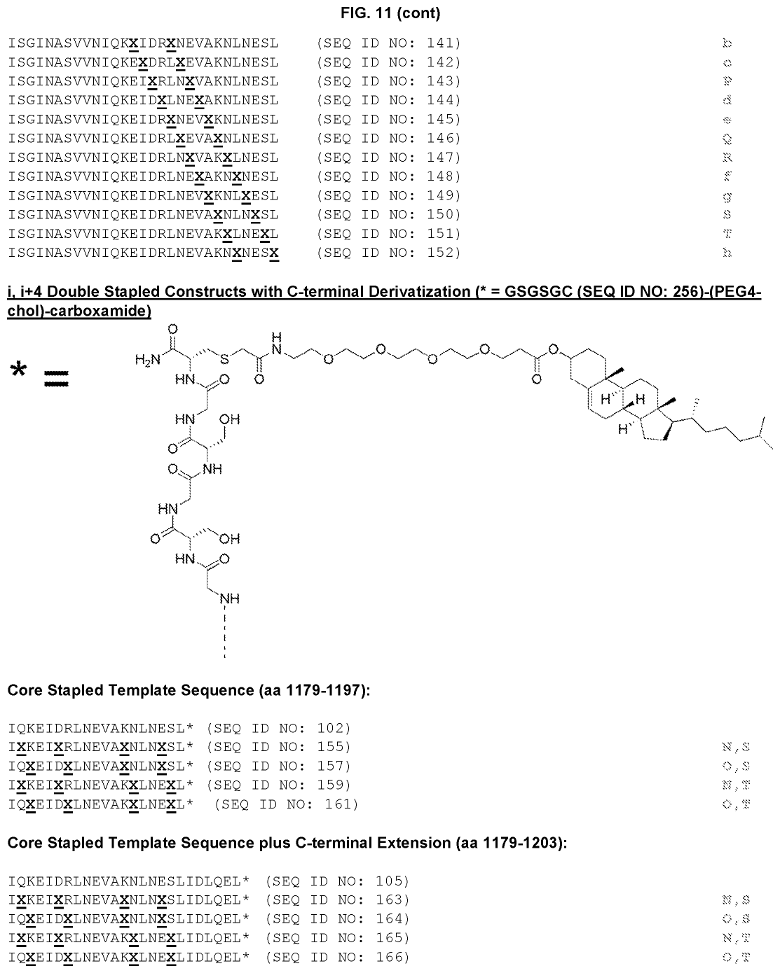

Linear peptides are susceptible to rapid proteolysis in vitro and in vivo, limiting the application of natural peptides for mechanistic analyses and therapeutic use. In contrast, amide bonds engaged in the hydrogen-bonding network of a structured peptide helix are poor enzymatic substrates, as are residues shielded by the hydrocarbon staple itself (Bird et al, PNAS, 2010). To evaluate the relative protease resistance conferred by hydrocarbon stapling, in vitro proteolytic degradation was measured by LC/MS (Agilent 1200) using the following parameters: 20 μL injection, 0.6 mL flow rate, 15 min run time consisting of a gradient of water (0.1% formic acid) to 20-80% acetonitrile (0.075% formic acid) over 10 min, 4 min wash to revert to starting gradient conditions, and 0.5 min post-time. The DAD signal was set to 280 nm with an 8 nm bandwidth and MSD set to scan mode with one channel at (M+2H)/2, +/−1 mass units and the other at (M+3H)/3, +/−1 mass units. Integration of each MSD signal yielded areas under the curve of >10⁸ counts. Reaction samples were composed of 5 μL peptide in DMSO (1 mM stock) and 195 μL of buffer consisting of 50 mM Tris HCl at pH 7.4. Upon injection of the 0 hr time point sample, 2 μL of 100 ng/μL proteinase K (New England Biolabs) was added and the amount of intact peptide quantitated by serial injection over time. An internal control of acetylated tryptophan carboxamide at a concentration of 100 μM is used to normalize each MSD data point. A plot of MSD area versus time yielded an exponential decay curve and half-lives were determined by nonlinear regression analysis using Prism software (GraphPad). FIGS. 13A and 13B show how insertion of double staples or stitches into the core template sequence (aa 1169-1197) conferred striking protease stability compare to the unstapled sequence, depending on the sequence, staple type, and staple location. FIG. 13A shows that double staples or a stitch (SEQ ID NOs 48 and 52) both conferred marked resistance to Proteinase K treatment (half-lives of >1000 min), whereas the unstapled sequence (SEQ ID NO: 10) was rapidly digested (half-life of 35 min). FIG. 13B shows that the longer unstapled HR2 sequence (SEQ ID NO:9) was rapidly digested by Proteinase K (half-life of 25 min) and insertion of double staples 0, S (SEQ ID NO:158) only mildly enhanced proteolytic resistance (half-life of 33 min), whereas insertion of double staples N,S (SEQ ID NO:177) into an alternate HR2-type sequence (SEQ ID NO:111) conferred significant proteolytic resistance to Proteinase K (half-life of 840 min). The protease resistance and stability of stapled peptides were also measured by use of a mouse plasma stability assay. Stapled peptide stability was tested in freshly drawn mouse plasma collected in lithium heparin tubes. Triplicate incubations were set up with 500 μl of plasma spiked with 10 μM of the individual peptides. Samples were gently shaken in an orbital shaker at 37° C. and 25 μl aliquots were removed at 0, 5, 15, 30, 60, 240, 360 and 480 min and added to 100 μl of a mixture containing 10% methanol:10% water:80% acetonitrile to stop further degradation of the peptides. The samples were allowed to sit on ice for the duration of the assay and then transferred to a MultiScreen Solvinert 0.45 μm low-binding hydrophilic PTFE plate (Millipore). The filtrate was directly analyzed by LC-MS/MS. The peptides were detected as double or triple charged ions using a Sciex 5500 mass spectrometer. The percentage of remaining peptide was determined by the decrease in chromatographic peak area and log transformed to calculate the half-life. FIGS. 14A and 14B show that two doubly stapled peptides of the core template sequence (aa 1169-1197), including SEQ ID NO:51 (Staples N,T) in FIG. 14A and SEQ ID NO:52 (Staples 0, T) in FIG. 14B, exhibited no degradation whatsoever over time upon incubation with mouse plasma.

### Example 4: Investigating SARS-CoV-2 Binding Activity of SAH-SARS-CoV-2 Peptides

To measure direct binding affinity for the SARS-CoV-2 fusogenic bundle, a direct fluorescence polarization assay (FPA) was performed using recombinant five-helix bundle protein and fluorescent SARS-CoV-2 HR2 peptides (having excitation wavelengths of 488 nm and emission wavelength of 522 nm), by appending FITC-bAla at the N-terminus of the sequences shown in Table 1. More specifically, a recombinant 5-helix bundle protein (SEQ ID NO:263) was designed containing five of the six helices that comprise the core of the SARS-CoV-2 trimer of hairpins, connected by short peptide linkers in accordance with the design of the SARS-CoV-2 5-helix bundle. Because the recombinant 5-helix bundle lacks the third HR2 helix but is otherwise soluble, stable, and helical, incorporation of the sixth HR2 peptide in the form of FITC-SARS-CoV-2 HR2₍₁₁₇₉₋₁₁₉₇₎ or -SARS-CoV-2 HR2₍₁₁₆₉₋₁₂₁₀₎ peptides, and derivatives thereof, yielded a stable complex, which can be monitored by FPA to measure the direct binding affinity. FPA assays were used to measure and compare the relative binding activities of distinct SARS-CoV-2 HR2 constructs for the 5-helix fusion bundle. FITC-SARS-CoV-2HR2 peptides were mixed with a serial dilution of recombinant 5-helix bundle protein to generate a binding isotherm. Fluorescence polarization (mP units) was measured on a SpectraMax fluorimeter, and EC50 values were calculated by nonlinear regression analysis of competition curves using Prism software (Graphpad).

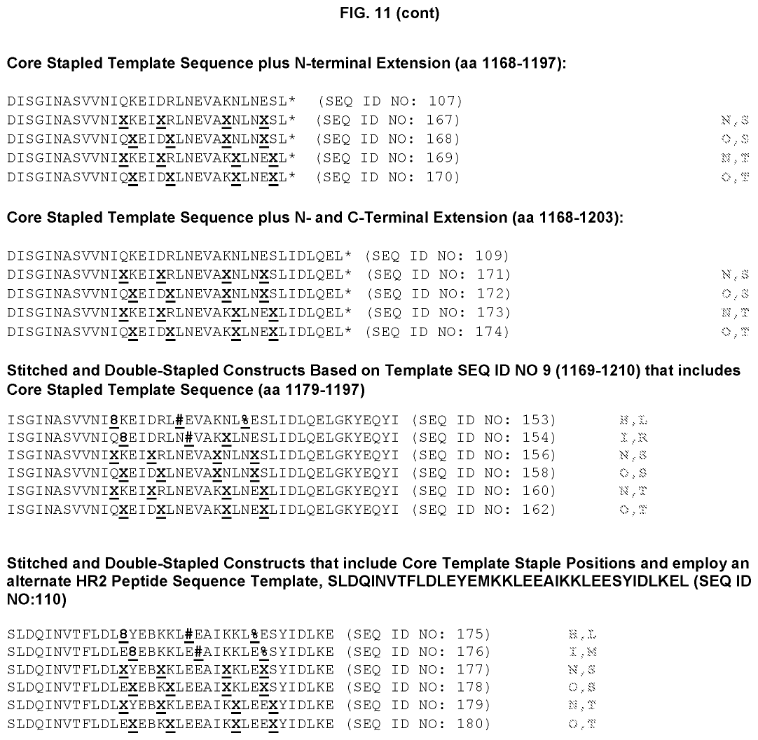

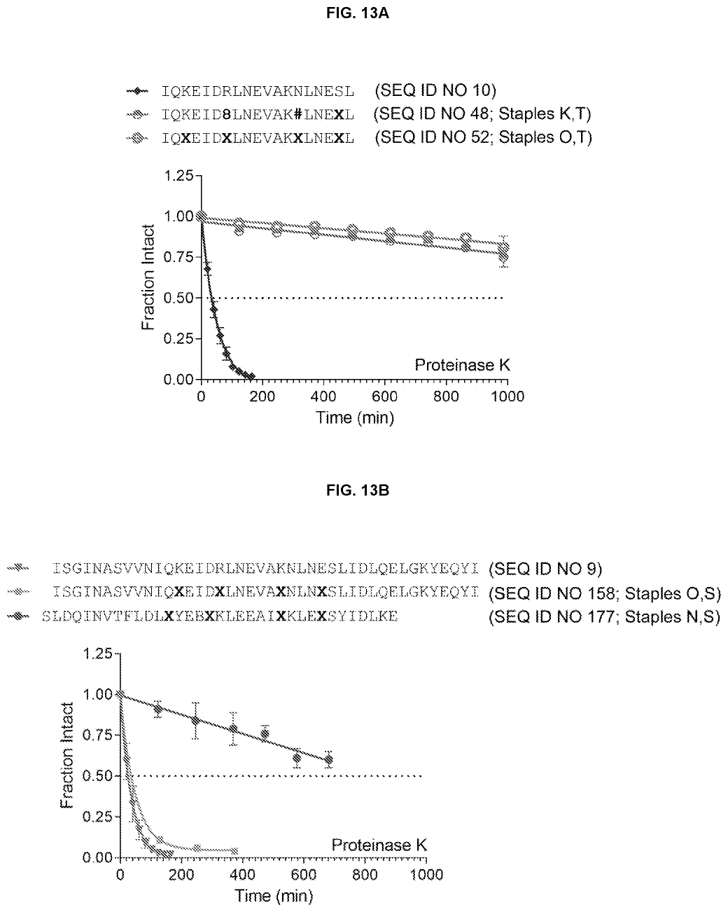

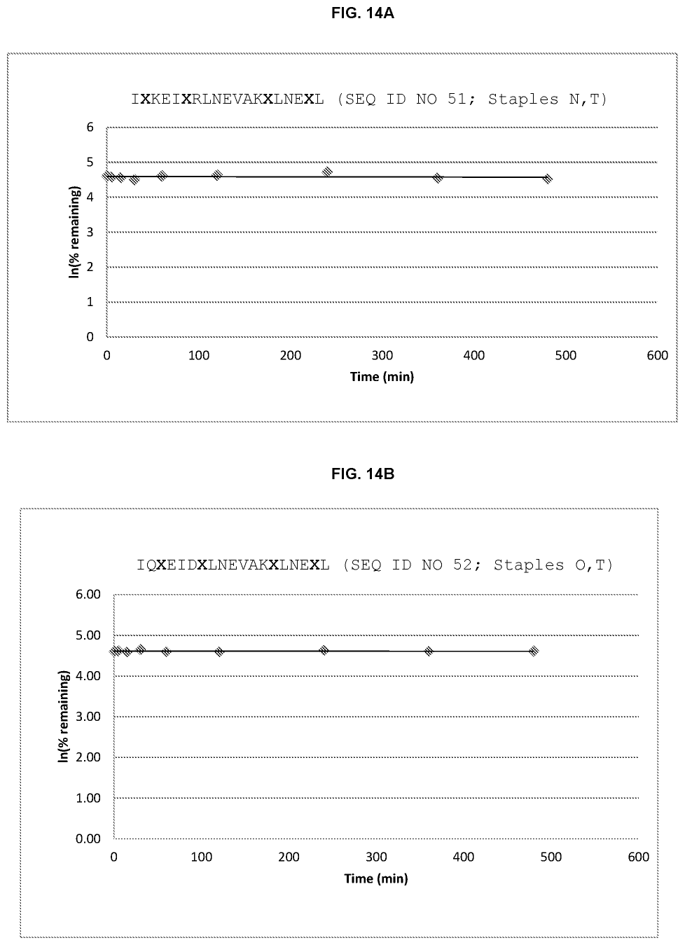

FIGS. 15A and 15B show the results of a direct fluorescence polarization binding assay using the recombinant SARS-CoV-2 5-helix binding protein and an N-terminal FITC derivatized i, i+4 staple scanning library of the core template sequence (aa 1179-1197, SEQ ID NO: 10). FIG. 15A illustrates the differential binding activities of the stapled peptides based on the i, i+4 staple location, as reflected by the change in fluorescence polarization (ΔmP) at 4 μM 5-HB protein concentration. FIG. 15B shows the dose-response curves for the fluorescent i, i+4 staple scanning library to the 5-HB protein, highlighting that depending on the particular staple position, the i, i+4 stapled peptides bind either better, similar to, or worse than the unstapled core template sequence. FIGS. 16A and 16B show the results of a direct fluorescence polarization binding assay using the recombinant SARS-CoV-2 5-helix binding protein and an N-terminal FITC derivatized i, i+7 staple scanning library of the core template sequence (aa 1179-1197, SEQ ID NO:10). FIG. 16A illustrates the differential binding activities of the stapled peptides based on the i, i+7 staple location, as reflected by the change in fluorescence polarization (ΔmP) at 4 μM 5-HB protein concentration. FIG. 16B shows the dose-response curves for the fluorescent i, i+7 staple scanning library to the 5-HB protein, highlighting that depending on the particular staple position, the i, i+7 stapled peptides bind either better, similar to, or worse than the unstapled core template sequence. FIG. 16C shows a helical wheel diagram depicting residues that participate in a favorable (light grey), unfavorable (dark grey), and intermediate (medium grey) i, i+7 staple. Residues that participate in two staples are shown as bisected circles with the leftward semicircle coloration representative of the staple's activity when that residue is incorporated at the N-terminal position of a staple and the rightward semicircle coloration representative of the staple's activity when that residue is incorporated at the C-terminal position of a staple; when a semicircle is colored white, the indicated residue position does not participate in either the N- or C-terminal position of a staple. Staple positions located at the hydrophobic surface disrupt 5-HB binding activity and, unexpectedly, staple positions located at the hydrophilic surface opposite to the 5-HB binding surface were also disfavored (marked by Xs). In contrast, select staple positions at the boundary between the hydrophobic binding surface and the hydrophilic surface were favored (marked by stars). FIGS. 17A and 17B show the results of a direct fluorescence polarization binding assay using the recombinant SARS-CoV-2 5-helix binding protein and N-terminal FITC derivatized double i, i+4 stapled peptides of the core template sequence (aa 1179-1197, SEQ ID NO:10). FIG. 17A illustrates the differential binding activities of the stapled peptides based on double staple locations, as reflected by the change in fluorescence polarization (ΔmP) at 4 μM 5-HB protein concentration. FIG. 17B shows the dose-response curves for the fluorescent double stapled peptides to the 5-HB protein, highlighting that in each example, insertion of the double staples leads to enhanced binding activity compared to the unstapled core template sequence. FIG. 18 shows the results of a direct fluorescence polarization binding assay using the recombinant SARS-CoV-2 5-helix binding protein and N-terminal FITC derivatized double i, i+4 stapled peptides within the context of the longer HR2 (SEQ ID NO:9) and alternate HR2-type (SEQ ID NO:110) sequences. The plot demonstrates the comparative binding activity of the double stapled peptides for the 5-HB of SARS-CoV-2. In each case, insertion of double staples yields stapled peptides with dose-responsive 5-HB binding activity.

Integrating the FPA data across i, i+4 and i, i+7 staple scans, and the evaluation of double stapled constructs across peptide templates of distinct length and sequence further reveals that (1) single stapled peptides with notable binding activity can maintain target affinity in the context of a double stapled peptide, even when the second staple may be less effective or ineffective as a single stapled peptide (e.g. compare single i, i+4 stapled N, T, and O peptides and i, i+4 double stapled N,T and O,T peptides); (2) two staples that each may be less effective or ineffective as single stapled peptides can combine to yield a peptide with improved binding activity in the context of a double stapled peptide (e.g. compare single i, i+4 stapled O and S peptides and i, i+4 double stapled O,S peptide); and (3) double staple combinations that yield favorable binding activity in the context of one HR2 template sequence can also produce favorable binding activity in the context of a distinct HR2-type template sequence (e.g. compare the similar and favorable binding activity of O,T double stapled peptides in the context of the HR2 and EK1 template sequences; FIG. 18). Thus, whereas such binding data can guide iterative peptide design, synthesis and testing of discrete constructs is ultimately required to identify and validate definitive, direct binders of the SARS-CoV-2 5-HB.

An alternative approach to measuring the binding activity of SARS-CoV-2 HR2 stapled peptides involved performing a competitive ELISA assay in which a serial dilution of stapled peptide competes with the long HR2 peptide for binding to the recombinant 5-helix bundle of SARS-CoV-2. Notably, this binding assay measures a distinct activity from the direct FPA in that the stapled peptide construct must be capable of competing with and disrupting the interaction between another HR2 peptide and the 5-HB protein target. Microwells were coated overnight at 4° C. with 50 μl of PBS containing neutravidin (4 μg/ml). Wells were washed twice with PBS containing 0.05% Tween 20 (PBS-T), and blocked with 4% BSA in PBS-T for 45 min at 37° C. Next, 50 μl of 250 nM biotinylated-PEG2-SARS-CoV-2 HR2 (SEQ ID NO:9) was added in PBS-T with 1% BSA and incubated with shaking for 1 hr followed by 4× washes with 300 μl of PBS-T. Then, a 1:2 serial dilution of SARS-CoV-2 peptides starting at 10 μM containing 50 nM of recombinant 5-HB in 50 μL of PBS-T with 1% BSA was added to the plate and shaken at room temperature for 2 hr followed by 4× washes with 300 μl of PBS-T. Finally, 50 μL of a 1:5000 dilution of goat polyclonal to 6× His tag-HRP conjugated was added. Following incubation at RT for 40 min, the wells were washed five times, and developed by adding 50 μl of tetramethylbenzidine (TMB) solution. After 20 min, wells containing TMB solution were stopped by adding 50 μl of H₂SO₄ (2 M), and the absorbance at 450 nm was read on a microplate reader (Molecular Devices). The concentration of competitor peptide corresponding to a half-maximal signal (IC50) was determined by interpolation of the resulting binding curve using Prism software (Graphpad). Each peptide competitor was tested in triplicate in at least two separate experiments.

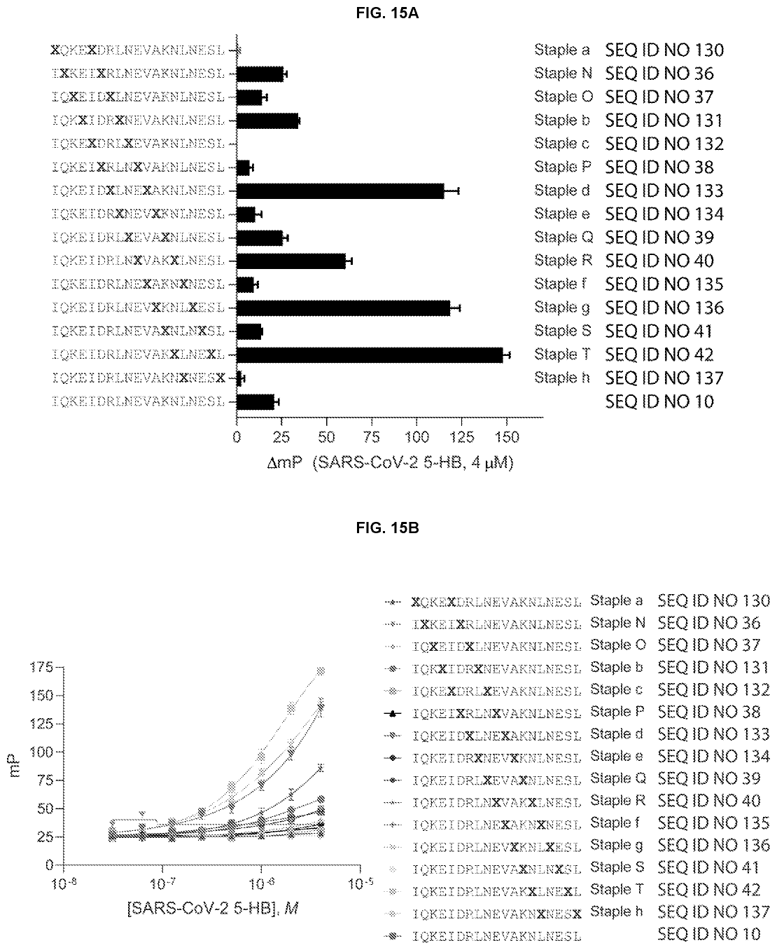

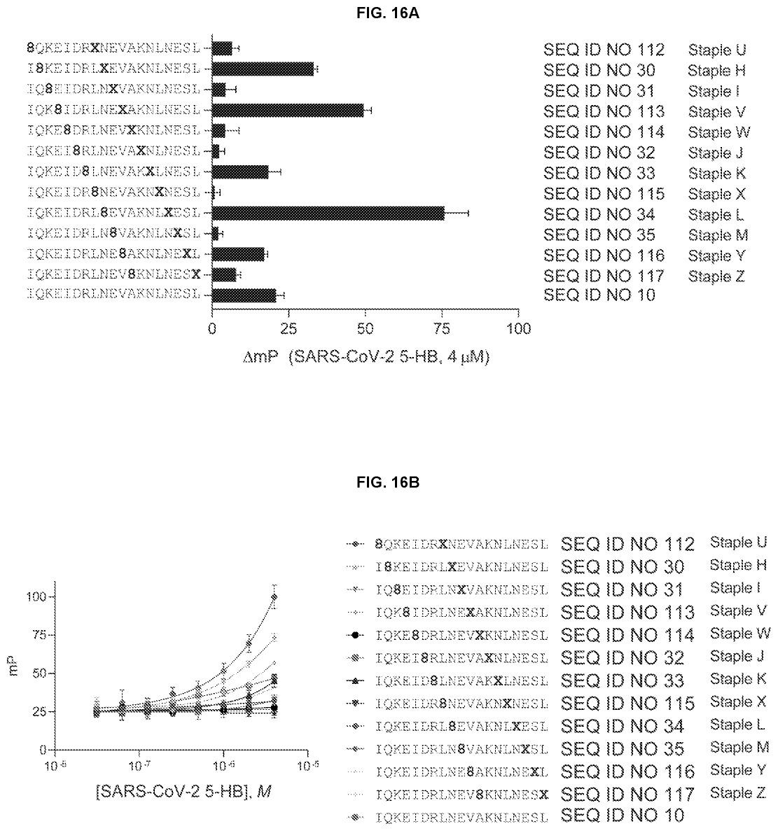

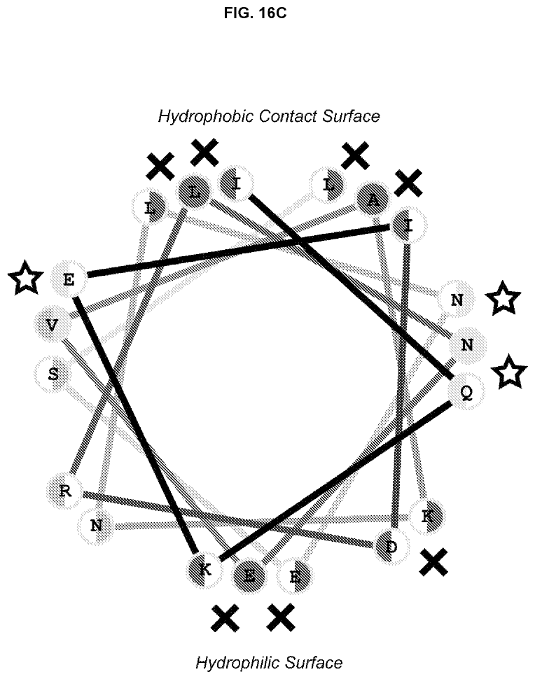

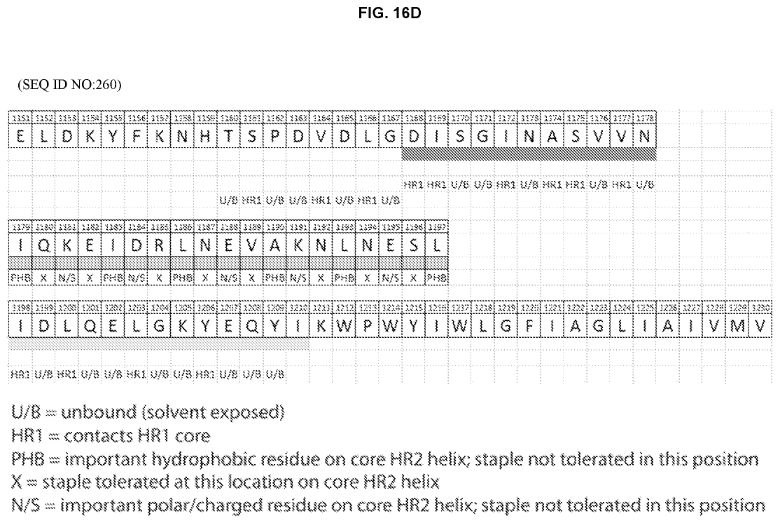

FIGS. 19A-19C show the results of a competitive ELISA binding assay in which the interaction between SARS-CoV-2 5-HB protein and the SARS-CoV-2 unstapled HR2 sequence corresponding to SEQ ID NO:9 was competed by a serial dilution of an i, i+4 staple scanning library of the core template sequence (SEQ ID NO:10) with an N-terminal extension (aa 1169-1178) (SEQ ID NO:103). FIG. 19A shows the full dose-response competitive binding curves and FIG. 19B and FIG. 19C highlight the comparative, competitive binding activity for each construct at 3 μM and 10 μM dosing, respectively. FIG. 20 shows the results of a competitive ELISA binding assay in which the interaction between SARS-CoV-2 5-HB protein and the SARS-CoV-2 unstapled HR2 sequence corresponding to SEQ ID NO:9 was competed by a fixed dose (10 μM) of double stapled and stitched peptides of the core template SARS-CoV-2 HR2 sequence corresponding to SEQ ID NO: 10. Whereas the unstapled core template sequence (SEQ ID NO:10) is unable to compete with the longer HR2 template sequence (SEQ ID NO:9) for binding to the 5-HB, select double stapled (staple combinations O,S and K,T) and stitched (staple combination H,L) peptides of the core template sequence were capable of partially disrupting the binding interaction at 10 μM dosing. FIG. 21 shows the results of a competitive ELISA binding assay in which the interaction between SARS-CoV-2 5-HB protein and the SARS-CoV-2 unstapled HR2 sequence corresponding to SEQ ID NO 9 was competed by dose-responsive treatment with double stapled and stitched peptides of the longer HR2 sequence corresponding to SEQ ID NO:9. The effectiveness at disrupting the 5-HB/HR2 interaction depended upon the staple type and staple positioning of the double staples and stitches in the core template sequence (SEQ ID NO:10) within the context of the longer HR2 peptide (SEQ ID NO:9). FIG. 22 shows the results of a competitive ELISA binding assay in which the interaction between SARS-CoV-2 5-HB protein and the SARS-CoV-2 unstapled HR2 sequence corresponding to SEQ ID NO:9 was competed by dose-responsive treatment with double stapled and stitched peptides of an alternative HR2 sequence corresponding to SEQ ID NO:110. The effectiveness at disrupting the 5-HB/HR2 interaction depended upon the staple type and staple positioning of the double staples and stitches of the core template sequence (SEQ ID NO:258) within the context of the longer HR2-type peptide (SEQ ID NO:110), with double staples N,S producing the most potent competitive inhibitor of this group.

Integrating the competitive ELISA data across the i, i+4 staple scan of the core template HR2 sequence (SEQ ID NO:10) bearing an N-terminal extension (SEQ ID NO: 103), and various double stapled and stitched constructs within the core template sequence (SEQ ID NO: 10), longer HR2 sequence (SEQ ID NO:9), and alternate HR-2 type sequence (SEQ ID NO:110), further reveals (1) the addition of N- or N- and C-terminal sequence to the stapled core template sequence can enhance competitive binding activity of the stapled peptides (compare the N,S double staple in the context of SEQ ID NO:10, SEQ ID NO:9, and SEQ ID NO:110); (2) in the context of SEQ ID NO:103, C-terminal staple positions are generally more favorable than N-terminal staple positions (FIG. 19B and FIG. 19C); and (3) several double staple positions show binding activity across both direct and competitive binding assays and in the context of alternate HR2 sequences (SEQ ID NO:9, SEQ ID NO:110) (see for example double staples N,S and O,S in FIGS. 18, 21, and 22).

### Example 5: Assessing Antiviral Activity of SARS-CoV-2-S HR2 Stapled Peptides

To test the capacity of SARS-CoV-2 HR2 stapled peptides to block SARS-CoV-2 infection of cultured cells, Vero E6 cells plated in 384-well format were treated for 1 hour with a serial dilution of stapled peptides (e.g. 10 μM starting dose), performed in triplicate, followed by 4 hour challenge with SARS-CoV-2 to achieve control infection of 10-20% cells (the pre-determined optimal infectivity to assess the dynamic range of test compounds in the assay). Infected cells were then washed, fixed with 4% paraformaldehyde, rewashed in PBS, immune-stained with anti-SARS-CoV-2 nucleocapsid monoclonal antibody followed by anti-mouse Ig secondary antibody (Alexa Fluor 488; Life Technologies), and cell bodies counterstained with HCS CellMask blue. Cells were imaged across the z-plane on a Nikon Ti Eclipse automated microscope, analyzed by CellProfiler software, and infection efficiency calculated by dividing infected by total cells. Control cytotoxicity assays were performed using Cell-Titer Glo (Promega) and LDH release (Roche) assays.

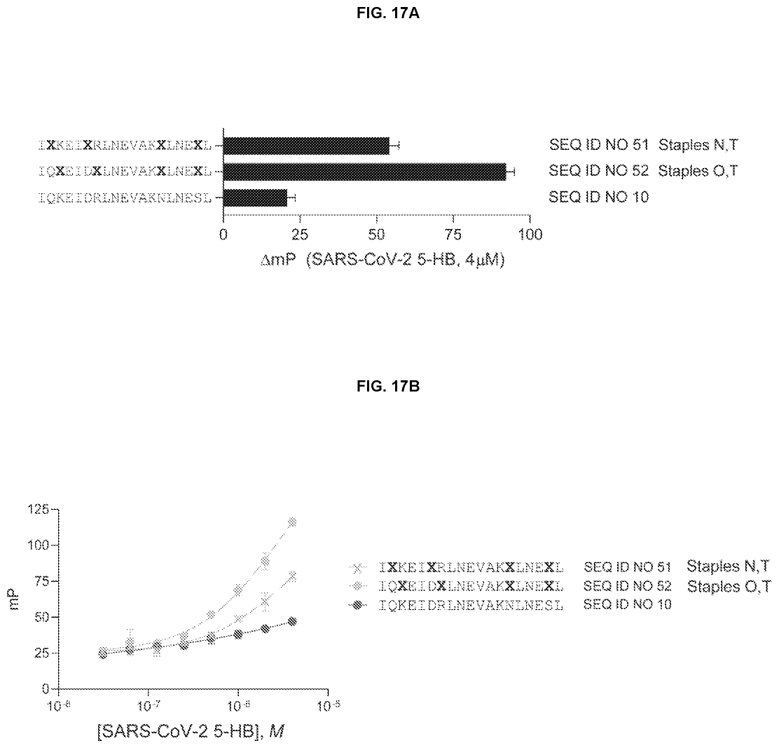

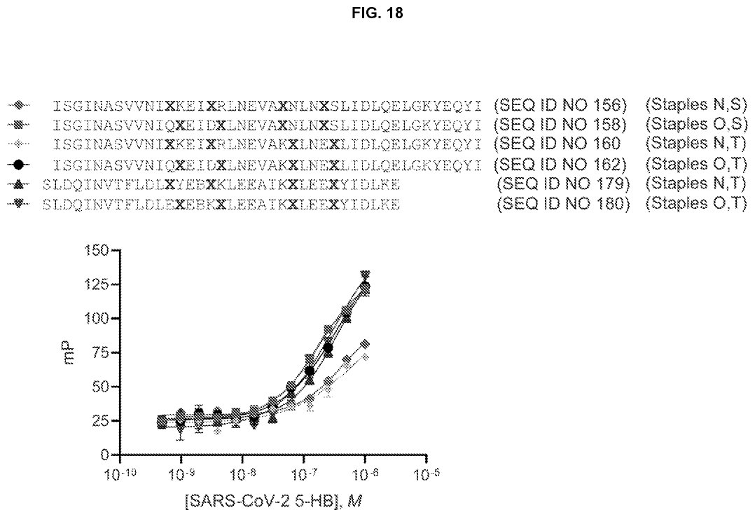

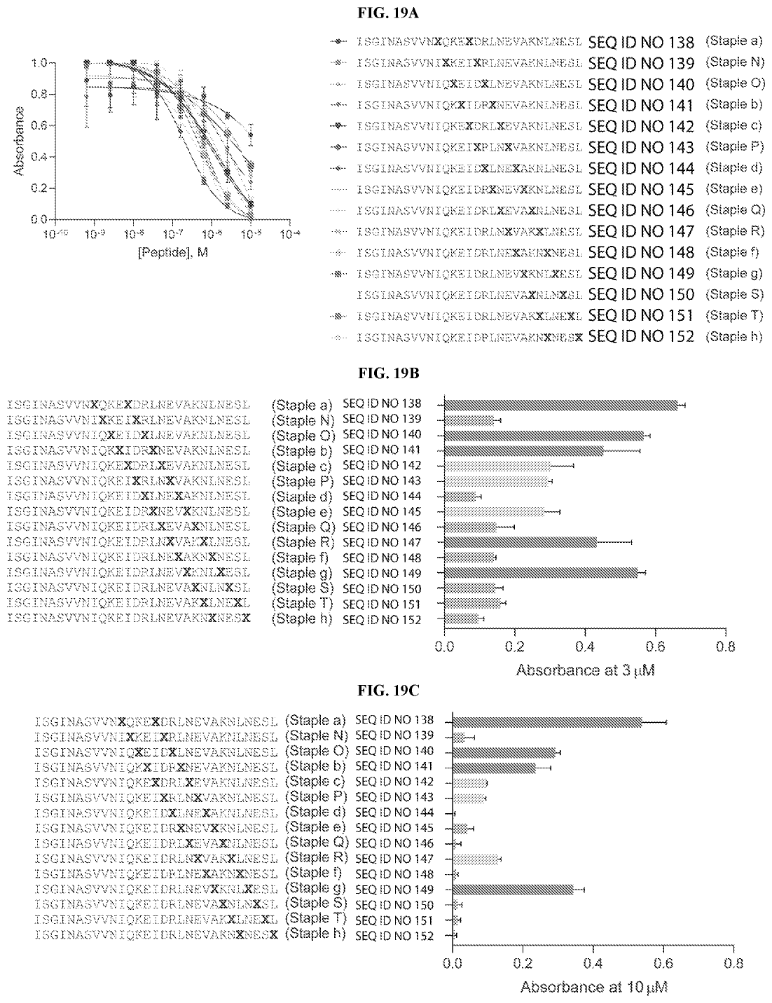

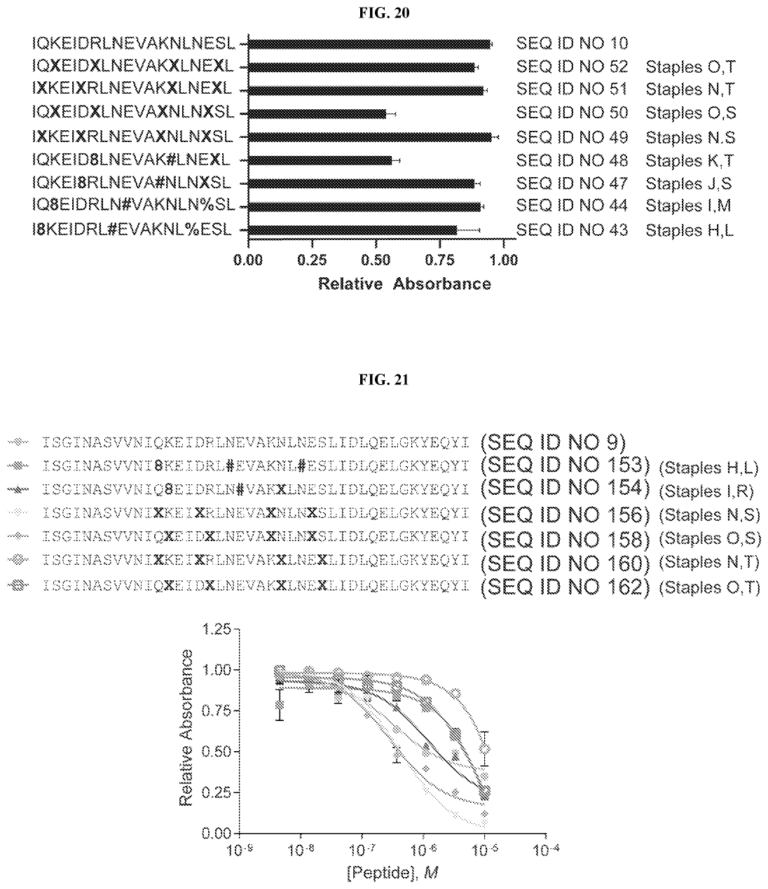

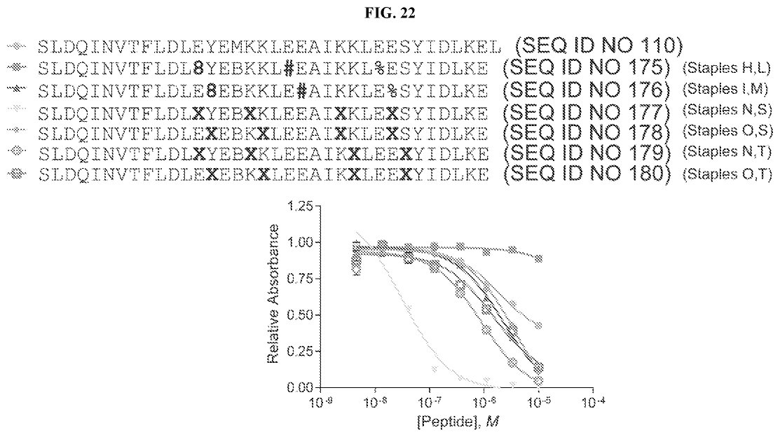

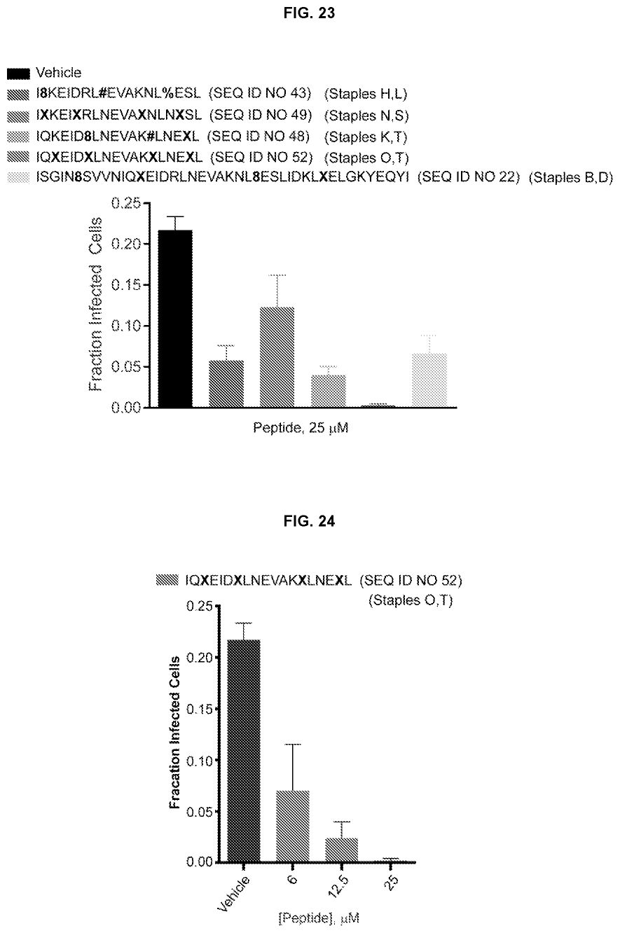

FIG. 23 shows the antiviral activity of exemplary double stapled and stitched peptides of the core template sequence SEQ ID NO:10 and a double stapled peptide of the longer HR2 sequence corresponding to SEQ ID NO:9. Peptides were screened at 25 μM for the capacity to block infection of Vero E6 cells by live, wild-type SARS-CoV-2 virus, with fraction infected cells plotted. In each case, the stapled peptides inhibited infection as compared to treatment with the vehicle control. FIG. 24 shows that hits from the peptide screen in Vero E6 cells subjected to SARS-CoV-2 infection were then subjected to further dose-response testing, as exemplified by the double stapled core template sequence bearing staples O,T (SEQ ID NO:52), which has an IC₅₀ below 6 μM for blocking SARS-CoV-2 in the assay. FIG. 25 shows the differential anti-viral activity of double stapled and stitched peptides of the core template sequence (SEQ ID NO: 10), as assessed in high-throughput by an antibody-based SARS-CoV-2 detection platform in infected Vero E6 cells. FIG. 26 shows that double i, i+7 stapling and stitching in the indicated positions outside of the core template sequence (SEQ ID NO:10) within the context of the longer HR2 peptide sequence (SEQ ID NO: 9) did not yield compounds with anti-viral activity. FIG. 27 shows the differential anti-viral activity of exemplary double stapled and stitched peptides of the core template sequence (SEQ ID NO:10) within the context of the longer HR2 peptide sequence corresponding to SEQ ID NO:9. The construct bearing double i, i+4 staples O,S had the most potent antiviral activity, followed by compounds bearing the O,T; I,R; and N,S staples, whereas the N,T and H,L constructs showed no effect in the assay. FIG. 28 shows the differential anti-viral activity of exemplary double stapled and stitched peptides of an alternate core template sequence in the context of its longer HR2-type peptide sequence corresponding to SEQ ID NO:110. The construct bearing double i, i+4 staples N,S had the most potent antiviral activity, followed by the peptide containing the N,T staples, whereas the other compounds in this group showed no significant effect.

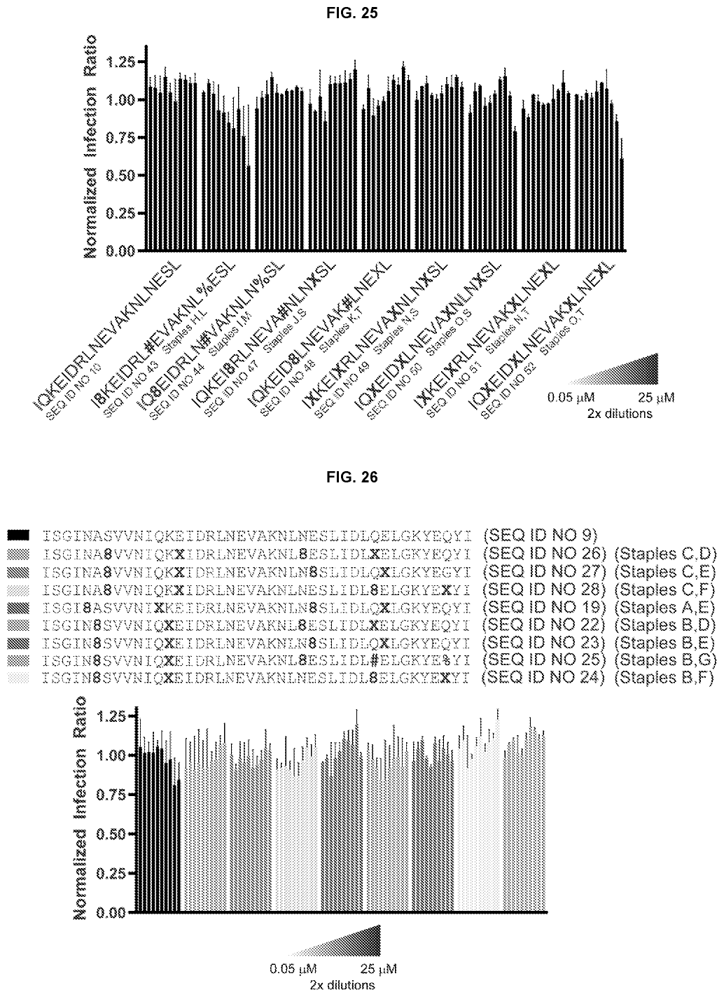

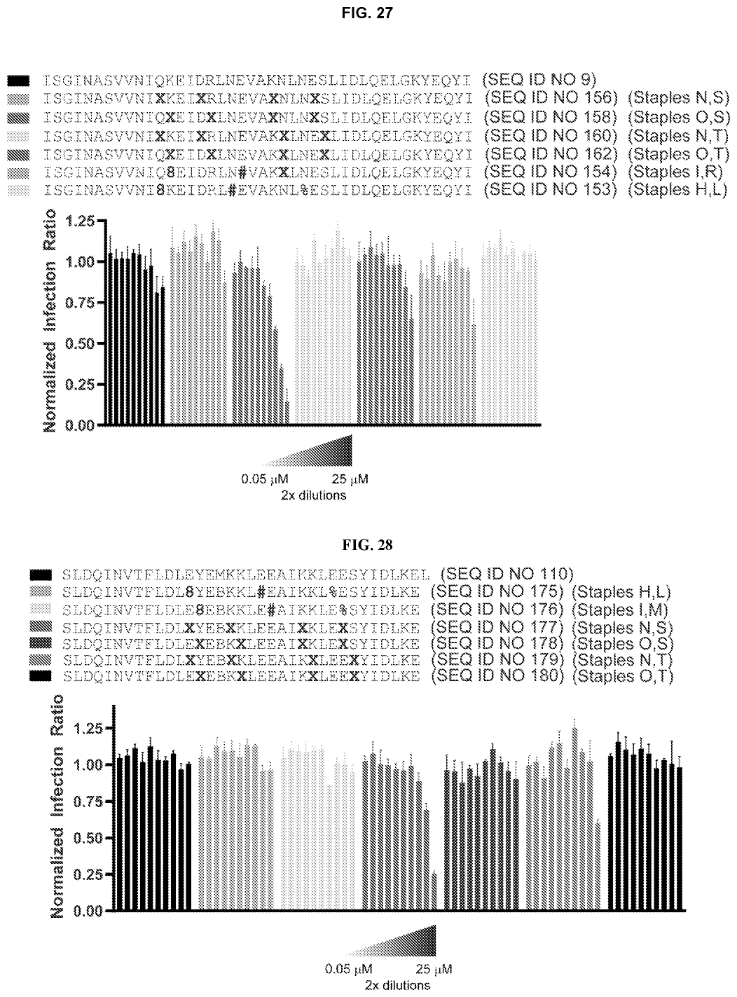

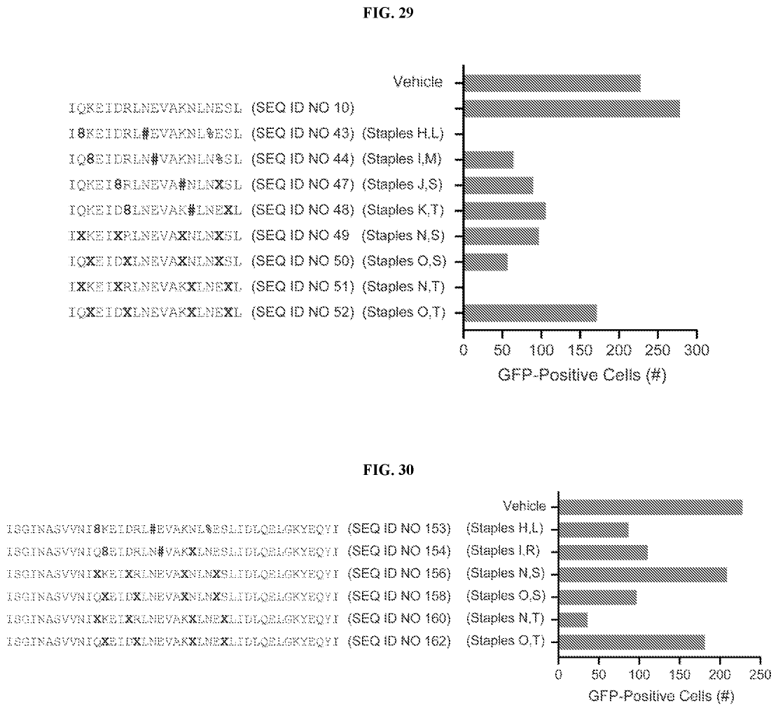

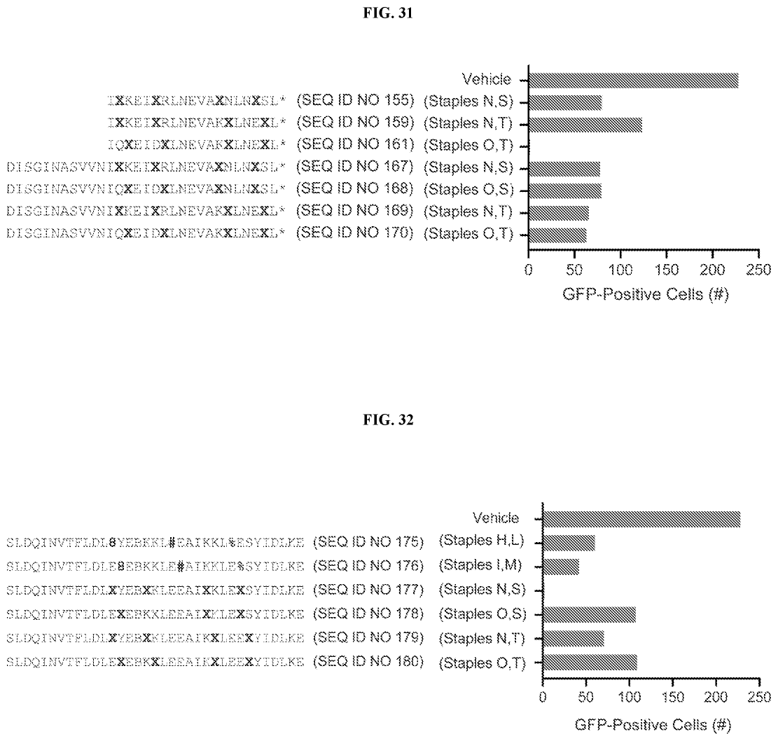

In an alternate antiviral assay system, SARS-CoV-2 pseudovirus was used instead of wild-type SARS-CoV-2 virus, and ACE2-expressing 293T cells were used in place of Vero E6 cells. The 293T-hsACE2 stable cell line (Cat #C-HA101) and the pseudotyped SARS-CoV-2 (Wuhan-Hu-1 strain) particles with GFP (Cat #RVP-701G, Lot #CG-113A) reporters were used (Integral Molecular). The neutralization assay was carried out according to the manufacturers' protocols. In brief, 5 μL of a single dose of peptide (5 μM final dose) was incubated with 5 μL pseudotyped SARS-CoV-2-GFP for 1 hr at 37° C. in a 384 well black clear bottom plate followed by addition of 30 μL of 1,000 293T-hsACE2 cells in 10% FBS DMEM, phenol red free media and placed in a humidified incubator for 48 or 72 hrs. Hoechst 33342 and DRAQ7 dyes were added and the plate imaged on a Molecular Devices ImageXpress Micro Confocal Laser at 10× magnification. GFP (+) cells were counted and plotted using Prism software (Graphpad). FIG. 29 shows the anti-viral activity of double stapled and stitched peptides of the core template sequence (SEQ ID NO:10) compared to the unstapled core template sequence that shows no anti-viral activity, as assessed by a SARS-CoV-2 pseudoviral assay in which the number of infected cells is counted by IXM microscopy based on the fluorescence of ACE2-expressing 293T cells infected by the GFP-expressing pseudovirus. FIG. 30 shows the differential anti-viral activity of double stapled and stitched peptides of the core template sequence (SEQ ID NO:10) in the context of its longer HR2 sequence (SEQ ID NO:9), as assessed by a SARS-CoV-2 pseudoviral assay in which the number of infected cells is counted by IXM microscopy based on the fluorescence of ACE2-expressing 293T cells infected by the GFP-expressing pseudovirus. FIG. 31 shows the differential anti-viral activity of double stapled peptides of the core template sequence (SEQ ID NO:10) with or without an N-terminal peptide extension (aa 1168-1176) and bearing a C-terminal derivatization with GSGSGC(SEQ ID NO:256)-(PEG4-chol)-carboxamide. FIG. 32 shows the differential anti-viral activity of double stapled and stitched peptides of an alternate core template sequence in the context of its longer HR2-type sequence (SEQ ID NO:110).

Integrating the antiviral data of various double stapled and stitched peptides across templates sequences of various length and composition, further revealed that (1) installing staples or stitches can transform an unstapled template sequence from a peptide with little to no activity into an active antiviral agent (see, for example, FIG. 27, FIG. 28 and FIG. 29); (2) the impact of installing staples can differentially impact the antiviral activity depending on the length of the template sequence and the alternate composition of the sequence template. For example, the N,S double staple yields the more active peptide in the context of SEQ ID NO:110, while the O,S double stapled has a greater benefit in the context of SEQ ID NO:9 (see FIGS. 27 and 28); (3) taking into consideration the distinctions among the direct FPA, competitive ELISA, live SARS-CoV-2 infectivity assay in Vero E6 cells, and SARS-CoV-2 pseudovirus assay in ACE2-expressing 293T cells, stapled constructs with direct or competitive binding activity likewise manifested antiviral activity in one or the other SARS-CoV-2 infectivity assay, including for example HR2 sequences bearing double staples N,S; O,S; and O,T. As another example, the N,T and N,S double staples conferred enhanced 5-HB competitive binding activity in the context of SEQ ID NO:110 relative to SEQ ID NO:9, and likewise showed enhanced antiviral activity against wild-type SARS-CoV-2 infectivity assay in Vero E6 cells (compare the double stapled N,T and N,S constructs in FIG. 21 vs. FIG. 22 and in FIG. 27 and FIG. 28); (4) double stapling or stitching outside of the core template sequence showed no antiviral effect, which stands in contrast to the beneficial effects of stapling within the core template sequence (compare FIG. 26 with FIG. 27); (5) staple type, staple location, the presence of one or more staples, template sequence length, and template sequence composition can influence the functional activity of stapled and stitched peptides of the SARS-CoV-2 HR2 domain.

### Example 6: Determining Whether SAH-SARS-CoV-2 Peptides Engage the Plasma Membrane and Colocalize with SARS-CoV-2 During Infection

FITC-labeled SAH-SARS-CoV-2 peptides are contacted with cultured cells (e.g. Vero, Huh770, Calu-371, 293T, primary nasal, lung epithelial, or alveolar cells), to determine whether they engage the plasma membrane and/or are taken up via the pinosomal pathway, which is tested by measuring accumulation of FITC-SAH-SARS-CoV-2 on the plasma membrane and/or in intracellular vesicles labeled with cytotracker red. Colocalization of FITC-SAH-SARS-CoV-2 peptide and Rhodamine (R₁₈)-labelled SARS-CoV-2 is also investigated during cellular contact and uptake, to determine the capacity of SAH-SARS-CoV-2 peptides to target SARS-CoV-2 during the infection process.

### Example 7: Investigating SAH-SARS-CoV-2 Inhibition of COVID-19 Infection In Vivo

To examine the capacity of SAH-SARS-CoV-2 peptides to inhibit SARS-CoV-2 infection in vivo, vehicle or SAH-SARS-CoV-2 peptide (e.g. 250 μM, 25 μL) is administered to anesthetized mice intranasally, and this is followed by trans-nasal infection with SARS-CoV-2 virus (e.g. USA-WA1/2020; Hongkon VM20001061) (104 PFU) 4-24 hours later. Mice are sacrificed 20 hours post-infection, and the nasal epithelium is cryosectioned, immunostained with anti-SARS-CoV-2 nucleocapsid antibody and fluorescent anti-mouse Ig secondary antibody, counterstained with DAPI, and imaged using a fluorescent microscope.

### Example 8: Assessing Whether SAH-SARS-CoV-2 Peptides Both Prevent and Treat COVID-19 Infection In Vitro

Vero E6 cells plated in 384-well format (60,000 cells/well) are exposed to (a) SARS-CoV-2 only; (b) SARS-CoV-2 for 4 hour followed by treatment with SAH-SARS-CoV-2; and (c) SAH-SARS-CoV-2 for 4 hour followed by SARS-CoV-2 infection. The Vero cells are then imaged 24 hour post-infection by anti-SARS-CoV-2 immunostaining and high-content fluorescence microscopy.

### Example 9: Photoreactive SAH-SARS-CoV-2 Peptides for Protein Capture and Binding Site Analysis

To identify and confirm SAH-SARS-CoV-2 targets in the context of cellular infection by SARS-CoV-2, stapled peptides derivatized for proteomic analyses are employed. First, photoreactive SAH-SARS-CoV-2 constructs are synthesized in which (1) a non-natural amino acid containing the photoreactive benzophenone functionality (Fmoc-Bpa) is substituted at discrete sites adjacent to the interaction surface of the HR2 domain and (2) the N-terminus of the peptide is capped with biotin for robust streptavidin-based target retrieval. Then, the photoreactive SAH-SARS-CoV-2 (pSAH-SARS-CoV-2) is added to cultured cells exposed to SARS-CoV-2 virus, and upon UV irradiation, the pSAH-SARS-CoV-2 intercalates into target protein(s). Infected cells are lysed, pelleted, and the isolated supernatant subjected to SA pull-down to retrieve pSAH-crosslinked proteins. The complexes are eluted by heating in load buffer and then trypsinized and subjected to MS-based identification using a reverse-phase nanoflow LC/MS/MS with an online LTQ-Orbitrap mass spectrometer (Thermo Scientific). MS data are processed using SEQUEST and Mascot software to catalogue protein targets.

Specific hits are defined as those proteins uniquely found in pSAH-SARS-CoV-2-treated and irradiated samples, but not in the unirradiated controls or in pSAH-SARS-CoV-2mutant-treated samples. This methodology allows identification of those amino acid residues in the target protein specifically modified by the pSAH-SARS-CoV-2, thus revealing the explicit site(s) of SAH-SARS-CoV-2 peptide interaction.

### Example 10: Structured Antigens for COVID-19 Vaccination

Structurally constrained-SARS-CoV-2 HR peptides are conjugated to protein carrier (e.g. KLH), followed by rabbit immunization, antisera collection, and ELISA-based immunogenicity testing. For a given structurally constrained SARS-CoV-2 HR construct, the unmodified template peptide and three alternatively conjugated stapled analogs are compared in a neutralizing immunogenicity study. Once pre-bled (˜5 mL serum), two NZW female rabbits (6-8 weeks old) per immunogen receive a primary intramuscular (IM) injection (250 μg with Freund's complete, CpG-ODN, or Ribi adjuvant) on day 1, followed by IM boosts (100 μg with corresponding adjuvant) on days 21, 42, 63, 84, and 105, and production bleeds on days 52, 73, 94, and 112. Direct ELISA assays are performed for each production bleed to monitor and compare specific antibody production titers. Briefly, 96-well microtiter plates are coated with individual SARS-CoV-2 HR immunogens (5 μg/mL) overnight at 4° C. The wells are washed twice with PBS containing 0.05% Tween 20 and blocked with 3% BSA for 45 min at 37° C. Serial dilutions of rabbit antisera are then added to the plate in triplicate and incubated at 37° C. for 2 hours. After washing three times, a 1:500 dilution of alkaline phosphatase-labeled goat anti-rabbit IgG in PBS/1% BSA is added, and the plate incubated for 40 min at room temperature. The wells are washed, exposed to alkaline phosphatase substrate for 30 minutes, and analyzed by microplate reader at 405 nm.

In addition to direct N-terminal conjugation of structured SARS-CoV-2 HR peptides (e.g. via thiol of installed cysteine) or installation of a lysine for conjugation on the non-interacting face of SAH-SARS-CoV-2 peptides, olefin derivatization of hydrocarbon staples also are performed so the proposed “neutralizing face” of the constructs is directed outward, maintaining the non-neutralizing face buried against the protein or lipid conjugate (e.g. KLH14, bovine serum albumin, cholera toxin, micelle). Catalytic osmium tetroxide is used to first dihydroxylate the olefin, followed by cyclization with thionyl chloride or carbonyl diimidazole. The electrophilic cyclic sulfite or carbonate is then reacted with sodium azide, which is reduced to an amine using phosphines. Reaction with the bifunctional reagent 3-thiopropionic acid installs a thiol, which is then used to attach the carrier (e.g. maleimide-KLH). As an alternative approach, the peptides are presented in the context of a lipid membrane, which may facilitate neutralizing antibody recognition. For example, the peptides are differentially conjugated to 1,3-dipalmitoyl-glycero-2-phosphoethanolamine, which is then combined with dodecylphosphocholine (DPC) to generate immunogen-tethered micelles.

A DNA prime-protein boost immunization strategy has been shown to be more effective than protein-alone or DNA-alone vaccination to yield HIV-1 neutralization antibodies. An analogous approach for COVID-19 is tested with lead structured SARS-CoV-2 HR conjugates replacing the timed protein boosts with structured peptide boosts according to the published immunization protocols.

### Example 11: Determining Whether Stapled SAH-SARS-CoV-2 Peptides Inhibit SARS-COV-2 Infection of Infected Cells in Culture

In this study, cells (e.g. Vero, Huh770, Calu-371, 293T, primary nasal, lung epithelial, or alveolar cells) are plated in a 24 well plate at 30,000 cells/well. The following day, the cells are treated with a serial dilution of (e.g. 10 μM starting dose) of the indicated stapled SAH-SARS-CoV-2 peptide or volume-equivalent DMSO vehicle, followed by SARS-CoV-2 infection within 2 h at 0.1 MOI. The infection medium is removed at 2 h post-infection and the medium is replaced with media containing 5% FBS with a serial dilution of the indicated SAH-SARS-CoV-2 peptide as above. Cells are then incubated at 37° C. and 24 hours later are harvested for determination of viral infectivity (e.g. antibody based detection or qPCR, as described above).

### Example 12: Assessing Whether Stapled SAH-SARS-CoV-2 Peptides Inhibit SARS-CoV-2 Induced Syncytia Formation

In this study, cells (e.g. Vero, Huh770, Calu-371, 293T, primary nasal, lung epithelial, or alveolar cells) are plated and treated as described in Example 11, except that the number of viral syncytia are counted at 48 hours post-infection. Syncytia are counted in three different wells at four discrete locations per well.

### Example 13: Investigating Whether Stapled SAH-SARS-CoV-2 Peptide Prevents Viral Infection in Culture

In this study, cells (e.g. Vero, Huh770, Calu-371, 293T, primary nasal, lung epithelial, or alveolar cells) are plated and treated The following day with a serial dilution (e.g. 10 μM starting dose) of the indicated SAH-SARS-CoV-2 peptide or volume-equivalent DMSO vehicle, followed by infection with SARS-CoV-2 virus within 30 minutes. The supernatant is collected 24 h post infection and is applied to cells that are plated the day prior on a 24 well plate at 60,000 cells/well. Plaque assays are performed using the collected supernatant and titers determined at 5 days post-infection.

### Example 14: Determining Whether Stapled SAH-SARS-CoV-2 Peptide Blocks Intranasal SARS-CoV-2 Infection in a Sequence-Specific Manner

Four groups (n=10 per group) of K18-hACE2 (Jackson Laboratory) mice are anesthetized and treated intranasally with stapled SAH-SARS-CoV-2, stapled SAH-SARS-CoV-2 negative control peptide (e.g. 125 μM in 1.2% DMSO), or volume-equivalent vehicle. One-hour post treatment, three groups of mice are inoculated with a single dose of SARS-CoV-2 at 10⁴ pfu/mouse intranasally, with the fourth group receiving a mock inoculation. Mice are sacrificed at 24 hours post infection and the noses are harvested, sectioned, immunostained for SARS-CoV-2, counterstained with DAPI, and imaged with a fluorescent microscope.

### Example 15: Assessing Whether Prophylactic Intranasal Treatment with Stapled SAH-SARS-CoV-2 Peptide Inhibits SARS-CoV-2 Lung Infection

In this study, four groups (n=10 per group) of K18-hACE2 (Jackson Laboratory) mice are anesthetized and treated intranasally with stapled SAH-SARS-CoV-2 or stapled SAH-SARS-CoV-2 negative control peptides (e.g. 125 μM in 1.2% DMSO), or volume-equivalent vehicle. 24 hour later, three groups of mice are inoculated with a single dose of SARS-CoV-2 at 10⁴ pfu/mouse intranasally. The fourth group is treated with volume-equivalent vehicle and mock-infected. Mice are euthanized 4 days later (peak of viremia) for evaluation by necropsy and viral load as quantitated by qPCR from supernatant samples of lung homogenates, prepared as described using a tissuelyzer (Qiagen). See Bao L et al. Nature. 2020. Epub 2020 May 8; doi: 10.1038/s41586-020-2312-y. left lung lobes are harvested from two of the mice from each group after 1% paraformaldehyde perfusion, followed by cryopreservation in OCT. Tissue sections (5 μm) are treated with anti-SARS-CoV-2 nucleocapsid antibody overnight followed by fluorescent anti-Ig secondary for 1 h. Sections are washed and mounted with medium containing DAPI (blue), viewed with an Olympus fluorescent microscope, and analyzed by ImageJ. To evaluate the capacity of lead stapled peptides to treat or mitigate established SARS-CoV-2 infection, K18-hACE2 mice (n=10 per arm; 5 male, 5 female) are inoculated intranasally at a viral dosage of 10⁴ PFU on day 1, followed by daily oropharyngeal, intraperitoneal, intravenous, or subcutaneous treatment with stapled peptide or vehicle for 10 days (days 2-12). In an alternate design, dosing is delayed until 3-5 days post-inoculation to simulate symptom- or positive test-driven initiation of therapy. Mice are continuously monitored to record body weights and clinical signs, with disease progression scored as >10% body weight loss, labored breathing, and/or failure to thrive. Doses for the most effective compound and route are then be refined in both prevention and treatment studies to determine the minimum dose to protect mice. The same experimental design is used except that the 4 treatment groups (n=10; 5 male, 5 female) receive the original dose and then 3 progressively lowered doses in 4-fold increments.

### Example 16: Investigating Whether Administration of Stapled SAH-SARS-CoV-2 Peptide as a Nanoparticle Preparation Increases Lung Delivery

In this study, three groups (n=10) of K18-hACE2 (Jackson Laboratory) mice are treated intratracheally with Cy5-labeled stapled SAH-SARS-CoV-2 administered alone (e.g. 100 μM) or in combination with nanoparticles (NP) formed of nanochitosan polymer (Zhang et al., Nature Medicine, 2005), (1:2.5, peptide:NP) in a 50 μl volume. The control group receives volume-equivalent vehicle. Mice are sacrificed at 24 hours post-treatment and lungs are harvested after 1% paraformaldehyde perfusion, followed by cryopreservation in OCT, tissue sectioning, and fluorescence detection of Cy5-labeled stapled peptide.

### Example 17: Assessing Whether Intratracheal Administration of Stapled SAH-SARS-CoV-2 Peptide as a Nanoparticle Preparation at 48 hours pre-SARS-CoV-2 Inoculation Markedly Suppresses Viral Infection of the Lung

In this study, four groups (n=10 per group) of K18-hACE2 (Jackson Laboratory) mice are anesthetized and treated intratracheally with volume-equivalent vehicle with stapled nanoparticles (NP); SAH-SARS-CoV-2 peptide alone (e.g. 250 μM peptide in 1.2% DMSO), SAH-SARS-CoV-2 peptide in combination with NP (1:2.5, peptide:NP), or volume-equivalent vehicle alone. Forty-eight hours after treatment, the four groups of mice are inoculated intranasally with a single dose of SARS-CoV-2 at 1×10⁴ pfu/mouse. A fifth treatment group (n=10) receives volume-equivalent vehicle intratracheally followed by mock inoculation 48 hours later. Mice are sacrificed four days post-infection and evaluated as described above in Example 15.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A structurally-stabilized polypeptide comprising an amino acid sequence that is at least 55%, 60%, 65%, 70%, 75%, 80%, 85%, 90%, or 94% identical to sequence set forth in SEQ ID NO:10 (IQKEIDRLNEVAKNLNESL), wherein amino acids at positions of SEQ ID NO:10 selected from (wherein position 1 is the N-terminal Isoleucine and position 19 is the C-terminal Leucine of SEQ ID NO:10):
(i) positions 7 and 11;
(ii) positions 10 and 14;
(iii) positions 12 and 16;
(iv) positions 14 and 18
(v) positions 2 and 9;
(vi) positions 4 and 11;
(vii) positions 9 and 16;
(viii) positions 2 and 6;
(ix) positions 8 and 12;
(x) positions 9 and 13;
(xi) positions 11 and 15;
(xii) positions 14 and 18;
(xiii) positions 15 and 19;
(xiv) positions 7 and 14;
(xv) positions 3 and 10;
(xvi) positions 6 and 13;
(xvii) positions 13 and 17;
(xviii) positions 3 and 7;
(xix) positions 3, 7, 13, and 17;
(xx) positions 3, 7, 14, and 18;
(xxi) positions 2, 6, 14, and 18;
(xxii) positions 2, 6, 13, and 17;
(xxiii) positions 3, 10, and 17;
(xiv) positions 2, 9, and 13;
(xv) positions 3, 10, and 14;
(xvi) positions 6, 13, and 17; or
(xvii) positions 7, 14, and 18,
are replaced by α, α-disubstituted non-natural amino acids with olefinic side chains; and
if the amino acid sequence includes additional substitution(s), those substitution(s) are based on either (A) or (B):
(A) wherein positions 4, 8, 10, 13, 15, 17, and 18 of SEQ ID NO:10, if not substituted by an α, α-disubstituted non-natural amino acid with olefinic side chains, are not substituted or substituted by a conservative amino acid substitution;
wherein positions 1, 5, 7, and 11 if substituted are substituted by conservative amino acid substitutions or an α, α-disubstituted non-natural amino acid with olefinic side chains; and
wherein the remaining positions in SEQ ID NO:10 can be substituted by any amino acid or an α, α-disubstituted non-natural amino acid with olefinic side chains; or
(B) wherein one or more of positions 1, 3, 5, 6, 8, 10, 12, 13, 15, 17, and 19 of SEQ ID NO:10, are not substituted, or if substituted are replaced by a conservative amino acid substitution; and
wherein one or more of positions 2, 4, 7, 9, 11, 14, 16, and 18 of SEQ ID NO:10 can be replaced by any amino acid or an α, α-disubstituted non-natural amino acid with olefinic side chains;
wherein the structurally-stabilized peptide is 15 to 100 amino acids in length, optionally 19 to 45 amino acids in length; and
wherein the structurally-stabilized peptide has one or more of the properties listed below: (i) binds a recombinant 5-helix bundle of SARS-CoV-2 S protein; (ii) disrupts the interaction between the 5 helix bundle and SEQ ID NO:10; (iii) is alpha-helical; (iv) is protease resistant; (v) inhibits fusion of SARS-CoV-2 with a host cell; and/or (vi) inhibits infection of a cell by SARS-CoV-2.

2. The structurally-stabilized polypeptide of claim 1, wherein the amino acid sequence is at least 70% identical to the sequence set forth in SEQ ID NO:10.

3. The structurally-stabilized polypeptide of claim 1, wherein the amino acid sequence is at least 80% identical to the sequence set forth in SEQ ID NO:10.

4. The structurally-stabilized polypeptide of any one of claims 1 to 3, wherein the amino acid sequence is SEQ ID NO:50.

5. The structurally-stabilized polypeptide of any one of claims 1 to 3, wherein the amino acid sequence comprises the sequence of SEQ ID NO:52.

6. The structurally-stabilized polypeptide of any one of claims 1 to 3, wherein the amino acid sequence comprises the sequence of SEQ ID NO:51.

7. The structurally-stabilized polypeptide of any one of claims 1 to 3, wherein the amino acid sequence comprises (i) a sequence that is at least 75%, at least 80%, at least 85%, at least 90% or at least 92% identical to the sequence of any one of the sequences of SEQ ID NOs.: 133, 40, 136, 42, 30, 113, 34, 36, 134, 39, 135, 42, 137, 50, 52, 51, 31-33, 37, 41, and 44-49; or (ii) the sequence of any one of the sequences of SEQ ID NOs.: 133, 40, 136, 42, 30, 113, 34, 36, 134, 39, 135, 42, 137, 50, 52, 51, 31-33, 37, 41, and 44-49.

8. The structurally-stabilized polypeptide of any one of claims 1 to 7, further comprising the amino acid sequence ISGINASVVN (SEQ ID NO:250) appended at the N-terminus of the amino acid sequence.

9. The structurally-stabilized polypeptide of any one of claims 1 to 7, further comprising the amino acid sequence DISGINASVVN (SEQ ID NO:251) appended at the N-terminus of the amino acid sequence.

10. The structurally-stabilized polypeptide of any one of claims 1 to 9, further comprising the amino acid sequence IDLQEL (SEQ ID NO:252) appended at the C-terminus of the amino acid sequence.

11. The structurally-stabilized polypeptide of any one of claims 1 to 9, further comprising the amino acid sequence IDLQELGKYEQYI (SEQ ID NO:253) appended at the C-terminus of the amino acid sequence.

12. The structurally-stabilized polypeptide of any one of claims 1 to 9, further comprising the amino acid sequence IDLQELGSGSGC (SEQ ID NO:254) appended at the C-terminus of the amino acid sequence.

13. The structurally-stabilized polypeptide of any one of claims 1 to 9, further comprising the amino acid sequence IDLQELGKYEQYIGSGSGC (SEQ ID NO:255) appended at the C-terminus of the amino acid sequence.

14. The structurally-stabilized polypeptide of any one of claims 1 to 13, further comprising polyethylene glycol.

15. The structurally-stabilized polypeptide of any one of claims 1 to 14, further comprising cholesterol.

16. The structurally-stabilized polypeptide of any one of claims 1 to 13, further comprising GSGSGC(SEQ ID NO:256)-(PEG4-chol)-carboxamide.

17. A structurally-stabilized polypeptide comprising an amino acid sequence that is at least 55%, 60%, 65%, 70%, 75%, 80%, 85%, 90%, or 94% identical to sequence set forth in SEQ ID NO:110 (SLDQINVTFLDLEYEMKKLEEAIKKLEESYIDLKEL), wherein amino acids at positions of SEQ ID NO:110 selected from (wherein position 1 is the N-terminal Serine and position 36 is the C-terminal Leucine):
(i) positions 13, 20, and 27
(ii) positions 14, 21, and 28;
(iii) positions 13, 17, 24, and 28;
(iv) positions 14, 18, 24, and 28;
(v) positions 13, 17, 25, and 29; or
(vi) positions 14, 18, 25, and 29,
are replaced by α, α-disubstituted non-natural amino acids with olefinic side chains, and if the amino acid sequence has additional substitution(s) they are based on (A) (A) wherein if one or more of positions 4, 8, 10, 13, 15, 17, and 18 of SEQ ID NO:110 if not substituted by an α, α-disubstituted non-natural amino acid with olefinic side chains are not substituted or substituted by a conservative amino acid substitution;
wherein one or more of positions 1, 5, 7, and 11 of SEQ ID NO:110 if substituted are substituted by conservative amino acid substitutions;
wherein one or more of the remaining positions in SEQ ID NO:110 can be substituted by any amino acid;
and
wherein the peptide is 15 to 100 amino acids in length; and
wherein the structurally-stabilized peptide has one or more of the properties listed below: (i) binds a recombinant 5-helix bundle of SARS-CoV-2 S protein; (ii) disrupts the interaction between the 5 helix bundle and SEQ ID NO:258; (iii) is alpha-helical; (iv) is protease resistant; (v) inhibits fusion of SARS-CoV-2 with a host cell; and/or (vi) inhibits infection of a cell by SARS-CoV-2.

18. The structurally-stabilized polypeptide of claim 17, wherein the amino acid sequence is at least 70% identical to the sequence set forth in SEQ ID NOs:175-180.

19. The structurally-stabilized polypeptide of claim 17, wherein the amino acid sequence is at least 70% identical to the sequence set forth in SEQ ID NO:177 or 179.

20. The structurally-stabilized polypeptide of claim 17, wherein the amino acid sequence is identical to the sequence set forth in SEQ ID NO:179.

21. The structurally-stabilized polypeptide of claim 17, wherein the amino acid sequence is identical to the sequence set forth in SEQ ID NO:177.

22. The structurally-stabilized polypeptide of any one of claims 17-21, further comprising the amino acid sequence GSGSGC (SEQ ID NO:256) appended at the C-terminus of the amino acid sequence.

23. The structurally-stabilized polypeptide of claim 17 to 21, further comprising polyethylene glycol.

24. The structurally-stabilized polypeptide of any one of claims 17 to 21, further comprising cholesterol.

25. The structurally-stabilized polypeptide of any one of claims 17 to 21, further comprising GSGSGC(SEQ ID NO:256)-(PEG4-chol)-carboxamide).

26. A peptide comprising an amino acid sequence set forth in SEQ ID NO:9 with at least two amino acids separated by 2, 3, or 6 amino acids replaced by α, α-disubstituted non-natural amino acids with olefinic side chains, wherein the peptide is no greater than 45 amino acids in length and wherein the peptide binds a recombinant 5-helix bundle COVID-19 S protein.

27. A peptide comprising an amino acid sequence set forth in SEQ ID NO:10 with at least two amino acids separated by 2, 3, or 6 amino acids replaced by α, α-disubstituted non-natural amino acids with olefinic side chains, wherein the peptide is at most 45 amino acids in length, and wherein the peptide binds a recombinant 5-helix bundle COVID-19 S protein.

28. A structurally-stabilized peptide comprising an amino acid sequence set forth in SEQ ID NO:9 with at least two amino acids separated by 2, 3, or 6 amino acids replaced by α, α-disubstituted non-natural amino acids with olefinic side chains, wherein the structurally-stabilized peptide is no longer than 45 amino acids in length, and wherein the structurally-stabilized peptide has one or more of the properties listed below: (i) binds a SARS-CoV-2 recombinant 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2.

29. The structurally-stabilized peptide of claim 28, wherein the amino acid sequence comprises a sequence set forth in any one of SEQ ID NOs: 11-29, 153, 154, 156, 158, 160, or 162.

30. The peptide of claim 26, comprising the amino acid sequence:
(a)
ISGI8ASVVNIXKEIDRLNEVAKNLNESLIDLQELGKYEQYI,
wherein each of 8 and X is independently a stapling amino acid (SEQ ID NO:11);
(b)
ISGIN8SVVNIQXEIDRLNEVAKNLNESLIDLQELGKYEQYI,
wherein each of 8 and X is independently a stapling amino acid (SEQ ID NO:12);
(c)
ISGINA8VVNIQKXIDRLNEVAKNLNESLIDLQELGKYEQYI,
wherein each of 8 and X is independently a stapling amino acid (SEQ ID NO:13);
(d)
ISGINASVVNIQKEIDRLNEVAKNL8ESLIDLXELGKYEQYI,
wherein each of 8 and X is independently a stapling amino acid (SEQ ID NO:14);
(e)
ISGINASVVNIQKEIDRLNEVAKNLN8SLIDLQXLGKYEQYI,
wherein each of 8 and X is independently a stapling amino acid (SEQ ID NO:15);
(f)
ISGINASVVNIQKEIDRLNEVAKNLNESLIDL8ELGKYEXYI,
wherein each of 8 and X is independently a stapling amino acid (SEQ ID NO:16);
(h)
ISGINASVVNIQKEIDRLNEVAKNL8ESLIDL#ELGKYE%YI,
wherein each of 8, #, and % is independently a stapling/stitching amino acid (SEQ ID NO:17);
(i)
ISGI8₁ASVVNIX₁KEIDRLNEVAKNL8₂ESLIDLX₂ELGKYEQYI,
wherein each of 8₁, X₁, 8₂, and X₂ is independently a stapling amino acid
(j)
ISGI8₁ASVVNIX₁KEIDRLNEVAKNLN8₂SLIDLQX₂LGKYEQYI,
wherein each of 8₁, X₁, 8₂, and X₂ is independently a stapling amino acid (SEQ ID NO:19);
(k)
ISGI8₁ASVVNIX₁KEIDRLNEVAKNLNESLIDL8₂ELGKYEX₂YI,
wherein each of 8₁, X₁, 8₂, and X₂ is independently a stapling amino acid (SEQ ID NO:20);
(l)
ISGI8₁ASVVNIXKEIDRLNEVAKNL8₂ESLIDL#ELGKYE%YI,
wherein each of 8₁, X₁, 8₂, #, and % is independently a stapling/stitching amino acid (SEQ ID NO:21);
(m)
ISGIN8₁SVVNIQX₁EIDRLNEVAKNL8₂ESLIDLX₂ELGKYEQYI,
wherein each of 8₁, X₁, 8₂, and X₂ is independently a stapling amino acid (SEQ ID NO:22);
(n)
ISGIN8₁SVVNIQX₁EIDRLNEVAKNLN8₂SLIDLQX₂LGKYEQYI,
wherein each of 8₁, X₁, 8₂, and X₂ is independently a stapling amino acid (SEQ ID NO:23);
(o)
ISGIN8₁SVVNIQX₁EIDRLNEVAKNLNESLIDL8₂ELGKYEX₂YI,
wherein each of 8₁, X₁, 8₂, and X₂ is independently a stapling amino acid (SEQ ID NO:24);
(p)
ISGIN8₁SVVNIQXEIDRLNEVAKNL8₂ESLIDL#ELGKYE%YI,
wherein each of 8₁, X₁, 8₂, #, and % is independently a stapling/stitching amino acid (SEQ ID NO:25);
(q)
ISGINA8₁VVNIQKX₁IDRLNEVAKNL8₂ESLIDLX₂ELGKYEQYI,
wherein each of 8₁, X₁, 8₂, and X₂ is independently a stapling amino acid (SEQ ID NO:26);
(r)
ISGINA8₁VVNIQKX₁IDRLNEVAKNLNESLIDL8₂ELGKYEX₂YI,
wherein each of 8₁, X₁, 8₂, and X₂ is independently a stitching amino acid (SEQ ID NO:27);
(s)
ISGINA8₁VVNIQKX₁IDRLNEVAKNL8₂ESLIDLX₂ELGKYEQYI,
wherein each of 8₁, X₁, 8₂, and X₂ is independently a stapling amino acid (SEQ ID NO:28);
(t)
ISGINA8₁VVNIQKXIDRLNEVAKNL8₂ESLIDL#ELGKYE%YI,
wherein each of 8₁, X₁, 8₂, #, and % is independently a stapling/stitching amino acid (SEQ ID NO:29);
(u)
ISGINASVVNI8KEIDRL#EVAKNL%ESLIDLQELGKYEQYI,
wherein each of 8, #, and % is independently a stapling/stitching amino acid (SEQ ID NO:153);
(v)
ISGINASVVNIQ8EIDRLN#VAKXLNESLIDLQELGKYEQYI,
wherein each of 8, #, and X is independently a stapling/stitching amino acid (SEQ ID NO:154);
(w)
ISGINASVVNIX₁KEIX₂RLNEVAX₃NLNX₄SLIDLQELGKYEQYI
wherein each of X₁, X₂, X₃, and X₄ is independently a stapling/stitching amino acid (SEQ ID NO:156);
(x)
SGINASVVNIQX₁EIDX₂LNEVAX₃NLNX₄SLIDLQELGKYEQYI,
wherein each of X₁, X₂, X₃, and X₄ is independently a stapling/stitching amino acid (SEQ ID NO:158);
(y)
ISGINASVVNIX₁KEIX₂RLNEVAKX₃LNEX₄LIDLQELGKYEQYI
wherein each of X₁, X₂, X₃, and X₄ is independently a stapling/stitching amino acid (SEQ ID NO:160); or
(z)
ISGINASVVNIQX₁EIDX₂LNEVAKX₃LNEX₄LIDLQELGKYEQYI
wherein each of X₁, X₂, X₃, and X₄ is independently a stapling/stitching amino acid (SEQ ID NO:162).

31. The structurally-stabilized peptide of claim 28, comprising the amino acid sequence:
(a)
ISGI8ASVVNIXKEIDRLNEVAKNLNESLIDLQELGKYEQYI,
wherein each of 8 and X is independently a stapling amino acid (SEQ ID NO:11); and wherein the sidechains of 8 and X are cross-linked to each other;
(b)
ISGIN8SVVNIQXEIDRLNEVAKNLNESLIDLQELGKYEQYI,
wherein each of 8 and X is independently a stapling amino acid (SEQ ID NO:12); and wherein the sidechains of 8 and X are cross-linked to each other;
(c)
ISGINA8VVNIQKXIDRLNEVAKNLNESLIDLQELGKYEQYI,
wherein each of 8 and X is independently a stapling amino acid (SEQ ID NO:13); and wherein the sidechains of 8 and X are cross-linked to each other;
(d)
ISGINASVVNIQKEIDRLNEVAKNL8ESLIDLXELGKYEQYI,
wherein each of 8 and X is independently a stapling amino acid (SEQ ID NO:14); and wherein the sidechains of 8 and X are cross-linked to each other;
(e)
ISGINASVVNIQKEIDRLNEVAKNLN8SLIDLQXLGKYEQYI,
wherein each of 8 and X is independently a stapling amino acid (SEQ ID NO:15); and wherein the sidechains of 8 and X are cross-linked to each other;
(f)
ISGINASVVNIQKEIDRLNEVAKNLNESLIDL8ELGKYEXYI,
wherein each of 8 and X is independently a stapling amino acid (SEQ ID NO:16); and wherein the sidechains of 8 and X are cross-linked to each other;
(h)
ISGINASVVNIQKEIDRLNEVAKNL8ESLIDL#ELGKYE%YI,
wherein each of 8, #, and % is independently a stapling/stitching amino acid (SEQ ID NO:17); and wherein the sidechains of 8 and # are cross-linked to each other and the sidechains of # and % are cross-linked to each other;
(i)
ISGI8₁ASVVNIX₁KEIDRLNEVAKNL8₂ESLIDLX₂ELGKYEQYI,
wherein each of 8₁, X₁, 8₂, and X₂ is independently a stapling amino acid (SEQ ID NO:18); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(j)
ISGI8₁ASVVNIX₁KEIDRLNEVAKNLN8₂SLIDLQX₂LGKYEQYI,
wherein each of 8₁, X₁, 8₂, and X₂ is independently a stapling amino acid (SEQ ID NO:19); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(k)
ISGI8₁ASVVNIX₁KEIDRLNEVAKNLNESLIDL8₂ELGKYEX₂YI,
wherein each of 8₁, X₁, 8₂, and X₂ is independently a stapling amino acid (SEQ ID NO:20); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(l)
ISGI8₁ASVVNIXKEIDRLNEVAKNL8₂ESLIDL#ELGKYE%YI,
wherein each of 8₁, X₁, 8₂, #, and % is independently a stapling/stitching amino acid (SEQ ID NO:21); wherein the sidechains of 8₁ and X are cross-linked to each other; wherein the sidechains of X and 8₂ are cross-linked to each other; and wherein the sidechains of 8₂ and # are cross-linked to each other;
(m)
ISGIN8₁SVVNIQX₁EIDRLNEVAKNL8₂ESLIDLX₂ELGKYEQYI,
wherein each of 8₁, X₁, 8₂, and X₂ is independently a stapling amino acid (SEQ ID NO:22); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(n)
ISGIN8₁SVVNIQX₁EIDRLNEVAKNLN8₂SLIDLQX₂LGKYEQYI,
wherein each of 8₁, X₁, 8₂, and X₂ is independently a stapling amino acid (SEQ ID NO:23); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(o)
ISGIN8₁SVVNIQX₁EIDRLNEVAKNLNESLIDL8₂ELGKYEX₂YI,
wherein each of 8₁, X₁, 8₂, and X₂ is independently a stapling amino acid (SEQ ID NO:24); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(p)
ISGIN8₁SVVNIQXEIDRLNEVAKNL8₂ESLIDL#ELGKYE%YI,
wherein each of 8₁, X₁, 8₂, #, and % is independently a stapling/stitching amino acid (SEQ ID NO:25); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(q)
ISGINA8₁VVNIQKX₁IDRLNEVAKNL8₂ESLIDLX₂ELGKYEQYI,
wherein each of 8₁, X₁, 8₂, and X₂ is independently a stapling amino acid (SEQ ID NO:26); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(r)
ISGINA8₁VVNIQKX₁IDRLNEVAKNLNESLIDL8₂ELGKYEX₂YI,
wherein each of 8₁, X₁, 8₂, and X₂ is independently a stitching amino acid (SEQ ID NO:27); wherein the sidechains of 8₁ and X₁ are cross-linked to each other; wherein the sidechains of X₁ and 8₂ are cross-linked to each other; and wherein the sidechains of 8₂ and X₂ are cross-linked to each other;
(s)
ISGINA8₁VVNIQKX₁IDRLNEVAKNL8₂ESLIDLX₂ELGKYEQYI,
wherein each of 8₁, X₁, 8₂, and X₂ is independently a stapling amino acid (SEQ ID NO:28); wherein the sidechains of 8₁ and X₁ are cross-linked to each other; wherein the sidechains of X₁ and 8₂ are cross-linked to each other; and wherein the sidechains of 8₂ and X₂ are cross-linked to each other; or
(t)
ISGINA8₁VVNIQKXIDRLNEVAKNL8₂ESLIDL#ELGKYE%YI,
wherein each of 8₁, X₁, 8₂, #, and % is independently a stapling/stitching amino acid (SEQ ID NO:29) wherein the sidechains of 8₁ and X are cross-linked to each other; wherein the sidechains of X and 8₂ are cross-linked to each other; and wherein the sidechains of 8₂ and # are cross-linked to each other.

32. A structurally-stabilized polypeptide comprising the amino acid sequence set forth in SEQ ID NO:10 with at least two amino acids separated by 2, 3, or 6 amino acids replaced by α, α-disubstituted non-natural amino acids with olefinic side chains, wherein the peptide is at most 45 amino acids in length, and wherein the structurally-stabilized peptide has one or more of the properties listed below: (i) binds a recombinant SARS-CoV-2 5-helix bundle S protein; (ii) inhibits the interactions between the 5 helix bundle and SARS-CoV-2 HR2 peptide (SEQ ID NO:9); (iii) is alpha-helical; (iv) is protease resistant; (v) inhibits fusion of SARS-CoV-2 with a host cell; and/or (vi) inhibits infection of a cell by SARS-CoV-2.

33. The structurally-stabilized peptide of claim 32, wherein the amino acid sequence has a sequence set forth in any one of SEQ ID NOs: 30-52, 112-117, 130-137, 157, 159, or 161.

34. The peptide of claim 27, comprising the amino acid sequence:
(a) I8KEIDRLXEVAKNLNESL, wherein each of 8 and X is independently a stapling amino acid (SEQ ID NO:30);
(b) IQ8EIDRLNXVAKNLNESL, wherein each of 8 and X is independently a stapling amino acid (SEQ ID NO:31);
(c) IQKEISRLNEVAXNLNESL, wherein each of 8 and X is independently a stapling amino acid (SEQ ID NO:32);
(d) IQKEIDSLNEVAKXLNESL, wherein each of 8 and X is independently a stapling amino acid (SEQ ID NO:33);
(e) IQKEIDRL8EVAKNLXESL, wherein each of 8 and X is independently a stapling amino acid (SEQ ID NO:34);
(f) IQKEIDRLNSVAKNLNXSL, wherein each of 8 and X is independently a stapling amino acid (SEQ ID NO:35);
(h) IX₁KEIX₂RLNEVAKNLNESL, wherein each of X₁ and X₂ is independently a stapling amino acid (SEQ ID NO:36);
(i) IQX₁EIDX₂LNEVAKNLNESL, wherein each of X₁ and X₂ is independently a stapling amino acid (SEQ ID NO:37);
(j) IQKEIX₁RLNX₂VAKNLNESL, wherein each of X₁ and X₂ is independently a stapling amino acid (SEQ ID NO:38);
(k) IQKEIDRLX₁EVAX₂NLNESL, wherein each of X₁ and X₂ is independently a stapling amino acid (SEQ ID NO:39);
(l) IQKEIDRLNX₁VAKX₂LNESL, wherein each of X₁ and X₂ is independently a stapling amino acid (SEQ ID NO:40);
(m) IQKEIDRLNEVAX₁NLNX₂SL, wherein each of X₁ and X₂ is independently a stapling amino acid (SEQ ID NO:41);
(n) IQKEIDRLNEVAKX₁LNEX₂L, wherein each of X₁ and X₂ is independently a stapling amino acid (SEQ ID NO:42);
(o) I8KEIDRL#EVAKNL % ESL, wherein each of 8, #, and % is independently a stapling/stitching amino acid (SEQ ID NO:43);
(p) IQ8EIDRLN#VAKNLN % SL, wherein each of 8, #, and % is independently a stapling/stitching amino acid (SEQ ID NO:44);
(q) I8KEIDRL#EVAXNLNESL, wherein each of 8, #, and X is independently a stapling/stitching amino acid (SEQ ID NO:45);
(r) IQ8EIDRLN#VAKXLNESL, wherein each of 8, #, and X is independently a stapling/stitching amino acid (SEQ ID NO:46);
(s) IQKEI8RLNEVA#NLNXSL, wherein each of 8, #, and X is independently a stapling/stitching amino acid (SEQ ID NO:47);
(t) IQKEID8LNEVAK#LNEXL, wherein each of 8, #, and X is independently a stapling/stitching amino acid (SEQ ID NO:48);
(q) IX₁KEIX₂RLNEVAX₃NLNX₄SL, wherein each of X₁, X₂, X₃, and X₄ is independently a stapling amino acid (SEQ ID NO:49);
(r) IQX₁EIDX₂LNEVAX₃NLNX₄SL, wherein each of X₁, X₂, X₃, and X₄ is independently a stapling amino acid (SEQ ID NO:50);
(s) IX₁KEIX₂RLNEVAKX₃LNEX₄L, wherein each of X₁, X₂, X₃, and X₄ is independently a stapling amino acid (SEQ ID NO:51); or
(t) IQX₁EIDX₂LNEVAKX₃LNEX₄L, wherein each of X₁, X₂, X₃, and X₄ is independently a stapling amino acid (SEQ ID NO:52).

35. The structurally-stabilized peptide of claim 32, comprising the amino acid sequence:
(a) ISKEIDRLXEVAKNLNESL, wherein each of 8 and X is independently a stapling amino acid (SEQ ID NO:30); and wherein the sidechains of 8 and X are cross-linked to each other;
(b) IQ8EIDRLNXVAKNLNESL, wherein each of 8 and X is independently a stapling amino acid (SEQ ID NO:31); and wherein the sidechains of 8 and X are cross-linked to each other;
(c) IQKEISRLNEVAXNLNESL, wherein each of 8 and X is independently a stapling amino acid (SEQ ID NO:32); and wherein the sidechains of 8 and X are cross-linked to each other;
(d) IQKEIDSLNEVAKXLNESL, wherein each of 8 and X is independently a stapling amino acid (SEQ ID NO:33); and wherein the sidechains of 8 and X are cross-linked to each other;
(e) IQKEIDRL8EVAKNLXESL, wherein each of 8 and X is independently a stapling amino acid (SEQ ID NO:34); and wherein the sidechains of 8 and X are cross-linked to each other;
(f) IQKEIDRLNSVAKNLNXSL, wherein each of 8 and X is independently a stapling amino acid (SEQ ID NO:35); and wherein the sidechains of 8 and X are cross-linked to each other;
(h) IX₁KEIX₂RLNEVAKNLNESL, wherein each of X₁ and X₂ is independently a stapling amino acid (SEQ ID NO:36); and wherein the sidechains of X₁ and X₂ are cross-linked to each other;
(i) IQX₁EIDX₂LNEVAKNLNESL, wherein each of X₁ and X₂ is independently a stapling amino acid (SEQ ID NO:37); and wherein the sidechains of X₁ and X₂ are cross-linked to each other;
(j) IQKEIX₁RLNX₂VAKNLNESL, wherein each of X₁ and X₂ is independently a stapling amino acid (SEQ ID NO:38); and wherein the sidechains of X₁ and X₂ are cross-linked to each other;
(k) IQKEIDRLX₁EVAX₂NLNESL, wherein each of X₁ and X₂ is independently a stapling amino acid (SEQ ID NO:39); and wherein the sidechains of X₁ and X₂ are cross-linked to each other;
(l) IQKEIDRLNX₁VAKX₂LNESL, wherein each of X₁ and X₂ is independently a stapling amino acid (SEQ ID NO:40); and wherein the sidechains of X₁ and X₂ are cross-linked to each other;
(m) IQKEIDRLNEVAX₁NLNX₂SL, wherein each of X₁ and X₂ is independently a stapling amino acid (SEQ ID NO:41); and wherein the sidechains of X₁ and X₂ are cross-linked to each other;
(n) IQKEIDRLNEVAKX₁LNEX₂L, wherein each of X₁ and X₂ is independently a stapling amino acid (SEQ ID NO:42); and wherein the sidechains of X₁ and X₂ are cross-linked to each other;
(o) I8KEIDRL#EVAKNL % ESL, wherein each of 8, #, and % is independently a stapling/stitching amino acid (SEQ ID NO:43); wherein the sidechains of 8 and # are cross-linked to each other; and wherein the sidechains of # and % are cross-linked to each other;
(p) IQ8EIDRLN#VAKNLN % SL, wherein each of 8, #, and % is independently a stapling/stitching amino acid (SEQ ID NO:44); wherein the sidechains of 8 and # are cross-linked to each other; and wherein the sidechains of # and % are cross-linked to each other;
(q) ISKEIDRL#EVAXNLNESL, wherein each of 8, #, and X is independently a stapling/stitching amino acid (SEQ ID NO:45); wherein the sidechains of 8 and # are cross-linked to each other; and wherein the sidechains of # and X are cross-linked to each other;
(r) IQ8EIDRLN#VAKXLNESL, wherein each of 8, #, and X is independently a stapling/stitching amino acid (SEQ ID NO:46); wherein the sidechains of 8 and # are cross-linked to each other; and wherein the sidechains of # and X are cross-linked to each other;
(s) IQKEISRLNEVA#NLNXSL, wherein each of 8, #, and X is independently a stapling/stitching amino acid (SEQ ID NO:47); wherein the sidechains of 8 and # are cross-linked to each other; and wherein the sidechains of # and X are cross-linked to each other;
(t) IQKEID8LNEVAK#LNEXL, wherein each of 8, #, and X is independently a stapling/stitching amino acid (SEQ ID NO:48); wherein the sidechains of 8 and # are cross-linked to each other; and wherein the sidechains of # and X are cross-linked to each other;
(q) IX₁KEIX₂RLNEVAX₃NLNX₄SL, wherein each of X₁, X₂, X₃, and X₄ is independently a stapling amino acid (SEQ ID NO:49); wherein the sidechains of X₁ and X₂ are cross-linked to each other; wherein the sidechains of X₂ and X₃ are cross-linked to each other; and wherein the sidechains of X₃ and X₄ are cross-linked to each other;
(r) IQX₁EIDX₂LNEVAX₃NLNX₄SL, wherein each of X₁, X₂, X₃, and X₄ is independently a stapling amino acid (SEQ ID NO:50); wherein the sidechains of X₁ and X₂ are cross-linked to each other; wherein the sidechains of X₂ and X₃ are cross-linked to each other; and wherein the sidechains of X₃ and X₄ are cross-linked to each other;
(s) IX₁KEIX₂RLNEVAKX₃LNEX₄L, wherein each of X₁, X₂, X₃, and X₄ is independently a stapling amino acid (SEQ ID NO:51); wherein the sidechains of X₁ and X₂ are cross-linked to each other; wherein the sidechains of X₂ and X₃ are cross-linked to each other; and wherein the sidechains of X₃ and X₄ are cross-linked to each other;
(t) IQX₁EIDX₂LNEVAKX₃LNEX₄L, wherein each of X₁, X₂, X₃, and X₄ is independently a stapling amino acid (SEQ ID NO:52); wherein the sidechains of X₁ and X₂ are cross-linked to each other; wherein the sidechains of X₂ and X₃ are cross-linked to each other; and wherein the sidechains of X₃ and X₄ are cross-linked to each other;
(u) IQK8IDRLNEXAKNLNESL, wherein each of 8 and X is independently a stapling amino acid (SEQ ID NO: 113); and wherein the sidechains of 8 and X are cross-linked to each other;
(v) IQKEIDX₁LNEX₂AKNLNESL, wherein each of X₁ and X₂ is independently a stapling amino acid (SEQ ID NO:133); and wherein the sidechains of X₁ and X₂ are cross-linked to each other;
(w) IQKEIDRX₁NEVX₂KNLNESL, wherein each of X₁ and X₂ is independently a stapling amino acid (SEQ ID NO:134); and wherein the sidechains of X₁ and X₂ are cross-linked to each other;
(x) IQKEIDRLNEX₁AKNX₂NESL, wherein each of X₁ and X₂ is independently a stapling amino acid (SEQ ID NO:135); and wherein the sidechains of X₁ and X₂ are cross-linked to each other;
(y) IQKEIDRLNEVX₁KNLX₂ESL, wherein each of X₁ and X₂ is independently a stapling amino acid (SEQ ID NO:136); and wherein the sidechains of X₁ and X₂ are cross-linked to each other; or
(z) IQKEIDRLNEVAKNX₁NESX₂, wherein each of X₁ and X₂ is independently a stapling amino acid (SEQ ID NO: 137); and wherein the sidechains of X₁ and X₂ are cross-linked to each other.

36. The structurally-stabilized peptide of any one of claim 27, or 32-35, which is 15 to 100 amino acids in length, optionally 19 to 45 amino acids in length.

37. A structurally-stabilized peptide comprising the formula: 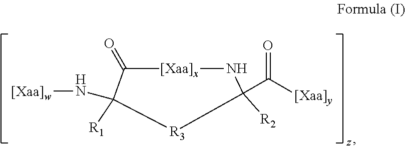
or a pharmaceutically acceptable salt thereof, wherein:
each R₁ and R₂ is H or a C₁ to C₁₀ alkyl, alkenyl, alkynyl, arylalkyl, cycloalkylalkyl, heteroarylalkyl, or heterocyclylalkyl, any of which is substituted or unsubstituted;
each R₃ is independently alkylene, alkenylene, or alkynylene, any of which is substituted or unsubstituted;
z is 1, 2, 3, 4, 5, 6, 7, 8, 9, or 10; and
(a) each [Xaa]_{w} is ISGI (SEQ ID NO:53), each [Xaa]ₓ is ASVVNI (SEQ ID NO:54), and each [Xaa]_{y} is KEIDRLNEVAKNLNESLIDLQELGKYEQYI (SEQ ID NO:55);
(b) each [Xaa]_{w} is ISGIN (SEQ ID NO:56), each [Xaa]ₓ is SVVNIQ (SEQ ID NO:57), and each [Xaa]_{y} is EIDRLNEVAKNLNESLIDLQELGKYEQYI (SEQ ID NO:58);
(c) each [Xaa]_{w} is ISGINA (SEQ ID NO:59), each [Xaa]ₓ is VVNIQK (SEQ ID NO:60), and each [Xaa]_{y} is IDRLNEVAKNLNESLIDLQELGKYEQYI (SEQ ID NO:61);
(d) each [Xaa]_{w} is ISGINASVVNIQKEIDRLNEVAKNL (SEQ ID NO:62), each [Xaa]ₓ is ESLIDL (SEQ ID NO:63), and each [Xaa]_{y} is ELGKYEQYI (SEQ ID NO:64);
(e) each [Xaa]_{w} is ISGINASVVNIQKEIDRLNEVAKNLN (SEQ ID NO:65), each [Xaa]ₓ is SLIDLQ (SEQ ID NO:66), and each [Xaa]_{y} is LGKYEQYI (SEQ ID NO:67);
(f) each [Xaa]_{w} is ISGINASVVNIQKEIDRLNEVAKNLNESLIDL (SEQ ID NO:68), each [Xaa]ₓ is ELGKYE (SEQ ID NO:69), and each [Xaa]_{y} is YI;
(g) each [Xaa]_{w} is I, each [Xaa]ₓ is KEIDRL (SEQ ID NO:70), and each [Xaa]_{y} is EVAKNLNESL (SEQ ID NO:71);
(h) each [Xaa]_{w} is IQ, each [Xaa]ₓ is EIDRLN (SEQ ID NO: 72), and each [Xaa]_{y} is VAKNLNESL (SEQ ID NO:73);
(i) each [Xaa]_{w} is IQKEI (SEQ ID NO:74), each [Xaa]ₓ is RLNEVA (SEQ ID NO:75), and each [Xaa]_{y} is NLNESL (SEQ ID NO:76);
(j) each [Xaa]_{w} is IQKEID (SEQ ID NO:77), each [Xaa]ₓ is LNEVAK (SEQ ID NO:78), and each [Xaa]_{y} is LNESL (SEQ ID NO:79);
(k) each [Xaa]_{w} is IQKEIDRL (SEQ ID NO:80), each [Xaa]ₓ is EVAKNL (SEQ ID NO:81), and each [Xaa]_{y} is ESL;
(l) each [Xaa]_{w} is IQKEIDRLN (SEQ ID NO:82), each [Xaa]ₓ is VAKNLN (SEQ ID NO:83), and each [Xaa]_{y} is SL;
(m) each [Xaa]_{w} is I, each [Xaa]ₓ is KEI, and each [Xaa]_{y} is RLNEVAKNLNESL (SEQ ID NO:84);
(n) each [Xaa]_{w} is IQ, each [Xaa]ₓ is EID, and each [Xaa]_{y} is LNEVAKNLNESL (SEQ ID NO:85);
(o) each [Xaa]_{w} is IQKEI (SEQ ID NO:74), each [Xaa]ₓ is RLN, and each [Xaa]_{y} is VAKNLNESL (SEQ ID NO:73);
(p) each [Xaa]_{w} is IQKEIDRL (SEQ ID NO:80), each [Xaa]ₓ is EVA, and each [Xaa]_{y} is NLNESL (SEQ ID NO:76);
(q) each [Xaa]_{w} is IQKEIDRLN (SEQ ID NO:82), each [Xaa]ₓ is VAK, and each [Xaa]_{y} is LNESL (SEQ ID NO:79);
(r) each [Xaa]_{w} is IQKEIDRLNEVA (SEQ ID NO:86), each [Xaa]ₓ is NLN, and each [Xaa]_{y} is SL;
(s) each [Xaa]_{w} is IQKEIDRLNEVAK (SEQ ID NO:87), each [Xaa]ₓ is LNE, and each [Xaa]_{y} is L;
(t) each [Xaa]_{w} is missing, each [Xaa]ₓ is QKEIDR (SEQ ID NO:228), and each [Xaa]_{y} is NEVAKNLNESL (SEQ ID NO:229);
(u) each [Xaa]_{w} is IQK, each [Xaa]ₓ is IDRLNE (SEQ ID NO:230), and each [Xaa]_{y} is AKNLNESL (SEQ ID NO:231);
(v) each [Xaa]_{w} is IQKE (SEQ ID NO:232), each [Xaa]ₓ is DRLNEV (SEQ ID NO:181), and each [Xaa]_{y} is KNLNESL (SEQ ID NO:182);
(w) each [Xaa]_{w} is IQKEIDR (SEQ ID NO:183), each [Xaa]ₓ is NEVAKN (SEQ ID NO:184), and each [Xaa]_{y} is NESL (SEQ ID NO:185);
(x) each [Xaa]_{w} is IQKEIDRLNE (SEQ ID NO:186), each [Xaa]ₓ is AKNLNE (SEQ ID NO:187), and each [Xaa]_{y} is L;
(y) each [Xaa]_{w} is IQKEIDRLNEV (SEQ ID NO:188), each [Xaa]ₓ is KNLNES (SEQ ID NO:189), and each [Xaa]_{y} is missing;
(z) each [Xaa]_{w} is QKE, each [Xaa]ₓ is DRLNEVAKNLNESL (SEQ ID NO:190), and each [Xaa]_{y} is missing;
(aa) each [Xaa]_{w} is IQK, each [Xaa]ₓ is IDR, and each [Xaa]_{y} is NEVAKNLNESL (SEQ ID NO:229);
(bb) each [Xaa]_{w} is IQK, each [Xaa]ₓ is IDR, and each [Xaa]_{y} is NEVAKNLNESL (SEQ ID NO:229);
(cc) each [Xaa]_{w} is IQKE (SEQ ID NO:232), each [Xaa]x is DRL, and each [Xaa]y is EVAKNLNESL (SEQ ID NO:71);
(dd) each [Xaa]_{w} is IQKEID (SEQ ID NO:77), each [Xaa]x is LNE, and each [Xaa]y is AKNLNESL (SEQ ID NO:231);
(ee) each [Xaa]_{w} is IQKEIDR (SEQ ID NO:183), each [Xaa]x is NEV, and each [Xaa]y is KNLNESL (SEQ ID NO:182);
(ff) each [Xaa]w is IQKEIDRLNE (SEQ ID NO:186), each [Xaa]x is AKN, and each [Xaa]y is NESL (SEQ ID NO:185);
(gg) each [Xaa]w is IQKEIDRLNEV (SEQ ID NO:188), each [Xaa]x is KNL, and each [Xaa]y is ESL;
(hh) each [Xaa]w is IQKEIDRLNEVAKN (SEQ ID NO:191), each [Xaa]x is NES, and each [Xaa]y is missing;
(ii) each [Xaa]w is ISGINASVVN (SEQ ID NO:192), each [Xaa]x is QKEIDR (SEQ ID NO: 228), and each [Xaa]y is NEVAKNLNESL (SEQ ID NO:229);
(jj) each [Xaa]w is ISGINASVVNI (SEQ ID NO:193), each [Xaa]x is KEIDRL (SEQ ID NO:70), and each [Xaa]y is EVAKNLNESL (SEQ ID NO:71);
(kk) each [Xaa]w is ISGINASVVNIQ (SEQ ID NO:194), each [Xaa]x is EIDRLN (SEQ ID NO:72), and each [Xaa]y is VAKNLNESL (SEQ ID NO:73);
(ll) each [Xaa]w is ISGINASVVNIQK (SEQ ID NO:195), each [Xaa]x is IDRLNE (SEQ ID NO:230), and each [Xaa]y is AKNLNESL (SEQ ID NO:231);
(mm) each [Xaa]w is ISGINASVVNIQKE (SEQ ID NO:196), each [Xaa]x is DRLNEV (SEQ ID NO:181), and each [Xaa]y is KNLNESL (SEQ ID NO:182);
(nn) each [Xaa]w is ISGINASVVNIQKEI (SEQ ID NO:197), each [Xaa]x is RLNEVA (SEQ ID NO:75), and each [Xaa]y is NLNESL (SEQ ID NO:76);
(oo) each [Xaa]w is ISGINASVVNIQKEID (SEQ ID NO:198), each [Xaa]x is LNEVAK (SEQ ID NO:78), and each [Xaa]y is LNESL (SEQ ID NO:79);
(pp) each [Xaa]w is ISGINASVVNIQKEIDR (SEQ ID NO:199), each [Xaa]x is NEVAKN (SEQ ID NO:184), and each [Xaa]y is NESL (SEQ ID NO:185);
(qq) each [Xaa]w is ISGINASVVNIQKEIDRL (SEQ ID NO:200), each [Xaa]x is EVAKNL (SEQ ID NO:81), and each [Xaa]y is ESL;
(rr) each [Xaa]w is ISGINASVVNIQKEIDRLN (SEQ ID NO:201), each [Xaa]x is VAKNLN (SEQ ID NO:83), and each [Xaa]y is SL;
(ss) each [Xaa]w is ISGINASVVNIQKEIDRLNE (SEQ ID NO:202), each [Xaa]x is AKNLNE (SEQ ID NO:187), and each [Xaa]y is L;
(tt) each [Xaa]w is ISGINASVVNIQKEIDRLNEV (SEQ ID NO:203), each [Xaa]x is KNLNES (SEQ ID NO:189), and each [Xaa]y is missing;
(uu) each [Xaa]w is ISGINASVVN (SEQ ID NO:192), each [Xaa]x is QKE, and each [Xaa]y is DRLNEVAKNLNESL (SEQ ID NO:190);
(vv) each [Xaa]w is ISGINASVVNI (SEQ ID NO:193), each [Xaa]x is KEI, and each [Xaa]y is RLNEVAKNLNESL (SEQ ID NO:84);
(ww) each [Xaa]w is ISGINASVVNIQ (SEQ ID NO:194), each [Xaa]x is EID, and each [Xaa]y is LNEVAKNLNESL (SEQ ID NO:85);
(xx) each [Xaa]w is ISGINASVVNIQK (SEQ ID NO:195), each [Xaa]x is IDR, and each [Xaa]y is NEVAKNLNESL (SEQ ID NO:229);
(yy) each [Xaa]w is ISGINASVVNIQKE (SEQ ID NO:196), each [Xaa]x is DRL, and each [Xaa]y is EVAKNLNESL (SEQ ID NO:71);
(zz) each [Xaa]w is ISGINASVVNIQKEI (SEQ ID NO:197), each [Xaa]x is RLN, and each [Xaa]y is VAKNLNESL (SEQ ID NO:73);
(aaa) each [Xaa]w is ISGINASVVNIQKEID (SEQ ID NO:198), each [Xaa]x is LNE, and each [Xaa]y is AKNLNESL (SEQ ID NO:231);
(bbb) each [Xaa]w is ISGINASVVNIQKEIDR (SEQ ID NO:199), each [Xaa]x is NEV, and each [Xaa]y is KNLNESL (SEQ ID NO:182);
(ccc) each [Xaa]w is ISGINASVVNIQKEIDRL (SEQ ID NO:200), each [Xaa]x is EVA, and each [Xaa]y is NLNESL (SEQ ID NO:76);
(ddd) each [Xaa]w is ISGINASVVNIQKEIDRLN (SEQ ID NO:201), each [Xaa]x is VAK, and each [Xaa]y is LNESL (SEQ ID NO:79);
(eee) each [Xaa]w is ISGINASVVNIQKEIDRLNE (SEQ ID NO:202), each [Xaa]x is AKN, and each [Xaa]y is NESL (SEQ ID NO:185);
(fff) each [Xaa]w is ISGINASVVNIQKEIDRLNEV (SEQ ID NO:203), each [Xaa]x is KNL, and each [Xaa]y is ESL;
(ggg) each [Xaa]w is ISGINASVVNIQKEIDRLNEVA (SEQ ID NO:204), each [Xaa]x is NLN, and each [Xaa]y is SL;
(hhh) each [Xaa]w is ISGINASVVNIQKEIDRLNEVAK (SEQ ID NO:205), each [Xaa]x is LNE, and each [Xaa]y is L; or
(iii) each [Xaa]w is ISGINASVVNIQKEIDRLNEVAKN (SEQ ID NO:206), each [Xaa]x is NES, and each [Xaa]y is missing; and
wherein the structurally-stabilized peptide has one or more of the properties listed below: (i) binds a recombinant 5-helix bundle of SARS-CoV-2 S protein; (ii) disrupts the interaction between the recombinant 5-helix bundle of SARS-CoV-2 S protein and SEQ ID NO; 9 (ii) is alpha-helical; (iii) is protease resistant; (iv) inhibits fusion of SARS-CoV-2 with a host cell; and/or (v) inhibits infection of a cell by SARS-CoV-2.

38. The structurally-stabilized peptide or pharmaceutically acceptable salt thereof of claim 37, wherein R₁ is an alkyl.

39. The structurally-stabilized peptide or pharmaceutically acceptable salt thereof of claim 37, wherein R₁ is a methyl group.

40. The structurally-stabilized peptide or pharmaceutically acceptable salt thereof of claim 37, wherein R₃ is an alkyl.

41. The structurally-stabilized peptide or pharmaceutically acceptable salt thereof of claim 37, wherein R₃ is a methyl group.

42. The structurally-stabilized peptide or pharmaceutically acceptable salt thereof of claim 37, wherein R₂ is an alkenyl.

43. A structurally-stabilized peptide comprising the formula: 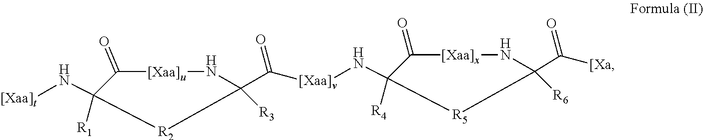
or a pharmaceutically acceptable salt thereof, wherein:
(a) [Xaa]ₜ is ISGI (SEQ ID NO:53), [Xaa]ᵤ is ASVVNI (SEQ ID NO:54), [Xaa]ᵥ is KEIDRLNEVAKNL (SEQ ID NO:88), [Xaa]ₓ is ESLIDL (SEQ ID NO:63), and [Xaa]_{y} is ELGKYEQYI (SEQ ID NO:64);
(b) [Xaa]ₜ is ISGI (SEQ ID NO:53), [Xaa]ᵤ is ASVVNI (SEQ ID NO:54), [Xaa]ᵥ is KEIDRLNEVAKNLN (SEQ ID NO:89), [Xaa]ₓ is SLIDLQ (SEQ ID NO:66), and [Xaa]_{y} is LGKYEQYI (SEQ ID NO:67);
(c) [Xaa]ₜ is ISGI (SEQ ID NO:53), [Xaa]ᵤ is ASVVNI (SEQ ID NO:54), [Xaa]ᵥ is KEIDRLNEVAKNLNESLIDL (SEQ ID NO:90), [Xaa]ₓ is ELGKYE (SEQ ID NO:69), and [Xaa]_{y} is YI;
(d) [Xaa]ₜ is ISGIN (SEQ ID NO:56), [Xaa]ᵤ is SVVNIQ (SEQ ID NO:57), [Xaa]ᵥ is EIDRLNEVAKNL (SEQ ID NO:91), [Xaa]ₓ is ESLIDL (SEQ ID NO:63), and [Xaa]_{y} is ELGKYEQYI (SEQ ID NO:64);
(e) [Xaa]ₜ is ISGIN (SEQ ID NO:56), [Xaa]ᵤ is SVVNIQ (SEQ ID NO:57), [Xaa]ᵥ is EIDRLNEVAKNLN (SEQ ID NO:92), [Xaa]ₓ is SLIDLQ (SEQ ID NO:66), and [Xaa]_{y} is LGKYEQYI (SEQ ID NO:67);
(f) [Xaa]ₜ is ISGIN (SEQ ID NO:56), [Xaa]ᵤ is SVVNIQ (SEQ ID NO:57), [Xaa]ᵥ is EIDRLNEVAKNLNESLIDL (SEQ ID NO:93), [Xaa]ₓ is ELGKYE (SEQ ID NO:69), and [Xaa]_{y} is YI;
(g) [Xaa]ₜ is ISGINA (SEQ ID NO:59), [Xaa]ᵤ is VVNIQK (SEQ ID NO:60), [Xaa]ᵥ is IDRLNEVAKNL (SEQ ID NO:94), [Xaa]ₓ is ESLIDL (SEQ ID NO:63), and [Xaa]_{y} is ELGKYEQYI (SEQ ID NO:64);
(h) [Xaa]ₜ is ISGINA (SEQ ID NO:59), [Xaa]ᵤ is VVNIQK (SEQ ID NO:60), [Xaa]ᵥ is IDRLNEVAKNLN (SEQ ID NO:95), [Xaa]ₓ is SLIDLQ (SEQ ID NO:66), and [Xaa]_{y} is LGKYEQYI (SEQ ID NO:67);
(i) [Xaa]ₜ is ISGINA (SEQ ID NO:59), [Xaa]ᵤ is VVNIQK (SEQ ID NO:60), [Xaa]ᵥ is IDRLNEVAKNLNESLIDL (SEQ ID NO:96), [Xaa]ₓ is ELGKYE (SEQ ID NO:69), and [Xaa]_{y} is YI;
(j) [Xaa]ₜ is I, [Xaa]ᵤ is KEI, [Xaa]ᵥ is RLNEVA (SEQ ID NO:97), [Xaa]ᵥ is NLN, and [Xaa]_{y} is SL;
(k) [Xaa]ₜ is IQ, [Xaa]ᵤ is EID, [Xaa]ᵥ is LNEVA (SEQ ID NO:98), [Xaa]ₓ is NLN, and [Xaa]_{y} is SL;
(l) [Xaa]ₜ is I, [Xaa]ᵤ is KEI, [Xaa]ᵥ is RLNEVAK (SEQ ID NO:99), [Xaa]ₓ is LNE, and [Xaa]_{y} is L;
(m)[Xaa]ₜ is IQ, [Xaa]ᵤ is EID, [Xaa]ᵥ is LNEVAK (SEQ ID NO:100), [Xaa]x is LNE, and [Xaa]_{y} is L;
(n) [Xaa]ₜ is I, [Xaa]ᵤ is KEI, [Xaa]ᵥ is RLNEVA (SEQ ID NO:75), [Xaa]ₓ is NLN, and [Xaa]_{y} is SL;
(o) [Xaa]ₜ is IQ, [Xaa]ᵤ is EID, [Xaa]ᵥ is LNEVA (SEQ ID NO:98), [Xaa]ₓ is NLN, and [Xaa]_{y} is SL;
(p) [Xaa]ₜ is I, [Xaa]ᵤ is KEI, [Xaa]ᵥ is RLNEVAK (SEQ ID NO:99), [Xaa]ₓ is LNE, and [Xaa]_{y} is L;
(q) [Xaa]ₜ is IQ, [Xaa]ᵤ is EID, [Xaa]ᵥ is LNEVAK (SEQ ID NO:78), [Xaa]ₓ is LNE, and [Xaa]_{y} is L;
(r) [Xaa]ₜ is I, [Xaa]ᵤ is KEI, [Xaa]ᵥ is RLNEVA (SEQ ID NO:75), [Xaa]x is NLN, and [Xaa]_{y} is SLIDLQEL (SEQ ID NO:207);
(s) [Xaa]ₜ is Q, [Xaa]ᵤ is EID, [Xaa]ᵥ is LNEVA (SEQ ID NO:98), [Xaa]ₓ is NLN, and [Xaa]_{y} is SLIDLQEL (SEQ ID NO:207);
(t) [Xaa]ₜ is I, [Xaa]ᵤ is KEI, [Xaa]ᵥ is RLNEVAK (SEQ ID NO:99), [Xaa]x is LNE, and [Xaa]_{y} is LIDLQEL (SEQ ID NO:208);
(u) [Xaa]ₜ is IQ, [Xaa]ᵤ is EID, [Xaa]ᵥ is LNEVAK (SEQ ID NO:78), [Xaa]x is LNE, and [Xaa]_{y} is LIDLQEL (SEQ ID NO:208);
(v) [Xaa]ₜ is DISGINASVVNI (SEQ ID NO:209), [Xaa]ᵤ is KEI, [Xaa]ᵥ is RLNEVA (SEQ ID NO:75), [Xaa]ₓ is NLN, and [Xaa]_{y} is SL;
(w) [Xaa]ₜ is DISGINASVVNIQ (SEQ ID NO:210), [Xaa]ᵤ is EID, [Xaa]ᵥ is LNEVA (SEQ ID NO:98), [Xaa]ₓ is NLN, and [Xaa]_{y} is SL;
(x) [Xaa]ₜ is DISGINASVVNI (SEQ ID NO:209), [Xaa]ᵤ is KEI, and [Xaa]ᵥ is RLNEVAK (SEQ ID NO:99), [Xaa]ₓ is LNE, and [Xaa]_{y} is L;
(y) [Xaa]ₜ is DISGINASVVNIQ (SEQ ID NO:210), [Xaa]ᵤ is EID, [Xaa]ᵥ is LNEVAK (SEQ ID NO:78), [Xaa]ₓ is LNE, and [Xaa]_{y} is L;
(z) [Xaa]ₜ is DISGINASVVNI (SEQ ID NO:209), [Xaa]ᵤ is KEI, [Xaa]ᵥ is RLNEVA (SEQ ID NO:75), [Xaa]ₓ is NLN, and [Xaa]_{y} is SLIDLQEL (SEQ ID NO:207);
(aa) [Xaa]ₜ is DISGINASVVNIQ (SEQ ID NO:210), [Xaa]ᵤ is EID, [Xaa]ᵥ is LNEVA (SEQ ID NO:98), [Xaa]ₓ is NLN, and [Xaa]_{y} is SLIDLQEL (SEQ ID NO:207);
(bb) [Xaa]ₜ is DISGINASVVNI (SEQ ID NO:209), [Xaa]ᵤ is KEI, [Xaa]ᵥ is RLNEVAK (SEQ ID NO:99), [Xaa]ₓ is LNE, and [Xaa]_{y} is LIDLQEL (SEQ ID NO:208);
(cc) [Xaa]ₜ is DISGINASVVNIQ (SEQ ID NO:210), [Xaa]ᵤ is EID, [Xaa]ᵥ is LNEVAK (SEQ ID NO:78), [Xaa]ₓ is LNE, and [Xaa]_{y} is LIDLQEL (SEQ ID NO:208) (dd) [Xaa]ₜ is ISGINASVVNI (SEQ ID NO:193), [Xaa]ᵤ is KEI, [Xaa]ᵥ is RLNEVA (SEQ ID NO:75), [Xaa]ₓ is NLN, and [Xaa]_{y} is SLIDLQELGKYEQYI (SEQ ID NO:211);
(ee) [Xaa]ₜ is ISGINASVVNIQ (SEQ ID NO:194), [Xaa]ᵤ is EID, [Xaa]ᵥ is LNEVA (SEQ ID NO:98), [Xaa]ₓ is NLN, and [Xaa]_{y} is SLIDLQELGKYEQYI (SEQ ID NO:211);
(ff) [Xaa]ₜ is ISGINASVVNI (SEQ ID NO: 193), [Xaa]ᵤ is KEI, [Xaa]ᵥ is RLNEVAK (SEQ ID NO:99), [Xaa]ₓ is LNE, and [Xaa]_{y} is LIDLQELGKYEQYI (SEQ ID NO:212);
(gg) [Xaa]ₜ is ISGINASVVNIQ (SEQ ID NO:194), [Xaa]ᵤ is EID, [Xaa]ᵥ is LNEVAK (SEQ ID NO:78), [Xaa]ₓ is LNE, and [Xaa]_{y} is LIDLQELGKYEQYI (SEQ ID NO:212);
(hh) [Xaa]ₜ is SLDQINVTFLDL (SEQ ID NO:213), [Xaa]ᵤ is YEB, [Xaa]ᵥ is KLEEAI (SEQ ID NO:214), [Xaa]ₓ is KLE, and [Xaa]_{y} is SYIDLKE (SEQ ID NO:215);
(ii) [Xaa]ₜ is SLDQINVTFLDLE (SEQ ID NO:216), [Xaa]ᵤ is EBK, [Xaa]ᵥ is LEEAI (SEQ ID NO:217), [Xaa]ₓ is KLE, and [Xaa]_{y} is SYIDLKE (SEQ ID NO:215);
(jj) [Xaa]ₜ is SLDQINVTFLDL (SEQ ID NO:213), [Xaa]ᵤ is YEB, [Xaa]ᵥ is KLEEAIK (SEQ ID NO:218), [Xaa]ₓ is LEE, and [Xaa]_{y} is YIDLKE (SEQ ID NO:219);
(kk) [Xaa]ₜ is SLDQINVTFLDLE (SEQ ID NO:216), [Xaa]ᵤ is EBK, [Xaa]ᵥ is LEEAIK (SEQ ID NO:220), [Xaa]ₓ is LEE, and [Xaa]_{y} is YIDLKE (SEQ ID NO:219),
wherein R₁, R₃, R₄, and R₆ is independently H, alkyl, alkenyl, alkynyl, arylalkyl, cycloalkylalkyl, heteroarylalkyl, or heterocyclylalkyl, any of which is substituted or unsubstituted;
wherein R₂ and R₅ is independently alkylene, alkenylene, or alkynylene, any of which is substituted or unsubstituted; and
wherein the structurally-stabilized peptide has one or more of the properties listed below: (i) binds a recombinant 5-helix bundle of SARS-CoV-2 S protein; (ii) disrupts the interaction between the recombinant 5-helix bundle of SARS-CoV-2 S protein and SEQ ID NO:9 or 10; (iii) is alpha-helical; (iv) is protease resistant; (v) inhibits fusion of SARS-CoV-2 with a host cell; and/or (vi) inhibits infection of a cell by SARS-CoV-2.

44. A structurally-stabilized peptide comprising the formula: 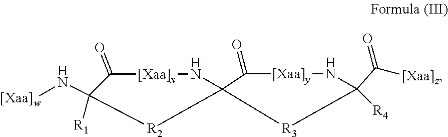
or a pharmaceutically acceptable salt thereof, wherein:
(a) [Xaa]_{w} is
(SEQ ID NO: 62)
ISGINASVVNIQKEIDRLNEVAKNL;
[Xaa]ₓ is
(SEQ ID NO: 63)
ESLIDL;
[Xaa]_{y} is
(SEQ ID NO: 69)
ELGKYE;
and
[Xaa]_{z} is
YI;
(b) [Xaa]_{w} is
I;
[Xaa]ₓ is
(SEQ ID NO: 70)
KEIDRL;
[Xaa]_{y} is
(SEQ ID NO: 81)
EVAKNL;
and
[Xaa]_{z} is
ESL;
(c) [Xaa]_{w} is
IQ;
[Xaa]ₓ is
(SEQ ID NO: 72)
EIDRLN;
[Xaa]_{y} is
(SEQ ID NO: 83)
VAKNLN;
and
[Xaa]_{z} is
SL;
(d) [Xaa]_{w} is
I;
[Xaa]ₓ is
(SEQ ID NO: 70)
KEIDRL;
[Xaa]_{y} is
EVA;
and
[Xaa]_{z} is
(SEQ ID NO: 76)
NLNESL;
(e) [Xaa]_{w} is
IQ;
[Xaa]ₓ is
(SEQ ID NO: 72)
EIDRLN;
[Xaa]_{y} is
VAK;
and
[Xaa]_{z} is
(SEQ ID NO: 79)
LNESL;
(f) [Xaa]_{w} is
(SEQ ID NO: 74)
IQKEI;
[Xaa]ₓ is
(SEQ ID NO: 75)
RLNEVA;
[Xaa]_{y} is
NLN;
and
[Xaa]_{z} is
SL;
(g) [Xaa]_{w} is
(SEQ ID NO: 77)
IQKEID;
[Xaa]ₓ is
(SEQ ID NO: 78)
LNEVAK;
[Xaa]_{y} is
LNE;
and
[Xaa]_{z} is
L;
(h) [Xaa]_{w} is
(SEQ ID NO: 193)
ISGINASVVNI;
[Xaa]ₓ is
(SEQ ID NO: 70)
KEIDRL;
[Xaa]_{y} is
(SEQ ID NO: 81)
EVAKNL;
and
[Xaa]_{z} is
(SEQ ID NO: 221)
ESLIDLQELGKYEQYI;
(i) [Xaa]_{w} is
(SEQ ID NO: 194)
ISGINASVVNIQ;
[Xaa]ₓ is
(SEQ ID NO: 72)
EIDRLN;
[Xaa]_{y} is
VAK;
and
[Xaa]_{z} is
(SEQ ID NO: 222)
LNESLIDLQELGKYEQYI;
(j) [Xaa]_{w} is
(SEQ ID NO: 213)
SLDQINVTFLDL;
[Xaa]ₓ is
(SEQ ID NO: 223)
YEBKKL;
[Xaa]_{y} is
(SEQ ID NO: 224)
EAIKKL;
and
[Xaa]_{z} is
(SEQ ID NO: 225)
ESYIDLKE;
or
(k) [Xaa]_{w} is
(SEQ ID NO: 216)
SLDQINVTFLDLE;
[Xaa]ₓ is
(SEQ ID NO: 226)
EBKKLE;
[Xaa]_{y} is
(SEQ ID NO: 227)
AIKKLE;
and
[Xaa]_{z} is
(SEQ ID NO: 215)
SYIDLKE;
wherein R₁ and R₄ is independently H, alkyl, alkenyl, alkynyl, arylalkyl, cycloalkylalkyl, heteroarylalkyl, or heterocyclylalkyl, any of which is substituted or unsubstituted;
wherein R₂ and R₃ is independently alkylene, alkenylene, or alkynylene, any of which is substituted or unsubstituted; and
wherein the structurally-stabilized peptide has one or more of the properties listed below: (i) binds a recombinant 5-helix bundle of SARS-CoV-2 S protein; (ii) is alpha-helical; (iii) is protease resistant; (iv) inhibits fusion of SARS-CoV-2 with a host cell; and/or (v) inhibits infection of a cell by SARS-CoV-2.

45. A structurally-stabilized peptide comprising the formula: 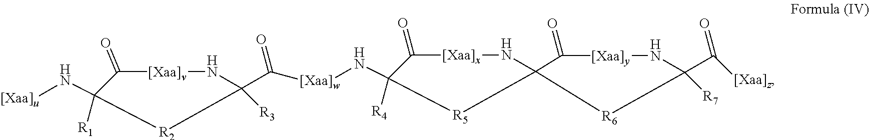
or a pharmaceutically acceptable salt thereof, wherein:
(a) [Xaa]ᵤ is ISGI (SEQ ID NO:53), [Xaa]ᵥ is ASVVNI (SEQ ID NO:54), [Xaa]_{w} is KEIDRLNEVAKNL (SEQ ID NO:88), [Xaa]ₓ is ESLIDL (SEQ ID NO:63), [Xaa]_{y} is ELGKYE (SEQ ID NO:69), and [Xaa]_{z} is YI;
(b) [Xaa]ᵤ is ISGIN (SEQ ID NO:56), [Xaa]ᵥ is SVVNIQ (SEQ ID NO:57), [Xaa]_{w} is EIDRLNEVAKNL (SEQ ID NO:91), [Xaa]ₓ is ESLIDL (SEQ ID NO:63), [Xaa]_{y} is ELGKYE (SEQ ID NO:69), and [Xaa]_{z} is YI; or
(c) [Xaa]ᵤ is ISGINA (SEQ ID NO:59), [Xaa]ᵥ is VVNIQK (SEQ ID NO:60), [Xaa]_{w} is IDRLNEVAKNL (SEQ ID NO:94), [Xaa]ₓ is ESLIDL (SEQ ID NO:63), [Xaa]_{y} is ELGKYE (SEQ ID NO:69), and [Xaa]_{z} is YI; and
wherein R₁, R₃, R₄, and R₇ is independently H, alkyl, alkenyl, alkynyl, arylalkyl, cycloalkylalkyl, heteroarylalkyl, or heterocyclylalkyl, any of which is substituted or unsubstituted;
wherein R₂, R₅, and R₆ is independently alkylene, alkenylene, or alkynylene, any of which is substituted or unsubstituted; and
wherein the structurally-stabilized peptide has one or more of the properties listed below: (i) binds a recombinant 5-helix bundle of SARS-CoV-2 S protein; ((ii) disrupts the interaction between the recombinant 5-helix bundle of SARS-CoV-2 S protein and of peptide of SEQ ID NO:9 or 10; (iii) is alpha-helical; (iv) is protease resistant; (v) inhibits fusion of SARS-CoV-2 with a host cell; and/or (vi) inhibits infection of a cell by SARS-CoV-2.

46. The structurally-stabilized peptide or pharmaceutically acceptable salt thereof of any one of claims 37 to 45, wherein R₁ is an alkyl.

47. The structurally-stabilized peptide or pharmaceutically acceptable salt thereof of any one of claims 37 to 45, wherein R₁ is a methyl group.

48. The structurally-stabilized peptide or pharmaceutically acceptable salt thereof of any one of claims 37 to 47, wherein R₄ is an alkyl.

49. The compound or pharmaceutically acceptable salt thereof of any one of claims 37 to 47, wherein R₄ is a methyl group.

50. The structurally-stabilized peptide or pharmaceutically acceptable salt thereof of any one of claims 37 to 49, wherein R₂ is an alkenyl.

51. The structurally-stabilized peptide or pharmaceutically acceptable salt thereof of any one of claims 37 to 50, wherein R₃ is an alkenyl.

52. The structurally-stabilized peptide or pharmaceutically acceptable salt thereof of any one of claims 37 to 51, which is at most 100 amino acids in length, optionally at most 45 amino acids in length.

53. A pharmaceutical compound comprising the structurally-stabilized peptide, the peptide, or pharmaceutically acceptable salt thereof of any one of claims 1 to 52 and a pharmaceutically acceptable carrier.

54. A method of treating a coronavirus infection in a human subject in need thereof, the method comprising administering to the human subject a therapeutically-effective amount of the structurally-stabilized peptide, the peptide, or pharmaceutically acceptable salt thereof of any one of claims 1 to 52.

55. A method of preventing a coronavirus infection in a human subject in need thereof, the method comprising administering to the human subject a therapeutically-effective amount of the structurally-stabilized peptide, the peptide, or pharmaceutically acceptable salt thereof of any one of claims 1 to 52.

56. The method of claim 54 or 55, wherein the coronavirus infection is by a betacoronavirus.

57. The method of any one of claims 54 to 56, wherein the coronavirus infection is caused by an infection by SARS-CoV-2.

58. A method of making a structurally-stabilized peptide, the method comprising: (a) providing a peptide having the sequence set forth in any one of SEQ ID NOs: 11-52, 112-180, or 258, and (b) cross-linking the peptide.

59. The method of claim 58, wherein cross-linking the peptide is by a ruthenium catalyzed metathesis reaction.

60. A nanoparticle composition comprising the structurally-stabilized peptide of any one of claims 1 to 52, optionally wherein the nanoparticle is a PLGA nanoparticle, and further optionally, wherein the lactic acid:glycolic acid ratio of the PLGA nanoparticle is in the range of 2:98 to 100:0.

61. The structurally-stabilized peptide of any one of claims 1 to 52, wherein
8, 8₁, and 8₂=(R)-α-(7′-octenyl)alanine or (R)-α-(4′-pentenyl)alanine;
X, X₁, X₂, X₃, and X₄=(S)-α-(4′-pentenyl)alanine;
#=α,α-Bis(4′-pentenyl)glycine or α,α-Bis(7′-octenyl)glycine; and
%=(S)-α-(7′-octenyl)alanine or (S)-α-(4′-pentenyl)alanine.

62. The structurally-stabilized peptide of any one of claims 1 to 52, wherein 8, 8₁, and 8₂=(R)-α-(7′-octenyl)alanine;
X, X₁, X₂, X₃, and X₄=(S)-α-(4′-pentenyl)alanine;
#=α,α-Bis(4′-pentenyl)glycine; and
% (S)-α-(7′-octenyl)alanine.


SEQUENCE LISTING (263 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 73 aa, Severe acute respiratory syndrome coronavirus 2
TQNVLYENQKLIANQFNSAIGKIQDSLSSTASALGKLQDVVNQNAQALNTLVKQLSSNFGAISSVLNDILSRL

SEQ ID NO: 3 - protein, 51 aa, Unknown
  source: /note=“Description of Unknown SARS and COVID-19 HR2 sequence”
DVDLGDISGINASVVNIQKEIDRLNEVAKNLNESLIDLQELGKYEQYIKWP

SEQ ID NO: 4 - protein, 46 aa, Severe acute respiratory syndrome coronavirus 2
DLGDISGINASVVNIQKEIDRLNEVAKNLNESLIDLQELGKYEQYI

SEQ ID NO: 5 - protein, 23 aa, Severe acute respiratory syndrome coronavirus 2
DLGDISGINASVVNIQKEIDRLN

SEQ ID NO: 6 - protein, 23 aa, Severe acute respiratory syndrome coronavirus 2
EVAKNLNESLIDLQELGKYEQYI

SEQ ID NO: 7 - protein, 19 aa, Severe acute respiratory syndrome coronavirus 2
IQKEIDRLNEVAKNLNESL

SEQ ID NO: 8 - protein, 73 aa, Unknown
  source: /note=“Description of Unknown SARS HR1 sequence”
TQNVLYENQKQIANQFNKAISQIQESLTTTSTALGKLQDVVNQNAQALNTLVKQLSSNFGAISSVLNDILSRL

SEQ ID NO: 9 - protein, 42 aa, Severe acute respiratory syndrome coronavirus 2
ISGINASVVNIQKEIDRLNEVAKNLNESLIDLQELGKYEQYI

SEQ ID NO: 10 - protein, 19 aa, Severe acute respiratory syndrome coronavirus 2
IQKEIDRLNEVAKNLNESL

SEQ ID NO: 11 - protein, 42 aa, Artificial Sequence
  source: /note=“Description of Artificial Sequence Synthetic polypeptide”
  MOD_RES (5)..(5): Any stapling amino acid
  SITE (5)..(12): /note=“May or may not be cross-linked via a side chain”
  MOD_RES (12)..(12): Any stapling amino acid
ISGIXASVVNIXKEIDRLNEVAKNLNESLIDLQELGKYEQYI

SEQ ID NO: 12 - protein, 42 aa, Artificial Sequence
  source: /note=“Description of Artificial Sequence Synthetic polypeptide”
  MOD_RES (6)..(6): Any stapling amino acid
  SITE (6)..(13): /note=“May or may not be cross-linked via a side chain”
  MOD_RES (13)..(13): Any stapling amino acid
ISGINXSVVNIQXEIDRLNEVAKNLNESLIDLQELGKYEQYI

SEQ ID NO: 13 - protein, 42 aa, Artificial Sequence
  source: /note=“Description of Artificial Sequence Synthetic polypeptide”
  MOD_RES (7)..(7): Any stapling amino acid
  SITE (7)..(14): /note=“May or may not be cross-linked via a side chain”
  MOD_RES (14)..(14): Any stapling amino acid
ISGINAXVVNIQKXIDRLNEVAKNLNESLIDLQELGKYEQYI

SEQ ID NO: 14 - protein, 42 aa, Artificial Sequence
  source: /note=“Description of Artificial Sequence Synthetic polypeptide”
  MOD_RES (26)..(26): Any stapling amino acid
  SITE (26)..(33): /note=“May or may not be cross-linked via a side chain”
  MOD_RES (33)..(33): Any stapling amino acid
ISGINASVVNIQKEIDRLNEVAKNLXESLIDLXELGKYEQYI

SEQ ID NO: 15 - protein, 42 aa, Artificial Sequence
  source: /note=“Description of Artificial Sequence Synthetic polypeptide”
  MOD_RES (27)..(27): Any stapling amino acid
  SITE (27)..(34): /note=“May or may not be cross-linked via a side chain”
  MOD_RES (34)..(34): Any stapling amino acid
ISGINASVVNIQKEIDRLNEVAKNLNXSLIDLQXLGKYEQYI

SEQ ID NO: 16 - protein, 42 aa, Artificial Sequence
  source: /note=“Description of Artificial Sequence Synthetic polypeptide”
  MOD_RES (33)..(33): Any stapling amino acid
  SITE (33)..(40): /note=“May or may not be cross-linked via a side chain”
  MOD_RES (40)..(40): Any stapling amino acid
ISGINASVVNIQKEIDRLNEVAKNLNESLIDLXELGKYEXYI

SEQ ID NO: 17 - protein, 42 aa, Artificial Sequence
  source: /note=“Description of Artificial Sequence Synthetic polypeptide”
  MOD_RES (26)..(26): Any stapling/stitching amino acid
  SITE (26)..(33): /note=“May or may not be cross-linked via a side chain”
  MOD_RES (33)..(33): Any stapling/stitching amino acid
  SITE (33)..(40): /note=“May or may not be cross-linked via a side chain”
  MOD_RES (40)..(40): Any stapling/stitching amino acid
ISGINASVVNIQKEIDRLNEVAKNLXESLIDLXELGKYEXYI

SEQ ID NO: 18 - protein, 42 aa, Artificial Sequence
  source: /note=“Description of Artificial Sequence Synthetic polypeptide”
  MOD_RES (5)..(5): Any stapling amino acid
  SITE (5)..(12): /note=“May or may not be cross-linked via a side chain”
  MOD_RES (12)..(12): Any stapling amino acid
  MOD_RES (26)..(26): Any stapling amino acid
  SITE (26)..(33): /note=“May or may not be cross-linked via a side chain”
  MOD_RES (33)..(33): Any stapling amino acid
ISGIXASVVNIXKEIDRLNEVAKNLXESLIDLXELGKYEQYI

SEQ ID NO: 19 - protein, 42 aa, Artificial Sequence
  source: /note=“Description of Artificial Sequence Synthetic polypeptide”
  MOD_RES (5)..(5): Any stapling amino acid
  SITE (5)..(12): /note=“May or may not be cross-linked via a side chain”
  MOD_RES (12)..(12): Any stapling amino acid
  MOD_RES (27)..(27): Any stapling amino acid
  SITE (27)..(34): /note=“May or may not be cross-linked via a side chain”
  MOD_RES (34)..(34): Any stapling amino acid
ISGIXASVVNIXKEIDRLNEVAKNLNXSLIDLQXLGKYEQYI

SEQ ID NO: 20 - protein, 42 aa, Artificial Sequence
  source: /note=“Description of Artificial Sequence Synthetic polypeptide”
  MOD_RES (5)..(5): Any stapling amino acid
  SITE (5)..(12): /note=“May or may not be cross-linked via a side chain”
  MOD_RES (12)..(12): Any stapling amino acid
  MOD_RES (33)..(33): Any stapling amino acid
  SITE (33)..(40): /note=“May or may not be cross-linked via a side chain”
  MOD_RES (40)..(40): Any stapling amino acid
ISGIXASVVNIXKEIDRLNEVAKNLNESLIDLXELGKYEXYI

SEQ ID NO: 21 - protein, 42 aa, Artificial Sequence
  source: /note=“Description of Artificial Sequence Synthetic polypeptide”
  MOD_RES (5)..(5): Any stapling/stitching amino acid
  SITE (5)..(12): /note=“May or may not be cross-linked via a side chain”
  MOD_RES (12)..(12): Any stapling/stitching amino acid
  MOD_RES (26)..(26): Any stapling/stitching amino acid
  SITE (26)..(33): /note=“May or may not be cross-linked via a side chain”
  MOD_RES (33)..(33): Any stapling/stitching amino acid
  SITE (33)..(40): /note=“May or may not be cross-linked via a side chain”
  MOD_RES (40)..(40): Any stapling/stitching amino acid
ISGIXASVVNIXKEIDRLNEVAKNLXESLIDLXELGKYEXYI

SEQ ID NO: 22 - protein, 42 aa, Artificial Sequence
  source: /note=“Description of Artificial Sequence Synthetic polypeptide”
  MOD_RES (6)..(6): Any stapling amino acid
  SITE (6)..(13): /note=“May or may not be cross-linked via a side chain”
  MOD_RES (13)..(13): Any stapling amino acid
  MOD_RES (26)..(26): Any stapling amino acid
  SITE (26)..(33): /note=“May or may not be cross-linked via a side chain”
  MOD_RES (33)..(33): Any stapling amino acid
ISGINXSVVNIQXEIDRLNEVAKNLXESLIDLXELGKYEQYI

SEQ ID NO: 23 - protein, 42 aa, Artificial Sequence
  source: /note=“Description of Artificial Sequence Synthetic polypeptide”
  MOD_RES (6)..(6): Any stapling amino acid
  SITE (6)..(13): /note=“May or may not be cross-linked via a side chain”
  MOD_RES (13)..(13): Any stapling amino acid
  MOD_RES (27)..(27): Any stapling amino acid
  SITE (27)..(34): /note=“May or may not be cross-linked via a side chain”
  MOD_RES (34)..(34): Any stapling amino acid
ISGINXSVVNIQXEIDRLNEVAKNLNXSLIDLQXLGKYEQYI

SEQ ID NO: 24 - protein, 42 aa, Artificial Sequence
  source: /note=“Description of Artificial Sequence Synthetic polypeptide”
  MOD_RES (6)..(6): Any stapling amino acid
  SITE (6)..(13): /note=“May or may not be cross-linked via a side chain”
  MOD_RES (13)..(13): Any stapling amino acid
  MOD_RES (33)..(33): Any stapling amino acid
  SITE (33)..(40): /note=“May or may not be cross-linked via a side chain”
  MOD_RES (40)..(40): Any stapling amino acid
ISGINXSVVNIQXEIDRLNEVAKNLNESLIDLXELGKYEXYI

SEQ ID NO: 25 - protein, 42 aa, Artificial Sequence
  source: /note=“Description of Artificial Sequence Synthetic polypeptide”
  MOD_RES (6)..(6): Any stapling/stitching amino acid
  SITE (6)..(13): /note=“May or may not be cross-linked via a side chain”
  MOD_RES (13)..(13): Any stapling/stitching amino acid
  MOD_RES (26)..(26): Any stapling/stitching amino acid
  SITE (26)..(33): /note=“May or may not be cross-linked via a side chain”
  MOD_RES (33)..(33): Any stapling/stitching amino acid
  SITE (33)..(40): /note=“May or may not be cross-linked via a side chain”
  MOD_RES (40)..(40): Any stapling/stitching amino acid
ISGINXSVVNIQXEIDRLNEVAKNLXESLIDLXELGKYEXYI

SEQ ID NO: 26 - protein, 42 aa, Artificial Sequence
  source: /note=“Description of Artificial Sequence Synthetic polypeptide”
  MOD_RES (7)..(7): Any stapling amino acid
  SITE (7)..(14): /note=“May or may not be cross-linked via a side chain”
  MOD_RES (14)..(14): Any stapling amino acid
  MOD_RES (26)..(26): Any stapling amino acid
  SITE (26)..(33): /note=“May or may not be cross-linked via a side chain”
  MOD_RES (33)..(33): Any stapling amino acid
ISGINAXVVNIQKXIDRLNEVAKNLXESLIDLXELGKYEQYI

SEQ ID NO: 27 - protein, 42 aa, Artificial Sequence
  source: /note=“Description of Artificial Sequence Synthetic polypeptide”
  MOD_RES (7)..(7): Any stitching amino acid
  SITE (7)..(14): /note=“May or may not be cross-linked via a side chain”
  MOD_RES (14)..(14): Any stitching amino acid
  MOD_RES (27)..(27): Any stitching amino acid
  SITE (27)..(34): /note=“May or may not be cross-linked via a side chain”
  MOD_RES (34)..(34): Any stitching amino acid
ISGINAXVVNIQKXIDRLNEVAKNLNXSLIDLQXLGKYEQYI

SEQ ID NO: 28 - protein, 42 aa, Artificial Sequence
  source: /note=“Description of Artificial Sequence Synthetic polypeptide”
  MOD_RES (7)..(7): Any stapling amino acid
  SITE (7)..(14): /note=“May or may not be cross-linked via a side chain”
  MOD_RES (14)..(14): Any stapling amino acid
  MOD_RES (33)..(33): Any stapling amino acid
  SITE (33)..(40): /note=“May or may not be cross-linked via a side chain”
  MOD_RES (40)..(40): Any stapling amino acid
ISGINAXVVNIQKXIDRLNEVAKNLNESLIDLXELGKYEXYI

SEQ ID NO: 29 - protein, 42 aa, Artificial Sequence
  source: /note=“Description of Artificial Sequence Synthetic polypeptide”
  MOD_RES (7)..(7): Any stapling/stitching amino acid
  SITE (7)..(14): /note=“May or may not be cross-linked via a side chain”
  MOD_RES (14)..(14): Any stapling/stitching amino acid
  MOD_RES (26)..(26): Any stapling/stitching amino acid
  SITE (26)..(33): /note=“May or may not be cross-linked via a side chain”
  MOD_RES (33)..(33): Any stapling/stitching amino acid
  SITE (33)..(40): /note=“May or may not be cross-linked via a side chain”
  MOD_RES (40)..(40): Any stapling/stitching amino acid
ISGINAXVVNIQKXIDRLNEVAKNLXESLIDLXELGKYEXYI

SEQ ID NO: 30 - protein, 19 aa, Artificial Sequence
  source: /note=“Description of Artificial Sequence Synthetic peptide”
  MOD_RES (2)..(2): Any stapling amino acid
  SITE (2)..(3): /note=“May or may not be cross-linked via a side chain”
  MOD_RES (9)..(9): Any stapling amino acid
IXKEIDRLXEVAKNLNESL

SEQ ID NO: 31 - protein, 19 aa, Artificial Sequence
  source: /note=“Description of Artificial Sequence Synthetic peptide”
  MOD_RES (3)..(3): Any stapling amino acid
  SITE (3)..(10): /note=“May or may not be cross-linked via a side chain”
  MOD_RES (10)..(10): Any stapling amino acid
IQXEIDRLNXVAKNLNESL

SEQ ID NO: 32 - protein, 19 aa, Artificial Sequence
  source: /note=“Description of Artificial Sequence Synthetic peptide”
  MOD_RES (6)..(6): Any stapling amino acid
  SITE (6)..(13): /note=“May or may not be cross-linked via a side chain”
  MOD_RES (13)..(13): Any stapling amino acid
IQKEIXRLNEVAXNLNESL

SEQ ID NO: 33 - protein, 19 aa, Artificial Sequence
  source: /note=“Description of Artificial Sequence Synthetic peptide”
  MOD_RES (7)..(7): Any stapling amino acid
  SITE (7)..(14): /note=“May or may not be cross-linked via a side chain”
  MOD_RES (14)..(14): Any stapling amino acid
IQKEIDXLNEVAKXLNESL

SEQ ID NO: 34 - protein, 19 aa, Artificial Sequence
  source: /note=“Description of Artificial Sequence Synthetic peptide”
  MOD_RES (9)..(9): Any stapling amino acid
  SITE (9)..(16): /note=“May or may not be cross-linked via a side chain”
  MOD_RES (16)..(16): Any stapling amino acid
IQKEIDRLXEVAKNLXESL

SEQ ID NO: 35 - protein, 19 aa, Artificial Sequence
  source: /note=“Description of Artificial Sequence Synthetic peptide”
  MOD_RES (10)..(10): Any stapling amino acid
  SITE (10)..(17): /note=“May or may not be cross-linked via a side chain”
  MOD_RES (17)..(17): Any stapling amino acid
IQKEIDRLNXVAKNLNXSL

SEQ ID NO: 36 - protein, 19 aa, Artificial Sequence
  source: /note=“Description of Artificial Sequence Synthetic peptide”
  MOD_RES (2)..(2): Any stapling amino acid
  SITE (2)..(6): /note=“May or may not be cross-linked via a side chain”
  MOD_RES (6)..(6): Any stapling amino acid
IXKEIXRLNEVAKNLNESL

SEQ ID NO: 37 - protein, 19 aa, Artificial Sequence
  source: /note=“Description of Artificial Sequence Synthetic peptide”
  MOD_RES (3)..(3): Any stapling amino acid
  SITE (3)..(7): /note=“May or may not be cross-linked via a side chain”
  MOD_RES (7)..(7): Any stapling amino acid
IQXEIDXLNEVAKNLNESL

SEQ ID NO: 38 - protein, 19 aa, Artificial Sequence
  source: /note=“Description of Artificial Sequence Synthetic peptide”
  MOD_RES (6)..(6): Any stapling amino acid
  SITE (6)..(10): /note=“May or may not be cross-linked via a side chain”
  MOD_RES (10)..(10): Any stapling amino acid
IQKEIXRLNXVAKNLNESL

SEQ ID NO: 39 - protein, 19 aa, Artificial Sequence
  source: /note=“Description of Artificial Sequence Synthetic peptide”
  MOD_RES (9)..(9): Any stapling amino acid
  SITE (9)..(13): /note=“May or may not be cross-linked via a side chain”
  MOD_RES (13)..(13): Any stapling amino acid
IQKEIDRLXEVAXNLNESL

SEQ ID NO: 40 - protein, 19 aa, Artificial Sequence
  source: /note=“Description of Artificial Sequence Synthetic peptide”
  MOD_RES (10)..(10): Any stapling amino acid
  SITE (10)..(14): /note=“May or may not be cross-linked via a side chain”
  MOD_RES (14)..(14): Any stapling amino acid
IQKEIDRLNXVAKXLNESL

SEQ ID NO: 41 - protein, 19 aa, Artificial Sequence
  source: /note=“Description of Artificial Sequence Synthetic peptide”
  MOD_RES (13)..(13): Any stapling amino acid
  SITE (13)..(17): /note=“May or may not be cross-linked via a side chain”
  MOD_RES (17)..(17): Any stapling amino acid
IQKEIDRLNEVAXNLNXSL

SEQ ID NO: 42 - protein, 19 aa, Artificial Sequence
  source: /note=“Description of Artificial Sequence Synthetic peptide”
  MOD_RES (14)..(14): Any stapling amino acid
  SITE (14)..(18): /note=“May or may not be cross-linked via a side chain”
  MOD_RES (18)..(18): Any stapling amino acid
IQKEIDRLNEVAKXLNEXL

SEQ ID NO: 43 - protein, 19 aa, Artificial Sequence
  source: /note=“Description of Artificial Sequence Synthetic peptide”
  MOD_RES (2)..(2): Any stapling/stitching amino acid
  SITE (2)..(9): /note=“May or may not be cross-linked via a side chain”
  MOD_RES (9)..(9): Any stapling/stitching amino acid
  SITE (9)..(16): /note=“May or may not be cross-linked via a side chain”
  MOD_RES (16)..(16): Any stapling/stitching amino acid
IXKEIDRLXEVAKNLXESL

SEQ ID NO: 44 - protein, 19 aa, Artificial Sequence
  source: /note=“Description of Artificial Sequence Synthetic peptide”
  MOD_RES (3)..(3): Any stapling/stitching amino acid
  SITE (3)..(10): /note=“May or may not be cross-linked via a side chain”
  MOD_RES (10)..(10): Any stapling/stitching amino acid
  SITE (10)..(17): /note=“May or may not be cross-linked via a side chain”
  MOD_RES (17)..(17): Any stapling/stitching amino acid
IQXEIDRLNXVAKNLNXSL

SEQ ID NO: 45 - protein, 19 aa, Artificial Sequence
  source: /note=“Description of Artificial Sequence Synthetic peptide”
  MOD_RES (2)..(2): Any stapling/stitching amino acid
  SITE (2)..(9): /note=“May or may not be cross-linked via a side chain”
  MOD_RES (9)..(9): Any stapling/stitching amino acid
  SITE (9)..(13): /note=“May or may not be cross-linked via a side chain”
  MOD_RES (13)..(13): Any stapling/stitching amino acid
IXKEIDRLXEVAXNLNESL

SEQ ID NO: 46 - protein, 19 aa, Artificial Sequence
  source: /note=“Description of Artificial Sequence Synthetic peptide”
  MOD_RES (3)..(3): Any stapling/stitching amino acid
  SITE (3)..(10): /note=“May or may not be cross-linked via a side chain”
  MOD_RES (10)..(10): Any stapling/stitching amino acid
  SITE (10)..(14): /note=“May or may not be cross-linked via a side chain”
  MOD_RES (14)..(14): Any stapling/stitching amino acid
IQXEIDRLNXVAKXLNESL

SEQ ID NO: 47 - protein, 19 aa, Artificial Sequence
  source: /note=“Description of Artificial Sequence Synthetic peptide”
  MOD_RES (6)..(6): Any stapling/stitching amino acid
  SITE (6)..(13): /note=“May or may not be cross-linked via a side chain”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
IQKEIXRLNEVAXNLNXSL

SEQ ID NO: 48 - protein, 19 aa, Artificial Sequence
  source: /note=“Description of Artificial Sequence Synthetic peptide”
  MOD_RES (7)..(7): Any stapling/stitching amino acid
  SITE (7)..(14): /note=“May or may not be cross-linked via a side chain”
  MOD_RES (14)..(14): Any stapling/stitching amino acid
  SITE (14)..(18): /note=“May or may not be cross-linked via a side chain”
  MOD_RES (18)..(18): Any stapling/stitching amino acid
IQKEIDXLNEVAKXLNEXL

SEQ ID NO: 49 - protein, 19 aa, Artificial Sequence
  source: /note=“Description of Artificial Sequence Synthetic peptide”
  MOD_RES (2)..(2): Any stapling amino acid
  SITE (2)..(6): /note=“May or may not be cross-linked via a side chain”
  MOD_RES (6)..(6): Any stapling amino acid
  MOD_RES (13)..(13): Any stapling amino acid
  SITE (13)..(17): /note=“May or may not be cross-linked via a side chain”
  MOD_RES (17)..(17): Any stapling amino acid
IXKEIXRLNEVAXNLNXSL

SEQ ID NO: 50 - protein, 19 aa, Artificial Sequence
  source: /note=“Description of Artificial Sequence Synthetic peptide”
  MOD_RES (3)..(3): Any stapling amino acid
  SITE (3)..(7): /note=“May or may not be cross-linked via a side chain”
  MOD_RES (7)..(7): Any stapling amino acid
  SITE (7)..(13): /note=“May or may not be cross-linked via a side chain”
  MOD_RES (13)..(13): Any stapling amino acid
  SITE (13)..(17): /note=“May or may not be cross-linked via a side chain”
  MOD_RES (17)..(17): Any stapling amino acid
IQXEIDXLNEVAXNLNXSL

SEQ ID NO: 51 - protein, 19 aa, Artificial Sequence
  source: /note=“Description of Artificial Sequence Synthetic peptide”
  MOD_RES (2)..(2): Any stapling amino acid
  SITE (2)..(6): /note=“May or may not be cross-linked via a side chain”
  MOD_RES (6)..(6): Any stapling amino acid
  MOD_RES (14)..(14): Any stapling amino acid
  SITE (14)..(18): /note=“May or may not be cross-linked via a side chain”
  MOD_RES (18)..(18): Any stapling amino acid
IXKEIXRLNEVAKXLNEXL

SEQ ID NO: 52 - protein, 19 aa, Artificial Sequence
  source: /note=“Description of Artificial Sequence Synthetic peptide”
  MOD_RES (3)..(3): Any stapling amino acid
  SITE (3)..(7): /note=“May or may not be cross-linked via a side chain”
  MOD_RES (7)..(7): Any stapling amino acid
  MOD_RES (14)..(14): Any stapling amino acid
  SITE (14)..(18): /note=“May or may not be cross-linked via a side chain”
  MOD_RES (18)..(18): Any stapling amino acid
IQXEIDXLNEVAKXLNEXL

SEQ ID NO: 53 - protein, 4 aa, Artificial Sequence
  source: /note=“Description of Artificial Sequence Synthetic peptide”
ISGI

SEQ ID NO: 54 - protein, 6 aa, Artificial Sequence
  source: /note=“Description of Artificial Sequence Synthetic peptide”
ASVVNI

SEQ ID NO: 55 - protein, 30 aa, Artificial Sequence
  source: /note=“Description of Artificial Sequence Synthetic polypeptide”
KEIDRLNEVAKNLNESLIDLQELGKYEQYI

SEQ ID NO: 56 - protein, 5 aa, Artificial Sequence
  source: /note=“Description of Artificial Sequence Synthetic peptide”
ISGIN

SEQ ID NO: 57 - protein, 6 aa, Artificial Sequence
  source: /note=“Description of Artificial Sequence Synthetic peptide”
SVVNIQ

SEQ ID NO: 58 - protein, 29 aa, Artificial Sequence
  source: /note=“Description of Artificial Sequence Synthetic peptide”
EIDRLNEVAKNLNESLIDLQELGKYEQYI

SEQ ID NO: 59 - protein, 6 aa, Artificial Sequence
  source: /note=“Description of Artificial Sequence Synthetic peptide”
ISGINA

SEQ ID NO: 60 - protein, 6 aa, Artificial Sequence
  source: /note=“Description of Artificial Sequence Synthetic peptide”
VVNIQK

SEQ ID NO: 61 - protein, 28 aa, Artificial Sequence
  source: /note=“Description of Artificial Sequence Synthetic peptide”
IDRLNEVAKNLNESLIDLQELGKYEQYI

SEQ ID NO: 62 - protein, 25 aa, Artificial Sequence
  source: /note=“Description of Artificial Sequence Synthetic peptide”
ISGINASVVNIQKEIDRLNEVAKNL

SEQ ID NO: 63 - protein, 6 aa, Artificial Sequence
  source: /note=“Description of Artificial Sequence Synthetic peptide”
ESLIDL

SEQ ID NO: 64 - protein, 9 aa, Artificial Sequence
  source: /note=“Description of Artificial Sequence Synthetic peptide”
ELGKYEQYI

SEQ ID NO: 65 - protein, 26 aa, Artificial Sequence
  source: /note=“Description of Artificial Sequence Synthetic peptide”
ISGINASVVNIQKEIDRLNEVAKNLN

SEQ ID NO: 66 - protein, 6 aa, Artificial Sequence
  source: /note=“Description of Artificial Sequence Synthetic peptide”
SLIDLQ

SEQ ID NO: 67 - protein, 8 aa, Artificial Sequence
  source: /note=“Description of Artificial Sequence Synthetic peptide”
LGKYEQYI

SEQ ID NO: 68 - protein, 32 aa, Artificial Sequence
  source: /note=“Description of Artificial Sequence Synthetic polypeptide”
ISGINASVVNIQKEIDRLNEVAKNLNESLIDL

SEQ ID NO: 69 - protein, 6 aa, Artificial Sequence
  source: /note=“Description of Artificial Sequence Synthetic peptide”
ELGKYE

SEQ ID NO: 70 - protein, 6 aa, Artificial Sequence
  source: /note=“Description of Artificial Sequence Synthetic peptide”
KEIDRL

SEQ ID NO: 71 - protein, 10 aa, Artificial Sequence
  source: /note=“Description of Artificial Sequence Synthetic peptide”
EVAKNLNESL

SEQ ID NO: 72 - protein, 6 aa, Artificial Sequence
  source: /note=“Description of Artificial Sequence Synthetic peptide”
EIDRLN

SEQ ID NO: 73 - protein, 9 aa, Artificial Sequence
  source: /note=“Description of Artificial Sequence Synthetic peptide”
VAKNLNESL

SEQ ID NO: 74 - protein, 5 aa, Artificial Sequence
  source: /note=“Description of Artificial Sequence Synthetic peptide”
IQKEI

SEQ ID NO: 75 - protein, 6 aa, Artificial Sequence
  source: /note=“Description of Artificial Sequence Synthetic peptide”
RLNEVA

SEQ ID NO: 76 - protein, 6 aa, Artificial Sequence
  source: /note=“Description of Artificial Sequence Synthetic peptide”
NLNESL

SEQ ID NO: 77 - protein, 6 aa, Artificial Sequence
  source: /note=“Description of Artificial Sequence Synthetic peptide”
IQKEID

SEQ ID NO: 78 - protein, 6 aa, Artificial Sequence
  source: /note=“Description of Artificial Sequence Synthetic peptide”
LNEVAK

SEQ ID NO: 79 - protein, 5 aa, Artificial Sequence
  source: /note=“Description of Artificial Sequence Synthetic peptide”
LNESL

SEQ ID NO: 80 - protein, 8 aa, Artificial Sequence
  source: /note=“Description of Artificial Sequence Synthetic peptide”
IQKEIDRL

SEQ ID NO: 81 - protein, 6 aa, Artificial Sequence
  source: /note=“Description of Artificial Sequence Synthetic peptide”
EVAKNL

SEQ ID NO: 82 - protein, 9 aa, Artificial Sequence
  source: /note=“Description of Artificial Sequence Synthetic peptide”
IQKEIDRLN

SEQ ID NO: 83 - protein, 6 aa, Artificial Sequence
  source: /note=“Description of Artificial Sequence Synthetic peptide”
VAKNLN

SEQ ID NO: 84 - protein, 13 aa, Artificial Sequence
  source: /note=“Description of Artificial Sequence Synthetic peptide”
RLNEVAKNLNESL

SEQ ID NO: 85 - protein, 12 aa, Artificial Sequence
  source: /note=“Description of Artificial Sequence Synthetic peptide”
LNEVAKNLNESL

SEQ ID NO: 86 - protein, 12 aa, Artificial Sequence
  source: /note=“Description of Artificial Sequence Synthetic peptide”
IQKEIDRLNEVA

SEQ ID NO: 87 - protein, 13 aa, Artificial Sequence
  source: /note=“Description of Artificial Sequence Synthetic peptide”
IQKEIDRLNEVAK

SEQ ID NO: 88 - protein, 13 aa, Artificial Sequence
  source: /note=“Description of Artificial Sequence Synthetic peptide”
KEIDRLNEVAKNL

SEQ ID NO: 89 - protein, 14 aa, Artificial Sequence
  source: /note=“Description of Artificial Sequence Synthetic peptide”
KEIDRLNEVAKNLN

SEQ ID NO: 90 - protein, 20 aa, Artificial Sequence
  source: /note=“Description of Artificial Sequence Synthetic peptide”
KEIDRLNEVAKNLNESLIDL

SEQ ID NO: 91 - protein, 12 aa, Artificial Sequence
  source: /note=“Description of Artificial Sequence Synthetic peptide”
EIDRLNEVAKNL

SEQ ID NO: 92 - protein, 13 aa, Artificial Sequence
  source: /note=“Description of Artificial Sequence Synthetic peptide”
EIDRLNEVAKNLN

SEQ ID NO: 93 - protein, 19 aa, Artificial Sequence
  source: /note=“Description of Artificial Sequence Synthetic peptide”
EIDRLNEVAKNLNESLIDL

SEQ ID NO: 94 - protein, 11 aa, Artificial Sequence
  source: /note=“Description of Artificial Sequence Synthetic peptide”
IDRLNEVAKNL

SEQ ID NO: 95 - protein, 12 aa, Artificial Sequence
  source: /note=“Description of Artificial Sequence Synthetic peptide”
IDRLNEVAKNLN

SEQ ID NO: 96 - protein, 18 aa, Artificial Sequence
  source: /note=“Description of Artificial Sequence Synthetic peptide”
IDRLNEVAKNLNESLIDL

SEQ ID NO: 97 - protein, 6 aa, Artificial Sequence
  source: /note=“Description of Artificial Sequence Synthetic peptide”
RLNEVA

SEQ ID NO: 98 - protein, 5 aa, Artificial Sequence
  source: /note=“Description of Artificial Sequence Synthetic peptide”
LNEVA

SEQ ID NO: 99 - protein, 7 aa, Artificial Sequence
  source: /note=“Description of Artificial Sequence Synthetic peptide”
RLNEVAK

SEQ ID NO: 100 - protein, 6 aa, Artificial Sequence
  source: /note=“Description of Artificial Sequence Synthetic peptide”
LNEVAK

SEQ ID NO: 101 - protein, 6 aa, Artificial Sequence
  source: /note=“Description of Artificial Sequence Synthetic 6xHis tag”
HHHHHH

SEQ ID NO: 102 - protein, 19 aa, Artificial Sequence
  source: /note=“Description of Artificial Sequence Synthetic peptide”
IQKEIDRLNEVAKNLNESL

SEQ ID NO: 103 - protein, 29 aa, Artificial Sequence
  source: /note=“Description of Artificial Sequence Synthetic peptide”
ISGINASVVNIQKEIDRLNEVAKNLNESL

SEQ ID NO: 104 - protein, 25 aa, Artificial Sequence
  source: /note=“Description of Artificial Sequence Synthetic peptide”
IQKEIDRLNEVAKNLNESLIDLQEL

SEQ ID NO: 105 - protein, 25 aa, Artificial Sequence
  source: /note=“Description of Artificial Sequence Synthetic peptide”
IQKEIDRLNEVAKNLNESLIDLQEL

SEQ ID NO: 106 - protein, 30 aa, Artificial Sequence
  source: /note=“Description of Artificial Sequence Synthetic polypeptide”
DISGINASVVNIQKEIDRLNEVAKNLNESL

SEQ ID NO: 107 - protein, 30 aa, Artificial Sequence; identical to SEQ ID NO: 106
  source: /note=“Description of Artificial Sequence Synthetic polypeptide”

SEQ ID NO: 108 - protein, 36 aa, Artificial Sequence
  source: /note=“Description of Artificial Sequence Synthetic polypeptide”
DISGINASVVNIQKEIDRLNEVAKNLNESLIDLQEL

SEQ ID NO: 109 - protein, 36 aa, Artificial Sequence; identical to SEQ ID NO: 108
  source: /note=“Description of Artificial Sequence Synthetic polypeptide”

SEQ ID NO: 110 - protein, 36 aa, Artificial Sequence
  source: /note=“Description of Artificial Sequence Synthetic polypeptide”
SLDQINVTFLDLEYEMKKLEEAIKKLEESYIDLKEL

SEQ ID NO: 111 - protein, 36 aa, Artificial Sequence; identical to SEQ ID NO: 110
  source: /note=“Description of Artificial Sequence Synthetic polypeptide”

SEQ ID NO: 112 - protein, 19 aa, Artificial Sequence
  source: /note=“Description of Artificial Sequence Synthetic peptide”
  MOD_RES (1)..(1): Any stapling/stitching amino acid
  SITE (1)..(8): /note=“May or may not be cross-linked via a side chain”
  MOD_RES (8)..(8): Any stapling/stitching amino acid
XQKEIDRXNEVAKNLNESL

SEQ ID NO: 113 - protein, 19 aa, Artificial Sequence
  source: /note=“Description of Artificial Sequence Synthetic peptide”
  MOD_RES (4)..(4): Any stapling/stitching amino acid
  SITE (4)..(11): /note=“May or may not be cross-linked via a side chain”
  MOD_RES (11)..(11): Any stapling/stitching amino acid
IQKXIDRLNEXAKNLNESL

SEQ ID NO: 114 - protein, 19 aa, Artificial Sequence
  source: /note=“Description of Artificial Sequence Synthetic peptide”
  MOD_RES (5)..(5): Any stapling/stitching amino acid
  SITE (5)..(12): /note=“May or may not be cross-linked via a side chain”
  MOD_RES (12)..(12): Any stapling/stitching amino acid
IQKEXDRLNEVXKNLNESL

SEQ ID NO: 115 - protein, 19 aa, Artificial Sequence
  source: /note=“Description of Artificial Sequence Synthetic peptide”
  MOD_RES (8)..(8): Any stapling/stitching amino acid
  SITE (8)..(15): /note=“May or may not be cross-linked via a side chain”
  MOD_RES (15)..(15): Any stapling/stitching amino acid
IQKEIDRXNEVAKNXNESL

SEQ ID NO: 116 - protein, 19 aa, Artificial Sequence
  source: /note=“Description of Artificial Sequence Synthetic peptide”
  MOD_RES (11)..(11): Any stapling/stitching amino acid
  SITE (11)..(18): /note=“May or may not be cross-linked via a side chain”
  MOD_RES (18)..(18): Any stapling/stitching amino acid
IQKEIDRLNEXAKNLNEXL

SEQ ID NO: 117 - protein, 19 aa, Artificial Sequence
  source: /note=“Description of Artificial Sequence Synthetic peptide”
  MOD_RES (12)..(12): Any stapling/stitching amino acid
  SITE (12)..(19): /note=“May or may not be cross-linked via a side chain”
  MOD_RES (19)..(19): Any stapling/stitching amino acid
IQKEIDRLNEVXKNLNESX

SEQ ID NO: 118 - protein, 29 aa, Artificial Sequence
  source: /note=“Description of Artificial Sequence Synthetic peptide”
  MOD_RES (11)..(11): Any stapling/stitching amino acid
  SITE (11)..(18): /note=“May or may not be cross-linked via a side chain”
  MOD_RES (18)..(18): Any stapling/stitching amino acid
ISGINASVVNXQKEIDRXNEVAKNLNESL

SEQ ID NO: 119 - protein, 29 aa, Artificial Sequence
  source: /note=“Description of Artificial Sequence Synthetic peptide”
  MOD_RES (12)..(12): Any stapling/stitching amino acid
  SITE (12)..(19): /note=“May or may not be cross-linked via a side chain”
  MOD_RES (19)..(19): Any stapling/stitching amino acid
ISGINASVVNIXKEIDRLXEVAKNLNESL

SEQ ID NO: 120 - protein, 29 aa, Artificial Sequence
  source: /note=“Description of Artificial Sequence Synthetic peptide”
  MOD_RES (13)..(13): Any stapling/stitching amino acid
  SITE (13)..(20): /note=“May or may not be cross-linked via a side chain”
  MOD_RES (20)..(20): Any stapling/stitching amino acid
ISGINASVVNIQXEIDRLNXVAKNLNESL

SEQ ID NO: 121 - protein, 29 aa, Artificial Sequence
  source: /note=“Description of Artificial Sequence Synthetic peptide”
  MOD_RES (14)..(14): Any stapling/stitching amino acid
  SITE (14)..(21): /note=“May or may not be cross-linked via a side chain”
  MOD_RES (21)..(21): Any stapling/stitching amino acid
ISGINASVVNIQKXIDRLNEXAKNLNESL

SEQ ID NO: 122 - protein, 29 aa, Artificial Sequence
  source: /note=“Description of Artificial Sequence Synthetic peptide”
  MOD_RES (15)..(15): Any stapling/stitching amino acid
  SITE (15)..(22): /note=“May or may not be cross-linked via a side chain”
  MOD_RES (22)..(22): Any stapling/stitching amino acid
ISGINASVVNIQKEXDRLNEVXKNLNESL

SEQ ID NO: 123 - protein, 29 aa, Artificial Sequence
  source: /note=“Description of Artificial Sequence Synthetic peptide”
  MOD_RES (16)..(16): Any stapling/stitching amino acid
  SITE (16)..(23): /note=“May or may not be cross-linked via a side chain”
  MOD_RES (23)..(23): Any stapling/stitching amino acid
ISGINASVVNIQKEIXRLNEVAXNLNESL

SEQ ID NO: 124 - protein, 29 aa, Artificial Sequence
  source: /note=“Description of Artificial Sequence Synthetic peptide”
  MOD_RES (17)..(17): Any stapling/stitching amino acid
  SITE (17)..(24): /note=“May or may not be cross-linked via a side chain”
  MOD_RES (24)..(24): Any stapling/stitching amino acid
ISGINASVVNIQKEIDXLNEVAKXLNESL

SEQ ID NO: 125 - protein, 29 aa, Artificial Sequence
  source: /note=“Description of Artificial Sequence Synthetic peptide”
  MOD_RES (18)..(18): Any stapling/stitching amino acid
  SITE (18)..(25): /note=“May or may not be cross-linked via a side chain”
  MOD_RES (25)..(25): Any stapling/stitching amino acid
ISGINASVVNIQKEIDRXNEVAKNXNESL

SEQ ID NO: 126 - protein, 29 aa, Artificial Sequence
  source: /note=“Description of Artificial Sequence Synthetic peptide”
  MOD_RES (19)..(19): Any stapling/stitching amino acid
  SITE (19)..(26): /note=“May or may not be cross-linked via a side chain”
  MOD_RES (26)..(26): Any stapling/stitching amino acid
ISGINASVVNIQKEIDRLXEVAKNLXESL

SEQ ID NO: 127 - protein, 29 aa, Artificial Sequence
  source: /note=“Description of Artificial Sequence Synthetic peptide”
  MOD_RES (20)..(20): Any stapling/stitching amino acid
  SITE (20)..(27): /note=“May or may not be cross-linked via a side chain”
  MOD_RES (27)..(27): Any stapling/stitching amino acid
ISGINASVVNIQKEIDRLNXVAKNLNXSL

SEQ ID NO: 128 - protein, 29 aa, Artificial Sequence
  source: /note=“Description of Artificial Sequence Synthetic peptide”
  MOD_RES (21)..(21): Any stapling/stitching amino acid
  SITE (21)..(28): /note=“May or may not be cross-linked via a side chain”
  MOD_RES (28)..(28): Any stapling/stitching amino acid
ISGINASVVNIQKEIDRLNEXAKNLNEXL

SEQ ID NO: 129 - protein, 29 aa, Artificial Sequence
  source: /note=“Description of Artificial Sequence Synthetic peptide”
  MOD_RES (22)..(22): Any stapling/stitching amino acid
  SITE (22)..(29): /note=“May or may not be cross-linked via a side chain”
  MOD_RES (29)..(29): Any stapling/stitching amino acid
ISGINASVVNIQKEIDRLNEVXKNLNESX

SEQ ID NO: 130 - protein, 19 aa, Artificial Sequence
  source: /note=“Description of Artificial Sequence Synthetic peptide”
  MOD_RES (1)..(1): Any stapling/stitching amino acid
  SITE (1)..(5): /note=“May or may not be cross-linked via a side chain”
  MOD_RES (5)..(5): Any stapling/stitching amino acid
XQKEXDRLNEVAKNLNESL

SEQ ID NO: 131 - protein, 19 aa, Artificial Sequence
  source: /note=“Description of Artificial Sequence Synthetic peptide”
  MOD_RES (4)..(4): Any stapling/stitching amino acid
  SITE (4)..(8): /note=“May or may not be cross-linked via a side chain”
  MOD_RES (8)..(8): Any stapling/stitching amino acid
IQKXIDRXNEVAKNLNESL

SEQ ID NO: 132 - protein, 19 aa, Artificial Sequence
  source: /note=“Description of Artificial Sequence Synthetic peptide”
  MOD_RES (5)..(5): Any stapling/stitching amino acid
  SITE (5)..(9): /note=“May or may not be cross-linked via a side chain”
  MOD_RES (9)..(9): Any stapling/stitching amino acid
IQKEXDRLXEVAKNLNESL

SEQ ID NO: 133 - protein, 19 aa, Artificial Sequence
  source: /note=“Description of Artificial Sequence Synthetic peptide”
  MOD_RES (7)..(7): Any stapling/stitching amino acid
  SITE (7)..(11): /note=“May or may not be cross-linked via a side chain”
  MOD_RES (11)..(11): Any stapling/stitching amino acid
IQKEIDXLNEXAKNLNESL

SEQ ID NO: 134 - protein, 19 aa, Artificial Sequence
  source: /note=“Description of Artificial Sequence Synthetic peptide”
  MOD_RES (8)..(8): Any stapling/stitching amino acid
  SITE (8)..(12): /note=“May or may not be cross-linked via a side chain”
  MOD_RES (12)..(12): Any stapling/stitching amino acid
IQKEIDRXNEVXKNLNESL

SEQ ID NO: 135 - protein, 19 aa, Artificial Sequence
  source: /note=“Description of Artificial Sequence Synthetic peptide”
  MOD_RES (11)..(11): Any stapling/stitching amino acid
  SITE (11)..(15): /note=“May or may not be cross-linked via a side chain”
  MOD_RES (15)..(15): Any stapling/stitching amino acid
IQKEIDRLNEXAKNXNESL

SEQ ID NO: 136 - protein, 19 aa, Artificial Sequence
  source: /note=“Description of Artificial Sequence Synthetic peptide”
  MOD_RES (12)..(12): Any stapling/stitching amino acid
  SITE (12)..(16): /note=“May or may not be cross-linked via a side chain”
  MOD_RES (16)..(16): Any stapling/stitching amino acid
IQKEIDRLNEVXKNLXESL

SEQ ID NO: 137 - protein, 19 aa, Artificial Sequence
  source: /note=“Description of Artificial Sequence Synthetic peptide”
  MOD_RES (15)..(15): Any stapling/stitching amino acid
  SITE (15)..(19): /note=“May or may not be cross-linked via a side chain”
  MOD_RES (19)..(19): Any stapling/stitching amino acid
IQKEIDRLNEVAKNXNESX

SEQ ID NO: 138 - protein, 29 aa, Artificial Sequence
  source: /note=“Description of Artificial Sequence Synthetic peptide”
  MOD_RES (11)..(11): Any stapling/stitching amino acid
  SITE (11)..(15): /note=“May or may not be cross-linked via a side chain”
  MOD_RES (15)..(15): Any stapling/stitching amino acid
ISGINASVVNXQKEXDRLNEVAKNLNESL

SEQ ID NO: 139 - protein, 29 aa, Artificial Sequence
  source: /note=“Description of Artificial Sequence Synthetic peptide”
  MOD_RES (12)..(12): Any stapling/stitching amino acid
  SITE (12)..(16): /note=“May or may not be cross-linked via a side chain”
  MOD_RES (16)..(16): Any stapling/stitching amino acid
ISGINASVVNIXKEIXRLNEVAKNLNESL

SEQ ID NO: 140 - protein, 29 aa, Artificial Sequence
  source: /note=“Description of Artificial Sequence Synthetic peptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
ISGINASVVNIQXEIDXLNEVAKNLNESL

SEQ ID NO: 141 - protein, 29 aa, Artificial Sequence
  source: /note=“Description of Artificial Sequence Synthetic peptide”
  MOD_RES (14)..(14): Any stapling/stitching amino acid
  SITE (14)..(18): /note=“May or may not be cross-linked via a side chain”
  MOD_RES (18)..(18): Any stapling/stitching amino acid
ISGINASVVNIQKXIDRXNEVAKNLNESL

SEQ ID NO: 142 - protein, 29 aa, Artificial Sequence
  source: /note=“Description of Artificial Sequence Synthetic peptide”
  MOD_RES (15)..(15): Any stapling/stitching amino acid
  SITE (15)..(19): /note=“May or may not be cross-linked via a side chain”
  MOD_RES (19)..(19): Any stapling/stitching amino acid
ISGINASVVNIQKEXDRLXEVAKNLNESL

SEQ ID NO: 143 - protein, 29 aa, Artificial Sequence
  source: /note=“Description of Artificial Sequence Synthetic peptide”
  MOD_RES (16)..(16): Any stapling/stitching amino acid
  SITE (16)..(20): /note=“May or may not be cross-linked via a side chain”
  MOD_RES (20)..(20): Any stapling/stitching amino acid
ISGINASVVNIQKEIXRLNXVAKNLNESL

SEQ ID NO: 144 - protein, 29 aa, Artificial Sequence
  source: /note=“Description of Artificial Sequence Synthetic peptide”
  MOD_RES (17)..(17): Any stapling/stitching amino acid
  SITE (17)..(21): /note=“May or may not be cross-linked via a side chain”
  MOD_RES (21)..(21): Any stapling/stitching amino acid
ISGINASVVNIQKEIDXLNEXAKNLNESL

SEQ ID NO: 145 - protein, 29 aa, Artificial Sequence
  source: /note=“Description of Artificial Sequence Synthetic peptide”
  MOD_RES (18)..(18): Any stapling/stitching amino acid
  SITE (18)..(22): /note=“May or may not be cross-linked via a side chain”
  MOD_RES (22)..(22): Any stapling/stitching amino acid
ISGINASVVNIQKEIDRXNEVXKNLNESL

SEQ ID NO: 146 - protein, 29 aa, Artificial Sequence
  source: /note=“Description of Artificial Sequence Synthetic peptide”
  MOD_RES (19)..(19): Any stapling/stitching amino acid
  SITE (19)..(23): /note=“May or may not be cross-linked via a side chain”
  MOD_RES (23)..(23): Any stapling/stitching amino acid
ISGINASVVNIQKEIDRLXEVAXNLNESL

SEQ ID NO: 147 - protein, 29 aa, Artificial Sequence
  source: /note=“Description of Artificial Sequence Synthetic peptide”
  MOD_RES (20)..(20): Any stapling/stitching amino acid
  SITE (20)..(24): /note=“May or may not be cross-linked via a side chain”
  MOD_RES (24)..(24): Any stapling/stitching amino acid
ISGINASVVNIQKEIDRLNXVAKXLNESL

SEQ ID NO: 148 - protein, 29 aa, Artificial Sequence
  source: /note=“Description of Artificial Sequence Synthetic peptide”
  MOD_RES (21)..(21): Any stapling/stitching amino acid
  SITE (21)..(25): /note=“May or may not be cross-linked via a side chain”
  MOD_RES (25)..(25): Any stapling/stitching amino acid
ISGINASVVNIQKEIDRLNEXAKNXNESL

SEQ ID NO: 149 - protein, 29 aa, Artificial Sequence
  source: /note=“Description of Artificial Sequence Synthetic peptide”
  MOD_RES (22)..(22): Any stapling/stitching amino acid
  SITE (22)..(26): /note=“May or may not be cross-linked via a side chain”
  MOD_RES (26)..(26): Any stapling/stitching amino acid
ISGINASVVNIQKEIDRLNEVXKNLXESL

SEQ ID NO: 150 - protein, 29 aa, Artificial Sequence
  source: /note=“Description of Artificial Sequence Synthetic peptide”
  MOD_RES (23)..(23): Any stapling/stitching amino acid
  SITE (23)..(27): /note=“May or may not be cross-linked via a side chain”
  MOD_RES (27)..(27): Any stapling/stitching amino acid
ISGINASVVNIQKEIDRLNEVAXNLNXSL

SEQ ID NO: 151 - protein, 29 aa, Artificial Sequence
  source: /note=“Description of Artificial Sequence Synthetic peptide”
  MOD_RES (24)..(24): Any stapling/stitching amino acid
  SITE (24)..(28): /note=“May or may not be cross-linked via a side chain”
  MOD_RES (28)..(28): Any stapling/stitching amino acid
ISGINASVVNIQKEIDRLNEVAKXLNEXL

SEQ ID NO: 152 - protein, 29 aa, Artificial Sequence
  source: /note=“Description of Artificial Sequence Synthetic peptide”
  MOD_RES (25)..(25): Any stapling/stitching amino acid
  SITE (25)..(29): /note=“May or may not be cross-linked via a side chain”
  MOD_RES (29)..(29): Any stapling/stitching amino acid
ISGINASVVNIQKEIDRLNEVAKNXNESX

SEQ ID NO: 153 - protein, 42 aa, Artificial Sequence
  source: /note=“Description of Artificial Sequence Synthetic polypeptide”
  MOD_RES (12)..(12): Any stapling/stitching amino acid
  SITE (12)..(19): /note=“May or may not be cross-linked via a side chain”
  MOD_RES (19)..(19): Any stapling/stitching amino acid
  SITE (19)..(26): /note=“May or may not be cross-linked via a side chain”
  MOD_RES (26)..(26): Any stapling/stitching amino acid
ISGINASVVNIXKEIDRLXEVAKNLXESLIDLQELGKYEQYI

SEQ ID NO: 154 - protein, 42 aa, Artificial Sequence
  source: /note=“Description of Artificial Sequence Synthetic polypeptide”
  MOD_RES (13)..(13): Any stapling/stitching amino acid
  SITE (13)..(20): /note=“May or may not be cross-linked via a side chain”
  MOD_RES (20)..(20): Any stapling/stitching amino acid
  SITE (20)..(24): /note=“May or may not be cross-linked via a side chain”
  MOD_RES (24)..(24): Any stapling/stitching amino acid
ISGINASVVNIQXEIDRLNXVAKXLNESLIDLQELGKYEQYI

SEQ ID NO: 155 - protein, 19 aa, Artificial Sequence
  source: /note=“Description of Artificial Sequence Synthetic peptide”
  MOD_RES (2)..(2): Any stapling/stitching amino acid
  SITE (2)..(6): /note=“May or may not be cross-linked via a side chain”
  MOD_RES (6)..(6): Any stapling/stitching amino acid
  MOD_RES (13)..(13): Any stapling/stitching amino acid
  SITE (13)..(17): /note=“May or may not be cross-linked via a side chain”
  MOD_RES (17)..(17): Any stapling/stitching amino acid
IXKEIXRLNEVAXNLNXSL

SEQ ID NO: 156 - protein, 42 aa, Artificial Sequence
  source: /note=“Description of Artificial Sequence Synthetic polypeptide”
  MOD_RES (12)..(12): Any stapling/stitching amino acid
  SITE (12)..(16): /note=“May or may not be cross-linked via a side chain”
  MOD_RES (16)..(16): Any stapling/stitching amino acid
  MOD_RES (23)..(23): Any stapling/stitching amino acid
  SITE (23)..(27): /note=“May or may not be cross-linked via a side chain”
  MOD_RES (27)..(27): Any stapling/stitching amino acid
ISGINASVVNIXKEIXRLNEVAXNLNXSLIDLQELGKYEQYI

SEQ ID NO: 157 - protein, 29 aa, Artificial Sequence
  source: /note=“Description of Artificial Sequence Synthetic peptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (23)..(23): Any stapling/stitching amino acid
  SITE (23)..(27): /note=“May or may not be cross-linked via a side chain”
  MOD_RES (27)..(27): Any stapling/stitching amino acid
ISGINASVVNIQXEIDXLNEVAXNLNXSL

SEQ ID NO: 158 - protein, 42 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (23)..(23): Any stapling/stitching amino acid
  SITE (23)..(27): /note=“May or may not be cross-linked via a side chain”
  MOD_RES (27)..(27): Any stapling/stitching amino acid
ISGINASVVNIQXEIDXLNEVAXNLNXSLIDLQELGKYEQYI

SEQ ID NO: 159 - protein, 19 aa, Artificial Sequence
  source: /note=“Description of Artificial Sequence Synthetic peptide”
  MOD_RES (2)..(2): Any stapling/stitching amino acid
  SITE (2)..(6): /note=“May or may not be cross-linked via a side chain”
  MOD_RES (6)..(6): Any stapling/stitching amino acid
  MOD_RES (14)..(14): Any stapling/stitching amino acid
  SITE (14)..(18): /note=“May or may not be cross-linked via a side chain”
  MOD_RES (18)..(18): Any stapling/stitching amino acid
IXKEIXRLNEVAKXLNEXL

SEQ ID NO: 160 - protein, 42 aa, Artificial Sequence
  source: /note=“Description of Artificial Sequence Synthetic polypeptide”
  MOD_RES (12)..(12): Any stapling/stitching amino acid
  SITE (12)..(16): /note=“May or may not be cross-linked via a side chain”
  MOD_RES (16)..(16): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
ISGINASVVNIXKEIXRLNEVAKXLNEXLIDLQELGKYEQYI

SEQ ID NO: 161 - protein, 19 aa, Artificial Sequence
  source: /note=“Description of Artificial Sequence Synthetic peptide”
  MOD_RES (3)..(3): Any stapling/stitching amino acid
  SITE (3)..(7): /note=“May or may not be cross-linked via a side chain”
  MOD_RES (7)..(7): Any stapling/stitching amino acid
  MOD_RES (14)..(14): Any stapling/stitching amino acid
  SITE (14)..(18): /note=“May or may not be cross-linked via a side chain”
  MOD_RES (18)..(18): Any stapling/stitching amino acid
IQXEIDXLNEVAKXLNEXL

SEQ ID NO: 162 - protein, 42 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
ISGINASVVNIQXEIDXLNEVAKXLNEXLIDLQELGKYEQYI

SEQ ID NO: 163 - protein, 25 aa, Artificial Sequence
  source: /note=“Description of Artificial Sequence Synthetic peptide”
  MOD_RES (2)..(2): Any stapling/stitching amino acid
  SITE (2)..(6): /note=“May or may not be cross-linked via a side chain”
  MOD_RES (6)..(6): Any stapling/stitching amino acid
  MOD_RES (13)..(13): Any stapling/stitching amino acid
  SITE (13)..(17): /note=“May or may not be cross-linked via a side chain”
  MOD_RES (17)..(17): Any stapling/stitching amino acid
IXKEIXRLNEVAXNLNXSLIDLQEL

SEQ ID NO: 164 - protein, 25 aa, Artificial Sequence
  source: /note=“Description of Artificial Sequence Synthetic peptide”
  MOD_RES (3)..(3): Any stapling/stitching amino acid
  SITE (3)..(7): /note=“May or may not be cross-linked via a side chain”
  MOD_RES (7)..(7): Any stapling/stitching amino acid
  MOD_RES (13)..(13): Any stapling/stitching amino acid
  SITE (13)..(17): /note=“May or may not be cross-linked via a side chain”
  MOD_RES (17)..(17): Any stapling/stitching amino acid
IQXEIDXLNEVAXNLNXSLIDLQEL

SEQ ID NO: 165 - protein, 25 aa, Artificial Sequence
  source: /note=“Description of Artificial Sequence Synthetic peptide”
  MOD_RES (2)..(2): Any stapling/stitching amino acid
  SITE (2)..(6): /note=“May or may not be cross-linked via a side chain”
  MOD_RES (6)..(6): Any stapling/stitching amino acid
  MOD_RES (14)..(14): Any stapling/stitching amino acid
  SITE (14)..(18): /note=“May or may not be cross-linked via a side chain”
  MOD_RES (18)..(18): Any stapling/stitching amino acid
IXKEIXRLNEVAKXLNEXLIDLQEL

SEQ ID NO: 166 - protein, 25 aa, Artificial Sequence
  source: /note=“Description of Artificial Sequence Synthetic peptide”
  MOD_RES (3)..(3): Any stapling/stitching amino acid
  SITE (3)..(7): /note=“May or may not be cross-linked via a side chain”
  MOD_RES (7)..(7): Any stapling/stitching amino acid
  MOD_RES (14)..(14): Any stapling/stitching amino acid
  SITE (14)..(18): /note=“May or may not be cross-linked via a side chain”
  MOD_RES (18)..(18): Any stapling/stitching amino acid
IQXEIDXLNEVAKXLNEXLIDLQEL

SEQ ID NO: 167 - protein, 30 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
DISGINASVVNIXKEIXRLNEVAXNLNXSL

SEQ ID NO: 168 - protein, 30 aa, Artificial Sequence
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (18)..(18): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
DISGINASVVNIQXEIDXLNEVAXNLNXSL

SEQ ID NO: 169 - protein, 30 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid
DISGINASVVNIXKEIXRLNEVAKXLNEXL

SEQ ID NO: 170 - protein, 30 aa, Artificial Sequence
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (18)..(18): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid
DISGINASVVNIQXEIDXLNEVAKXLNEXL

SEQ ID NO: 171 - protein, 36 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
DISGINASVVNIXKEIXRLNEVAXNLNXSLIDLQEL

SEQ ID NO: 172 - protein, 36 aa, Artificial Sequence
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (18)..(18): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
DISGINASVVNIQXEIDXLNEVAXNLNXSLIDLQEL

SEQ ID NO: 173 - protein, 36 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid
DISGINASVVNIXKEIXRLNEVAKXLNEXLIDLQEL

SEQ ID NO: 174 - protein, 36 aa, Artificial Sequence
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (18)..(18): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid
DISGINASVVNIQXEIDXLNEVAKXLNEXLIDLQEL

SEQ ID NO: 175 - protein, 35 aa, Artificial Sequence
  source: /note=“Description of Artificial Sequence Synthetic polypeptide”
  MOD_RES (13)..(13): Any stapling/stitching amino acid
  SITE (13)..(20): /note=“May or may not be cross-linked via a side chain”
  MOD_RES (16)..(16): Norleucine
  MOD_RES (20)..(20): Any stapling/stitching amino acid
  SITE (20)..(27): /note=“May or may not be cross-linked via a side chain”
  MOD_RES (27)..(27): Any stapling/stitching amino acid
SLDQINVTFLDLXYEXKKLXEAIKKLXESYIDLKE

SEQ ID NO: 176 - protein, 35 aa, Artificial Sequence
  source: /note=“Description of Artificial Sequence Synthetic polypeptide”
  MOD_RES (14)..(14): Any stapling/stitching amino acid
  SITE (14)..(21): /note=“May or may not be cross-linked via a side chain”
  MOD_RES (16)..(16): Norleucine
  MOD_RES (21)..(21): Any stapling/stitching amino acid
  SITE (21)..(28): /note=“May or may not be cross-linked via a side chain”
  MOD_RES (28)..(28): Any stapling/stitching amino acid
SLDQINVTFLDLEXEXKKLEXAIKKLEXSYIDLKE

SEQ ID NO: 177 - protein, 35 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (16)..(16): Norleucine
  MOD_RES (17)..(17): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
SLDQINVTFLDLXYEXXKLEEAIXKLEXSYIDLKE

SEQ ID NO: 178 - protein, 35 aa, Artificial Sequence
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (16)..(16): Norleucine
  MOD_RES (18)..(18): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid
SLDQINVTFLDLEXEXKXLEEAIXKLEXSYIDLKE

SEQ ID NO: 179 - protein, 35 aa, Artificial Sequence
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (16)..(16): Norleucine
  MOD_RES (17)..(17): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid
SLDQINVTFLDLXYEXXKLEEAIKXLEEXYIDLKE

SEQ ID NO: 180 - protein, 35 aa, Artificial Sequence
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (16)..(16): Norleucine
  MOD_RES (18)..(18): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid
SLDQINVTFLDLEXEXKXLEEAIKXLEEXYIDLKE

SEQ ID NO: 181 - protein, 6 aa, Artificial Sequence
  source: /note=“Description of Artificial Sequence Synthetic peptide”
DRLNEV

SEQ ID NO: 182 - protein, 7 aa, Artificial Sequence
  source: /note=“Description of Artificial Sequence Synthetic peptide”
KNLNESL

SEQ ID NO: 183 - protein, 7 aa, Artificial Sequence
  source: /note=“Description of Artificial Sequence Synthetic peptide”
IQKEIDR

SEQ ID NO: 184 - protein, 6 aa, Artificial Sequence
  source: /note=“Description of Artificial Sequence Synthetic peptide”
NEVAKN

SEQ ID NO: 185 - protein, 4 aa, Artificial Sequence
  source: /note=“Description of Artificial Sequence Synthetic peptide”
NESL

SEQ ID NO: 186 - protein, 10 aa, Artificial Sequence
  source: /note=“Description of Artificial Sequence Synthetic peptide”
IQKEIDRLNE

SEQ ID NO: 187 - protein, 6 aa, Artificial Sequence
  source: /note=“Description of Artificial Sequence Synthetic peptide”
AKNLNE

SEQ ID NO: 188 - protein, 11 aa, Artificial Sequence
  source: /note=“Description of Artificial Sequence Synthetic peptide”
IQKEIDRLNEV

SEQ ID NO: 189 - protein, 6 aa, Artificial Sequence
  source: /note=“Description of Artificial Sequence Synthetic peptide”
KNLNES

SEQ ID NO: 190 - protein, 14 aa, Artificial Sequence
  source: /note=“Description of Artificial Sequence Synthetic peptide”
DRLNEVAKNLNESL

SEQ ID NO: 191 - protein, 14 aa, Artificial Sequence
  source: /note=“Description of Artificial Sequence Synthetic peptide”
IQKEIDRLNEVAKN

SEQ ID NO: 192 - protein, 10 aa, Artificial Sequence
  source: /note=“Description of Artificial Sequence Synthetic peptide”
ISGINASVVN

SEQ ID NO: 193 - protein, 11 aa, Artificial Sequence
  source: /note=“Description of Artificial Sequence Synthetic peptide”
ISGINASVVNI

SEQ ID NO: 194 - protein, 12 aa, Artificial Sequence
  source: /note=“Description of Artificial Sequence Synthetic peptide”
ISGINASVVNIQ

SEQ ID NO: 195 - protein, 13 aa, Artificial Sequence
  source: /note=“Description of Artificial Sequence Synthetic peptide”
ISGINASVVNIQK

SEQ ID NO: 196 - protein, 14 aa, Artificial Sequence
  source: /note=“Description of Artificial Sequence Synthetic peptide”
ISGINASVVNIQKE

SEQ ID NO: 197 - protein, 15 aa, Artificial Sequence
  source: /note=“Description of Artificial Sequence Synthetic peptide”
ISGINASVVNIQKEI

SEQ ID NO: 198 - protein, 16 aa, Artificial Sequence
  source: /note=“Description of Artificial Sequence Synthetic peptide”
ISGINASVVNIQKEID

SEQ ID NO: 199 - protein, 17 aa, Artificial Sequence
  source: /note=“Description of Artificial Sequence Synthetic peptide”
ISGINASVVNIQKEIDR

SEQ ID NO: 200 - protein, 18 aa, Artificial Sequence
  source: /note=“Description of Artificial Sequence Synthetic peptide”
ISGINASVVNIQKEIDRL

SEQ ID NO: 201 - protein, 19 aa, Artificial Sequence
  source: /note=“Description of Artificial Sequence Synthetic peptide”
ISGINASVVNIQKEIDRLN

SEQ ID NO: 202 - protein, 20 aa, Artificial Sequence
  source: /note=“Description of Artificial Sequence Synthetic peptide”
ISGINASVVNIQKEIDRLNE

SEQ ID NO: 203 - protein, 21 aa, Artificial Sequence
  source: /note=“Description of Artificial Sequence Synthetic peptide”
ISGINASVVNIQKEIDRLNEV

SEQ ID NO: 204 - protein, 22 aa, Artificial Sequence
  source: /note=“Description of Artificial Sequence Synthetic peptide”
ISGINASVVNIQKEIDRLNEVA

SEQ ID NO: 205 - protein, 23 aa, Artificial Sequence
  source: /note=“Description of Artificial Sequence Synthetic peptide”
ISGINASVVNIQKEIDRLNEVAK

SEQ ID NO: 206 - protein, 24 aa, Artificial Sequence
  source: /note=“Description of Artificial Sequence Synthetic peptide”
ISGINASVVNIQKEIDRLNEVAKN

SEQ ID NO: 207 - protein, 8 aa, Artificial Sequence
  source: /note=“Description of Artificial Sequence Synthetic peptide”
SLIDLQEL

SEQ ID NO: 208 - protein, 7 aa, Artificial Sequence
  source: /note=“Description of Artificial Sequence Synthetic peptide”
LIDLQEL

SEQ ID NO: 209 - protein, 12 aa, Artificial Sequence
  source: /note=“Description of Artificial Sequence Synthetic peptide”
DISGINASVVNI

SEQ ID NO: 210 - protein, 13 aa, Artificial Sequence
  source: /note=“Description of Artificial Sequence Synthetic peptide”
DISGINASVVNIQ

SEQ ID NO: 211 - protein, 15 aa, Artificial Sequence
  source: /note=“Description of Artificial Sequence Synthetic peptide”
SLIDLQELGKYEQYI

SEQ ID NO: 212 - protein, 14 aa, Artificial Sequence
  source: /note=“Description of Artificial Sequence Synthetic peptide”
LIDLQELGKYEQYI

SEQ ID NO: 213 - protein, 12 aa, Artificial Sequence
  source: /note=“Description of Artificial Sequence Synthetic peptide”
SLDQINVTFLDL

SEQ ID NO: 214 - protein, 6 aa, Artificial Sequence
  source: /note=“Description of Artificial Sequence Synthetic peptide”
KLEEAI

SEQ ID NO: 215 - protein, 7 aa, Artificial Sequence
  source: /note=“Description of Artificial Sequence Synthetic peptide”
SYIDLKE

SEQ ID NO: 216 - protein, 13 aa, Artificial Sequence
  source: /note=“Description of Artificial Sequence Synthetic peptide”
SLDQINVTFLDLE

SEQ ID NO: 217 - protein, 5 aa, Artificial Sequence
  source: /note=“Description of Artificial Sequence Synthetic peptide”
LEEAI

SEQ ID NO: 218 - protein, 7 aa, Artificial Sequence
  source: /note=“Description of Artificial Sequence Synthetic peptide”
KLEEAIK

SEQ ID NO: 219 - protein, 6 aa, Artificial Sequence
  source: /note=“Description of Artificial Sequence Synthetic peptide”
YIDLKE

SEQ ID NO: 220 - protein, 6 aa, Artificial Sequence
  source: /note=“Description of Artificial Sequence Synthetic peptide”
LEEAIK

SEQ ID NO: 221 - protein, 16 aa, Artificial Sequence
  source: /note=“Description of Artificial Sequence Synthetic peptide”
ESLIDLQELGKYEQYI

SEQ ID NO: 222 - protein, 18 aa, Artificial Sequence
  source: /note=“Description of Artificial Sequence Synthetic peptide”
LNESLIDLQELGKYEQYI

SEQ ID NO: 223 - protein, 6 aa, Artificial Sequence
  source: /note=“Description of Artificial Sequence Synthetic peptide”
  MOD_RES (3)..(3): Norleucine
YEXKKL

SEQ ID NO: 224 - protein, 6 aa, Artificial Sequence
  source: /note=“Description of Artificial Sequence Synthetic peptide”
EAIKKL

SEQ ID NO: 225 - protein, 8 aa, Artificial Sequence
  source: /note=“Description of Artificial Sequence Synthetic peptide”
ESYIDLKE

SEQ ID NO: 226 - protein, 6 aa, Artificial Sequence
  source: /note=“Description of Artificial Sequence Synthetic peptide”
  MOD_RES (2)..(2): Norleucine
EXKKLE

SEQ ID NO: 227 - protein, 6 aa, Artificial Sequence
  source: /note=“Description of Artificial Sequence Synthetic peptide”
AIKKLE

SEQ ID NO: 228 - protein, 6 aa, Artificial Sequence
  source: /note=“Description of Artificial Sequence Synthetic peptide”
QKEIDR

SEQ ID NO: 229 - protein, 11 aa, Artificial Sequence
  source: /note=“Description of Artificial Sequence Synthetic peptide”
NEVAKNLNESL

SEQ ID NO: 230 - protein, 6 aa, Artificial Sequence
  source: /note=“Description of Artificial Sequence Synthetic peptide”
IDRLNE

SEQ ID NO: 231 - protein, 8 aa, Artificial Sequence
  source: /note=“Description of Artificial Sequence Synthetic peptide”
AKNLNESL

SEQ ID NO: 232 - protein, 4 aa, Artificial Sequence
  source: /note=“Description of Artificial Sequence Synthetic peptide”
IQKE

SEQ ID NO: 233 - protein, 19 aa, Artificial Sequence
  source: /note=“Description of Artificial Sequence Synthetic peptide”
  MOD_RES (1)..(1): Any stapling/stitching amino acid
  SITE (1)..(8): /note=“May or may not be cross-linked via a side chain”
  MOD_RES (8)..(8): Any stapling/stitching amino acid
XQKEIDRXNEVAKNLNESL

SEQ ID NO: 234 - protein, 29 aa, Artificial Sequence
  source: /note=“Description of Artificial Sequence Synthetic peptide”
  MOD_RES (15)..(15): Any stapling/stitching amino acid
  SITE (15)..(22): /note=“May or may not be cross-linked via a side chain”
  MOD_RES (22)..(22): Any stapling/stitching amino acid
ISGINASVVNIQKEXDRLNEVXKNLNESL

SEQ ID NO: 235 - protein, 36 aa, Artificial Sequence; identical to SEQ ID NO: 174
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (18)..(18): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid

SEQ ID NO: 236 - protein, 42 aa, Artificial Sequence; identical to SEQ ID NO: 153
  source: /note=“Description of Artificial Sequence Synthetic polypeptide”
  MOD_RES (12)..(12): Any stapling/stitching amino acid
  SITE (12)..(19): /note=“May or may not be cross-linked via a side chain”
  MOD_RES (19)..(19): Any stapling/stitching amino acid
  SITE (19)..(26): /note=“May or may not be cross-linked via a side chain”
  MOD_RES (26)..(26): Any stapling/stitching amino acid

SEQ ID NO: 237 - protein, 42 aa, Artificial Sequence; identical to SEQ ID NO: 154
  source: /note=“Description of Artificial Sequence Synthetic polypeptide”
  MOD_RES (13)..(13): Any stapling/stitching amino acid
  SITE (13)..(20): /note=“May or may not be cross-linked via a side chain”
  MOD_RES (20)..(20): Any stapling/stitching amino acid
  SITE (20)..(24): /note=“May or may not be cross-linked via a side chain”
  MOD_RES (24)..(24): Any stapling/stitching amino acid

SEQ ID NO: 238 - protein, 42 aa, Artificial Sequence; identical to SEQ ID NO: 156
  source: /note=“Description of Artificial Sequence Synthetic polypeptide”
  MOD_RES (12)..(12): Any stapling/stitching amino acid
  SITE (12)..(16): /note=“May or may not be cross-linked via a side chain”
  MOD_RES (16)..(16): Any stapling/stitching amino acid
  MOD_RES (23)..(23): Any stapling/stitching amino acid
  SITE (23)..(27): /note=“May or may not be cross-linked via a side chain”
  MOD_RES (27)..(27): Any stapling/stitching amino acid

SEQ ID NO: 239 - protein, 42 aa, Artificial Sequence; identical to SEQ ID NO: 158
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (23)..(23): Any stapling/stitching amino acid
  SITE (23)..(27): /note=“May or may not be cross-linked via a side chain”
  MOD_RES (27)..(27): Any stapling/stitching amino acid

SEQ ID NO: 240 - protein, 42 aa, Artificial Sequence; identical to SEQ ID NO: 160
  source: /note=“Description of Artificial Sequence Synthetic polypeptide”
  MOD_RES (12)..(12): Any stapling/stitching amino acid
  SITE (12)..(16): /note=“May or may not be cross-linked via a side chain”
  MOD_RES (16)..(16): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid

SEQ ID NO: 241 - protein, 42 aa, Artificial Sequence; identical to SEQ ID NO: 162
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid

SEQ ID NO: 242 - protein, 35 aa, Artificial Sequence; identical to SEQ ID NO: 175
  source: /note=“Description of Artificial Sequence Synthetic polypeptide”
  MOD_RES (13)..(13): Any stapling/stitching amino acid
  SITE (13)..(20): /note=“May or may not be cross-linked via a side chain”
  MOD_RES (16)..(16): Norleucine
  MOD_RES (20)..(20): Any stapling/stitching amino acid
  SITE (20)..(27): /note=“May or may not be cross-linked via a side chain”
  MOD_RES (27)..(27): Any stapling/stitching amino acid

SEQ ID NO: 243 - protein, 35 aa, Artificial Sequence; identical to SEQ ID NO: 176
  source: /note=“Description of Artificial Sequence Synthetic polypeptide”
  MOD_RES (14)..(14): Any stapling/stitching amino acid
  SITE (14)..(21): /note=“May or may not be cross-linked via a side chain”
  MOD_RES (16)..(16): Norleucine
  MOD_RES (21)..(21): Any stapling/stitching amino acid
  SITE (21)..(28): /note=“May or may not be cross-linked via a side chain”
  MOD_RES (28)..(28): Any stapling/stitching amino acid

SEQ ID NO: 244 - protein, 35 aa, Artificial Sequence; identical to SEQ ID NO: 177
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (16)..(16): Norleucine
  MOD_RES (17)..(17): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid

SEQ ID NO: 245 - protein, 35 aa, Artificial Sequence; identical to SEQ ID NO: 178
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (16)..(16): Norleucine
  MOD_RES (18)..(18): Any stapling/stitching amino acid
  MOD_RES (24)..(24): Any stapling/stitching amino acid
  SITE (24)..(28): /note=“May or may not be cross-linked via a side chain”
  MOD_RES (28)..(28): Any stapling/stitching amino acid

SEQ ID NO: 246 - protein, 35 aa, Artificial Sequence; identical to SEQ ID NO: 179
  source: /note=“Description of Artificial Sequence Synthetic polypeptide”
  MOD_RES (13)..(13): Any stapling/stitching amino acid
  SITE (13)..(17): /note=“May or may not be cross-linked via a side chain”
  MOD_RES (16)..(16): Norleucine
  MOD_RES (17)..(17): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid

SEQ ID NO: 247 - protein, 35 aa, Artificial Sequence; identical to SEQ ID NO: 180
  source: /note=“Description of Artificial Sequence Synthetic polypeptide”
  MOD_RES (14)..(14): Any stapling/stitching amino acid
  SITE (14)..(18): /note=“May or may not be cross-linked via a side chain”
  MOD_RES (16)..(16): Norleucine
  MOD_RES (18)..(18): Any stapling/stitching amino acid
  MOD_RES (25)..(25): Any stapling/stitching amino acid
  SITE (25)..(29): /note=“May or may not be cross-linked via a side chain”
  MOD_RES (29)..(29): Any stapling/stitching amino acid

SEQ ID NO: 248 - protein, 19 aa, Artificial Sequence
  source: /note=“Description of Artificial Sequence Synthetic peptide”
  MOD_RES (3)..(3): Any stapling/stitching amino acid
  SITE (3)..(7): /note=“May or may not be cross-linked via a side chain”
  MOD_RES (7)..(7): Any stapling/stitching amino acid
  MOD_RES (13)..(13): Any stapling/stitching amino acid
  SITE (13)..(17): /note=“May or may not be cross-linked via a side chain”
  MOD_RES (17)..(17): Any stapling/stitching amino acid
IQXEIDXLNEVAXNLNXSL

SEQ ID NO: 249 - protein, 35 aa, Artificial Sequence
  source: /note=“Description of Artificial Sequence Synthetic polypeptide”
SLDQINVTFLDLEYEMKKLEEAIKKLEESYIDLKE

SEQ ID NO: 250 - protein, 10 aa, Artificial Sequence
  source: /note=“Description of Artificial Sequence Synthetic peptide”
ISGINASVVN

SEQ ID NO: 251 - protein, 11 aa, Artificial Sequence
  source: /note=“Description of Artificial Sequence Synthetic peptide”
DISGINASVVN

SEQ ID NO: 252 - protein, 6 aa, Artificial Sequence
  source: /note=“Description of Artificial Sequence Synthetic peptide”
IDLQEL

SEQ ID NO: 253 - protein, 13 aa, Artificial Sequence
  source: /note=“Description of Artificial Sequence Synthetic peptide”
IDLQELGKYEQYI

SEQ ID NO: 254 - protein, 12 aa, Artificial Sequence
  source: /note=“Description of Artificial Sequence Synthetic peptide”
IDLQELGSGSGC

SEQ ID NO: 255 - protein, 19 aa, Artificial Sequence
  source: /note=“Description of Artificial Sequence Synthetic peptide”
IDLQELGKYEQYIGSGSGC

SEQ ID NO: 256 - protein, 6 aa, Artificial Sequence
  source: /note=“Description of Artificial Sequence Synthetic peptide”
GSGSGC

SEQ ID NO: 257 - protein, 29 aa, Artificial Sequence
  source: /note=“Description of Artificial Sequence Synthetic peptide”
  MOD_RES (13)..(13): Any stapling/stitching amino acid
  SITE (13)..(17): /note=“May or may not be cross-linked via a side chain”
  MOD_RES (17)..(17): Any stapling/stitching amino acid
  MOD_RES (23)..(23): Any stapling/stitching amino acid
  SITE (23)..(27): /note=“May or may not be cross-linked via a side chain”
  MOD_RES (27)..(27): Any stapling/stitching amino acid
ISGINASVVNIQXEIDXLNEVAXNLNXSL

SEQ ID NO: 258 - protein, 19 aa, Artificial Sequence
  source: /note=“Description of Artificial Sequence Synthetic peptide”
  MOD_RES (5)..(5): Norleucine
LEYEXKKLEEAIKKLEESY

SEQ ID NO: 259 - protein, 36 aa, Middle East respiratory syndrome-related coronavirus
SLTQINTTLLDLTYEMLSLQQVVKALNESYIDLKEL

SEQ ID NO: 260 - protein, 80 aa, Severe acute respiratory syndrome coronavirus 2
ELDKYFKNHTSPDVDLGDISGINASVVNIQKEIDRLNEVAKNLNESLIDLQELGKYEQYIKWPWYIWLGFIAGLIAIVMV

SEQ ID NO: 261 - protein, 147 aa, Artificial Sequence
  source: /note=“Description of Artificial Sequence Synthetic polypeptide”
MNQFNSAIGKIQDSLSSTASALGKLQDGGSGGIQKEIDRLNEVAKNLNESLGSSGGNQFNSAIGKIQDSLSSTASALGKLQDGGSGGIQKEIDRLNEVAKNLNESLGSSGGNQFNSAIGKIQDSLSSTASALGKLQDSSGGHHHHHH

SEQ ID NO: 262 - protein, 250 aa, Artificial Sequence
  source: /note=“Description of Artificial Sequence Synthetic polypeptide”
MQKLIANQFNSAIGKIQDSLSSTASALGKLQDVVNQNAQALNTLVKQLSSGGSGGDISGINASVVNIQKEIDRLNEVAKNLNESLIDLQELGSSGGQKLIANQFNSAIGKIQDSLSSTASALGKLQDVVNQNAQALNTLVKQLSSGGSGGDISGINASVVNIQKEIDRLNEVAKNLNESLIDLQELGSSGGQKLIANQFNSAIGKIQDSLSSTASALGKLQDVVNQNAQALNTLVKQLSSSSGGHHHHHH

SEQ ID NO: 263 - protein, 358 aa, Artificial Sequence
  source: /note=“Description of Artificial Sequence Synthetic polypeptide”
MVTQNVLYENQKLIANQFNSAIGKIQDSLSSTASALGKLQDVVNQNAQALNTLVKQLSSNFGAISSVLNDILSRLDKVESGGRGGPDVDLGDISGINASVVNIQKEIDRLNEVAKNLNESLIDLQELGKYGGRGGVTQNVLYENQKLIANQFNSAIGKIQDSLSSTASALGKLQDVVNQNAQALNTLVKQLSSNFGAISSVLNDILSRLDKVESGGRGGPDVDLGDISGINASVVNIQKEIDRLNEVAKNLNESLIDLQELGKYGGRGGVTQNVLYENQKLIANQFNSAIGKIQDSLSSTASALGKLQDVVNQNAQALNTLVKQLSSNFGAISSVLNDILSRLDKVESGGSSHHHHHH